(19) United States
(12) Patent Application Publication
(10) Pub. No.: US 2025/0135092 A1
(43) Pub. Date: May 1, 2025

(54) APPARATUS FOR SUPPLYING FLUID TO A TISSUE AREA

(71) Applicant: Fisher & Paykel Healthcare Limited, Auckland (NZ)

(72) Inventors: Thomas Heinrich BARNES, Auckland (NZ); Dexter Chi Lun CHEUNG, Auckland (NZ); Margaret Shi LU, Auckland (NZ); Howard Kuo-Hao CHIU, Auckland (NZ); Eglantine Penelope Mary LAYEC, Auckland (NZ)

(21) Appl. No.: 18/684,136

(22) PCT Filed: Aug. 17, 2022
(86) PCT No.: PCT/IB2022/057691

§ 371 Date: Feb. 15, 2024

(30) Foreign Application Priority Data
Aug. 17, 2021 (AU) 2021218054
Oct. 22, 2021 (AU) 2021903382
May 18, 2022 (AU) 2022901333

(51) Int. Cl. A61M 1/00; A61F 13/00; A61F 13/05

(52) U.S. Cl. CPC A61M 1/94; A61F 13/05; A61F 2013/00093; A61F 2013/00174; A61F 2013/00246

(57) ABSTRACT

An apparatus is provided for supplying fluid to tissue of a patient. The apparatus includes an inflatable receptacle positionable at a tissue area. The receptacle comprises one or more walls and is adapted to receive a fluid. The one or more walls comprise at least one section adapted to allow molecules within the fluid to diffuse from the receptacle to the tissue area. The apparatus further includes a cover positionable over the receptacle to form a compartment substantially bounded by the cover, the receptacle and the tissue area.

### TECHNICAL FIELD

The present invention relates to an apparatus for supplying fluid to a tissue area.

### BACKGROUND

The application of topical oxygen to a tissue area can improve tissue health. For example, in cases where the tissue area comprises a wound, the application of topical oxygen can aid healing. This is because healing can involve increased cell metabolic activity (which demands a large amount of oxygen). The application of topical oxygen to the wound can help to meet this demand. Exposing the wound surface to a negative-pressure environment can also aid healing. The negative-pressure environment helps to contract the wound and remove exudate from the wound.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure describes embodiments of an apparatus for supplying fluid to a tissue area. At least one receptacle, which includes at least a section made from a material adapted to allow molecules within the fluid to diffuse from the receptacle to the tissue area, may be positioned against the tissue area. The tissue area may include, for example, a wound. The material may be, for example, a membrane. The material allow fluid molecules, for example oxygen gas molecules, inside of the receptacle, to diffuse from the inside of the receptacle to the wound. At the same time, the material substantially provides a barrier to liquid. An outer surface of the material through which molecules diffuse may contact the tissue to be treated and/or adjacent tissue, for example a wound and/or the healthy tissue that surrounds the wound. Because the membrane of the receptacle is in contact with the wound surface during use, the delivery of fluid to the wound is not significantly affected by the amount of exudate withdrawn from the wound. Some embodiments of the apparatus include an absorbent layer positioned away from the wound. Positioning the absorbent layer away from the wound reduces the possibility of tissue cells growing into the absorbent layer and pieces of absorbent layer being left in the wound when the device is removed.

According to an aspect, an apparatus for supplying fluid to a tissue area comprises an inflatable receptacle positionable at a tissue area, the receptacle comprising one or more walls and being adapted to receive a fluid, the one or more walls comprising at least one section adapted to allow molecules within the fluid to diffuse from the receptacle to the tissue area; and a cover positionable over the receptacle to form a compartment substantially bounded by the cover, the receptacle, and the tissue area. The arrangement of the apparatus may facilitate the simultaneous application of therapeutic fluid molecules to the tissue area and the application of negative pressure treatment without significantly impacting the application of the therapeutic fluid molecules to the tissue area. The tissue area may comprise a wound area.

In some embodiments, the receptacle is moveable within the compartment substantially bounded by the cover, the receptacle and the tissue area.

In some embodiments, the receptacle may be in the form of a bag. The receptacle is inflatable, and depends on the presence of fluid to maintain its size and shape. However, the receptacle need not be fully inflated and the apparatus is operable with a partially inflated receptacle. The receptacle may be configured to contact the tissue area. At least the at least one section of the receptacle may be conformable with a surface of the tissue area. The at least one section of the receptacle may comprise a stretchable material. The at least one section of the one or more walls of the receptacle may be configured to have an undulating outer surface when at least partially inflated. The inflatable receptacle may provide a substantially even concentration of fluid within the receptacle and a substantially even diffusion of molecules through the section through which molecules diffuse to the tissue. It may also provide a substantially even pressure distribution of molecules over the tissue. The receptacle may act as a reservoir and contain volumes of fluid. The fluid in the receptacle may be used in the event of a disruption to the supply of fluid to the receptacle e.g. if the fluid source becomes disconnected from the receptacle.

In some embodiments, the one or more walls of the receptacle other than the at least one section allow molecules within the fluid to diffuse from the receptacle to the tissue area.

The receptacle may be connected to the cover at a single connection point or at two connection points. This minimal connection between the receptacle and the cover enables the receptacle to be movable within the compartment bounded by the cover, the receptacle, and the tissue area, which may help it to conform to tissue with complex geometries and/or varying topologies.

The apparatus may further comprise a fluid inlet conduit arranged in fluid communication with the receptacle for delivery of fluid to the receptacle. In some embodiments, the cover may have a first opening through which the fluid inlet conduit passes or to which the fluid inlet conduit is connected. The receptacle may be connected to the cover via the fluid inlet conduit. The fluid inlet conduit may include a pressure relief valve. In some embodiments, the compartment substantially bounded by the cover, the receptacle, and the tissue area may also be bounded by additional components of the apparatus that may be present and still be substantially bounded by the cover, the receptacle and the tissue area. For example, in some embodiments, the compartment may be further bounded by one or more fluid inlet conduits and/or fluid outlet conduits. In these embodiments, the compartment substantially bounded by the cover, the receptacle and the tissue area may be further bounded by the fluid inlet conduit, including the pressure relief valve if present inside the cover.

In some embodiments, the apparatus may further comprise at least one fastener for securing the receptacle in place at the tissue area. The fastener may comprise adhesive, such as adhesive tape. It may also comprise a compression dressing that presses and secures the receptacle in place.

In some embodiments, the cover comprises one or more walls and the one or more walls of the cover are independent of the one or more walls of the receptacle. This arrangement may enable the receptacle to be movable within the compartment, which may help it to conform to tissue having complex geometries and/or varying topologies.

Alternatively, the receptacle and the cover may share a common wall.

The receptacle may be fillable with the fluid during use of the apparatus. The receptacle may be filled with the fluid prior to positioning the receptacle at the tissue area. Alternatively, the receptacle may be pre-filled with the fluid during manufacture.

The one or more walls of the receptacle may define one or more through-holes through the receptacle. Where the tissue includes a wound, these ‘through-holes’ allow exudate from the wound to be drawn through the holes, in particular when the compartment is under negative pressure. This may result in more exudate being drawn away from the wound.

In some embodiments, the apparatus may include a plurality of the receptacles. Multiple receptacles can provide additional conformability to the surface of the tissue area for complex geometries and/or varying tissue topologies.

The apparatus may further comprise structures associated with the at least one section of the receptacle, the structures being configured to contact the tissue area. The contact may encourage cell growth. The structures may be microstructures. The microstructures may exert forces on the tissue. These forces may result in micro-stresses that stretch the underlying cells and cause them to take on the same signalling pathways as those affected by growth factors. This can encourage cell growth. The microstructures may comprise formations which protrude from the one or more walls of the receptacle. In some embodiments, molecules diffuse through the at least one section of the receptacle, including the microstructures, into the tissue area.

The microstructures may consist of formations that protrude from a tissue facing surface of the one or more walls of the receptacle. Each formation may have one or more of a parabolic, trapezoidal, semi-spherical, spherical, pyramidal, conical, or frustoconical shape. Each formation may be a different shape to the other formations. Each formation may be the same shape as one or more of the other formations. The microstructures may have rounded edges and/or corners. Accordingly, in this embodiment there are no sharp edges on the microstructures which could potentially damage cells.

In some embodiments, the at least one section of the receptacle may comprise a membrane adapted to allow molecules within the fluid to diffuse through it. More specifically, the receptacle may comprise a membrane adapted to allow molecules within the fluid in the receptacle to diffuse through to reach the tissue area. The membrane may be a film, sheet, flexible wall, or the like. The membrane adapted to allow molecules within the fluid to diffuse from the receptacle to the tissue area may be substantially pore-free. In some embodiments, the membrane comprises substantially no discontinuities or pores that are visible with an optical or electron microscope at a specified magnification. The specified resolution may be on the order of 1 micrometre. This produces a substantially smooth outer surface to the membrane that helps to prevent exudate from entering the receptacle. It may also discourage the growth of skin cells into pores of the membrane. Molecules within a liquid may only pass from one side of the membrane to the other via diffusion, which further helps to prevent exudate from entering the receptacle. The membrane may also be hydrophobic.

In some embodiments, the membrane adapted to allow molecules within the fluid to diffuse from the receptacle to the tissue area may comprise at least one of the following: liquid silicone rubber, pre-formed silicone sheet, silicone hydrogel, linear low-density polyethylene treated with calcium carbonate, silicone coatings, and high consistency rubber silicone.

The membrane adapted to allow molecules within the fluid to diffuse from the receptacle to the tissue area may be configured to contact the tissue area. The at least one section may be conformable to the surface of the tissue. It may have a wall thickness of between about 10 micrometres and about 150 micrometres, for example between approximately 20 micrometres and 140 micrometres, or between approximately 30 micrometres and 130 micrometres, or between approximately 35 micrometres and 100 micrometres, or between approximately 40 micrometres and 80 micrometres, or between approximately 40 micrometres to 70 micrometres, or between approximately 30 micrometres to 60 micrometres, or between approximately 20 to 60 micrometres, or between approximately 45 micrometres and 55 micrometres. In some embodiments, the wall thickness is approximately 60 micrometres. In some embodiments, the wall thickness is approximately 50 micrometres.

In some embodiments, the one or more walls of the receptacle, including the at least one section, comprise the membrane adapted to allow molecules within the fluid to diffuse from the receptacle to the tissue area. In other embodiments, the one or more walls of the receptacle, other than the at least one section, comprise a material that is adapted to allow molecules to pass through via molecular diffusion at only a negligible rate of diffusion.

The cover may be configured to secure the receptacle to the tissue surface. The cover may be configured to press the receptacle onto or into the tissue surface. In some embodiments, the cover may be a separate component that is positionable over the receptacle once the receptacle has been placed on the tissue. Alternatively, the cover may be combined with the receptacle to form a single dressing. The cover may be integrally formed with the receptacle.

The cover may comprise a dressing, for example a bandage. The bandage may be wrapped around a body part, for example a limb, of the patient to form the cover. The cover may comprise an adhesive dressing or polyurethane film. Alternatively, the bandage may be a compression bandage.

In some embodiments, the cover may comprise an absorbent material for absorbing exudate from the tissue area. The absorbent material may be wrapped around a limb of a patient and the bandage may be wrapped around the absorbent material to form the cover.

In some embodiments, the cover may comprise an absorbent material for absorbing exudate from the tissue area. The absorbent material may be placed upon or within the wound area of a patient. The cover may hold the absorbent material in place.

The fluid may be deliverable to the receptacle via the first opening in the cover. The first opening may be adapted for receiving the fluid inlet conduit there through. The cover may have a second opening via which the fluid is receivable from the receptacle. The apparatus may further comprise a first fluid outlet conduit arranged in fluid communication with the receptacle, wherein the second opening is adapted for receiving the first fluid outlet conduit. The cover may have a third opening via which the fluid is receivable from the compartment bounded by the cover, the receptacle, and the tissue area. The apparatus may comprise a second fluid outlet conduit arranged in fluid communication with the compartment substantially bounded by the cover, the receptacle, and the tissue area, wherein the third opening is adapted for receiving the second fluid outlet conduit. In these embodiments, the compartment substantially bounded by the cover, the receptacle and the tissue area may be further bounded by one or more of the fluid inlet conduit and the first fluid outlet conduit.

The receptacle may comprise a first fluid outlet conduit via which fluid is receivable from the receptacle.

The apparatus may include a pressure relief valve in communication with the first opening and/or the receptacle, operable to relieve pressure within the first opening and/or receptacle

In some embodiments, at least one of the fluid inlet conduit, the first fluid outlet conduit and the second fluid outlet conduit may pass into the compartment adjacent an outer edge of the cover. At least one of the fluid inlet conduit, the first fluid outlet conduit and the second fluid outlet conduit may pass into the compartment past an outer edge of the cover.

At least two of the fluid inlet conduit, the first fluid outlet conduit, and the second fluid outlet conduit may be formed as a single coaxial conduit. In some embodiments, the fluid inlet conduit and the first fluid outlet conduit may be formed as a co-axial conduit or as a combined conduit.

The cover may be sealable to the tissue area, for example to healthy skin adjacent to a wound. In some embodiments, the cover may be hermetically sealable to the tissue area. The seal may comprise one of an adhesive seal or a suction seal.

The cover may comprise one or more walls of a material that is impermeable to bulk transport of fluid, e.g. gas and/or liquid. For example, a compression bandage dressing may be impermeable to bulk transport of gas and liquid or multiple layers of the compression dressing can substantially reduce permeability to bulk transport of gas relative to a single layer of the compression bandage dressing.

The apparatus may comprise a negative pressure source arranged in fluid communication with the compartment for applying a negative pressure to the compartment. In some embodiments, the negative pressure source is a pump. In some embodiments, the compartment substantially bounded by the cover, the receptacle, and the tissue area is pressurised during use to form a negative pressure compartment. The compartment may be altered to be between approximately 50 mmHg to 150 mmHg below atmospheric pressure. For example, the pressure may be altered to between approximately 80 mmHg to 125 mmHg, or to between approximately 90 mmHg to 110 mmHg.

In some embodiments, the fluid may comprise a gas, such as oxygen gas and/or carbon dioxide gas and/or carbon monoxide gas. When the tissue comprises a wound, the application of oxygen may aid healing, while the application of carbon dioxide may induce hypoxia and help to trigger angiogenesis.

The apparatus may further comprise a fluid source. For example, the fluid may be a gas and the gas source may comprise a wall source, gas bottle, gas concentrator, or other gas generator.

The receptacle may form a reservoir adapted for storing fluid therein. The reservoir may be fillable with the fluid, for example by a clinician prior to positioning the receptacle 10 on the tissue area of the patient or during use of the apparatus. Alternatively, the reservoir may be pre-filled with fluid during manufacture of the apparatus 100. A pressure within the reservoir may be maintainable at a positive pressure. At the same time, the compartment substantially bounded by the cover, the receptacle and the tissue area may be maintainable at a pressure that is below ambient pressure.

The apparatus may be configured for simultaneous delivery of fluid to tissue of a patient via the receptacle and application of a negative pressure to the compartment. The receptacle may be adapted for operation at a positive pressure simultaneously as a negative pressure is applied to the compartment.

According to another aspect, an apparatus for supplying fluid to a tissue area comprises a receptacle positionable at a tissue area, the receptacle comprising one or more walls adapted to receive a fluid, the one or more walls comprising at least one section adapted to allow fluid to pass from the receptacle to the tissue area; and a cover positionable over the receptacle to form a compartment substantially bounded by the cover, the receptacle, and the tissue area; a first opening in the cover via which fluid is deliverable to the receptacle; a second opening in the cover via which fluid is receivable from the receptacle; and a third opening in the cover via which fluid is receivable from the compartment bounded by the cover, the receptacle, and the tissue area. The tissue area may comprise a wound area.

The receptacle may comprise multiple layers, including a first layer configured to form a tissue facing layer, and a second layer. The second layer may have a first thickness and the first layer has a second thickness, the first thickness being greater than the second thickness. The second layer may comprise an upper layer of the receptacle.

The apparatus may comprise a tubular fluid compartment having a fluid inlet and a fluid outlet.

In some embodiments, the at least one section is adapted to allow molecules within the fluid to diffuse from the receptacle to the tissue area or the fluid may pass from the receptacle to the tissue area by pore flow.

The fluid may comprise a gas, such as oxygen gas and/or carbon dioxide gas and/or carbon monoxide gas.

According to a further aspect, an apparatus for supplying fluid to a tissue area comprises a receptacle positionable at a tissue area, the receptacle comprising one or more walls adapted to receive a fluid, the one or more walls comprising at least one section adapted to allow molecules within the fluid to diffuse from the receptacle to the tissue area; and a cover positionable over the receptacle to form a compartment substantially bounded by the cover, the receptacle, and the tissue area, wherein the receptacle is moveable within the compartment substantially bounded by the cover, the receptacle and the tissue area. The receptacle may be inflatable. The tissue area may comprise a wound area.

The receptacle may comprise multiple layers, including a first layer configured to form a tissue facing layer, and a second layer. The second layer may have a first thickness and the first layer has a second thickness, the first thickness being greater than the second thickness. The second layer may comprise an upper layer of the receptacle.

According to a further aspect, an apparatus for supplying fluid to a tissue area comprises a plurality of receptacles positionable at a tissue area, each receptacle comprising one or more walls adapted to receive a fluid, the one or more walls comprising at least one section adapted to allow fluid to pass from the receptacle to the tissue area. The tissue area may comprise a wound area.

In some embodiments, the one or more walls of any one of the plurality of receptacles are independent of the one or more walls of the other receptacles of the plurality of receptacles. Each receptacle of the plurality of receptacles may be individually positionable at the tissue area. Each receptacle of the plurality of receptacles may be associated with a separate fluid supply. The apparatus may comprise at least one manifold for providing fluid to each receptacle of the plurality of receptacles.

The apparatus may comprise a plurality of fluid inlet conduits, wherein each receptacle of the plurality of receptacles is in fluid communication with a respective fluid inlet conduit of the plurality of fluid inlet conduits, via which fluid is supplied to the respective receptacle. Each of the receptacles may be arranged in fluid communication with at least one other of the receptacles. One or more of the receptacles of the plurality of receptacles may have a plurality of microstructures formed thereon. The apparatus may further comprise a cover positionable over the plurality of receptacles to form a compartment substantially bounded by the cover, the plurality of receptacles, and the tissue area. The compartment substantially bounded by the cover, the receptacle and the tissue area may be further bounded by the plurality of fluid inlet conduits.

In some embodiments, the plurality of fluid inlet conduits are in fluid communication with the manifold and wherein the manifold has a fluid supply conduit that passes through a single opening in the cover. Alternatively, each fluid inlet conduit of the plurality of fluid inlet conduits passes through a respective opening in the cover. In still further embodiments, each fluid inlet conduit of the plurality of fluid inlet conduits extends past an outer edge of the cover to an associated fluid supply. The compartment substantially bounded by the cover, the receptacle and the tissue area may be further bounded by the plurality of fluid inlet conduits, the manifold and the fluid supply conduit.

In some embodiments, the at least one section allows molecules within the fluid to diffuse from the receptacle to the tissue area. In some embodiments, the at least one section allows the fluid pass from the receptacle to the tissue area by pore flow.

The receptacles may each comprise multiple layers, including a first layer configured to form a tissue facing layer, and a second layer. The second layer may have a first thickness and the first layer has a second thickness, the first thickness being greater than the second thickness. The second layer may comprise an upper layer of the receptacle.

The fluid may comprise a gas, such as oxygen gas or carbon dioxide gas.

According to a further aspect, an apparatus for supplying fluid to a tissue area comprises a receptacle positionable at a tissue area, the receptacle comprising one or more walls adapted to receive a fluid, the one or more walls comprising at least one section adapted to allow the fluid to pass from the receptacle to the tissue area; a fluid inlet in fluid communication with the receptacle; and a pressure relief valve in communication with the fluid inlet and/or the receptacle and operable to relieve pressure within the fluid inlet and/or receptacle.

The receptacle may be inflatable. The tissue area may comprise a wound area.

In some embodiments, the at least one section allows molecules within the fluid to diffuse from the receptacle to the tissue area. In some embodiments, the at least one section allows the fluid pass from the receptacle to the tissue area by pore flow.

The receptacle may comprises multiple layers, including a first layer configured to form a tissue facing layer, and a second layer. The second layer may have a first thickness and the first layer has a second thickness, the first thickness being greater than the second thickness. The second layer may comprise an upper layer of the receptacle.

The fluid may comprise a gas, such as oxygen gas or carbon dioxide gas.

In some embodiments, the apparatus may further comprise a cover positionable over the receptacle to form a compartment substantially bounded by the cover, the receptacle, and the tissue area, wherein the pressure relief valve may be positioned inside the compartment. In this embodiment, the compartment substantially bounded by the cover, the receptacle and the tissue area may be further bounded by the pressure relief valve. In other embodiments, the pressure relief valve may be positioned externally of the compartment. The fluid inlet and/or the fluid outlet may comprise an opening formed in the cover. The tissue area may comprise a wound area.

According to a further aspect, an apparatus for supplying fluid to a tissue area comprises a fluid delivery device configured to be positioned over a tissue site, wherein the fluid delivery device comprises a head portion that is configured to be positioned over the tissue area, a tail portion that is configured to extend away from the tissue area and a fluid inlet conduit arranged in fluid communication with the tail portion.

The fluid inlet conduit may be connected to the tail portion. The head portion may be in fluid communication with the tail portion such that the fluid inlet conduit is configured to supply fluid to the head portion via the tail portion. The tail portion may be longer than it is wide. The tail portion may have a width that is less than the width of the head portion

The fluid inlet conduit may pass through a wall of the tail portion. The wall through which the fluid inlet conduit may pass may be at an end of the tail portion that is furthest from the head portion.

The fluid delivery device may comprise a receptacle. The receptacle may be inflatable. In some embodiments, the receptacle is a bag. The receptacle may comprise one or more walls and is adapted to receive a fluid. The walls may comprise at least one section adapted to allow molecules within the fluid to move from inside of the receptacle to the tissue area. The one or more walls may be adapted to allow the molecules to move through the wall via diffusion. The one or more walls may be substantially pore-free such that molecules do not move through the wall via pore flow. The one or more walls may be substantially impermeable to bulk transport of fluid.

The apparatus may comprise a pressure spreading device. The fluid inlet conduit may be bonded to the pressure spreading device. The pressure spreading device may encapsulate at least a portion of the fluid inlet conduit between the fluid delivery device and a fluid source.

The pressure spreading device may be shaped to reduce a pressure exerted on the patient by the fluid inlet conduit. In some embodiments, the pressure spreading device may have a width that is greater than an external diameter of the fluid inlet conduit.

The tail portion and the pressure spreading device may be formed integrally with one another or they may comprise separate components. The tail portion of the fluid delivery device and the pressure spreading device may be bonded together. The pressure spreading device and the fluid delivery device may be made from the same material.

The pressure spreading device may have a generally elliptical cross-section. In some embodiments, it may have a cross-section that is shaped like a convex lens.

The apparatus may comprise a negative pressure compartment.

The receptacle may comprise multiple layers, including a first layer configured to form a tissue facing layer, and a second layer. The second layer may have a first thickness and the first layer has a second thickness, the first thickness being greater than the second thickness. The second layer may comprise an upper layer of the receptacle.

The tissue area may comprise a wound area. The wound area may comprise a wound and skin adjacent to the wound. The tissue site may comprise a wound. The fluid may comprise a gas, such as oxygen gas or carbon dioxide gas.

According to a further aspect, an apparatus for supplying fluid to a tissue area comprises a fluid delivery device configured to be positioned over a tissue site, a fluid inlet conduit for delivery of fluid to the fluid delivery device, and a pressure spreading device which encapsulates at least a portion of the fluid inlet conduit and is adjacent the fluid delivery device.

The fluid inlet conduit may be connected to the fluid delivery device. The fluid inlet conduit may be bonded to the pressure spreading device. The pressure spreading device may be shaped to reduce a pressure exerted on the patient by the fluid inlet conduit. The pressure spreading device may have a width that is greater than an external diameter of the fluid inlet conduit. For example, the width may be about 2 to 10 times greater than the external diameter, or about 3 to 9 times greater than the external diameter, or about 4 to 8 times greater than the external diameter, or about 5 to 7 times greater than the external diameter of the fluid inlet conduit.

The pressure spreading device and the fluid delivery device may be made from the same material. The pressure spreading device may have an elliptical cross-section. In some embodiments, the pressure spreading device may have a cross-section that is shaped like a convex lens.

The fluid delivery device may comprise a receptacle. The receptacle may be inflatable. In some embodiments, the receptacle is a bag. The receptacle may comprise one or more walls and is adapted to receive a fluid. The walls may comprise at least one section adapted to allow molecules within the fluid to move from inside of the receptacle to the tissue area. The one or more walls may be adapted to allow the molecules to move through the wall via diffusion. The one or more walls may be substantially pore-free such that molecules do not move through the wall via pore flow. The one or more walls may be substantially impermeable to bulk transport of fluid.

The receptacle may have a head portion that is configured to be positioned over the tissue area. The receptacle may have a tail portion that is configured to extend away from the tissue area. The fluid inlet conduit may be connected to the tail portion.

The head portion of the receptacle may be in fluid communication with the tail portion such that the fluid inlet conduit is configured to supply fluid to the head portion via the tail portion.

The tail portion may be longer than it is wide. The tail portion may have a width that is less than a width of the head portion when the receptacle is in a non-inflated configuration.

The tail portion of the fluid delivery device and the pressure spreading device may be formed integrally with one another or as separate components. The tail portion and the pressure spreading device may be bonded together.

The fluid inlet conduit may pass through a wall of the tail portion. The wall through which the fluid inlet conduit may pass may be at an end of the tail portion that is furthest from the head portion.

The apparatus may comprise a negative pressure compartment.

The receptacle may comprise multiple layers, including a first layer configured to form a tissue facing layer, and a second layer. The second layer may have a first thickness and the first layer has a second thickness, the first thickness being greater than the second thickness. The second layer may comprise an upper layer of the receptacle.

The tissue area may comprise a wound area. The wound area may comprise a wound and skin adjacent to the wound. The tissue site may comprise a wound. The fluid may comprise a gas, such as oxygen gas or carbon dioxide gas.

According to a further aspect, a tissue care dressing comprises a receptacle comprising at least one flexible wall having at least one inner surface; wherein at least a portion of the at least one inner surface comprises a rough surface adapted for reducing adherence of the at least a portion of the at least one inner surface to another portion of the inner surface and/or to another inner surface of the receptacle. The receptacle may be inflatable and/or in the form of a bag.

The rough surface may be irregular and may vary in height. It may include a plurality of raised and/or recessed elements that vary in width. The rough surface may be present on a non-wound contacting surface of the receptacle.

The receptacle may be adapted for connection to a fluid source. The receptacle may be configured to deliver therapy to a wound upon connection of the receptacle to the fluid source. The receptacle may be positionable at a tissue area and adapted to receive a fluid from the fluid source, the at least one flexible wall comprising at least one section adapted to allow the fluid to pass from the receptacle to the tissue area. The at least one section of the at least one flexible wall may be adapted to allow molecules within the fluid to diffuse from the receptacle to the tissue area. The at least one section of the at least one flexible wall may be adapted to allow the fluid to pass from the receptacle to the tissue area by pore flow.

The receptacle may be configured to contact the tissue area. The at least one section of the receptacle may be conformable with a surface of the tissue area. The at least one section of the at least one flexible wall may comprise a stretchable material. The at least one section of the at least one flexible wall may comprise a membrane. The membrane may be adapted to allow molecules to diffuse from the receptacle to the tissue area. The membrane may be substantially pore-free. The membrane may be impermeable to bulk transport of fluid. The at least one section of the receptacle may be configured to have an undulating outer surface when at least partially inflated.

The tissue care dressing may further comprise structures associated with the at least one section of the receptacle, the structures being configured to contact the tissue area. The structures may be configured for encouraging cell growth. The structures may be microstructures.

The tissue care dressing may comprise ahead portion that is configured to be positioned over the tissue area, a tail portion that is configured to extend away from the tissue area and a fluid inlet conduit arranged in fluid communication with the tail portion. The fluid inlet conduit may be connected to the tail portion. The head portion may be in fluid communication with the tail portion such that the fluid inlet conduit is configured to supply fluid to the head portion via the tail portion. The tail portion is longer than it is wide. The tail portion has a width that is less than the width of the head portion. The head portion may include the rough surface. Alternatively or additionally, the tail portion may include the rough surface.

The receptacle may comprise multiple layers, including a first layer configured to form a tissue facing layer, and a second layer. The second layer may have a first thickness and the first layer has a second thickness, the first thickness being greater than the second thickness. The second layer may comprise an upper layer of the receptacle.

The tissue area may comprise a wound area. The tissue care dressing may comprise a plurality of the receptacles. The fluid may comprise a therapeutic gas, for example oxygen and/or carbon dioxide.

According to a further aspect, a tissue care dressing comprises a receptacle being positionable at a tissue area of a patient and comprising at least one flexible wall including a first wall portion and a second wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path whilst mitigating indentation of the at least one ridge on the tissue area of the patient, upon a pressing force being applied to the tissue care dressing.

The receptacle may comprise a head portion that is configured to be positioned over the tissue area, and a tail portion in fluid communication with the head portion and that is configured to extend away from the tissue area; wherein the at least one ridge is positioned in the tail portion and/or in the head portion. The at least one ridge may be configured to maintain the fluid flow path at least partially open upon compression of the tail portion and/or the head portion of the receptacle in use.

The at least one ridge may extend along a longitudinal axis of the tail portion of the receptacle. The at least one ridge may extend along a longitudinal axis of the head portion of the receptacle. The at least one ridge may be substantially straight. Alternatively, the at least one ridge may have a shape, when viewed in plan view, that is substantially non-straight. For example, when viewed in plan view, the at least one ridge may be one or more of curved, zigzag shaped or irregularly shaped.

The at least one ridge may have a height dimension of between about 100 to 200 micrometres, or between about 125 to 175 micrometres, or approximately 150 micrometres. The at least one ridge may be formed from a yielding material.

The at least one ridge may comprise two or more ridges arranged substantially parallel to one another.

The tissue care dressing may comprise at least one ridge on the inner surface of the first wall portion of the receptacle and at least one ridge on the inner surface of the second wall portion of the receptacle.

The at least one ridge on the inner surface of the first wall portion may be offset from the at least one ridge on the inner surface of the second wall portion.

The at least one ridge may adjoin the inner surface of the respective first wall portion or second wall portion with a substantially non-filleted corner. The substantially non-filleted corner may be a sharp corner.

The at least one ridge may adjoin the inner surface of the respective first wall portion or second wall portion with a fillet radius of less than or equal to about 30% or less than or equal to about 25% or less than or equal to about 20% or less than or equal to about 10% of a height dimension of the ridge. A thickness dimension of the first wall portion and/or the second wall portion may be less than or approximately equal to a height dimension of the ridge.

The first wall portion and/or the second wall portion may have a wall thickness of between about 10 micrometres and about 150 micrometres, for example between approximately 20 micrometres and 140 micrometres, or between about 30 micrometres and 130 micrometres, or between about 35 micrometres and 100 micrometres, or between about 40 micrometres and 80 micrometres, or between approximately 40 micrometres to 70 micrometres, or between approximately 30 micrometres to 60 micrometres, or between approximately 20 to 60 micrometres, or between about 45 micrometres and 55 micrometres. In some embodiments, the wall thickness is approximately 60 micrometres. The first wall portion and/or the second wall portion may have a wall thickness of approximately 50 micrometres.

The inner surface of the at least one of the first wall portion and/or the second wall portion may comprise a plurality of the ridges.

The first wall portion and the second wall portion may be part of a single common wall. Alternatively, the first wall portion may be a portion of a first wall and the second wall portion may be a portion of a second wall.

The first wall portion and/or the second wall portion may comprise a rough surface adapted for reducing adherence of the first wall portion to the second wall portion.

The receptacle may be inflatable. The receptacle may be in the form of a bag.

The receptacle may be adapted for connection to a fluid source. The receptacle may be configured to deliver therapy to a wound upon connection of the receptacle to the fluid source. The receptacle may be adapted to receive a fluid, the at least one flexible wall comprising at least one section adapted to allow the fluid to pass from the receptacle to the tissue area.

The at least one section of the at least one flexible wall may be adapted to allow molecules within the fluid to diffuse from the receptacle to the tissue area. Alternatively, the at least one section of the at least one flexible wall may be adapted to allow the fluid to pass from the receptacle to the tissue area by pore flow.

The receptacle may be configured to contact the tissue area. The at least one section of the receptacle may be conformable with a surface of the tissue area.

The at least one section of the at least one flexible wall and/or the first wall portion and/or the second wall portion may comprise a stretchable material. The at least one section and/or the first wall portion and/or the second wall portion of the at least one flexible wall may comprise a membrane. The membrane may be adapted to allow molecules to diffuse from the receptacle to the tissue area. The membrane may be substantially pore-free. The membrane may be impermeable to bulk transport of fluid.

The at least one section of the receptacle may be configured to have an undulating outer surface when at least partially inflated.

The tissue care dressing may further comprise structures associated with the at least one section of the receptacle, the structures being configured to contact the tissue area. The structures may be configured for encouraging cell growth. The structures may be microstructures.

The tissue care dressing may comprise ahead portion that is configured to be positioned over the tissue area, a tail portion that is configured to extend away from the tissue area and a fluid inlet conduit arranged in fluid communication with the tail portion. The fluid inlet conduit may be connected to the tail portion. The head portion is in fluid communication with the tail portion such that the fluid inlet conduit is configured to supply fluid to the head portion via the tail portion.

The tail portion may be longer than it is wide. The tail portion may have a width that is less than the width of the head portion. The head portion may include the rough surface. Alternatively or additionally, the tail portion may include the rough surface.

The receptacle may comprise multiple layers, including a first layer configured to form a tissue facing layer, and a second layer. The second layer may have a first thickness and the first layer has a second thickness, the first thickness being greater than the second thickness. The second layer may comprise an upper layer of the receptacle.

According to a further aspect, an apparatus for supplying fluid to a tissue comprises: a fluid receptacle positionable at a tissue area, the fluid receptacle comprising one or more walls, a fluid inlet for receiving fluid into the fluid receptacle, and a fluid outlet for the passage of fluid from the fluid receptacle; and a pressure regulator associated with the fluid outlet.

The pressure regulator may be positioned in spaced relation from the fluid receptacle. The apparatus may further comprise a fluid outlet conduit arranged in fluid communication with the fluid receptacle, wherein the pressure regulator is disposed on the fluid outlet conduit. The pressure regulator may be selectively actuable to an at least partially open position to permit fluid to pass there through, and is configured, when in the at least partially open position, to vent the fluid passing there through to the atmosphere.

A portion of the fluid receptacle may define a tissue facing portion, configured to face the tissue of the patient in use of the apparatus.

A fluid flow path through the fluid receptacle may be defined by the one or more walls of the fluid receptacle, the fluid inlet and the fluid outlet. The one or more walls of the fluid receptacle may be made of a polymer material. A boundary of the fluid flow path through the fluid receptacle may be independent of the tissue of the patient. The one or more walls of the receptacle may be present between the fluid flow path and the tissue of the patient in use.

At least a portion of the tissue facing portion may comprise a membrane adapted to allow molecules within the receptacle to pass through the membrane to the tissue of the patient. At least a portion of the tissue facing portion may comprise a membrane adapted to allow fluid within the receptacle to pass through the membrane to the tissue of the patient.

The fluid inlet may be adapted for providing a positive pressure within the fluid receptacle. The one or more walls of the fluid receptacle may define a positive pressure receptacle. The fluid receptacle may be inflatable.

The pressure regulator may comprise a pressure relief valve. The pressure regulator may be configurable to relieve pressure within the receptacle when the pressure within the receptacle reaches a threshold value. The pressure regulator may be configurable to adjust the pressure threshold value. The pressure threshold value is an upper bound of the pressure. The pressure regulator may be passively and/or mechanically actuable to permit fluid to pass there through. Alternatively, the pressure regulator may be actively actuable to permit fluid to pass there through.

The fluid receptacle may comprises multiple layers, including a first layer configured to form a tissue facing layer, and a second layer. The second layer may have a first thickness and the first layer has a second thickness, the first thickness being greater than the second thickness. The second layer may comprise an upper layer of the receptacle. The upper layer and the tissue facing layer may be permeable, the upper layer having a lower permeability than the tissue facing layer. The second layer may include a plurality of apertures configured to permit fluid within the fluid receptacle to pass there through to exit the fluid receptacle. The plurality of apertures may be configured to provide pressure relief within the fluid receptacle.

The fluid receptacle may be adapted to allow fluid to enter into the fluid receptacle at the fluid inlet. The apparatus may further comprise a fluid source in fluid communication with the fluid inlet and adapted to provide a substantially continuous supply of fluid to the fluid receptacle. The apparatus may be further adapted to apply a negative pressure to the tissue of the patient. The apparatus may further comprise a cover positionable over the fluid receptacle at a tissue area of the patient to form a compartment substantially bounded by the cover, the receptacle and the tissue area, the cover comprising a negative pressure port.

The fluid may comprise a therapeutic gas, for example oxygen and/or carbon dioxide and/or carbon monoxide.

The apparatus may comprise structures associated with the tissue facing portion of the receptacle, the structures being configured to face the tissue of the patient. The structures may be microstructures and/or may comprise formations which protrude from the one or more walls of the receptacle.

The one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

According to a further aspect, an apparatus comprises: a receptacle comprising one or more walls, a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet. The apparatus may for treating tissue. The apparatus may be for supplying fluid to tissue of a patient.

The one or more walls may include a tissue facing portion; and the internal flow guide may have a first flow guide surface and a second opposing flow guide surface; wherein the internal flow guide is disposed in the receptacle such that only its first flow guide surface faces the tissue facing portion.

Alternatively, the one or more walls may include a tissue facing portion; and the internal flow guide may have a first flow guide surface and a second opposing flow guide surface; wherein the internal flow guide is disposed in the receptacle such that the tissue facing portion is disposed adjacent each of the first flow guide surface and the second flow guide surface.

The internal flow guide may comprise an internal wall of the receptacle.

The one or more walls of the receptacle may include at least a section comprising a membrane adapted to allow molecules within the fluid to pass from the receptacle to the tissue of the patient. The receptacle may be sealed such that fluid may enter the receptacle substantially only through the fluid inlet; and fluid may exit the receptacle substantially only through the fluid outlet or as a result of molecules within the fluid passing through the membrane. The membrane may be adapted to allow molecules within the fluid to diffuse from the receptacle to the tissue of the patient. The membrane may comprise the tissue facing portion, configured to allow fluid within the receptacle to pass through the tissue facing portion to the tissue of the patient.

The membrane may be impermeable to bulk transport of fluid. The membrane may be hydrophobic.

The at least a portion of the internal flow guide may have a length, wherein the fluid flow path extends along the length prior to exiting the receptacle. The internal flow guide may be configured to prolong a residence time in which fluid entering the fluid inlet spends within the receptacle prior to exiting the receptacle through the fluid outlet.

The apparatus may have a longitudinal axis, wherein the internal flow guide is configured to extend at least partially along or substantially parallel to the longitudinal axis prior to use. The internal flow guide may be disposed substantially orthogonal to the tissue facing portion, prior to use.

The apparatus may have a longitudinal axis, wherein the internal flow guide is configured to extend at least partially along the longitudinal axis of the dressing.

The apparatus may have a longitudinal axis, wherein the internal flow guide is configured to extend at least partially along the longitudinal axis of the dressing, wherein the internal flow guide is disposed in the receptacle such that only its first flow guide surface faces the tissue facing portion or wherein the internal flow guide is disposed in the receptacle such that the tissue facing portion is disposed adjacent each of the first flow guide surface and the second flow guide surface.

The internal flow guide may divide the receptacle into a first compartment and a second compartment, the first compartment and the second compartment being fluidly interconnected. The internal flow guide may have a fluid flow path feature adapted for allowing fluid to flow from the first compartment to the second compartment. The fluid flow path feature may be one or more of an opening, an aperture, a permeable membrane or a perforation.

The fluid inlet and the fluid outlet may be disposed substantially adjacent one another. The fluid inlet may be disposed to allow fluid to enter into the first compartment, the fluid outlet may be disposed to allow fluid in the second compartment to exit the receptacle, and wherein the internal flow guide is configured to substantially separate the first compartment and the second compartment. The internal flow guide may be configured to cause fluid within the first compartment to flow past at least a portion of the internal flow guide prior to exiting the receptacle via the outlet.

The internal flow guide may be configured to permit fluid within the receptacle to pass through it. Alternatively, the internal flow guide may comprise a material adapted to substantially prevent molecules within the fluid from passing through it. It may have a thickness that is one of greater than or substantially the same as a thickness of the one or more walls of the receptacle.

The receptacle may be inflatable. The internal flow guide may be disposed inside the inflatable receptacle. The receptacle may be divided into a first compartment and a second compartment, wherein the first and second compartments of the receptacle are substantially hollow.

The apparatus may further comprise a pressure regulator associated with the fluid outlet. The pressure regulator may be a pressure relief valve. The pressure regulator may be configurable to relieve pressure within the receptacle when the pressure within the receptacle reaches a threshold value. The pressure regulator may be passively and/or mechanically actuable to permit fluid to pass there through. The pressure regulator may be disposed in spaced relation from the fluid receptacle.

According to a further aspect, an apparatus for supplying fluid to tissue of a patient comprises: a fluid receptacle positionable at a tissue area, the fluid receptacle comprising one or more walls defining a first compartment and a second compartment of the fluid receptacle; a fluid inlet disposed for receiving fluid into the first compartment of the fluid receptacle, and a fluid outlet disposed for the passage of fluid from the second compartment of the fluid receptacle to exit the receptacle; wherein the one or more walls of the receptacle, the fluid inlet and the fluid outlet are configured to define a fluid flow path whereby fluid entering the receptacle at the fluid inlet is caused to pass from the first compartment into the second compartment.

A fluid flow path through the fluid receptacle may be defined by the one or more walls of the fluid receptacle, the internal flow guide, the fluid inlet and the fluid outlet. A boundary of the fluid flow path through the fluid receptacle may be independent of the tissue of the patient. The one or more walls of the receptacle may be present between the fluid flow path and the tissue of the patient in use.

The receptacle may be in the form of a bag.

In some embodiments, the apparatus may comprise a cover positionable over the receptacle to form a compartment substantially bounded by the cover, the receptacle and the tissue area. The compartment substantially bounded by the cover, the receptacle and the tissue area may be pressurised during use to form a negative pressure compartment. The apparatus may comprise a negative pressure source arranged in fluid communication with the compartment substantially bounded by the cover, the receptacle and the tissue area for applying a negative pressure to the compartment.

The fluid may comprise a therapeutic gas. The gas may comprise oxygen and/or carbon dioxide and/or carbon monoxide.

The fluid outlet may be configured to allow cycling of fluid out of the fluid receptacle.

The apparatus may comprise structures associated with the tissue facing portion of the receptacle, the structures being configured to face the tissue of the patient. The structures may be microstructures and/or may comprise formations which protrude from the one or more walls of the receptacle.

The one or more walls may include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

A thickness dimension of the first wall portion and/or the second wall portion may be less than or approximately equal to a height dimension of the ridge. A thickness dimension of the first wall portion and/or the second wall portion may be greater than a height dimension of the ridge.

The first wall portion and the second wall portion may be part of a single common wall. Alternatively, the first wall portion is a portion of a first wall and the second wall portion is a portion of a second wall.

The one or more walls of the fluid receptacle may include an internal wall configured to divide the receptacle into the first compartment and the second compartment. The one or more walls of the receptacle may include at least a section of each of the first compartment and/or the second compartment comprising a membrane adapted to allow molecules within the fluid to pass from the receptacle to the tissue of the patient. The membrane may be adapted to allow molecules within the fluid to diffuse from the receptacle to the tissue of the patient. The membrane may comprise a tissue facing portion, configured to allow fluid within the receptacle to pass through to the tissue of the patient.

The at least a section of the one or more walls of the receptacle may form the tissue facing portion.

At least a portion of the internal wall has a length, and the fluid flow path may extend along the length and fluid flows along the fluid flow path in contact with tissue facing portion such that molecules from the fluid can pass through the tissue facing portion prior to exiting the receptacle. The internal wall may be configured to prolong a residence time in which fluid entering the fluid inlet spends within the receptacle prior to exiting the receptacle through the fluid outlet.

The apparatus may have a longitudinal axis, wherein the internal wall may be configured to extend at least partially along or substantially parallel to the longitudinal axis.

The internal wall may have a first flow guide surface and a second opposing flow guide surface and may be disposed such that the tissue facing portion is disposed adjacent each of the first flow guide surface and the second flow guide surface during use.

The apparatus may have a longitudinal axis, wherein the internal wall is configured to extend at least partially along the longitudinal axis of the dressing, prior to use.

The apparatus may have a longitudinal axis, wherein the internal wall is configured to extend at least partially along the longitudinal axis of the dressing, and wherein the internal wall has a first flow guide surface and a second opposing flow guide surface; wherein the internal wall is either disposed in the receptacle such that only its first flow guide surface faces the tissue facing portion during use or disposed such that the tissue facing portion is disposed adjacent each of the first flow guide surface and the second flow guide surface during use.

The fluid outlet may be configured for flushing fluid out of the fluid receptacle.

At least a periphery of each of the first compartment and the second compartment may be adjoining and may form the internal wall. The internal wall may have a fluid flow path feature adapted for allowing fluid to flow from the first compartment to the second compartment. The fluid flow path feature may be one or more of an opening, an aperture, a permeable membrane, a connector or a perforation.

The fluid inlet and the fluid outlet may be disposed substantially adjacent one another. The fluid inlet may be disposed to allow fluid to enter into the first compartment, the fluid outlet may be disposed to allow fluid in the second compartment to exit the receptacle, and the internal wall may be configured to substantially separate the first compartment and the second compartment. The internal wall may be configured to cause fluid within the first compartment to flow past at least a portion of the internal flow guide prior to exiting the receptacle via the outlet.

The internal wall may be configured to permit fluid within the receptacle to pass through it. Alternatively, the internal wall may comprise a material adapted to substantially prevent molecules within the fluid from passing through it. The internal wall may have a thickness that is one of greater than or substantially the same as a thickness of the one or more walls of the receptacle.

The first and second compartments may be inflatable. The internal wall may be disposed inside the inflatable receptacle.

The first and second compartments of the receptacle may be substantially hollow.

The apparatus may further comprise a pressure regulator associated with the fluid outlet. The pressure regulator may be a pressure relief valve. The pressure regulator may be configurable to relieve pressure within the receptacle when the pressure within the receptacle reaches a threshold value. The pressure regulator may be passively and/or mechanically actuable to permit fluid to pass there through. The pressure regulator may be disposed in spaced relation from the fluid receptacle.

A fluid flow path through the fluid receptacle may be defined by the one or more walls of the fluid receptacle, the at least a portion of the internal wall, the fluid inlet and the fluid outlet.

According to a further aspect, an apparatus for supplying fluid to tissue of a patient comprises: a fluid receptacle comprising one or more outer walls and adapted to receive a fluid, and having an internal wall configured to divide the receptacle into a first region and a second region, the first region and the second region being fluidly interconnected; and a fluid inlet disposed for receiving fluid into the first region of the fluid receptacle, and a fluid outlet disposed for the passage of fluid from the second region of the fluid receptacle, wherein the one or more outer walls of the receptacle, the internal wall, the fluid inlet and the fluid outlet are configured to define a fluid flow path whereby fluid entering the receptacle at the fluid inlet is caused to pass from the first region into the second region and to exit the second region of the receptacle at the fluid outlet.

The internal wall may include a fluid flow path feature adapted for allowing fluid to flow from the first region to the second region. The fluid flow path feature may be one or more of an opening, an aperture, a permeable membrane, connector or a perforation.

The one or more outer walls may be adapted to allow molecules within the fluid to pass from the receptacle to the tissue of the patient. The one or more outer walls may be adapted to allow molecules within the fluid to diffuse from the receptacle to the tissue of the patient. The one or more outer walls may comprise a membrane. At least one section of the one or more outer walls may form a tissue facing portion. The tissue facing portion may be configured to allow fluid within the receptacle to pass through the tissue facing portion to the tissue of the patient.

The receptacle may be sealed such that fluid may enter the receptacle substantially only through the fluid inlet; and fluid may exit the receptacle substantially only through the fluid outlet or as a result of molecules within the fluid passing through the tissue facing portion.

At least a portion of the internal wall may have a length, the fluid flow path extends along the length, and wherein fluid within the receptacle is caused to pass along at least a portion of the length and in fluid communication with the tissue of the patient via the tissue facing portion.

The one or more outer walls of the receptacle may comprise first and second walls. The first wall may form the tissue facing portion. A periphery of the second wall may be substantially sealed against a periphery of the first wall to form a substantially sealed receptacle. The internal wall may be a flow guide. The internal wall may at least partially divide the sealed receptacle into the first region and the second region. The flow guide may be configured to prolong a residence time in which fluid entering the fluid inlet spends within the receptacle prior to exiting the receptacle through the fluid outlet.

The apparatus may have a longitudinal axis, wherein the flow guide may be configured to extend at least partially along or substantially parallel to the longitudinal axis, prior to use.

The flow guide may have a first flow guide surface and an opposing second flow guide surface, and the flow guide may be disposed such that the tissue facing portion is disposed adjacent each of the first flow guide surface and the second flow guide surface during use.

The apparatus may have a longitudinal axis, wherein the flow guide may be configured to extend at least partially along the longitudinal axis of the dressing, prior to use.

The apparatus may have a longitudinal axis, wherein the flow guide is configured to extend at least partially along the longitudinal axis of the dressing, and wherein the flow guide has a first flow guide surface and a second opposing flow guide surface; wherein the flow guide may be either disposed in the receptacle such that only its first flow guide surface faces the tissue or disposed such that the tissue facing portion is disposed adjacent each of the first flow guide surface and the second flow guide surface during use.

The fluid inlet and the fluid outlet may be disposed substantially adjacent one another.

The fluid inlet may be disposed to allow fluid to enter into the first region, the fluid outlet may be disposed to allow fluid in the second region to exit the receptacle, and the internal wall may be configured to substantially separate the first region and the second region. The internal wall may be configured to cause fluid entering the receptacle at the fluid inlet in the first region to flow past at least a portion of the internal wall prior to exiting the receptacle via the adjacent outlet.

The internal wall may be configured to permit fluid within the receptacle to pass through it. Alternatively, the internal wall may comprise a material adapted to substantially prevent molecules within the fluid from passing through it. The internal wall may have a thickness that is one of greater than or substantially the same as a thickness of the one or more walls of the receptacle.

The receptacle may be inflatable. The receptacle may be substantially hollow.

A pressure regulator may be associated with the fluid outlet. The pressure regulator may be a pressure relief valve.

According to a further aspect, an apparatus comprises: a fluid receptacle comprising one or more walls and adapted to receive a fluid therein, the one or more walls having a fluid inlet for receiving fluid into the receptacle and a fluid outlet through which fluid within the receptacle may pass to upon exiting the receptacle; wherein the fluid inlet and the fluid outlet are disposed substantially adjacent one another. The apparatus may be for treating tissue. The apparatus may be for supplying fluid to tissue of a patient.

The apparatus may further comprise a fluid inlet conduit arranged in fluid communication with the fluid inlet and a fluid outlet conduit arranged in fluid communication with the fluid outlet, wherein the fluid inlet conduit and the fluid outlet conduit adjoin the receptacle parallel to one another.

An end portion of the fluid inlet conduit and an end portion of the fluid outlet conduit may be disposed substantially adjacent one another at a portion of the one or more walls of the receptacle. The fluid inlet conduit and the fluid outlet conduit may be arranged substantially parallel with one another at the interface portion of the one or more walls of the receptacle. The fluid inlet conduit and the fluid outlet conduit may be at least partially adjoined so as to extend from the portion of the one or more walls of the receptacle in a common direction. At least the end portion of the fluid inlet conduit and the fluid outlet conduit may be arranged substantially parallel with the tissue of a patient during use. At least the end portion of the fluid inlet conduit and the fluid outlet conduit may be configured to lie substantially adjacent and in close spaced relation from the tissue of a patient during use. At least the end portion of the fluid inlet conduit and the fluid outlet conduit may be configured to lie substantially adjacent and in contact with the tissue of a patient during use.

Prior to use, the receptacle may have a longitudinal axis, and at least the end portion of the fluid inlet conduit and the fluid outlet conduit may be arranged to extend from the interface of the receptacle in a direction along or substantially parallel to the longitudinal axis.

The receptacle may be inflatable.

The fluid outlet may be configured for cycling fluid out of the fluid receptacle. The fluid outlet may be configured to allow cycling of the fluid from the receptacle to maintain a concentration of the fluid within the receptacle.

The receptacle may comprise an internal wall configured to divide the receptacle into a first region and a second region, wherein the fluid inlet is disposed to allow fluid to enter into the first region and the fluid outlet is disposed to allow fluid to exit the receptacle from the second region. The internal wall may be configured to substantially separate the first region and the second region.

The internal wall may be configured to cause fluid entering the receptacle at the fluid inlet in the first region to flow past at least a portion of the internal wall prior to exiting the receptacle via the adjacent outlet.

At least a portion of the one or more walls may form a tissue facing portion. The tissue facing portion may be adapted to allow fluid molecules within the fluid to pass from the receptacle to the tissue of the patient. The tissue facing surface may be adapted to allow molecules within the fluid to diffuse from the receptacle to the tissue of the patient.

A fluid flow path through the fluid receptacle may be defined by the one or more walls of the fluid receptacle, the at least a portion of the internal wall, the fluid inlet and the fluid outlet. A boundary of the fluid flow path through the fluid receptacle may be independent of tissue of the patient. The one or more walls of the receptacle may be present between the fluid flow path and the tissue of the patient in use.

The apparatus may further comprise a pressure spreading device adapted for contacting the tissue of the patient. The pressure spreading dressing may encapsulate at least a portion of the fluid inlet conduit and the fluid outlet conduit.

The receptacle may comprise ahead portion and a tail portion, wherein the fluid inlet and the fluid outlet are disposed at the tail portion. The fluid inlet conduit and the fluid outlet conduit may each have a tail end that terminates at the tail portion of the receptacle to provide fluid communication between the respective one of the fluid inlet conduit and the fluid outlet conduit and the receptacle.

The apparatus may further comprise a pressure regulator associated with the fluid outlet. The pressure regulator may be a pressure relief valve.

The tissue area may comprise a wound area. The apparatus may comprise a plurality of the receptacles. The fluid may comprise a therapeutic gas. The gas may comprise oxygen and/or carbon dioxide and/or carbon monoxide.

According to a further aspect, a tissue care dressing, comprises a receptacle comprising at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

The receptacle may be inflatable. The rough surface may be irregular. It may vary in height. The rough surface may include a plurality of raised and/or recessed elements that vary in width. The rough surface may be a non-wound contacting surface of the receptacle.

The receptacle may be adapted for connection to a fluid source. It may be configured to deliver therapy to a wound upon connection of the receptacle to the fluid source.

The receptacle may be positionable at a tissue area and adapted to receive a fluid, the at least one flexible wall comprising at least one section adapted to allow the fluid to pass from the receptacle to the tissue area. The at least one section of the at least one flexible wall may be adapted to allow molecules within the fluid to diffuse from the receptacle to the tissue area. The at least one section of the at least one flexible wall may be adapted to allow the fluid to pass from the receptacle to the tissue area by pore flow. The at least one section of the at least one flexible wall may be adapted to allow molecules to diffuse from the receptacle to the tissue area is substantially pore free.

A tissue facing portion of the at least one flexible wall may be impermeable to bulk transport of fluid. The tissue facing portion may be hydrophobic.

The receptacle may be in the form of a bag.

The receptacle may be configured to contact the tissue area. The at least one section of the receptacle may be conformable with a surface of the tissue area.

The at least one section of the at least one flexible wall may comprise a membrane. The membrane may be adapted to allow molecules to diffuse from the receptacle to the tissue area. The membrane may be substantially pore-free. The membrane may be impermeable to bulk transport of fluid.

In some embodiments, the tissue care dressing may comprise structures associated with the at least one section of the receptacle, the structures being configured to contact the tissue area. The structures may be configured for encouraging cell growth. The structures may be microstructures. The structures may comprise formations which protrude from the one or more walls of the receptacle. Molecules within the fluid may diffuse through the tissue facing portion of the receptacle and the structures into the tissue of the patient.

The tissue care dressing may comprise a head portion that is configured to be positioned over the tissue area, and a tail portion that is configured to extend over and/or away from the tissue area. The tissue care dressing may further comprise a fluid inlet conduit arranged in fluid communication with the tail portion and/or connected to the tail portion.

The head portion may be in fluid communication with the tail portion such that the fluid inlet conduit is configured to supply fluid to the head portion via the tail portion.

The tail portion may be longer than it is wide. The tail portion may have a width that is less than the width of the head portion. The head portion may include the rough surface. The tail portion may include the rough surface.

The at least one flexible wall may include a first wall portion and a second wall portion opposite the first wall portion, the first wall portion and the second wall portion forming a fluid flow path there between, at least one ridge disposed on an inner surface of at least one of the first wall portion and the second wall portion. The at least one ridge may be configured to at least partially maintain the fluid flow path upon a pressing force being applied to the tissue care dressing.

The at least one ridge may have one or more properties configured to at least partially maintain the fluid flow path whilst mitigating indentation of the at least one ridge on the tissue area of the patient, upon a pressing force being applied to the tissue care dressing.

In some embodiments, the at least one ridge is substantially straight. In some embodiments, the at least one ridge may have a longitudinal form, when viewed in plan view, that is substantially non-straight.

The one or more properties of the at least one ridge may include that the at least one ridge has a height dimension of between about 100 to 200 micrometres, or between about 125 to 175 micrometres, or approximately 150 micrometres. The at least one ridge may be formed from a yielding material. The at least one ridge may comprise two or more ridges arranged substantially parallel to one another.

The tissue care dressing may comprise at least one ridge on the inner surface of the first wall portion of the receptacle and at least one ridge on the inner surface of the second wall portion of the receptacle. The at least one ridge on the inner surface of the first wall portion may be offset from the at least one ridge on the inner surface of the second wall portion.

The at least one ridge may adjoin the inner surface of the respective first wall portion or second wall portion with a substantially non-

The at least one ridge may adjoin the inner surface of the respective first wall portion or second wall portion with a fillet having a fillet radius of less than or equal to about 30% or less than or equal to about 20% or less than or equal to about 10% of a height dimension of the ridge.

A thickness dimension of the first wall portion and/or the second wall portion may be less than or approximately equal to a height dimension of the ridge. A thickness dimension of the first wall portion and/or the second wall portion may be greater than a height dimension of the ridge. the inner surface of the at least one of the first wall portion and/or the second wall portion comprises a plurality of the ridges.

The first wall portion and the second wall portion may be part of a single common wall. The first wall portion may be a portion of a first wall and the second wall portion may be a portion of a second wall.

The fluid may comprise a therapeutic gas. The gas may comprise oxygen and/or carbon dioxide and/or carbon monoxide.

The tissue care dressing may comprise a fluid outlet for the passage of fluid from the fluid receptacle; and a pressure regulator associated with the fluid outlet. The pressure regulator may comprise a pressure relief valve.

According to a further aspect, a tissue care dressing comprises a receptacle being positionable at a tissue area of a patient and comprising at least one flexible wall including a first wall portion and a second wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the tissue care dressing.

The at least one ridge may have one or more properties configured to at least partially maintain the fluid flow path whilst mitigating indentation of the at least one ridge on the tissue area of the patient, upon a pressing force being applied to the tissue care dressing. the receptacle comprises a head portion that is configured to be positioned over the tissue area, and a tail portion in fluid communication with the head portion and that is configured to extend away from the tissue area.

The at least one ridge may be positioned in the tail portion and/or in the head portion. The at least one ridge may have one or more properties configured to maintain the fluid flow path at least partially open upon compression of the tail portion and/or the head portion of the receptacle in use.

The at least one ridge may extend along a longitudinal axis of the tail portion of the receptacle. The at least one ridge may extend along a longitudinal axis of the head portion of the receptacle.

In some embodiments, the at least one ridge is substantially straight. In some embodiments, the at least one ridge may have a longitudinal form, when viewed in plan view, that is substantially non-straight.

The one or more properties of the at least one ridge may include that the at least one ridge has a height dimension of between about 100 to 200 micrometres, or between about 125 to 175 micrometres, or approximately 150 micrometres. The at least one ridge may be formed from a yielding material. The at least one ridge may comprise two or more ridges arranged substantially parallel to one another.

The tissue care dressing may comprise at least one ridge on the inner surface of the first wall portion of the receptacle and at least one ridge on the inner surface of the second wall portion of the receptacle. The at least one ridge on the inner surface of the first wall portion may be offset from the at least one ridge on the inner surface of the second wall portion.

The at least one ridge may adjoin the inner surface of the respective first wall portion or second wall portion with a substantially non-filleted corner. The substantially non-filleted corner may be a sharp corner.

The at least one ridge may adjoin the inner surface of the respective first wall portion or second wall portion with a fillet having a fillet radius of less than or equal to about 30% or less than or equal to about 20% or less than or equal to about 10% of a height dimension of the ridge.

A thickness dimension of the first wall portion and/or the second wall portion may be less than or approximately equal to a height dimension of the ridge. A thickness dimension of the first wall portion and/or the second wall portion may be greater than a height dimension of the ridge. the inner surface of the at least one of the first wall portion and/or the second wall portion comprises a plurality of the ridges.

The first wall portion and the second wall portion may be part of a single common wall. The first wall portion may be a portion of a first wall and the second wall portion may be a portion of a second wall.

The first wall portion and/or the second wall portion may comprise a rough surface adapted for reducing adherence of the first wall portion to the second wall portion.

The receptacle may be inflatable. The receptacle may be in the form of a bag.

The receptacle may be configured to contact the tissue area. The at least one section of the receptacle may be conformable with a surface of the tissue area.

The at least one section of the at least one flexible wall may comprise a membrane. The membrane may be adapted to allow molecules to diffuse from the receptacle to the tissue area. The membrane may be substantially pore-free. The membrane may be impermeable to bulk transport of fluid.

In some embodiments, the tissue care dressing may comprise structures associated with the at least one section of the receptacle, the structures being configured to contact the tissue area. The structures may be configured for encouraging cell growth. The structures may be microstructures. The structures may comprise formations which protrude from the one or more walls of the receptacle. Molecules within the fluid may diffuse through the tissue facing portion of the receptacle and the structures into the tissue of the patient.

The tissue care dressing may comprise ahead portion that is configured to be positioned over the tissue area, and a tail portion that is configured to extend over and/or away from the tissue area. The tissue care dressing may further comprise a fluid inlet conduit arranged in fluid communication with the tail portion and/or connected to the tail portion.

The head portion may be in fluid communication with the tail portion such that the fluid inlet conduit is configured to supply fluid to the head portion via the tail portion.

The tail portion may be longer than it is wide. The tail portion may have a width that is less than the width of the head portion. The head portion may include the rough surface. The tail portion may include the rough surface.

The receptacle may be adapted for connection to a fluid source. The receptacle may be configured to deliver therapy to a wound upon connection of the receptacle to the fluid source.

The fluid may comprise a therapeutic gas. The gas may comprise oxygen and/or carbon dioxide and/or carbon monoxide.

The tissue care dressing may comprise a fluid outlet for the passage of fluid from the fluid receptacle; and a pressure regulator associated with the fluid outlet. The pressure regulator may comprise a pressure relief valve.

According to a further aspect, an apparatus comprises a receptacle having: one or more walls comprising a first wall portion and a second wall portion opposite the first wall portion; an internal wall extending between the first wall portion and the second wall portion; the internal wall having an opening for passage of fluid there through; a fluid inlet; a fluid outlet; wherein the internal wall is configured as a barrier between the fluid inlet and the fluid outlet.

The internal wall may be configured to extend at least partially along a longitudinal axis of the receptacle. The internal wall may be configured to extend from an inner surface of the first wall portion to an inner surface of the second wall portion. The internal wall may be configured to divide the receptacle into a first compartment and a second compartment.

The fluid inlet and the fluid inlet outlet are disposed substantially adjacent one another. The fluid inlet may be disposed to allow fluid to enter into the first compartment and the fluid outlet may be disposed to allow fluid in the second compartment to exit the receptacle.

The opening of the internal wall may be positioned distally of the fluid inlet.

The apparatus may be configured to provide a fluid flow path from the fluid inlet into the first compartment, through the opening in the internal wall, into the second compartment and out of the fluid outlet.

The receptacle may be inflatable.

According to a further aspect, an apparatus for supplying fluid to a tissue area comprises: a receptacle positionable at a tissue area, the receptacle comprising one or more walls and being adapted to receive a fluid, the one or more walls comprising at least one section adapted to allow the fluid to pass from the receptacle to the tissue area; an inlet via which fluid is deliverable to the receptacle; an outlet via which fluid is receivable from the receptacle; and a cover positionable over the receptacle to form a compartment substantially bounded by the cover, the receptacle and the tissue area, the cover having a cover outlet via which fluid is receivable from the compartment substantially bounded by the cover, the receptacle and the tissue area.

It will be appreciated that the various aspects, embodiments and features of the apparatus disclosed herein may be combinable with one another.

### BRIEF DESCRIPTION OF DRAWINGS

One or more embodiments of the present disclosure will now be described by way of specific example(s) with reference to the accompanying drawings, in which:

FIG. 1 is a cross-sectional schematic view of an embodiment of an apparatus for supplying fluid to a tissue area;

FIG. 1A is a view of an embodiment of an apparatus for supplying fluid to a tissue area;

FIG. 2 is a cross-sectional schematic view of an embodiment of an apparatus for supplying fluid to a tissue area;

FIG. 3 is a cross-sectional schematic view of an embodiment of an apparatus for supplying fluid to a tissue area, having a fluid inlet conduit and first and second fluid outlet conduits;

FIG. 4 is a cross-sectional schematic view of an embodiment of an apparatus for supplying fluid to a tissue area, having multiple receptacles;

FIG. 5 is a cross-sectional schematic view of an embodiment of an apparatus for supplying fluid to a tissue area, having multiple receptacles and a manifold;

FIG. 6 is a cross-sectional schematic view of another embodiment of an apparatus for supplying fluid to a tissue area, having multiple receptacles and a manifold;

FIG. 7 is a cross-sectional schematic view of a further embodiment of an apparatus for supplying fluid to a tissue area;

FIG. 8 is a cross-sectional schematic view of an embodiment of an apparatus for supplying fluid to a tissue area, having a pressure relief valve;

FIG. 9 is a schematic plan view of an embodiment of the apparatus including a through-hole in the receptacle;

FIG. 10 is a schematic plan view of an embodiment of the apparatus including a plurality of through-holes in the receptacle;

FIG. 11 is a schematic plan view of an apparatus having multiple receptacles;

FIG. 12 is a cross-sectional schematic view of a further embodiment of the apparatus including fasteners;

FIG. 13 is a cross-sectional schematic view of a further embodiment of the apparatus including an absorbent material layer;

FIG. 14a and FIG. 14b are a schematic plan view and schematic cross-sectional view of an embodiment of the apparatus having combined fluid conduits;

FIG. 15a and FIG. 15b are a schematic plan view and schematic cross-sectional view of another embodiment of the apparatus having combined fluid conduits;

FIG. 16a and FIG. 1b are a schematic plan view and schematic cross-sectional view of a further embodiment of the apparatus having combined fluid conduits;

FIG. 17a and FIG. 17b are a schematic plan view and schematic cross-sectional view of a still further embodiment of the apparatus having combined fluid conduits;

FIG. 18 is a schematic cross-sectional view of another apparatus for supplying fluid to a tissue area having a receptacle;

FIG. 19 is a schematic cross-sectional view of the apparatus of FIG. 18, including a dressing;

FIG. 20 is a schematic cross-sectional view of another apparatus for supplying fluid to a tissue area having a receptacle;

FIG. 20A is a view of another apparatus for supplying fluid to a tissue area having a receptacle;

FIG. 21 is a schematic cross-sectional view of the apparatus of FIG. 20, including a dressing;

FIG. 21A is a view of a pressure spreading device, fluid inlet conduit and first fluid outlet conduit of an embodiment of the apparatus, protruding from a cover wrapped around a model of a leg of a patient;

FIG. 21B is a view of a receptacle, fluid inlet conduit and first fluid outlet conduit of an embodiment of the apparatus in situ on a wound on a model of a leg of a patient;

FIG. 21C is an enlarged view of a receptacle of an embodiment of the apparatus in situ on a wound on a model of a leg of a patient;

FIG. 22 is a schematic cross-sectional view of another apparatus for supplying fluid to a tissue area having multiple receptacles;

FIG. 23 is a schematic cross-sectional view of the apparatus of FIG. 22, including a dressing;

FIG. 24 is a schematic cross-sectional view of another apparatus for supplying fluid to a tissue, including a pressure relief valve;

FIG. 25 is a schematic cross-sectional view of the apparatus of FIG. 24, including a dressing;

FIG. 26 is a schematic plan view of a receptacle according to an embodiment of the apparatus;

FIG. 27 is a schematic cross-sectional view through the line A-A of FIG. 26;

FIG. 28 is a schematic cross-sectional view through the line B-B of FIG. 26 in i) an inflated configuration and ii) a configuration in which the receptacle is not inflated;

FIG. 28A is a view of a receptacle in i) an inflated configuration and ii) a configuration in which the receptacle is not inflated;

FIG. 29 is a schematic cross-sectional view through the line B-B of FIG. 26 with a) first rough surface and b) a first rough surface and a second rough surface, in accordance with an embodiment;

FIG. 30 is a close-up view of an example embodiment of the first rough surface of FIG. 29a and FIG. 29b;

FIG. 31 is a schematic cross sectional view of an inner surface of a receptacle with a) a plurality of ridges and b) a second wall portion pressed onto the ridge of a first wall portion of a receptacle according to an embodiment;

FIG. 32 is a schematic cross sectional view of the embodiment of FIG. 31, with i) overlapping ridges at a first wall portion and a second wall portion and ii) non-overlapping ridges at the first wall portion and the second wall portion;

FIG. 33 is a partial schematic cross-sectional representation of a ridge according to the embodiment of FIG. 31 adjoining a first and/or second wall portion with a) a small fillet radius; b) a medium fillet radius; c) a large fillet radius; and d) an overhanging corner;

FIG. 34 is a schematic plan view of a receptacle according to an embodiment showing embodiments of ridge configurations: a) ridges extend through tail portion; b) ridges extend through tail portion and head portion; c) non-straight ridges;

FIG. 35 is a schematic cross-sectional view of a further receptacle according to an embodiment of the apparatus;

FIG. 35A is a schematic cross-sectional view of a further receptacle according to an embodiment of the apparatus in a (i) unfolded configuration and (ii) folded configuration;

FIG. 35B is a schematic cross-sectional view of a further receptacle according to an embodiment of the apparatus in a (i) unfolded configuration and (ii) folded configuration;

FIG. 36 is a schematic cross-sectional view of an embodiment of the apparatus with a pressure relief valve at a fluid outlet port;

FIG. 37 is an embodiment of a receptacle with a pressure relief valve at a fluid outlet port;

FIG. 38 is an embodiment of a receptacle having a thicker material layer;

FIG. 39 is an embodiment of a receptacle having a plurality of apertures in an upper layer;

FIG. 40 is a schematic cross-sectional view of a further embodiment of the apparatus;

FIG. 41 is a schematic plan view of the embodiment of FIG. 40;

FIG. 42 is an enlarged schematic view of an opening of an internal flow guide of the embodiment of FIGS. 40 and 41;

FIG. 43 i) to iv) are example embodiments of cross-sectional shapes of a surround of the opening of FIGS. 40-42;

FIG. 43A to FIG. 43C are views of a receptacle showing overlap of an opening surround and ridges;

FIG. 43D is a schematic plan view of a circulation opening and opening surround of an internal flow guide of the embodiment of FIGS. 40 and 41;

FIG. 43E is a schematic plan view of the circulation opening and opening surround of an internal flow guide of FIG. 43D including ridges;

FIG. 43F is a schematic plan view of another circulation opening and opening surround of an internal flow guide of the embodiment of FIGS. 40 and 41;

FIG. 44 a) is a schematic cross-sectional view of the embodiment of FIG. 40 showing the attachment of the fluid inlet and outlet conduits to the receptacle;

FIG. 44 b) is an enlarged detail view of the attachment of the fluid inlet and outlet conduits to the receptacle;

FIG. 45 is schematic cross-sectional view of a further embodiment of the apparatus;

FIG. 46 is a further schematic plan view of the embodiment of the apparatus of FIG. 45;

FIG. 47 is a schematic plan view of a further embodiment of the apparatus;

FIG. 48 is a further cross-sectional side view of the embodiment of the apparatus of FIG. 47;

FIG. 49 is a schematic cross-sectional view of a further embodiment of the apparatus;

FIG. 50 a) is a schematic cross-sectional view of a further embodiment of the apparatus in which the fluid inlet and outlet conduits are disposed side by side;

FIG. 50 b) is a further schematic cross sectional view of the embodiment of the apparatus of FIG. 50 a), looking in the direction shown at line A-A in FIG. 50 a);

FIG. 51 a) and b) is a schematic diagram of example embodiments of internal ridges of the embodiments of FIGS. 41 to 50;

FIG. 52 is a schematic cross-sectional view of a further embodiment of the apparatus;

FIG. 53 is a schematic cross-sectional view of the tail portion and pressure spreading device of a receptacle and fluid inlet and outlet conduits of an embodiment of the apparatus;

FIG. 54 is another schematic view of the tail portion and pressure spreading device of a receptacle and fluid inlet and outlet conduits of an embodiment of the apparatus;

FIG. 55 is an exploded view of a pressure spreading device and tail portion of a receptacle, of an embodiment of the apparatus;

FIG. 56 is a partial section view of the pressure spreading device and tail portion of FIG. 55, including a cavity in the pressure spreading device, inlet conduit channel and outlet conduit channel;

FIG. 57 is a partial section view of the pressure spreading device and tail portion of FIG. 55, including a cavity in the pressure spreading device, inlet conduit channel and outlet conduit channel;

FIG. 58 is a cross-section view of the pressure spreading device of FIG. 55, showing a conduit locating feature of the inlet conduit channel;

FIGS. 59(a)-(d) are schematic plan views of alternative receptacle shapes;

FIGS. 60(a)-(d) are schematic plan views of alternative receptacle shapes that have a tail portion and a pressure spreading device;

FIG. 61 is a schematic representation of an embodiment of the apparatus, including an optional regulator and optional calibrated leak orifice upstream of the fluid inlet; and

FIG. 62 is a schematic partial representation of a variation of the embodiment of the apparatus of FIG. 61, including a conduit connector and holder.

### DESCRIPTION OF EMBODIMENTS

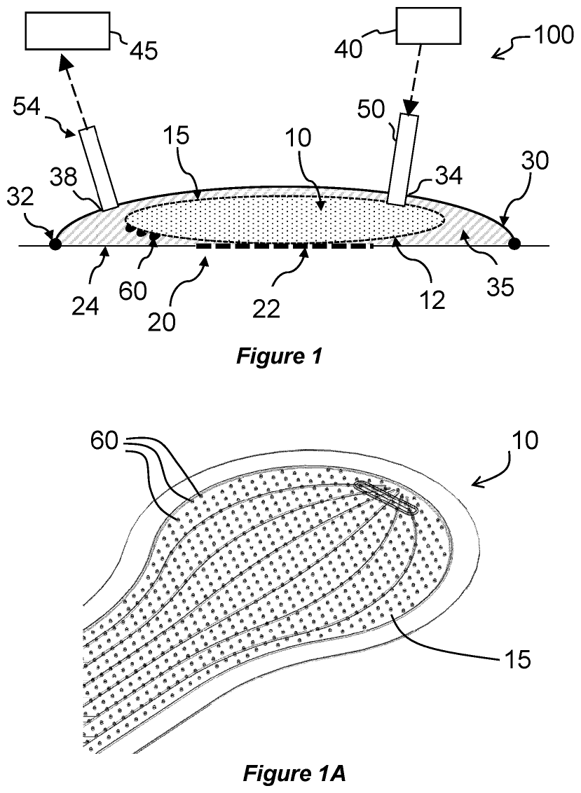

FIG. 1 shows an embodiment of an apparatus for supplying fluid to a tissue area. The tissue area may include target tissue that is to be treated and an area of tissue surrounding the target tissue. The target tissue may include one or more of healthy tissue, recently healed wound tissue, scar tissue, muscle, bone and the like. The apparatus may supply fluid to tissue on an external surface of the patient (e.g. skin) or it may supply fluid to tissue on an internal surface of the patient (e.g. it may supply fluid to an intestinal wall or to the wall of an internal organ during surgery). One example of the tissue that the apparatus may be applied to is a wound. The embodiments described herein are described with reference to a wound. However, it will be appreciated that they are, in general, applicable to other tissue areas as described above.

The apparatus 100 includes a receptacle 10 that is positioned at a wound area 20, for example a chronic wound as may be found on a lower limb of a diabetic patient. The wound area 20 includes the wound 22 and an area of healthy skin 24 that surrounds the wound 22. The wound area 20 is also referred to as the tissue area throughout this specification. The apparatus further includes a cover 30 that is positionable over the receptacle to form a compartment 35 that is substantially bounded by the cover 30, the receptacle 10, and the wound area 20. In some embodiments of the apparatus described in this disclosure, the compartment may also be bounded by additional components of the apparatus that may be present and still be substantially bounded by the cover 30, the receptacle 10 and the wound area 20. For example, in some embodiments of the disclosure described herein, the compartment may be further bounded by one or more fluid inlet conduits and/or fluid outlet conduits as will be described herein.

The receptacle 10 consists of an inflatable, hollow bag that is fluidly connectable to a fluid source 40 for inflating the receptacle 10 with a fluid. The receptacle 10 may be connected to a fluid inlet conduit 50 for delivery of the fluid from the fluid source 40 to the receptacle 10. The connection of the fluid source 40 to the fluid inlet conduit 50 is shown schematically in the Figures and may not be shown directly. It will be appreciated that one or more additional conduits (not shown) may be included in the connection between the fluid inlet conduit 50 and the fluid source 40. The receptacle 10 may be for single use on a single patient. It may be used continuously at the wound for up to several days (e.g. 7 days or 10 days), and may be changed as and when a dressing, for example a bandage, is changed.

In each of the embodiments described in this disclosure, the fluid may comprise a gas. For example, the fluid may comprise oxygen gas, carbon dioxide gas, carbon monoxide gas, aerosols and gases at different humidity levels and/or other therapeutic gases that may be beneficial in treating the wound and/or assisting the wound to heal. The fluid may also comprise a liquid, for example it may comprise water, water with molecularly dispersed substances, or saline solution. The fluid source 40 may be a gas source such as a wall source, oxygen or carbon dioxide or carbon monoxide bottle, oxygen or carbon dioxide or carbon monoxide concentrator, oxygen or carbon dioxide or carbon monoxide pump or the like, suitable to meet a predetermined concentration and/or pressure and/or flow rate of oxygen or other therapeutic fluid as will be described further below. The fluid source 40 may comprise a flow and/or pressure regulator for controlling a flow rate and/or pressure of fluid issuing from the fluid source 40. The fluid source 40 is reusable, as is the flow and/or pressure regulator.

The receptacle 10, and each of the receptacles 110, 210, 210A, 310, 410 described in this disclosure, may be formed of one or more walls 15 of a flexible material. For example, it may be formed from a single wall or two or more walls attached together via heat sealing or otherwise. The receptacle 10 may contain oxygen gas, however other therapeutic fluids, for example carbon dioxide gas or carbon monoxide gas may also be used as described herein. The one or more walls 15 of the receptacle 10 has at least one section that is made from a material that is adapted to allow molecules within the fluid in the receptacle 10 to diffuse to outside of the receptacle 10. In some embodiments, the entire receptacle 10 may be formed of the material that allows molecules within the fluid in the receptacle to diffuse through it, whereas in other embodiments the receptacle 10 (other than the at least one section) may be made of a material of a different thickness to the material of the at least one section, or it may be made of a different material. The material section(s) may have a thin wall. The material may have a thickness of between approximately 10 micrometres and 150 micrometres, for example between approximately 20 micrometres and 140 micrometres, or between approximately 30 micrometres and 130 micrometres, or between approximately 35 micrometres and 100 micrometres, or between approximately 40 micrometres and 80 micrometres, or between approximately 40 micrometres to 70 micrometres, or between approximately 30 micrometres to 60 micrometres, or between approximately 20 micrometres to 60 micrometres, or between approximately 45 micrometres and 55 micrometres. In some embodiments, the wall thickness is approximately 60 micrometres. In some embodiments, the wall thickness is approximately 50 micrometres. The thinness of the material allows the wall 15 of the receptacle 10 to be flexible. This flexibility may help to maximise the surface contact between the receptacle 10 and the wound area, by allowing the wall of the receptacle 10 to conform to the wound surface, regardless of the wound topology. The material section(s) may be made of a stretchable material (that may deform elastically). The material section(s) may stretch in response to the amount of fluid in the receptacle. The stretchable material may help to maximise the surface contact between the receptacle 10 and the wound area, by allowing the wall of the receptacle 10 to conform to the wound surface, regardless of the wound topology.

As used in this specification, the terms ‘diffuse’ and ‘diffusion’ refer to the process of molecular diffusion. Molecular diffusion is a process by which molecules within a fluid can pass from an upstream side to a downstream side of a material (e.g. a polymer film). Molecular diffusion involves three stages: 1) sorption—the molecules within the fluid on the upstream side of the material are adsorbed onto the upstream surface of the material and then absorbed into the material; 2) diffusion—the molecules diffuse through the material. The direction of diffusion is dependent on a concentration gradient of the molecules within the material. The diffusion stage may be facilitated by the opening and closing of free-volume elements in the material; 3) desorption—the molecules may be desorbed from the downstream surface of the material into the fluid on the downstream side of the material. If the fluid into which the molecules desorb is a gas, then the molecules may be in a gas phase, or dispersed in the gas, after desorption. If the fluid into which the molecules desorb is a liquid, then the molecules may be in a liquid phase, or dispersed in the liquid, after desorption. The molecules will not remain in a liquid phase or a gas phase throughout the diffusion stage. Instead, during this stage, the molecules are considered to be molecularly dispersed in the material. The number of molecules that pass from the upstream side to the downstream side per unit time via molecular diffusion may be a function of, for example: the partial pressures on the upstream and downstream sides, the concentrations on the upstream and downstream sides, the thickness of the material, and the area of the material through which the molecular diffusion can occur.

As used in this specification, the term ‘pore’ refers to pores through which fluid molecules may move from an upstream side towards a downstream side of a material (e.g. a porous foam). The movement of molecules through pores, from an upstream side towards a downstream side of a material, is known as pore flow. For molecules to pass completely through a material via pore flow alone, there must be one or more pores that provide a continuous pathway from the upstream side to the downstream side. When molecules move from the upstream side to the downstream side of a material via pore flow, they may remain in a particular phase (e.g. liquid or gas) throughout.

The material may be a membrane that is adapted to allow molecules within the fluid to diffuse through the membrane. The membrane may be hydrophobic. Alternatively the material can be treated so that it repels water. In some embodiments, the membrane is substantially pore-free. Accordingly, in those embodiments the membrane material may have no visible discontinuities or pores that are visible with an optical or electron microscope, for example a scanning electron microscope such as a Jeol IT300, at a resolution on the order of 1 micrometre. Transport of fluid through the membrane does not substantially take place via porosity of the membrane. The diffusion transport mechanism provides a more even distribution of fluid molecules diffusing from the receptacle to the wound area when compared with materials having micropores or porous membranes. This is because, some of the pores may become blocked, by exudate for example, causing an uneven distribution of molecules across the membrane. The substantially pore-free membrane helps to prevent wound exudate from clogging or entering the receptacle 10. However, transport by porosity may occasionally take place, for example, due to the possibility of manufacturing defects occurring in the membrane. The membrane is substantially impermeable to bulk transport (also referred to herein as bulk flow) of fluid. That is, the molecules within the fluid cannot pass from one side of the membrane to the other side of the membrane without becoming molecularly diffused, and therefore cannot pass through the membrane in the fluid form in which they enter it e.g. gas or liquid form. The membrane being impermeable to bulk transport (or bulk flow) of fluid further helps to prevent exudate from entering the receptacle 10.

Suitable materials for the membrane include any silicone liquid rubber, such as any one of, or a combination including, a liquid silicone rubber having a 70 Shore A hardness elastomer (Silopren® LSR 4070 silicone from Momentive) or a 40 Shore A hardness elastomer (Silopren® LSR 4840 silicone from Momentive); pre-formed silicone sheet or film; silicone hydrogel; multilayer, blown, LLDPE/CaCO3 films, for example resin LLDP/CaCO3 from Reifenhauser (BF110, BF106); materials having silicone coatings; high consistency rubber silicone (HCR); KEG-2000-60-A/B (Shore A hardness of approximately 60) or KEG-2000-40-A/B (Shore A hardness of approximately 40) from Shin Etsu; and a copolymer that comprises polyethylene and poly(ethylene oxide).

In some embodiments, the membrane is made of a liquid silicone rubber having a 60 Shore A hardness that forms sheets having a thickness of 60 micrometres or of 50 micrometres. In some embodiments, the membrane is made of a liquid silicone rubber having a 40 Shore A hardness that forms sheets having a thickness of 60 micrometres or of 50 micrometres. These materials have been found to have suitable durability and suitable ability to hold their shape. The fluid inlet conduit 50 may also be made of this material. In this case, the fluid inlet conduit 50 may be integrally formed with the receptacle 10 or it may be connected to it via a seal. Alternatively, the fluid inlet conduit 50 may be made of soft, pliable material such as silicone tubing, for example Versilic® silicone tubing.

The gas supply to the receptacle 10, or any one of the other receptacles 110, 210, 210A, 310 or 410 described in this disclosure, may be continuous or it may be intermittent, i.e. supplied periodically. The gas may have a concentration of up to 100%, for example 99% or 95% or 90% and/or a pressure of between about 6 mmHg and 50 mmHg above atmospheric pressure, for example between about 10 mmHg to 40 mmHg or about 20 mmHg to 30 mmHg.

The receptacle 10, or any of the receptacles 110, 210, 210A, 310 or 410 described in this disclosure, may come in various sizes according to the size of the wound. When inflated it may have any reasonable shape, for example a spheroid, ovoid, cuboid or cylindrical shape. The one or more walls include a tissue facing portion that faces the tissue of the patient in use. As seen in FIG. 1, the receptacle 10 has a flattened ovoid cross-sectional shape, such that the tissue facing portion of the one or more walls provides a substantial tissue-facing surface, which in this example is a wound-facing surface 12 for contacting the wound when the receptacle is at least partially inflated. The wound facing surface 12 of the receptacle 10 is made of the membrane adapted to allow molecules to diffuse from the receptacle to the wound 22. The wound facing surface 12 of the receptacle 10 is pressed against the wound surface and forces exudate to move outwards to the periphery of the wound 22. This displacement of exudate from the wound is due to the pressure that the receptacle 10 applies to the wound 22. The wound 22 ‘experiences’ the highest pressure from the receptacle 10 in the radial centre and the lowest pressure from the receptacle 10 at the radial extreme due to the shape of the receptacle 10. This pressure gradient can force exudate to move outwards. Forcing the exudate to move outwards may help to prevent or mitigate pooling of exudate between the wound facing surface 12 of the receptacle 10 and the wound 22. Preventing or mitigating this pooling may be beneficial as it may help to prevent exudate from impeding the movement of molecules from the inside of the receptacle 10 to the wound 22. Whilst exudate is forced outwards from the wound 22, the wound 22 may remain moist. Maintaining a moist environment within and around a wound can be important as a dry environment within and around the wound may be detrimental to the healing process. The receptacle configuration of FIG. 1 also provides a substantially even distribution of fluid to the wound 22 as a result of the substantial area of the wound facing surface 12. The diffusion of the fluid molecules through the membrane adapted to allow molecules to diffuse from the receptacle 10 to the wound 22 further provides for a substantially even concentration of fluid being applied across the wound 22.

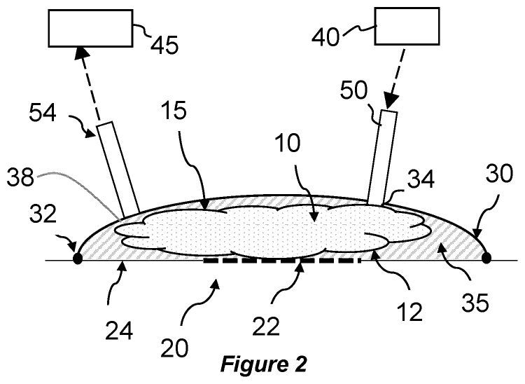

In the embodiment of FIG. 2, the receptacle 10 is shown in use, at least partially inflated, and has an undulating surface when at least partially inflated that may conform particularly well to complex, three-dimensional wound surfaces such that a larger proportion of the wound 22 is contacted by the wound-facing surface 12 of the membrane. The undulating surface may also help facilitate exudate flow away from the wound 22. This embodiment is described with reference to a wound. However, it will be appreciated that it is, in general, applicable to other tissue areas as described elsewhere in this disclosure.

In some embodiments, the receptacle 10, or any one of the receptacles 110, 210, 210A, 310 or 410 described in this disclosure, may function as a fluid reservoir in which a volume of fluid may be stored. This can be beneficial if the fluid supply to the receptacle 10 is interrupted, due, for example, to a disconnection of the fluid source 40 from the fluid inlet conduit 50 or a malfunction of the fluid source 40. In some embodiments, the receptacle 10 is filled with fluid to ensure a fluid supply to the wound 22 in the event of disruption of the fluid supply from the fluid source 40. The filling of the receptacle 10 may be carried out by a clinician prior to positioning the receptacle 10 at the wound area 20 of the patient or during use of the apparatus. Alternatively, the receptacles 10 may be pre-filled with the fluid during manufacture of the apparatus, for example at the site of manufacture.

As shown in FIG. 1, embodiments of the receptacle 10 include a plurality of structures, for example microstructures 60, arranged on the wound facing surface 12 of the receptacle 10. Whilst the microstructures 60 are shown on only a portion of the receptacle 10, they may cover one or more other portions of the one or more walls 15 of the receptacle 10, or of any of the receptacles 110, 210, 210A, 310, 410 described in this disclosure. In an embodiment, the microstructures may be present on each of the one or more walls 15 of the receptacle 10. A benefit of the receptacle 10 having microstructures 60 that may cover one or more other portions of the one or more walls 15 of the receptacle 10 is that the receptacle 10 (or any of the receptacles 110, 210, 210A, 310, 410) can be applied to the wound 22 in any orientation and the microstructures will contact the wound 22.

Whilst FIG. 1 shows a schematic representation of the microstructures 60, FIG. 1A shows a view of the receptacle 10 having a plurality of microstructures 60 on a wall 15 of the receptacle. The microstructures 60 are part of the receptacle 10 and whilst the microstructures are shown arranged on the receptacle 10 of the embodiment of FIG. 1, they may be included in any of the embodiments of the receptacle(s) described and illustrated in this disclosure. The microstructures are structures of microscale dimensions that are configured to contact the wound area 20 of the patient. The microstructures 60 may be made of the same material as the membrane adapted to allow molecules to diffuse through it and from the receptacle 10 to the wound 22. The microstructures 60 are positioned on and protrude from the wound facing surface 12. The microstructures may exert forces on the wound. These forces may result in micro-stresses that stretch the underlying cells and cause them to take on the same signalling pathways as those affected by growth factors. This can encourage cell growth. The molecules within the fluid in the receptacle 10 may diffuse through the membrane of the receptacle 10 and into the wound 22, and/or through the membrane of the receptacle, through the microstructures 60, and into the wound 22.

The microstructures 60 may be of a semi-spherical, spherical, pyramidal, conical, domed or frustoconical shape, or have a trapezoidal, semi-circular or parabolic shape in profile, or may be formed as small dimples in the surface of the membrane. Any corners or edges of the microstructures 60 may be rounded as sharp edges can cause damage to cells. The microstructures 60 may be formed as elongate structures having e.g. a semi-circular, parabolic, domed or trapezoidal cross-sectional profile as described above, or they may comprise discrete structures, each having e.g. a pyramidal or semi-spherical or parabolic, domed or conical or frustoconical shape. In some embodiments the microstructures 60 are configured in arrays. The microstructures 60 may have a base dimension, e.g. a width or a diameter of between approximately 100 to 400 micrometres, for example between approximately 250 to 350 micrometres, or between approximately 280 to 330 micrometres. The microstructures 60 may be spaced from one another at a distance of between about 0.5 mm to 3 mm between the bases of adjacent microstructures 60. The microstructures 60 may have a height dimension of between approximately 100 to 200 micrometres, for example between approximately 125 to 175 micrometres. In some embodiments, the microstructures may have a base dimension of approximately 310 micrometres and a height dimension of approximately 150 micrometres. The cells typically have a height dimension of between approximately 7 micrometres and 15 micrometres, for example 10 micrometres. It will be appreciated by the skilled person that the microstructures 60 illustrated in FIG. 1 are not to scale.

Whilst the features of the microstructures 60 are described with reference to the receptacle 10, it will be apparent that one or more of the features may also be applied to any of the other receptacles 110, 210, 210A, 310 or 410 described in this disclosure.

The cover 30 is configured to at least partially enclose the receptacle 10 to form a compartment 35 substantially bounded by the cover 30, the receptacle 10 and the wound area 20. In some embodiments, for example as shown in FIG. 1, the cover 30 is positionable over the receptacle 10 to fully enclose the receptacle 10 to form the compartment 35. The cover 30 may also be applied to any of the receptacles 110, 210, 210A, 310 or 410 described in this disclosure.

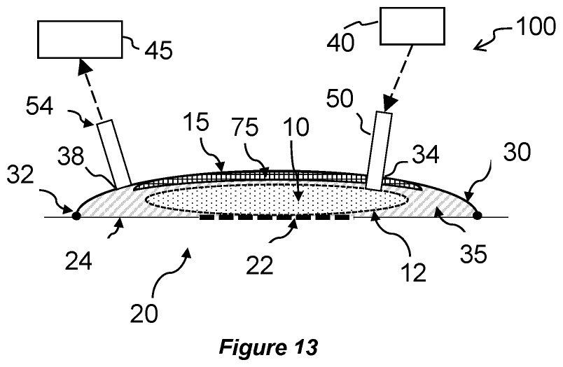

In some embodiments, the cover 30 consists of a wall or walls configured to enclose the receptacle 10 and to seal to the healthy skin 24 adjacent the wound 22. A seal 32 for this purpose may comprise an adhesive in the form of an adhesive bead or strip at a sealing surface of the cover 30 or it may be a suction seal. The cover 30 may include a layer of absorbent material 75, seen in FIG. 13, such that any exudate from the wound that reaches the cover is absorbed into the layer of absorbent material 75. The absorbent material 75 may be a fibrous fabric. The absorbent material 75 may form part of the cover 30, or it may be provided as a separate layer for use with the cover 30. For example, the absorbent material 75 may be wrapped around a limb or other body part of the patient that has the wound. The cover 30 may be placed over the absorbent material 75. The absorbent material 75 forms part of the cover 30 such that the compartment substantially bounded by the cover 30, the receptacle 10 and the wound area 20 includes the absorbent material 75. The absorbent material 75 may allow exudate to evaporate to the ambient environment. Alternatively, the cover 30 may be impermeable to the bulk flow of fluid. This arrangement is in contrast to some known solutions in which the absorbent material 75 is provided directly over the wound for collecting exudate. In these known solutions, the exudate may saturate and ‘clog’ the absorbent material 75. If this occurs, it may impede movement of molecules from the inside of the receptacle to the wound surface, or lead to inconsistent and/or unevenly distributed application of the molecules to the wound surface. In addition to this, there is also a chance that pieces of the absorbent material 75 may end up in the wound (which may increase the chance of infection). Alternatively, as the wound heals, tissue can grow into the voids of the absorbent (e.g. foam) material. This may make it painful to remove the absorbent material. An advantage of the absorbent material 75 forming part of the cover 30 or being provided for use with the cover 30 is that it avoids these issues, as it allows for absorption of the exudate into the absorbent material 75 away from the wound 22 and away from the receptacle 10. Even if exudate saturates or clogs the absorbent material 75, the movement of molecules from the inside of the receptacle 10 to the wound 22 will not be impeded by the absorbent material.

The cover 30 may be separate to the receptacle 10 or it may be combined with (i.e. connected to) the receptacle 10 to form a single dressing that can be placed at the wound area 20.

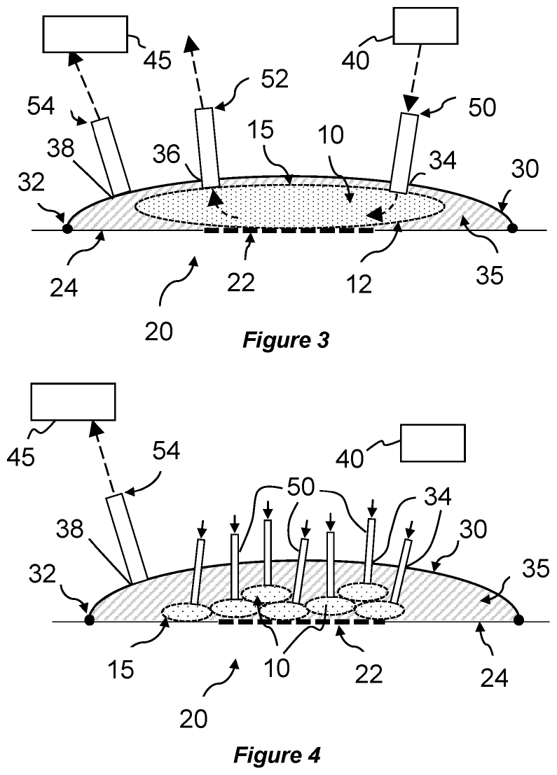

With reference to the embodiment of FIG. 3, the cover 30 includes a first opening 34 through which the fluid inlet conduit 50 passes to enter the compartment 35 to provide fluid to the receptacle 10. The cover 30 further includes a second opening 36 via which fluid in the receptacle 10 may exit the receptacle 10. The second opening 36 receives a first fluid outlet conduit 52. The first fluid outlet conduit 52 is integral with or connected to the receptacle 10 and passes through the second opening 36. The cover 30 further includes a third opening 38 through which fluid in the compartment 35 may exit the compartment 35. A second fluid outlet conduit 54 is connected at the third opening 38 of the cover 30 for transporting fluid out of the compartment 35. The first and second fluid outlet conduits 52, 54 may be made from soft, pliable material such as silicone tubing, for example Versilic® silicone tubing. The fluid inlet conduit 50 and the fluid outlet conduits 52, 54 may be for single use on a single patient and may be changed after up to several days (e.g. 7 days or 10 days) continuous use on a patient. In this embodiment, as described elsewhere in this disclosure, the compartment 35 substantially bounded by the cover 30, the receptacle 10 and the wound area 20 is further bounded by the fluid inlet conduit 50 and the first fluid outlet conduit 52.

In this embodiment, the at least one section of the wall 15 of the receptacle 10 may allow molecules within the fluid to diffuse from the receptacle 10 to the wound 22, or the fluid to pass from the receptacle 10 to the wound 22 via pore flow, or alternatively it may allow molecules within the fluid to both diffuse from the receptacle 10 to the wound 22 and also to allow the fluid to pass from the receptacle 10 to the wound 22 via pore flow. The receptacle 10 may include a plurality of the microstructures 60 arranged on the wound facing surface 12 of the receptacle 10. The fluid may comprise a gas, such as oxygen gas or carbon dioxide gas or carbon monoxide gas.

The provision of the fluid inlet conduit 50 and the first fluid outlet conduit 52 in fluid communication with the receptacle 10 allows for circulation of fluid through the receptacle 10. Fluid enters the receptacle 10 via the fluid inlet conduit 50. Fluid exits the receptacle 10 via the first fluid outlet conduit 52. Fresh fluid can be cycled into and out of the receptacle 10 as shown schematically in FIG. 3, either constantly or periodically. As the fluid is cycled out of the receptacle 10, it is flushed out, for example to the atmosphere, and not returned to the receptacle 10. This applies to all instances of fluid cycling through the receptacle 10 disclosed in this specification. The cycling enables a fluid concentration, for example an oxygen gas concentration, within the receptacle 10 to be maintained at a desired level, even if nitrogen and/or other fluid molecules enter the receptacle 10 through the membrane of the receptacle 10. The cycling also enables the fluid, from which molecules diffuse through the wall 15 of the receptacle 10, or which passes through the wall 15 via pore flow, to be replenished. The fluid concentration may be precisely controlled via a controller, for example a controller of the fluid source 40. Additionally, other fluids, including drugs could be cycled through the receptacle 10 and applied to the wound 22 via diffusion through the wound facing surface 12. As will be appreciated, the cycling may be applied to any one of the receptacles 10, 110, 210, 210A, 310 or 410 described in this disclosure that have a fluid inlet conduit 50 and a first fluid outlet conduit 52.

The apparatus may include a negative pressure source 45 for drawing fluid out of the compartment 35. Negative pressure refers to a pressure below that of the ambient atmosphere around the patient. The pressure in the compartment 35 may be altered to be between approximately 50 mmHg to 150 mmHg or between approximately 80 mmHg to 125 mmHg or between 90 mmHg to 110 mmHg below atmospheric pressure. The negative pressure source may be a pump. The second fluid outlet conduit 54 may connect the compartment 35 to the negative pressure source 45 at the third opening 38 of the cover 30. The connection of the negative pressure source 45 to the second fluid outlet conduit 54 is shown schematically in the Figures and may not be shown directly. It will be appreciated that one or more additional conduits (not shown) may be included in the connection between the second fluid outlet conduit 54 and the negative pressure source 45.

In use, the receptacle 10 is positioned on the wound 22 and may be held in place by the cover 30 operating as a negative pressure dressing. The cover 30 seals to the healthy skin 24 of the patient surrounding the wound 22 and, as the negative pressure source 45 draws fluid from the compartment 35, forms a negative pressure compartment enclosing the receptacle 10. The fluid inlet conduit 50 and/or the second fluid outlet conduit 54 may extend through the cover 30 or under an outer edge of the cover 30.

As shown in the embodiment of FIG. 3, fluid may be cycled through the receptacle 10 at the same time as a negative pressure is applied to the compartment 35. The negative pressure is applied to the compartment 35 by the negative pressure source 45, via the second fluid conduit 54 connected to the cover 30 at the third opening 38. The fluid in the receptacle 10 may diffuse through the membrane or pass through it via pore flow at the wound facing surface 12 of the receptacle 10 as the negative pressure is applied to the compartment 35.

This embodiment is described with reference to a wound. However, it will be appreciated that it is, in general, applicable to other tissue areas as described elsewhere in this disclosure.

In the embodiments of FIGS. 1 and 2, the cover 30 includes only the first opening 34 and the third opening 38. In these embodiments, fluid is supplied into the receptacle 10 via the fluid inlet conduit 50 and exits the receptacle only via diffusion through the membrane adapted to allow molecules within the fluid to diffuse from the wound facing surface 12 of the receptacle 10.

Simultaneously, a negative pressure may be applied to the compartment 35 via the second fluid outlet conduit 54 connected to the cover 30 at the third opening 38. In these embodiments, the compartment 35 substantially bounded by the cover 30, the receptacle 10 and the tissue area is further bounded by the fluid inlet conduit 50. This arrangement of the apparatus 100 is advantageous over prior solutions that allow a therapeutic fluid (e.g. oxygen) and negative pressure to be applied to a wound simultaneously. Many of these existing solutions rely on an arrangement that, when sealed to the heathy skin surrounding a wound, defines a single compartment (a compartment bounded by the inside of the bandage and the wound area). Oxygen is pumped into the compartment while the fluid within the compartment is removed (by a negative pressure pump) to generate the negative pressure environment. One issue with these existing, single-compartment arrangements is that the negative pressure tends to draw oxygen out of the wound environment before it has a chance to contact the wound. Furthermore, as the negative pressure draws exudate from the wound and into the compartment, this exudate may impede the movement of molecules from the inside of the receptacle to the wound surface. The dual compartment arrangement of the apparatus 100 allows negative pressure to be applied to the wound 22 and exudate to be removed into the compartment 35 and through the second fluid outlet conduit 54 without significantly affecting the movement of molecules from the receptacle 10, through the membrane, to the surface of the wound 22. The receptacle 10 of this embodiment may include a plurality of the microstructures 60 arranged on the wound facing surface 12 of the receptacle 10.

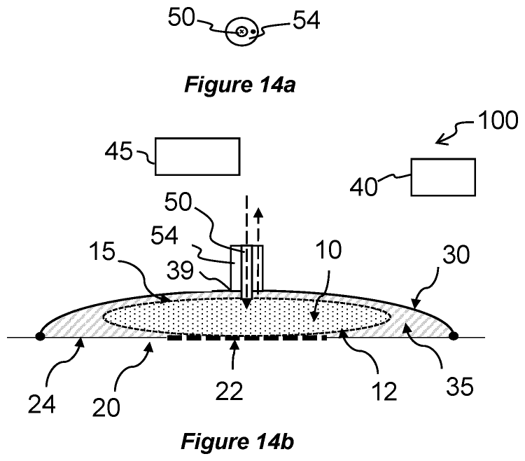

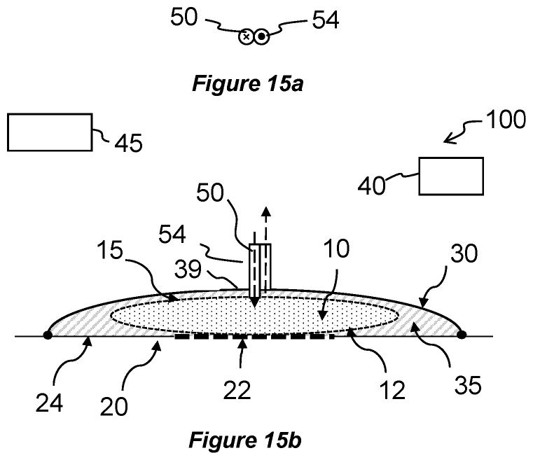

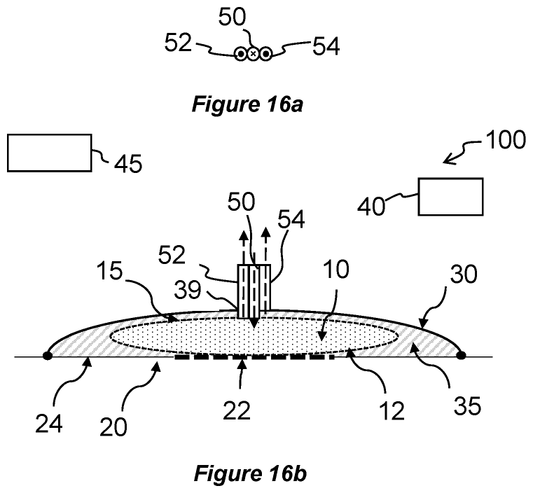

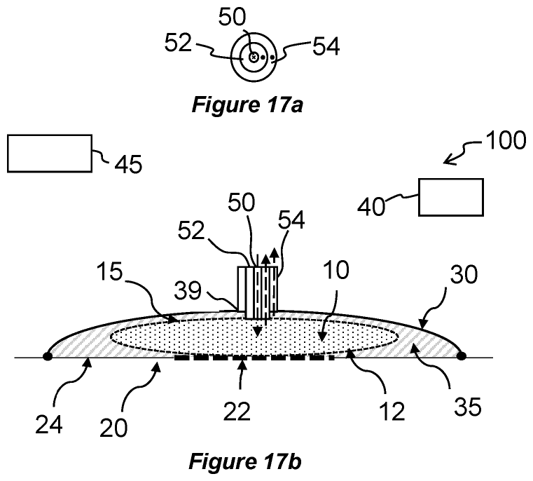

In some embodiments, shown in FIGS. 14 to 17, two or more of the fluid inlet conduit 50, the first fluid outlet conduit 52 and the second fluid outlet conduit 54 may be combined into a single conduit that passes through only a single opening 39 in the cover 30. For example, in FIG. 14a and FIG. 14b, the fluid inlet conduit 50 and the second fluid outlet conduit 54 may be arranged coaxially. FIG. 14a shows the coaxial arrangement of the fluid inlet conduit 50 and the second fluid outlet conduit 54 in plan view. FIG. 14b shows the coaxial arrangement of the conduits 50, 54 in use of the apparatus 100. FIG. 15a and FIG. 15b show another arrangement in which the fluid inlet conduit 50 and the second fluid outlet conduit 54 may be arranged adjacent one another so as to pass through the single opening 39 in the cover 30. In the arrangement of FIG. 16a and FIG. 16b, the apparatus includes the fluid inlet conduit 50, the first fluid outlet conduit 52 and the second fluid outlet conduit 54, arranged adjacent one another as a combined conduit that passes through the single opening 39. In FIGS. 17a and 17b, the fluid inlet conduit 50, first fluid outlet conduit 52 and second fluid outlet conduit 54 are arranged coaxially. These arrangements may improve the seal between the apparatus 100 and the skin of the patient or other tissue surface, due to multiple conduits passing through a single opening in the cover rather than through multiple openings. This is because multiple openings may compromise the seal in comparison with a single opening. Whilst a single opening may compromise the seal to some extent, multiple openings will compromise the seal to a greater extent. The single opening 39 provides a larger cover surface area for sealing against the tissue of the patient than would be the case with the first, second and third openings 34, 36, 38.

In the embodiments of FIGS. 1 to 6 and also FIG. 8 and FIGS. 12 to 17, the cover 30 and the receptacle 10 do not share any common walls; they are separate components and the walls of the receptacle 10 and the cover 30 are independent of one another. The cover 30 may be connected to the receptacle 10 via the fluid inlet conduit 50, at the first opening 34. The cover 30 may also be connected to the receptacle 10 via the first fluid outlet conduit 52 at the second opening 36. These one or two connection points may be the only points of connection between the receptacle 10 and the cover 30. This minimal connection between the receptacle 10 and the cover 30 enables the receptacle 10 to be movable within the compartment 35 while also allowing exudate to move out of the wound 22, which may help the receptacle 10 to conform to wounds having a complex geometry and/or topology. Alternatively, the fluid inlet conduit 50, first fluid outlet conduit 52 and/or the second fluid outlet conduit 54 may not be connected to the cover 30 but may protrude from a gap in the cover 30. In each of these embodiments, the receptacle(s) 10 may include a plurality of the microstructures 60 arranged on the wound facing surface 12 of the receptacle(s) 10.

In some embodiments of the apparatus 100, the cover 30 may be formed from wrapping a dressing, for example a compression bandage around a body part, for example a limb, of the patient and over the receptacle 10. The compression bandage holds the receptacle 10 in place on the wound 22 and can absorb exudate and/or allow evaporation of exudate. The compression bandage material allows the bulk transport of gases, however it will be appreciated that it may be wrapped about the patient forming enough layers that it provides a substantial barrier to the bulk flow of gases. The fluid, for example oxygen, is supplied to the receptacle 10 at a positive pressure for effective oxygen delivery, even when used under a compression bandage that may compress the receptacle 10 against the wound 22. As the receptacle 10 is impermeable to the bulk flow of fluid, a positive pressure can be maintained in the receptacle 10 even as it is positioned within a negative pressure compartment. This means that the apparatus 100 can be used to simultaneously deliver topical oxygen therapy and negative pressure therapy. The fluid inlet conduit 50, first fluid outlet conduit 52 and/or the second fluid outlet conduit 54 may not be connected to the compression bandage but may protrude from a gap in the wrapped layers. In a variation of this embodiment, the dressing may be an adhesive dressing that adheres to the healthy skin 24 of the patient over the receptacle 10. This embodiment may or may not utilise the negative pressure source 45 to form a negative pressure compartment. Whilst this embodiment is described with reference to a wound, it will be appreciated that it is, in general, applicable to other tissue areas as described elsewhere in this disclosure. In this embodiment, the compartment 35 substantially bounded by the cover 30, the receptacle 10 and the tissue area is further bounded by one or more of the fluid inlet conduit 50 and the first fluid outlet conduit 52. Whilst this embodiment of the cover 30 is described with reference to the receptacle 10, it will be appreciated that it may be applicable to any of the other receptacles 110, 210, 210A, 310 or 410 described in this disclosure.

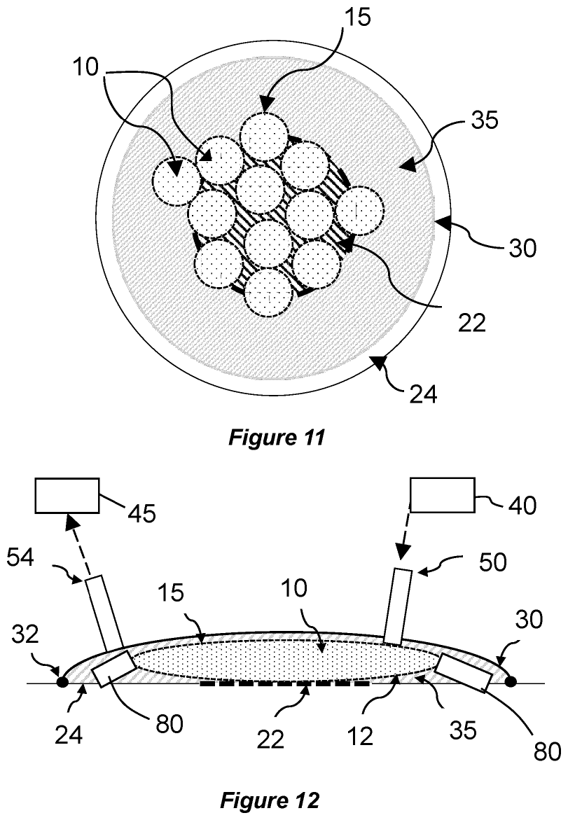

FIG. 4 shows a further embodiment of the apparatus 100 that includes a plurality of the receptacles 10 positioned at the wound area 20. The plurality of receptacles 10 may be distributed over the wound 22 as is also shown schematically in the plan view of FIG. 11. In the embodiments of FIG. 4 and FIG. 11, the at least one section of the wall 15 of each of the plurality of receptacles 10 may allow molecules within the fluid to diffuse from the plurality of receptacles 10 (also referred to herein as multiple receptacles 10) to the wound 22, or to pass from the multiple receptacles 10 to the wound 22 via pore flow, or to allow molecules within the fluid to diffuse from the multiple receptacles 10 to the wound 22 and also to pass from the multiple receptacles 10 to the wound 22 via pore flow. The fluid may comprise a gas, such as oxygen gas or carbon dioxide gas or carbon monoxide gas. The plurality of receptacles 10 may equally be a plurality of any of the receptacles 110, 210, 210A, 310 or 410 described in this disclosure.

Multiple receptacles 10 can provide additional conformability to the wound for complex and/or varying wound topologies. For example, if a wound is small or deep, multiple receptacles 10 may be more easily manipulated to optimise delivery of therapeutic fluid and/or negative pressure therapy. Accordingly, if a wound is deep and/or has an irregular surface, it may be possible to achieve greater surface area contact between the section(s) of the wall(s) 15 through which the molecules can diffuse or pass via pore flow and the wound by using multiple receptacles 10, relative to the contact that could be achieved using one receptacle 10. A large amount of surface area contact can be beneficial as it can result in a more even distribution of therapeutic molecules to the wound. Multiple receptacles 10 can also be used to treat a large wound.

Multiple receptacles 10 may also allow for more localised wound healing. For example, if some parts of a wound heal faster than others, one or more of the receptacles 10 can be positioned to promote healing of a wound region that is less healed rather than remaining at the more healed parts. Each receptacle 10 is moveable within the compartment 35 independently of any of the other receptacles 10. Each receptacle 10 may include a plurality of the microstructures 60 arranged on the wound facing surface 12 of the receptacle 10.

As shown in FIG. 4, each of the receptacles 10 has its own fluid inlet conduit 50 associated with it. In the embodiment of FIG. 4, each of the fluid inlet conduits passes through a separate respective first opening 34 in the cover 30, for connection to a respective separate fluid supply or fluid source. This arrangement allows different fluids to be provided in different receptacles for variable, customisable control of fluid molecule delivery over the wound area 20. It will be appreciated that any reasonable number of multiple receptacles 10 and fluid inlet conduits 50 may be positioned at the wound area 20 and that the number of receptacles is not limited to the number shown in FIG. 4. For example, the number of receptacles 10 may depend on the surface area of the wound 22. Examples include from two, three, four and all numbers of receptacles that may be required to treat the tissue area concerned. Whilst this embodiment is described with reference to a wound, it will be appreciated that it is, in general, applicable to other tissue areas as described elsewhere in this disclosure. In this embodiment, the compartment 35 substantially bounded by the cover 30, the receptacle 10 and the tissue area is further bounded by the each of the fluid inlet conduits 50.

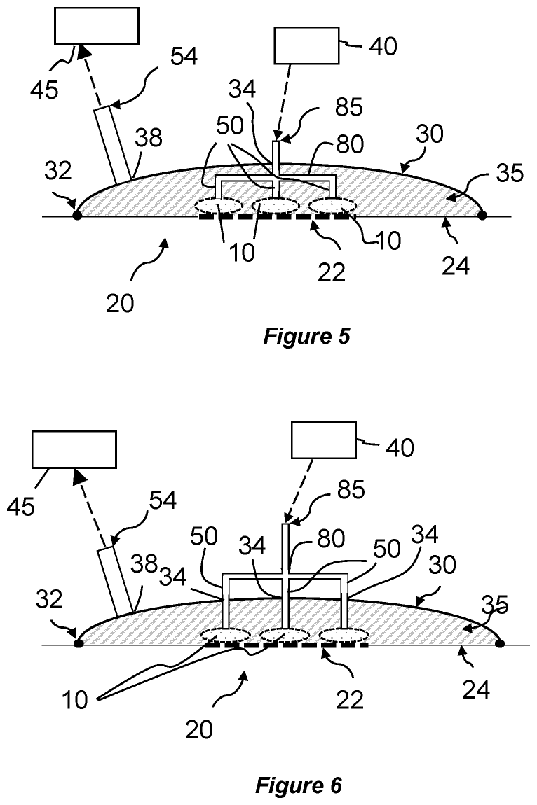

In the embodiments of FIG. 5 and FIG. 6, the fluid inlet conduits 50 are connected together in fluid communication with one another via a manifold 80, the manifold being supplied with fluid via a single fluid supply conduit 85. The manifold 80 joins together the three fluid inlet conduits 50 shown in FIG. 5 inside the compartment 35. The manifold 80 joins together the three fluid inlet conduits 50 shown in FIG. 6 outside the compartment 35. The FIG. 5 arrangement may improve sealing of the cover 30 against the skin when compared with the embodiment of FIG. 6, as it requires fewer openings in the cover 30. It will be appreciated that any reasonable number of multiple receptacles 10 and fluid inlet conduits 50 may be joined together at the manifold 80 and that the number of receptacles is not limited to the number shown in FIG. 5 and FIG. 6. For example, the number of receptacles 10 may depend on the surface area of the wound 22. Examples include from two, three, four and all numbers of receptacles that may be required to treat the tissue area concerned. The single fluid supply conduit 85 passes through the first opening 34 in the cover 30 for receiving fluid therein from a single fluid source 40. The manifold 80 may include a valve (not shown) on each fluid inlet conduit 50 for individual control of fluid flow through the respective fluid inlet conduits 50. Although not shown in FIG. 5, respective first fluid outlet conduits 52 may be used to cycle fluid through the fluid receptacles 10 and to exit the receptacles 10 via the respective first fluid outlet conduits 52. In these embodiments, the compartment 35 substantially bounded by the cover 30, the receptacles 10 and the tissue area is further bounded by the fluid inlet conduits 50, the manifold 80 and the fluid supply conduit 85, and the respective fluid outlet conduits 52 if present.

In the embodiment of FIG. 6, the manifold 80 joins together the three fluid inlet conduits 50 outside of the compartment 35. In these embodiments, the compartment 35 substantially bounded by the cover 30, the receptacles 10 and the tissue area is further bounded by the fluid inlet conduits 50. It will be appreciated that any reasonable number of receptacles 10 and fluid inlet conduits 50 may be joined together at the manifold 80. The three fluid inlet conduits 50 pass through respective first openings 34 in the cover 30 for receiving fluid therein from a single fluid source 40. The manifold 80 may include a valve (not shown) on each fluid inlet conduit 50 for individual control of fluid flow through the respective fluid inlet conduits 50. Although not shown in FIG. 6, respective first fluid outlet conduits 52 may be used to cycle fluid through the receptacles 10. The fluid may enter the receptacles 10 via the fluid supply conduit 85 and the respective fluid inlet conduits 50 and exit the receptacles 10 via the respective first fluid outlet conduits 52.

It is noted that FIGS. 5 and 6 are schematic illustrations of the manifold 80, fluid inlet conduits 50 and fluid supply conduit 85. The manifold 80, fluid inlet conduits 50 and fluid supply conduit 85 may be made from soft, pliable material such as silicone tubing, for example Versilic® silicone tubing. They may be formed integrally with the receptacles 10, so that they are conformable and comfortable against the wound 22. This embodiment is described with reference to a wound. However, it will be appreciated that it is, in general, applicable to other tissue areas as described elsewhere in this disclosure.

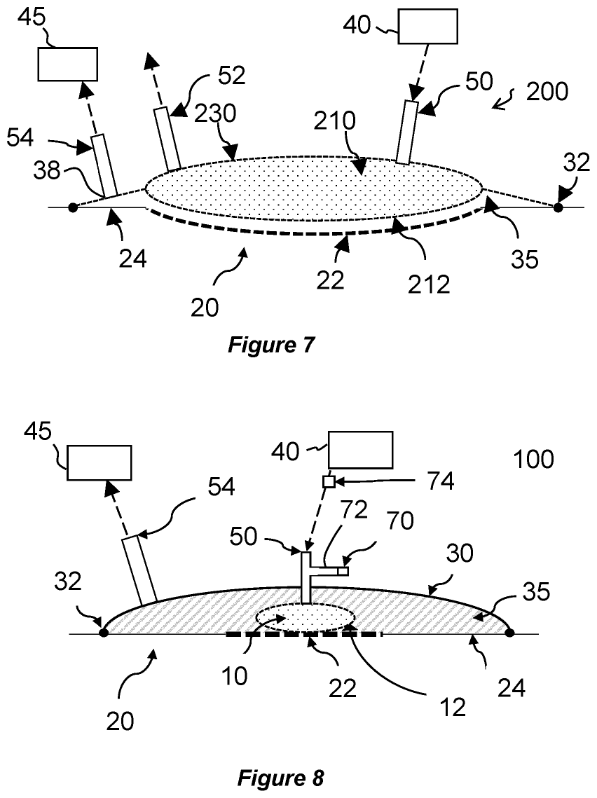

FIG. 7 shows an apparatus 200 in which the receptacle 210 is not enclosed within the negative pressure compartment 35. The receptacle 210 may be formed from two sheets of material, for example a wound facing sheet 212 and a cover sheet 230 that are pressed and sealed together at the edges of the receptacle 210. The excess pressed material around the receptacle 210 provides the functionality of the cover 30 of previous embodiments and includes the seal 32 for sealing to the healthy skin 24 of the patient around the wound 22 or other tissue area. The two sheets of material 212, 230 that are pressed and sealed together may be made of the same material. For example, the wound facing sheet 212 and the cover sheet 230 may both be made from the membrane that allows molecules to pass through it. Alternatively, the two sheets that are pressed and sealed together may be made of different material. For example, the wound facing sheet 212 may be made from the membrane that allows molecules to pass through it. The membrane may allow molecules within the fluid to diffuse from the receptacle 210 to the wound 22 or to pass through the membrane via pore flow, or it may allow molecules within the fluid to both diffuse from the receptacle 210 to the wound 22 and to pass from the receptacle 210 to the wound 22 via pore flow. The cover sheet 230 may not allow molecules within the fluid to pass through it. Alternatively, the cover sheet 230 may allow molecules within the fluid to pass through via diffusion or via pore flow, or it may allow molecules within the fluid to both diffuse from the receptacle 210 to the wound 22 and to pass from the receptacle 210 to the wound 22 via pore flow. If the cover sheet 230 is made from the membrane that allows molecules to pass through, then molecules may be able to move from the compartment 35 to the outside of the cover sheet 230. The apparatus 200 may further comprise a negative pressure compartment 35 substantially bounded by the cover sheet 230, the wound facing sheet 212 of the receptacle 10 and the wound area 20. In this construction, the negative pressure compartment 35 is substantially bounded by the cover 230, the wound facing surface 212 of the receptacle 210 and the wound area 20. In this embodiment, the fluid may enter the receptacle 210 via the fluid inlet conduit 50 and, although not shown in FIG. 7, it may also be cycled through the receptacle 210 and exit the receptacle 210 via the first fluid outlet 52. The negative pressure compartment 35 is formed by connecting the third opening 38 in the cover sheet 230 to the negative pressure source 45. Alternatively, a cover 30 could be added, for example glued, onto the receptacle 210, over the cover sheet 230, after the receptacle has been formed. The addition of the cover 30 allows a negative pressure compartment 35 to be formed as described in relation to the embodiment of FIG. 1 above. The addition of the cover 30 may also allow the wound facing sheet 212 and the cover sheet 230 of the receptacle 210 to be made from the same material, and for a cover 30 made from material that is impermeable to bulk flow of fluid to be glued or otherwise added over the top of the receptacle 210. The cover 30 may have all of the features of the cover 30 described earlier in this disclosure. For example, it may include an absorbent layer such as the absorbent material 75 described in FIG. 13. Alternatively, this embodiment may be used without a negative pressure compartment, in which case the first fluid outlet conduit 54 and the negative pressure source 45 need not be included. The receptacle 210 may include a plurality of the microstructures 60 arranged on the wound facing sheet 212 of the receptacle 210. The fluid may comprise a gas, such as oxygen gas or carbon dioxide gas or carbon monoxide gas. The apparatus 200 may require less material to manufacture than the apparatus 100.

If a fluid source 40 (e.g. a pump) or its associated controller malfunctions, the pressure within the associated oxygen compartment(s) may increase to an undesirable level. This could cause the receptacle(s) 10, 110, 210, or any of the other receptacles 210A, 310, 410 described in this disclosure, to rupture. In some embodiments, the apparatus 100 includes a pressure regulator, for example a pressure relief valve 70, that can be configured to open to relieve pressure if the pressure within the receptacle(s) 10 exceeds a threshold value. As seen in FIG. 8, the pressure relief valve 70 may be connected to the fluid inlet conduit 50 either at a wall of the fluid inlet conduit 50 or at a wall of a branch line 72 extending from and in fluid communication with the fluid inlet conduit 50. The pressure relief valve 70 may be positioned outside of the compartment 35 formed by the cover 30 as shown in FIG. 8, or inside the compartment 35 formed by the cover 30 (not shown). In the latter embodiment, the cover 30 may include a vent to atmosphere, for example an outlet port, a negative pressure port or may be a permeable cover 30 such as a compression bandage, such that gas released from the pressure relief valve 70 on the fluid inlet conduit 50 inlet may be vented from underneath the cover 30 to prevent the compartment 35 formed by the cover 30 from bursting.

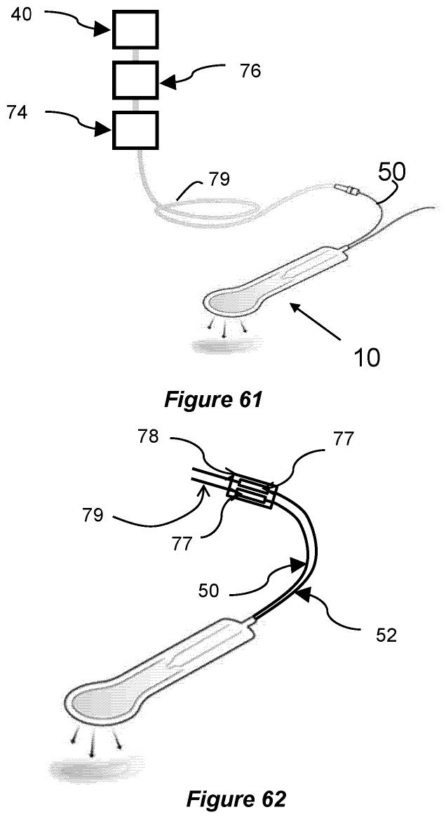

When open, the pressure relief valve 70 may exhaust fluid to the ambient environment (if positioned outside of the cover 30), or exhaust fluid into the compartment formed by the cover 30 (if positioned inside of the compartment 35). In this embodiment, the compartment 35 substantially bounded by the cover 30, the receptacle 10 and the tissue area is further bounded by the fluid inlet conduit 50, branch line 72 if present and the pressure relief valve 70, if present inside the cover 30. As shown in FIG. 8 and also in FIG. 61, a calibrated leak orifice 74 may be included upstream of the fluid inlet conduit 50 and downstream of the fluid source 40. A calibrated leak orifice is a mechanical device that may be calibrated to produce a specific flow rate in response to a certain pressure. The receptacle 10 of the embodiment of FIG. 8 is supplied with fluid from a fluid source 40 configured to supply fluid into the fluid inlet conduit 50. The calibrated leak orifice 74 may be used in addition to or in place of the fluid source regulator 76 (shown in FIG. 61) to control the flow rate of fluid passing from the fluid source 40 and into the fluid inlet conduit 50. The calibrated leak orifice 74 may be reusable or it may be for single use on a single patient.

In an alternative embodiment, the pressure relief valve 70 or other pressure regulator is not present at the fluid inlet conduit 50, however a calibrated leak orifice may be disposed on the fluid inlet conduit 50, between the fluid source 40 and the receptacle 10.

Any of the pressure relief valve 170 or other pressure regulator or calibrated leak orifice 74 may be present at any of the receptacles 10, 110, 210, 210A, 310 or 410 described in this disclosure.

As shown in FIG. 62, the fluid inlet conduit 50 is connected to a fluid source conduit 79 with a conduit connector 77. The first fluid outlet conduit 52 may also be connected to the pressure relief valve 70 with a further conduit connector (not seen in FIG. 62). The conduit connectors 77 may be any type of appropriate connector, for example barb connectors, double-ended barb connectors, luer-type connectors, or other connectors that click into place. Each conduit connector 77 may be held by a holder 78. A holder 78 may hold multiple conduit connectors 77 or each connector 77 may be held in a separate holder 78. Each holder 78 may be attachable to the limb, torso, neck or other part or portion of the body. The holder 78 may be attachable to the patient by, for example a strap with hook and loop or an attachment comprising adhesive, or a clip that attaches to the clothing or belt of a patient, or a strap with a buckle fastening. A single connector 77 may have two connection points such that the fluid inlet conduit 50 and the first fluid outlet connector 52 are connected to the holder 78 at a multi-conduit connector 77, as shown in FIG. 62.

In the embodiment of FIGS. 7 and 8, the at least one section of the wall 15 of the receptacle 10 may allow molecules within the fluid to diffuse from the receptacle 10 to the wound 22 or to pass from the receptacle 10 to the wound 22 via pore flow, or it may allow molecules within the fluid to both diffuse from the receptacle 10 to the wound 22 and to pass from the receptacle 10 to the wound 22 via pore flow. The fluid may comprise a gas, such as oxygen gas or carbon dioxide gas or carbon monoxide gas.

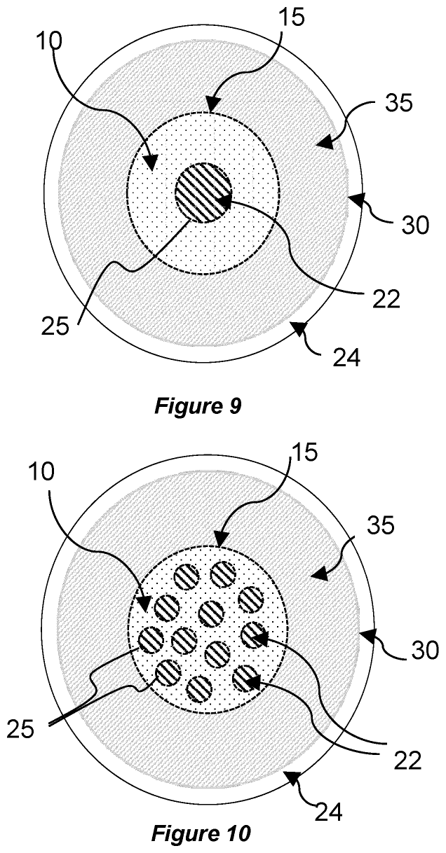

FIGS. 9 and 10 show embodiments of the apparatus 100 in which the receptacle 10 has a substantially annular or doughnut shape in plan view so as to include a through-hole 25. In use of the apparatus 100, the through-hole 25 provides an additional path for exudate to flow away from the wound surface. It provides additional areas of fluid communication between the wound 22 and the cover 30 or the absorbent material 75 (seen in FIG. 13) of the cover 30. The through hole 25 may therefore help to facilitate the flow of exudate away from the wound 22, particularly when the compartment 35 is subject to a negative pressure to form a negative pressure compartment. In the embodiment of FIG. 10, the receptacle 10 includes multiple through-holes 25, providing multiple additional paths for exudate to flow from the wound surface, which may further increase the flow of exudate away from the wound 22. These embodiments are described with reference to a wound, and its effect may be most advantageous when used at a wound. However, it will be appreciated that it is, in general, applicable to other tissue areas as described elsewhere in this disclosure. The receptacle 10 may include a plurality of the microstructures 60 arranged on the wound facing surface 12 (not shown in the plan views of FIGS. 9 and 10) of the receptacle 10.

The one or more receptacles 10 are movable by a clinician when being positioned on the wound area 20 on the patient and also in situ, in response to patient movement. However, in some circumstances it may be required to secure the receptacles in place. The apparatus 100 may further include one or more fasteners 80 for securing the receptacle(s) 10 in place at the wound area 20 once they are positioned. The fastener 80 may comprise adhesive, such as adhesive tape as schematically shown in FIG. 12 and may be used to secure the receptacle 10 and/or one or more of the fluid inlet conduit 50 and the first fluid outlet conduit 52 (seen in FIG. 3), 54 to the healthy skin 24 of the patient or to the cover 30. Securing the receptacle 10 and/or one or more the fluid inlet conduit 50, the first fluid outlet conduit 52, and the second fluid outlet conduit 54 in place may reduce movement of the components within the wound area 20, including the wound 22.

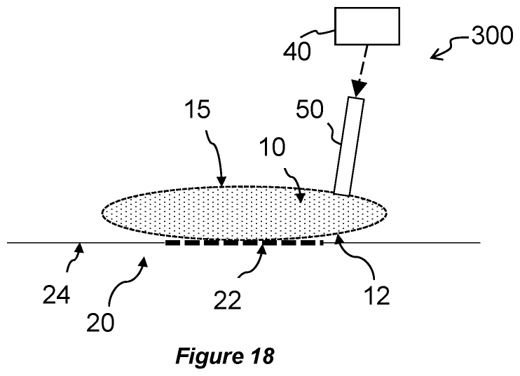

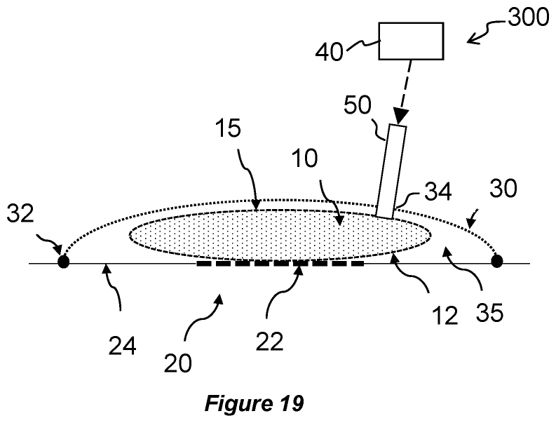

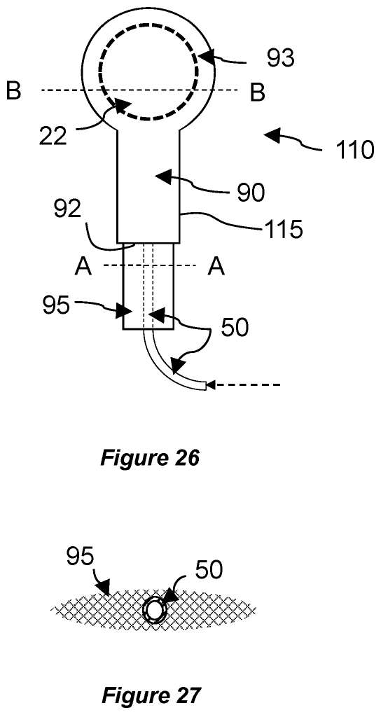

FIGS. 18 to 25 show embodiments of the apparatus 300 that do not include a negative pressure compartment. Whilst shown and described with reference to the receptacle 10, it may apply to any of the receptacles 10, 110, 210, 210A, 310, 410 described in this disclosure. These embodiments are described with reference to a wound. However, it will be appreciated that they are, in general, applicable to other tissue areas as described elsewhere in this disclosure. The components of these embodiments are as described in respect of the apparatus 100, 200 and like components are given like reference numbers. In each of these embodiments, as with the other embodiments disclosed herein, the fluid may comprise a gas, such as oxygen gas or carbon dioxide gas or carbon monoxide gas. In its simplest form as shown in FIG. 18, the apparatus 300 has a receptacle 10 and a fluid inlet conduit 50 through which fluid is introduced into the receptacle 10 from a fluid source 40. The receptacle 10 is formed of one or more walls 15 having at least one section made of the membrane adapted to allow molecules within the fluid to diffuse from the receptacle 10 to the wound area 20. The receptacle 10 is placed in contact with the wound 22, with the wound facing surface 12 of the receptacle 10 facing the wound 22. A schematic representation of a receptacle having a fluid inlet conduit 50 is shown in FIG. 26. In the embodiment of FIG. 19, an optional cover 30 in the form of a bandage may be wrapped around the body part containing the wound 22 such that it is wrapped over the receptacle 10 to provide pressure to the receptacle 10 and press it into contact with the wound 22. The cover 30 contacts the healthy skin 24 of the patient that surrounds the wound at the wound area 20. An optional seal 32, for example an adhesive seal, may be used to fasten the cover 30 to the healthy skin 24 to keep the cover 30 in place. Where the bandage is used as the cover 30, the fluid inlet conduit 50 may pass through the first opening 34 that is formed in between adjacent wrappings of the bandage. In this embodiment, the compartment 35 substantially bounded by the cover 30 in the form of the bandage, the receptacle 10 and the tissue area may be further bounded by the fluid inlet conduit 50.

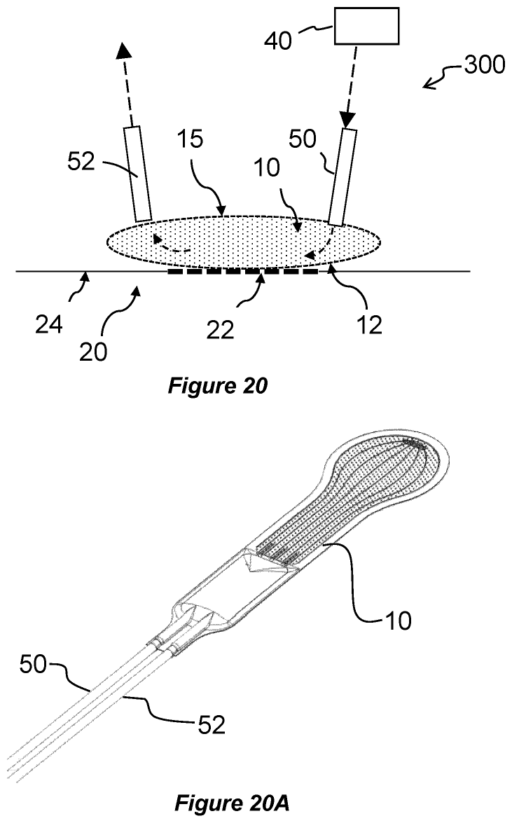

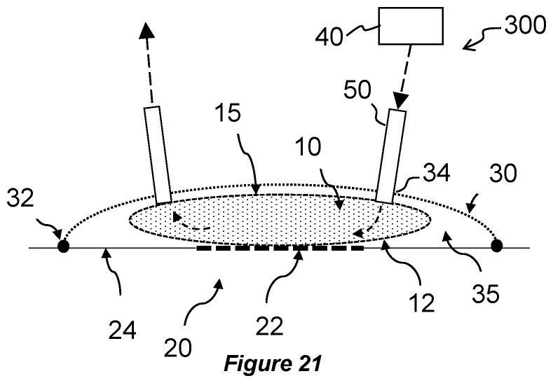

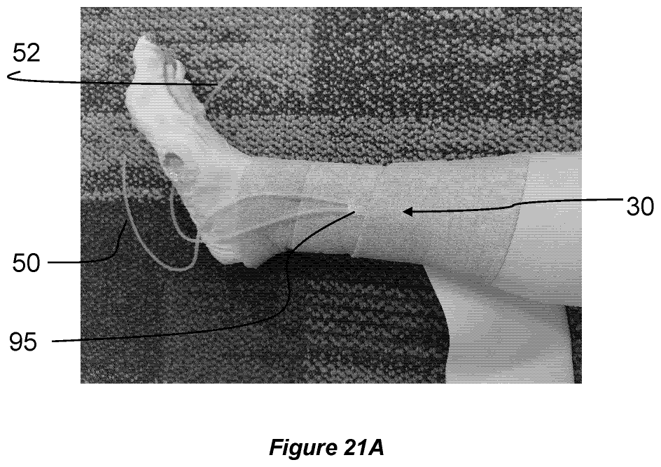

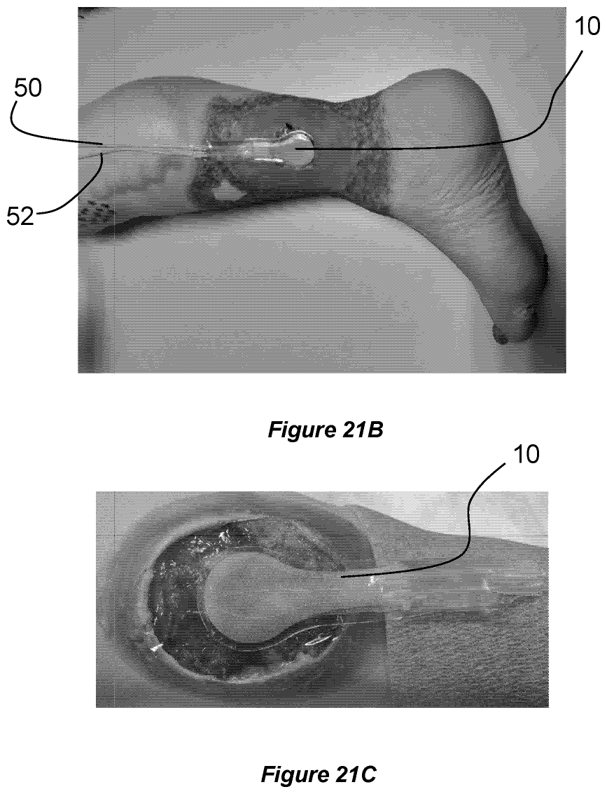

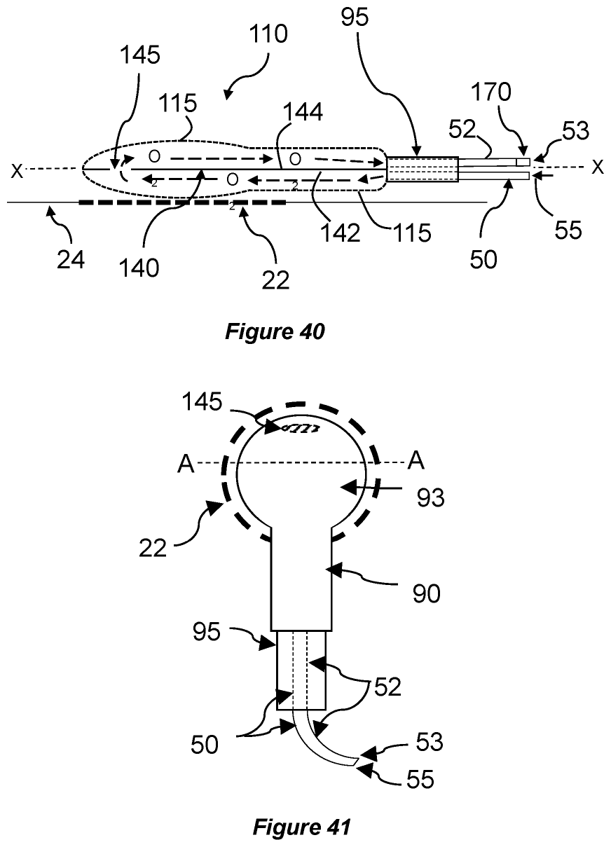

FIG. 20 shows an embodiment that is very similar to that of FIG. 18, with the addition of the first fluid outlet conduit 52. Accordingly, the fluid that enters the receptacle 10 through the fluid inlet conduit 50 may cycle through the receptacle 10 and exit the receptacle 10 through the first fluid outlet conduit 52. A view of a receptacle 10 having a fluid inlet conduit 50 and a first fluid outlet conduit 52 is shown in FIG. 20A. In FIG. 21, as with the embodiment of FIG. 19, the optional cover 30 in the form of a bandage may be wrapped around the body part of the patient having the wound 22 and around the receptacle 10 placed on the wound 22. An optional seal 32, which may be an adhesive seal, may be used to fasten the cover 30 to the healthy skin 24 to keep the cover 30 in place. Where the cover 30 in the form of a bandage is used, the fluid inlet conduit 50 and the first fluid outlet conduit 52 may pass through the first opening 34 and the second opening 36 that is formed in between adjacent wrappings of the bandage. Alternatively, the fluid inlet conduit 50 and the first fluid outlet conduit 52 may pass through the bandage as coaxial conduits or adjacent conduits as in the embodiment of FIG. 14 to 17, or they may be substantially adjacent conduits as described in relation to FIGS. 40, 41, 45-50 and 52 elsewhere in this disclosure. In this embodiment, the compartment 35 substantially bounded by the cover 30, the receptacle 10 and the tissue area may be further bounded by the fluid inlet conduit 50 and the first fluid outlet conduit 52.

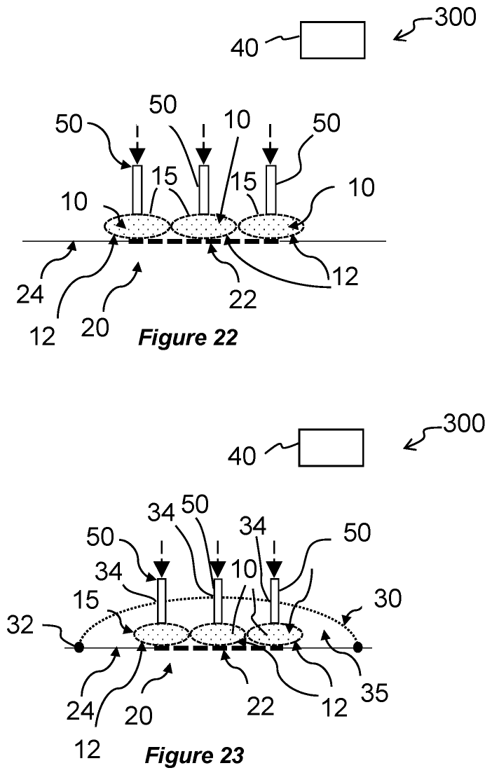

FIG. 22 shows an arrangement that has multiple receptacles 10, in this case three receptacles. As with the apparatus 100, the number of receptacles 10 shown in FIG. 22 is non-limiting and any reasonable number of receptacles 10 may be used depending on the area of the wound surface. Each receptacle 10 has a respective fluid inlet conduit 50 that is supplied with the fluid from the fluid source 40. The receptacles 10 are independent of one another and may be placed on the wound 22 where fluid is required. The receptacles 10 may be moved from time to time to treat other parts of the wound 22, for example a part that is not healing as quickly. FIG. 23 shows this arrangement with the optional cover 30 in the form of a bandage wrapped around the body part of the patient having the wound 22 and around the receptacles 10. The optional seal 32, which may be an adhesive seal, may be used to fasten the cover 30 to the healthy skin 24 to keep the cover 30 in place. Where the cover 30 in the form of a bandage is used, the respective fluid inlet conduits 50 may pass through respective first openings 34 that are formed in between adjacent wrappings of the bandage. In some embodiments, the respective fluid inlet conduits 50 may pass through a single first opening 34. In this embodiment, the compartment 35 substantially bounded by the cover 30, the receptacle 10 and the tissue area may be further bounded by the fluid inlet conduits 50.

In the embodiments of FIGS. 22 and 23, the at least one section of the wall 15 of the receptacle 10 may allow molecules within the fluid to diffuse from the receptacles 10 to the wound 22 or to pass from the receptacles 10 to the wound 22 via pore flow, or it may allow molecules within the fluid to both diffuse from the receptacle 10 to the wound 22 and to pass from the receptacle 10 to the wound 22 via pore flow.

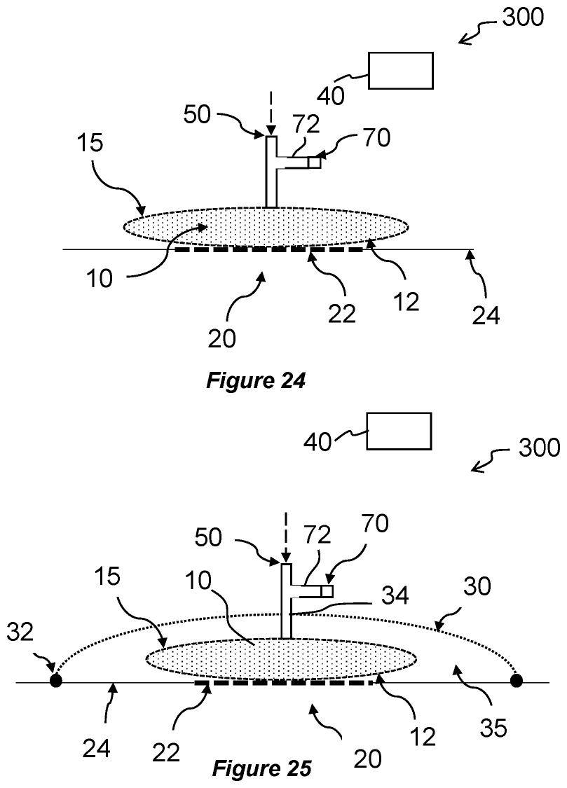

FIGS. 24 and 25 show variations of the arrangements of FIGS. 18 and 19 that include a pressure relief valve 70 either in the fluid inlet conduit 50 or in a branch line 72 of the fluid inlet conduit 50 as shown in FIGS. 24 and 25. In the embodiments of FIGS. 24 and 25, the at least one section of the wall 15 of the receptacle 10 may allow molecules within the fluid to diffuse from the receptacle 10 to the wound 22 or to pass from the receptacle 10 to the wound 22 via pore flow, or it may allow molecules within the fluid to both diffuse from the receptacle 10 to the wound 22 and to pass from the receptacle 10 to the wound 22 via pore flow. The arrangements are otherwise identical to the arrangements of FIGS. 18 and 19. In the embodiment of FIG. 25, the compartment 35 substantially bounded by the cover 30, the receptacle 10 and the tissue area may be further bounded by the fluid inlet conduit 50. In a further embodiment, the branch line 72 and pressure relief valve 70 may be present inside the cover 30. The cover 30 may include a vent to atmosphere, for example an outlet port, or the cover 30 may be a permeable cover 30 such as a compression bandage, such that gas released from the pressure relief valve 70 on the fluid inlet conduit 50 inlet may be vented from underneath the cover 30 to prevent the compartment 35 formed by the cover 30 from bursting. In this embodiment, the compartment 35 substantially bounded by the cover 30, the receptacle 10 and the tissue area may be further bounded by the fluid inlet conduit 50

In each of the embodiments of FIGS. 18 to 25, the receptacle(s) may include a plurality of microstructures 60 arranged on the wound facing surface 12 of the receptacle 10, or on any other portion of the wall 15 as shown in FIG. 1A.

As discussed elsewhere in this disclosure, some of the dressings that cover the receptacle(s) 10 may press the receptacle 10 against the wound 22 and the healthy skin 24 around the wound 22. Such dressings may also press some length of the fluid inlet conduit 50 or first fluid outlet conduit 52 connected to the receptacle 10 against the healthy skin 24 around the wound 22 and even the wound 22 itself, depending on the size and position of the receptacle 10 relative to the wound 22. When the outer diameter of the conduits 50, 52 is small, for example 5 mm or less, the patient may find it uncomfortable to have the conduit 50, 52 pressed against them in this way. Furthermore, when the apparatus 100, 200, 300 is removed, the patient may be left with a temporary yet uncomfortable indentation in the shape of the conduit 50, 52. FIG. 26 shows a schematic plan view of a variation of a receptacle 110 that is similar to the receptacle 10 in that it has a generally circular shaped head portion 93 that is placed over the wound 22 and further includes an additional tail portion 90 that is formed integrally with and extends from the generally circular shaped head portion 93. Alternatively, the tail portion and the head portion 93 may be formed as two separate components and joined together. The tail portion 90 has a generally elongate rectangular shape when viewed in plan view and it is inflatable with the circular head portion 93 of the receptacle 110. The tail portion 90 may be longer than it is wide. The tail portion 90 may have a width that is less than the width of the head portion 93. In an alternate embodiment, the tail portion 90 may be wider that is long. It may have a shape, when viewed in plan view, that is rectangular, square, elliptical or any other suitable shape. The fluid inlet conduit 50 connects to a distal end wall 92 of the tail portion 90 that is furthest from the head portion 93 such that it is in fluid communication with the tail portion 90. The fluid inlet conduit 50 is configured to supply fluid to the head portion 93 via the tail portion 90.

The tail portion 90 may extend beyond the wound 22 such that the interface between the fluid inlet conduit 50 and the receptacle 110 is spaced apart from the wound surface, reducing the likelihood of the fluid inlet conduit 50 being pressed into the wound 22 or other tissue site of the patient by the cover 30 in the form of a bandage or other dressing. Furthermore, the tail portion 90 may extend beyond the wound 22 such that the interface between the fluid inlet conduit 50 and the receptacle 110 is beyond the pressing force of the bandage 30 or other dressing, reducing the likelihood of the fluid inlet conduit 50 being pressed into the tissue of the patient by the bandage 30 or other dressing. In cases where the pressing force includes a portion of the tail portion 90 having the fluid inlet conduit 50, the pressing force may press the tail portion 90 against the patient rather than the fluid inlet conduit 50. This will be more comfortable for the patient. Whilst not shown in FIG. 26, a first fluid outlet conduit 52 may also protrude into the distal edge 92 of the tail portion 90. In embodiments that include a first fluid outlet conduit 52, see for example the embodiments of FIGS. 40 to 54 described later in this disclosure, the tail portion 90 allows the conduits 50, 52 to terminate distally from the wound 22 so that ends of the conduits 50, 52 will not impact the wound 22; that is, the application of a cover 30 in the form of a bandage or other dressing over the receptacle 110 will not press the conduits 50, 52 into the wound. In some cases, the tail portion 90 may lie over a portion of the wound 22 that is not covered by the head portion 93, particularly where the wound 22 is large and/or of an irregular shape. The cover 30 in the form of a bandage or other dressing may therefore be wrapped over the tail portion 90 rather than directly over the tissue or a wound of the patient. Furthermore, tissue bordering the wound 22 may also be compromised or at a different stage of healing and may be sensitive and/or easily damaged. Wrapping the cover 30 over the tail portion 90 rather than directly over the wound or the tissue in these cases may help to reduce patient discomfort. The user of the receptacle 10 may also find it more straight forward to seal a negative pressure dressing or cover 30 to an upper surface of the tail portion 90 rather than over the top of multiple, separate conduits. The receptacle 110 may be inflatable. In some embodiments, the receptacle 110 is a bag. The receptacle 110 may comprise one or more walls 115 and is adapted to receive a fluid. The one or more walls of the tail portion 90 may be formed from the same material as the one or more walls of the head portion 93. As such, the one or more walls 115 of the head portion 93 and the tail portion 90 of the receptacle 110 may have the same properties as the one or more walls 15 described in this disclosure, and may be flexible and conformable such that they can conform to the wound topology. The wall 115 of the tail portion 90 may be formed continuously with the wall 115 of the head portion 93; that is, they may be the same wall 115. Alternatively, the wall 115 of the tail portion 90 may not be formed continuously with the wall 115 of the head portion 93; that is, they may not be the same wall 115. The one or more walls 115 of the head portion 93 and the tail portion 90 may comprise at least one section adapted to allow molecules within the fluid to move from inside of the receptacle 110 to the tissue area. The one or more walls 115 of the head portion 93 and the tail portion 90 may be adapted to allow the molecules to move through the wall via diffusion. The one or more walls 115 may be substantially pore-free such that molecules do not move through the wall via pore flow. The one or more walls 115 may be substantially impermeable to bulk transport of fluid. However, in some embodiments, the one or more walls 115 that comprise the tail portion 90 of the receptacle 110 may or may not be adapted to allow the molecules to move through the wall via diffusion. The head portion 93 and the tail portion 90 may be formed as separate components that may be pneumatically connected. The tail portion 90 may be inflatable. Alternatively, the tail portion 90 may not be inflatable. The tail portion 90 may be made of a different material to the head portion 93, for example it may be made of a foam. The tail portion 90 may comprise of a foam having a coating that is impermeable to bulk transport of fluid and/or diffusion. Alternatively, it may be made of a material that is adapted to substantially prevent molecules within the fluid from passing through it. The receptacle 110 may include a plurality of microstructures 60 arranged on a wound facing surface 12 of the receptacle 110. The tail portion 90 of the receptacle 110 may include a plurality of the microstructures 60. However, an alternative embodiment of the tail portion 90 of the receptacle 110 may not include the plurality of microstructures 60. Embodiments of the tail portion 90 may provide a bridge between the head portion 93 of the receptacle 110 and the ends of the fluid inlet conduit 50 and first fluid outlet conduit 52 to which the fluid source 40 is connected. For the receptacle 110, 210, 210A, 310, 410 and all embodiments of the receptacle described in this disclosure that may include a tail portion 90, the tail portion 90 allows fluid supplied into the fluid inlet conduit 50 to be transported to the head portion 93 for treatment at the wound 22. All other features of the receptacle 110 are the same as for the receptacle 10. The fluid may comprise a gas, such as oxygen gas or carbon dioxide gas or carbon monoxide gas.

Whilst the disclosure of the features of the tail portion 90 are described with reference to the receptacle 110, one or more of the features may apply to any of the receptacles 10, 210, 210A, 310 or 410 described in this disclosure.

Furthermore, as shown in FIGS. 26 and 27, the fluid inlet conduit 50 is encapsulated in a pressure spreading device 95 that is adjacent the receptacle 110. The pressure spreading device 95 is a silicone component that is moulded with the fluid inlet conduit 50 so as to surround it, as shown in the cross-sectional view of FIG. 27. The pressure spreading device 95 spreads the load of the fluid inlet conduit 50 over a greater portion of the healthy skin 24 of the patient adjacent the wound 22 to reduce indentation during and after use of the receptacle 110.

The fluid inlet conduit 50 may be bonded to the pressure spreading device 95. The pressure spreading device 95 may encapsulate at least a portion of the fluid inlet conduit 50 between the fluid delivery device and a fluid source. The pressure spreading device 95 may be shaped to reduce a pressure exerted on the patient by the fluid inlet conduit 50. In some embodiments, the pressure spreading device 95 may have a width that is greater than an external diameter of the fluid inlet conduit 50. For example, the width may be about 2 to 10 times greater than the external diameter, or about 3 to 9 times greater than the external diameter, or about 4 to 8 times greater than the external diameter, or about 5 to 7 times greater than the external diameter of the fluid inlet conduit 50.

Whist some embodiments of the receptacles 10, 110, 210, 210A, 310, 410 may have a tail portion 90 and a pressure spreading device 95, some embodiments of the receptacles 10, 110, 210, 210A, 310, 410 may have a tail portion 90 and no pressure spreading device 95, or they may have a pressure spreading device 95 and no tail portion 90.

The tail portion 90 and the pressure spreading device 95 may be formed integrally with one another or they may comprise separate components. The tail portion 90 and the pressure spreading device 95 may be bonded together. The pressure spreading device 95 and the receptacle 110 may be made from the same material. The pressure spreading device 95 may have a generally elliptical cross-section. In some embodiments, it may have a cross-section that is shaped like a convex lens as shown in FIG. 27. The fluid inlet conduit 50 may be bonded to the pressure spreading device 95.

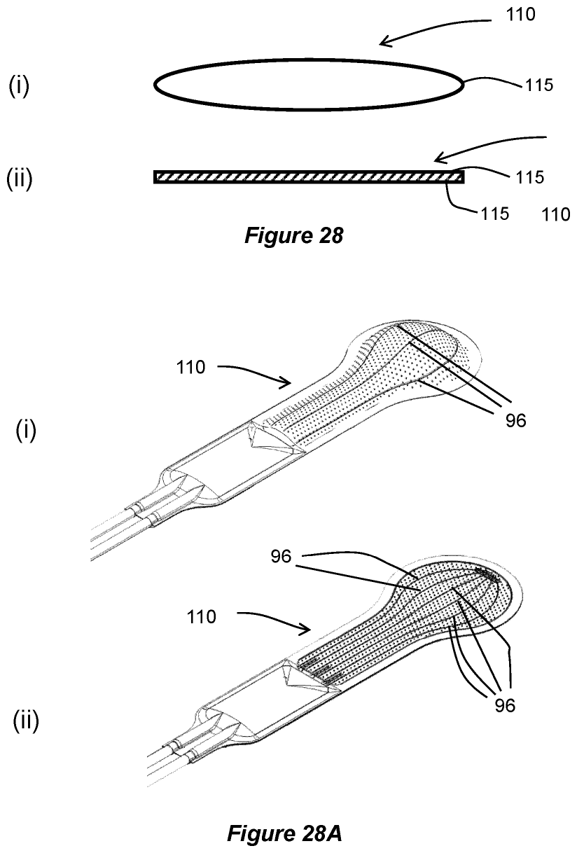

During use of the receptacle 110, fluid passes from the fluid inlet conduit 50, into the tail portion 90, through the tail portion 90, and into the head portion 93. This flow of fluid is intended to at least partially inflate both the tail portion 90 and the head portion 93 in order to press the outside of the receptacle 110 to the target tissue, which helps to maximise the surface contact between the receptacle 110 and the wound 22 for delivery of molecules within the fluid to the target tissue, including the wound 22. Inflation of the receptacle 110 is schematically shown in the cross-sectional view of FIG. 28(i) and in CAD form in the view of the inflated receptacle 110 of FIG. 28A(i). If the receptacle 110 is prevented from at least partially inflating, fluid flow through the receptacle may be impaired or even prevented, reducing or preventing the delivery of molecules within the fluid to the wound 22. For example, if the receptacle 110 is made of a smooth silicone material, one or more sections of the inner surface(s) of the one or more walls 115 of the receptacle 110 may adhere together such that the receptacle is unable to inflate. A non-inflated receptacle 110 is shown schematically in FIG. 28(ii), and in the view of FIG. 28A(ii).

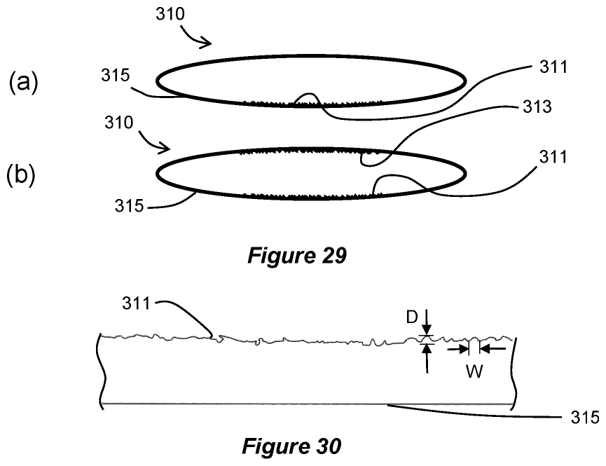

In order to reduce the adherence together of the one or more sections of the inner surface(s) of the wall(s) 115 of the receptacle 110, surface contact between the one or more sections may be reduced. In some embodiments of the receptacle 110, the surface contact may be reduced by roughening the one or more sections of the inner surface(s) of the wall(s) 115. FIG. 29 shows a cross-sectional view of an embodiment of a receptacle 310 in which an inner surface of one or more sections of the wall(s) 315. i.e. a non-wound contacting surface, is roughened to form a rough surface. In FIG. 29a, an inner surface of a wall 315 has a roughened section forming a first rough surface 311. As shown in FIG. 29b, multiple sections of the wall(s) 315 can be roughened; the inner surface of the wall 315 has a first roughened section forming a first rough surface 311 and a second roughened section forming a second rough surface 313. However, the wall(s) 315 may have further roughened sections forming further rough surfaces. Whilst FIG. 29(a) and FIG. 29(b) show roughening of a central section of the inner surface of the wall(s) 315, the roughening may be applied over other sections of the wall(s) 315 or even over the entirety of the inner surface of the wall(s) 315. In FIG. 29b, the first rough surface 311 and the second rough surface 313 are opposite one another, however they may also be on sections of the inner surface of the wall(s) 315 that are not opposite one another. A close-up view of a cross-section of the rough surface 311 of the wall 315 is shown in FIG. 30, however the features are equally applicable to the rough surface 313. The rough surface 311 has a rough texture. The rough surface 311 may be irregular; it may vary in height (D) and it may include a plurality of raised and/or recessed elements that vary in width (W) as shown in FIG. 30. For example, the height (D) of the elements may be between about 0.2 micrometres to 200 micrometres, or between about 0.2 micrometres and 150 micrometres, or between about 0.2 micrometres and 100 micrometres, or between about 0.3 micrometres and 50 micrometres, or between about 0.5 micrometres and 10 micrometres. As a result of the rough surface 311, there is less surface contact between the opposing inner surfaces of the wall(s) 315, so a lower likelihood of the inner surfaces of the wall(s) 315 adhering together to prevent inflation of the receptacle 310.

Whilst the rough surface is described with respect to the receptacle 310, it will be apparent that it may be applied to any of the receptacles 10, 110, 210, 210A, 310 or 410 described in this disclosure. Accordingly, the rough surface may be combined with one or more features of the receptacles 110, 210, 210A, 310 and 410.

If the receptacle 110 or 310, or any one of the receptacles 10, 210, 210A, 410 described in this disclosure, is covered with a cover in the form of a dressing, (e.g. a compression dressing), the pressure from the dressing may also press walls of the receptacle 10, 110, 210, 210A, 310, 410 together in such a way as to block the flow of fluid through the tail portion 90. Patient movement or position may also cause this kind of blockage if, for example, a patient rolls on top of the receptacle 10, 110, 210, 210A, 310, 410. Such a blockage may stop fluid getting to the head portion 93. If this occurs, the patient will not receive therapy. To reduce the chance of such a blockage, a feature can be incorporated to help maintain a flow path along the tail portion 90, regardless of the pressure applied, such as by dressings, patient movement or patient position.

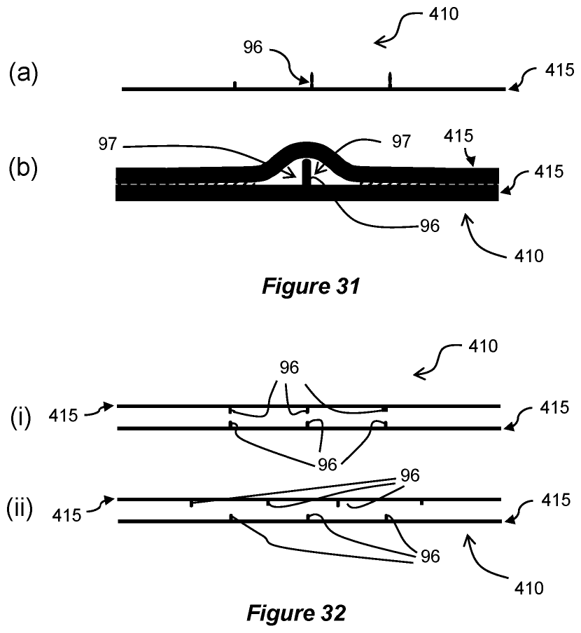

FIG. 31a shows a cross sectional view, such as at line B-B of FIG. 26 of an embodiment of the inside surface of a wall 415 of a receptacle 410. The inner surface of the wall 415 includes one or more small, soft, protruding ridges 96. When two portions of the one or more walls 415 are pressed together, the wall 415 opposing the ridge 96 drapes over the ridge 96, leaving gaps 97 on either side of it as shown in FIG. 3 lb. The gaps 97 will generally remain even when tight dressings are applied over the receptacle 410 in use or regardless of patient movement. The ridges 96 are also shown in the views of the receptacle 415 of FIG. 28A.

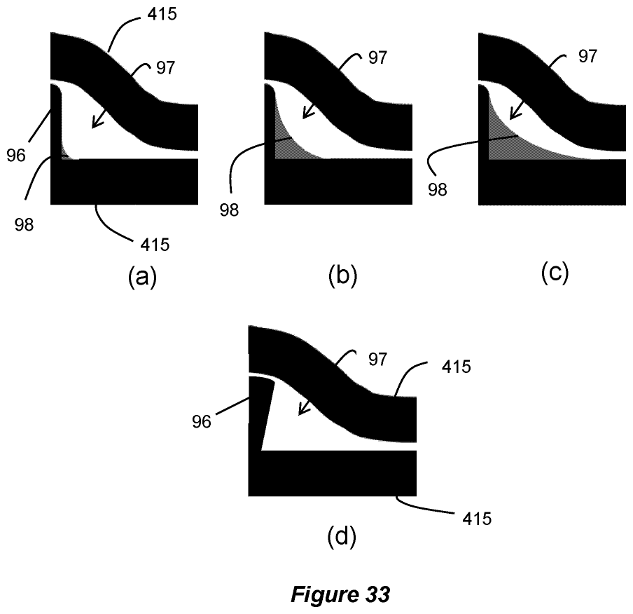

The gaps 97 are formed due to the shape of the corner between each ridge 96 and the section of wall 415 from which it protrudes. The corners may be sharp such that it is substantially non-filleted, or it may have a fillet 98 that is small as shown in FIG. 33a. For a substantially non-filleted corner, the corner may have a fillet that is so small as to be negligible, within the bounds of manufacturing capabilities. If the size of the fillet 98 is small in comparison with the height of the ridge 96, the likelihood that an opposing section of wall 415, pressed against the section with the ridges 96, will be able to deform in such a way that it fills that gaps 97, is reduced. For example, the fillet radius may be not more than about 30%, for example not more than about 25% or not more than about 20% or not more than about 10% of the height of the ridge 96.

The importance of the sharp corners and fillets 98 is illustrated by the four alternatives shown in FIG. 33a-c. Each of the alternatives shows the gap 97, between a first wall portion of the wall 415 and a second wall portion which can be an opposing wall portion of the wall 415 or another wall 415, on one side of a ridge 96. In FIG. 33a, the fillet 98 is very small, so the corner is very sharp. Therefore, in this alternative, it is unlikely that the opposing wall would deform in such a way as to fill the gaps 97 when pressed onto the ridge 96. In FIG. 33b, the fillet 98 is larger, so the corner is less sharp. Although this geometry may still result in gaps 97 being present when the opposing wall 415 presses down on the ridge 96, the gaps 97 would not be as large as they could be if the fillet 98 was smaller. In this case, fluid flow through the receptacle 410 would be more restricted in FIG. 33b than in FIG. 33a. In FIG. 33c, the fillet 98 is large. In this alternative, it is possible that the opposing wall 415 would deform to fill the gaps 97. This would likely result in a blockage within the receptacle 410, which would compromise the therapy. The illustrative examples of the fillets 98 are shown in FIG. 33a-c as having a concave fillet shape. However, the fillets 98 may also be convex or mitre shaped. Alternatively, as shown in FIG. 33d, the ridge 96 may overhang the corner to produce a sharp corner. In this embodiment, the corner may be substantially non-filleted, within the bounds of manufacturing capabilities.

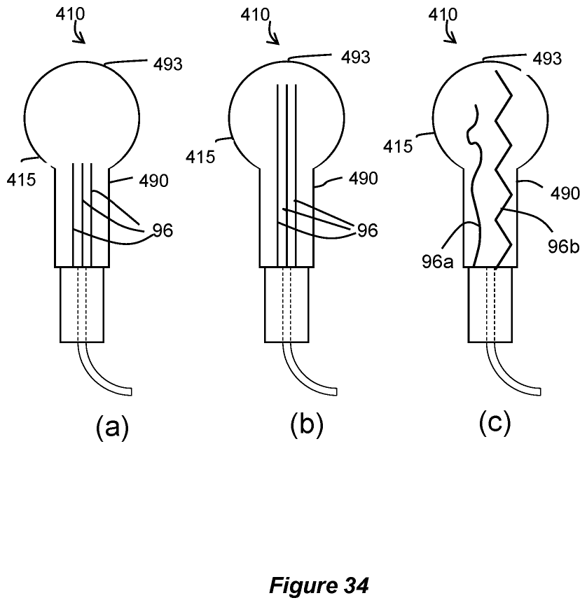

The one or more ridges 96 may extend through the tail portion 490 of the receptacle 410, as far along the tail portion 490 as is necessary to maintain an open flow path inside the receptacle 410. For example, a plurality of the ridges 96 may extend through the entire tail portion 490 as shown in FIG. 34a. FIG. 34a shows three ridges 96. However, it will be appreciated that any appropriate number of ridges 96 may be included inside the tail portion 490. The ridges 96 may extend across some proportion of the head portion 493 as shown in FIG. 34b. The ridges 96 may be straight as in the examples of FIG. 34a and FIG. 34b. The one or more ridges 96 may comprise two or more ridges arranged substantially parallel to one another as in the examples of FIG. 34a and FIG. 34b. Alternatively, the ridges 96 may be non-straight, for example they may have a longitudinal form that is a regular or irregular curve 96a, or zigzag 96b as illustrated in the example of FIG. 34c. The ridges 96, 96a, 96b maintain a flow path between the entry point of the fluid conduit 50 to the tail portion 490 and the head portion 493. FIG. 28A shows views of an embodiment of the receptacle 110 showing the ridges 96, in both an inflated configuration of the receptacle 110 (FIG. 28A(i) and a non-inflated configuration (FIG. 28A(ii). Whilst the ridges 96 are shown for the receptacle 110, it will be apparent that the ridges apply also to any of the receptacles 10, 110, 210, 210A, 310, 310, 410 described in this disclosure.

The ridges 96 may be formed of a soft, yielding material such as silicone. The yielding material is not rigid and may give way under pressure. It will deform to an extent under pressure but will regain its original configuration once the pressure is removed. The extent to which the ridges 96 may yield is a balance of the requirements for softer, more yielding ridges to help reduce indentation on the patient tissue while maintaining enough rigidity to maintain the flow path and prevent blockage. The one or more walls 415 of the receptacle 410 may include the first wall portion and the second wall portion opposite the first wall portion. The ridges 96 may be formed integrally with the first wall portion and/or the second wall portion respectively of the receptacle 410. The ridge 96 may have a rounded tip as shown in FIG. 31b. A height of each ridge 96 may be similar to the height of one of the microstructures 60 described elsewhere in this disclosure. Each ridge 96 may have a height of between approximately 50 to 500 micrometres, for example 75 to 250 micrometres, for example 100 to 200 micrometres, for example between approximately 125 to 175 micrometres. An embodiment of the ridge 96 has a height of 150 micrometres. The height of the ridge 96 is a balance of the requirement for a taller ridge to help maintain a sufficient gap 97 and thereby prevent blockage of the flow path, whilst avoiding an overly tall ridge that may cause indentation in the patient tissue and which may render the ridge 96 susceptible to buckling. A width of each ridge 96 may be similar to the base diameter of the one of the microstructures 60. Each ridge 96 may have a width of between approximately 50 to 500 micrometres, for example 100 to 400 micrometres, for example between approximately 250 to 350 micrometres, or between approximately 280 to 330 micrometres. The ridge 96 increases the thickness of the wall 415 at the particular location of the ridge 96, which may affect the ability of molecules within the fluid to diffuse through the wall 415 at that location. Thus, whilst a larger number of ridges 96 may be included in the receptacle 110 to maintain a fluid flow path in the event the receptacle 110 is crushed, the number of ridges 96 should be balanced with the requirement that the molecules within the fluid can diffuse through the wall 415.

The thickness of the wall portion opposing the first wall portion or second wall portion of the wall 415 having the ridge 96 formed therewith is also a factor in the size of the gap 97 that is formed as the opposing first wall portion or second wall portion of the wall 415 is pressed onto the ridge 96. A thicker wall portion is less likely to deform into the gap 97, whereas a thinner wall portion is more flexible and thus more likely to deform and fill a gap 97. A thinner wall portion 415 may also have higher rates of diffusion across the wall than a thicker wall portion 415. The wall(s) 415 of the receptacle 410 may be the same material and thickness as those described in respect of receptacle 10 and may be flexible and conformable such that they can conform to the wound topology. Hence, it is important to balance these desirable properties of the first wall portion or second wall portion of the wall(s) 415 with the likelihood of blockage and choose the dimensions of the ridge 96 accordingly e.g. decrease fillet size or increase ridge height to ensure a suitable gap 97 is always present. For example, a thickness dimension of the first wall portion and/or the second wall portion may be less than or approximately equal to a height dimension of the ridge 96. Alternatively, the thickness dimension of the first wall portion and/or the second wall portion may be greater than a height dimension of the ridge 96.

The first wall portion and/or the second wall portion of the wall(s) 415 may have a wall thickness of between about 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between about 45 micrometres and 55 micrometres. In an embodiment, the first wall portion and/or the second wall portion may have a wall thickness of approximately 60 micrometres. In another embodiment, the first wall portion and/or the second wall portion may have a wall thickness of approximately 50 micrometres.

In the embodiment of FIG. 32, ridges 96 are provided on each of an opposing first wall portion and second wall portion of the wall(s) 415. In FIG. 32(i), the ridges 96 are directly opposed when the surfaces are pressed together. In FIG. 32(ii), the ridges 96 on opposing inner surfaces of the first wall portion and second wall portion of the wall(s) 415 are offset from one another when the surfaces are pressed together and are not directly opposed. Either embodiment may be used, however the use of non-opposing ridges may be less likely to indent the patient's tissue when the receptacle 410 is pressed onto the wound 22 and/or surrounding tissue.

Whilst the features of the ridges 96 are described with respect to the receptacle 410, it will be apparent one or more of the features may also be applied to any of the receptacles 10, 110, 210, 210A, 310 or 410 described in this disclosure. For example, the ridges 96 may be used in combination with the rough surface 311, 313 of the receptacle 310 to mitigate indentation of the patient tissue in conjunction with reducing adherence of the inner surfaces of the receptacle 310 to one another. One or more ridges 96 extending through the tail portion 90 into the head portion 93 of the receptacles 110, 310 may contribute to the prevention of the inner surfaces adhering together.

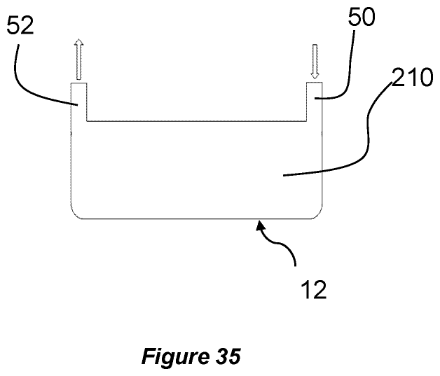

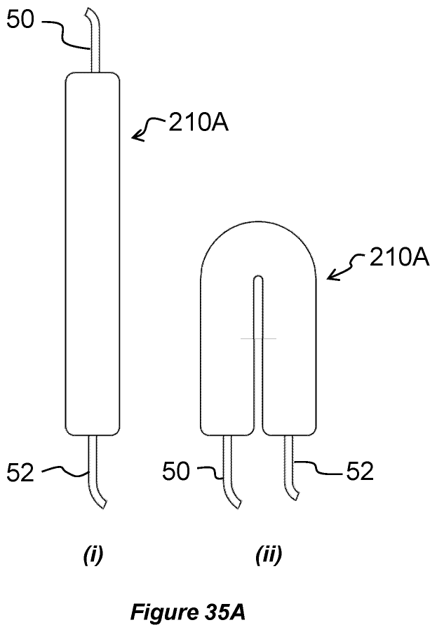

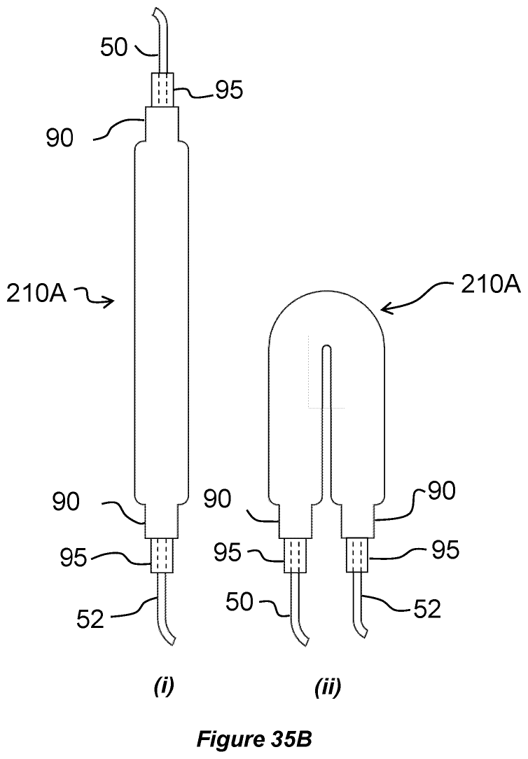

FIG. 35 shows a further variation of the receptacle 210 that is formed to have an elongated tubular shape through which fluid may be cycled. The receptacle 210 has a fluid inlet conduit 50 at one end of the elongate tube shape, for admission of fluid into the receptacle 210. It also has a first fluid outlet conduit 52 through which fluid may exit the receptacle 210 as with other embodiments of the receptacle 210. All other features of the receptacle 210 are the same as for the receptacle 10. This embodiment of the receptacle 210 may be particularly suitable for use on wounds or other tissue surfaces of a certain shape, for example elongate and/or narrow wound or other tissue area shapes. The receptacle 210 may include a plurality of microstructures 60 arranged on the wound facing surface 12 of the receptacle 10. The fluid may comprise a gas, such as oxygen gas or carbon dioxide gas or carbon monoxide gas.

A variation of the embodiment of FIG. 35 is shown in FIG. 35A and FIG. 35B. As shown in FIG. 35A(i), the receptacle 210A is formed to have an elongated tubular shape through which fluid may be cycled. The receptacle 210A has a fluid inlet conduit 50 at one end of the elongate tube shape, for admission of fluid into the receptacle 210A. It also has a first fluid outlet conduit 52 at an opposite end of the receptacle 210A to the fluid inlet conduit 50 through which fluid may exit the receptacle 210A as with other embodiments of the receptacle 210. All other features of the receptacle 210A are the same as for the receptacle 10. The elongate tube shape of the receptacle 210A allows a user to fold the receptacle 210A along its length as shown in FIG. 35A(ii), such that the fluid inlet condit 50 is positioned close to the first fluid outlet conduit 52 when in use on a patient. This arrangement means that fluid entering the receptacle 210A at the fluid inlet conduit 50 must pass along the length of the receptacle 210A to reach the first fluid outlet conduit 52, which may prolong the residence time that the fluid spends inside the receptacle 210A. The receptacle 210A may include one or more ridges extending from the fluid inlet conduit 50 to the first fluid outlet conduit 52, as described in respect of the embodiments of FIG. 31a to FIG. 34. The ridges 96 may at least partially maintain open a fluid flow path from the fluid inlet 50, through the receptacle 210A to the first fluid outlet 52 when the receptacle 210A is folded. As shown in FIG. 35B, the receptacle 210A may include a tail portion 90 and/or pressure spreading device 95 at the fluid inlet conduit end of the receptacle 210A and/or the first fluid outlet conduit end of the receptacle 210A.

For some wounds, such as wounds with complex shapes or tunneling wounds, it may be more effective to insert a receptacle into the wound rather than place it on the wound. Insertion of the receptacle may result in a greater surface contact between the one or more walls of the receptacle and the patient tissue. This may assist both fluid delivery to the wound and exudate management. To insert the receptacle into the wound, the user may fold, twist, scrunch or otherwise manipulate the receptacle so that it can be inserted into the wound. In this event, the ridges 96 help to maintain open the fluid flow path from the fluid inlet conduit 50 to the first fluid outlet conduit 52. The receptacle 210A is an example of a receptacle that is easily folded or otherwise manipulated for insertion into a wound, however other receptacles 10, 110, 210, 310, 410 described in this disclosure may also be user manipulated for this purpose.

In all embodiments of the apparatus 100, 200, 300, 1100, 1200, 1300 and/or the receptacles 10, 110, 210, 210A, 310, 410 the fluid source 40 may be controlled to maintain a set pressure in the receptacle 10, 110, 210, 210A, 310, 410. That is, the controller may compensate over time for loss of fluid from the receptacle 10, 110, 210, 210A, 310, 410 due to e.g. fluid diffusion out of the receptacle 10, 110, 210, 210A, 310, 410 via the wound facing surface 12 to the wound 22. As such, the section of the wall 15 or the entire wall 15 that is made of the membrane experiences an even pressure, which results in an even pressure of fluid applied at the wound 22.

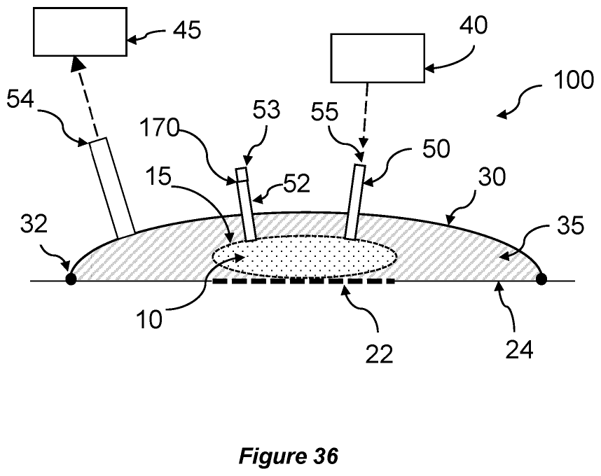

FIGS. 36 to 39 show further embodiments of the apparatus 100 having a pressure regulator associated with the fluid outlet 53. However, the features of the apparatus of any one of the embodiments of FIGS. 1 to 35A may feature also in the embodiment of FIGS. 36 to 39. The embodiments of FIGS. 36 to 39 may assist in achieving a substantially consistent concentration of the fluid within the receptacle 10, for example an oxygen concentration within the receptacle 10, by facilitating the cycling of the fluid through the receptacle 10.

In the embodiment of FIG. 36, the fluid inlet conduit 50 has a fluid inlet 55 that is positioned at an end of the fluid inlet conduit 50 that is distal from the receptacle 10. The first fluid outlet conduit 52 of the receptacle 10 has a fluid outlet 53 that is positioned at an end of the first fluid outlet conduit 52 that is distal from the receptacle 10. The fluid inlet 55 and the fluid outlet 53 each provide fluid communication with the receptacle 10, via the fluid inlet conduit 50 and the first fluid outlet conduit 52, respectively, and may comprise a port, connector, or other fitting. A fluid flow path through the receptacle 10 may be defined by the one or more walls of the receptacle 10, the fluid inlet 55 and the fluid outlet 53. A boundary of the fluid flow path through the receptacle 10 may be independent of the tissue of the patient. That is, the tissue of the patient does not form part of the fluid flow path through the receptacle 10. Whether the wall 15 of the receptacle 10 allows molecules within the fluid inside the receptacle 10 to diffuse through the wall 15 to the wound 22, or to pass from the receptacle 10 to the wound 22 via pore flow, the wall 15 is present between the fluid and the wound 22 as the fluid is delivered to the receptacle 10.

A pressure regulator in the form of a pressure relief valve 170 is associated with, that is, disposed at the fluid outlet 53. The pressure regulator is disposed in fluid communication with the fluid outlet 53 and thus also with the receptacle 10. The pressure regulator, for example the pressure relief valve 170, may be operable to relieve pressure within the fluid outlet 53 and/or the receptacle 10. The pressure relief valve 170 may be positioned in spaced relation from, that is, at a distance away from the receptacle 10, e.g. at the fluid outlet 53 that allows the pressure relief valve 170 to be moved relative to the receptacle 10. For example, the first fluid outlet conduit 52 may have a length, at the distal end of which is the fluid outlet 53. The pressure relief valve 170 may be positioned on the first fluid outlet conduit 52 at the fluid outlet 53, distally from the receptacle 10, which allows it to be positioned outside of any cover 30 As such, the fluid inlet conduit 50 or the first fluid outlet conduit, with the fluid outlet 53 and the pressure relief valve 170, can be moved for ease of wrapping a bandage or other dressing over the receptacle 10. The arrangement allows a cover 30 in the form of a bandage or other dressing to be wrapped over the receptacle 10 without having to also wrap the bandage around the additional bulk of a pressure relief valve 170 or other pressure regulator. As shown in FIG. 36, the pressure relief valve 170 is placed outside of the cover 30. Alternatively, for embodiments in which no negative pressure source 45 and no cover 30 is present, the pressure relief valve 170 may be placed close to, or even in contact with, the receptacle 10. In such an embodiment, the first fluid outlet conduit 52 may be omitted.

During use of the apparatus 100, the receptacle 10 may be maintained at a positive pressure (e.g. the receptacle 10 may be inflated) to encourage molecules within the fluid inside the receptacle 10 to diffuse through the membrane of the receptacle 10 to the wound 22 by generating a pressure gradient across the membrane. The apparatus 100 may be set up so that a constant, low flow of fluid enters the receptacle 10 via the fluid inlet conduit 50. The constant fluid flow, along with the pressure regulator on the fluid outlet 53, maintains a positive pressure within the receptacle 10. However, the constant fluid flow in through the fluid inlet 55 also means that there must be a regular fluid flow out through the fluid outlet 53 as the rate of diffusion through the membrane is not high enough to empty the receptacle 10 of all of the fluid entering the receptacle 10 at the fluid inlet 55. The regular fluid flow out through the fluid outlet 53 may not necessarily be constant due to operation of the pressure relief valve 170. Furthermore, in the initial stages of use of the receptacle 10 or at low fluid flow rates, there may be no need for the pressure relief valve 170 to open, such that no fluid flows out through the fluid outlet 53 unless a threshold pressure within the receptacle 10 is reached that causes the pressure relief valve 170 to open. At a steady state, the constant or regular flow of fluid in and out of the receptacle 10 cycles the fluid through the receptacle 10 so that the fluid, e.g. oxygen, within the receptacle 10 is maintained at a high concentration. If there was no cycling of the fluid, the fluid in the receptacle 10 could be diluted by other fluids, e.g. nitrogen, that may diffuse in to the receptacle 10 through the membrane from the outside.

The pressure regulator on the fluid outlet 53 is important to the cycling process, as it allows cycling of the fluid to be achieved in a controlled manner. In particular, the pressure regulator may be configured to ensure cycling does not occur at too high a rate that may lead to wasted fluid, e.g. oxygen, in addition to maintaining a positive pressure within the receptacle 10 as described above. The pressure relief valve 170 is configurable to relieve pressure within the receptacle 10 when the pressure within the receptacle 10 reaches a threshold value. In some embodiments, the pressure relief valve 170 may be configurable to adjust the pressure threshold value. The pressure threshold value may be an upper pressure bound, above which the pressure relief valve is to open. By having an upper pressure bound threshold, cycling of the fluid through the receptacle 10 may only occur if the upper bound is exceeded, therefore a positive pressure may be maintained within the receptacle, providing a pressure gradient between the receptacle 10 and the wound 22. This pressure gradient may aid with diffusion of molecules within the fluid across the membrane from a high pressure side (within the receptacle 10) to a low pressure side outside of the receptacle 10, e.g. at the wound 22. That is, if the pressure gradient results in a high partial pressure of fluid, e.g. oxygen, within the receptacle 10 and a low partial pressure of fluid, e.g. oxygen, at the wound 22, the pressure gradient may drive diffusion of molecules within the oxygen across the membrane to the wound 22. In contrast, a high partial pressure of nitrogen outside of the receptacle 10 and a low partial pressure of nitrogen within the receptacle 10 may cause nitrogen to diffuse into the receptacle 10 as discussed above, hence cycling of fluid through the receptacle 10 helps to maintain a high concentration of oxygen within the receptacle 10. The purpose of the cycling is to regularly refresh the fluid within the receptacle 10 with fluid of a high oxygen concentration by cycling out fluid already within the receptacle 10 including any other fluids such as nitrogen, so that more of the high oxygen concentration fluid reaches the wound 22.

The pressure relief valve 170 may comprise any suitable type of valve such as a spring-loaded check valve with a disc, a spring-loaded check valve with a ball, a diaphragm valve, a lift valve, a butterfly valve or a duckbill valve. Each of these valve types may be set to permit fluid to pass through the pressure relief valve 170 and out of the fluid outlet 53 when a threshold fluid pressure is reached upstream of the pressure relief valve 170.

The pressure relief valve 170 may be selectively actuable to an at least partially open position to permit fluid to pass therethrough, and is configured, when in the at least partially open position, to vent the fluid passing there through to the atmosphere, via the fluid outlet 53. In some embodiments, the pressure relief valve 170 may be either open or closed, however the pressure relief valve 170 valve may also include at least one partially open position between the open and closed positions. The open position may correspond to a threshold fluid pressure at which the pressure relief valve 170 opens. Where used, the at least one partially open position may correspond to at least one other threshold fluid pressure at which the pressure relief valve 170 partially opens.

The pressure relief valve 170 may be passively actuated, or it may be actively actuated. Where the pressure relief valve 170 is actively actuated, a sensor, for example a pressure sensor or a flow sensor, may be disposed to sense a parameter of the fluid from which a fluid pressure can be determined. A controller is disposed in communication with the sensor. The fluid pressure may be provided to the controller. The controller may be configured to control the pressure relief valve 170 such that if the pressure sensor detects a fluid pressure that is greater than or equal to one of the threshold fluid pressures, the pressure relief valve 170 will open to the open position or to the at least one partially open position.

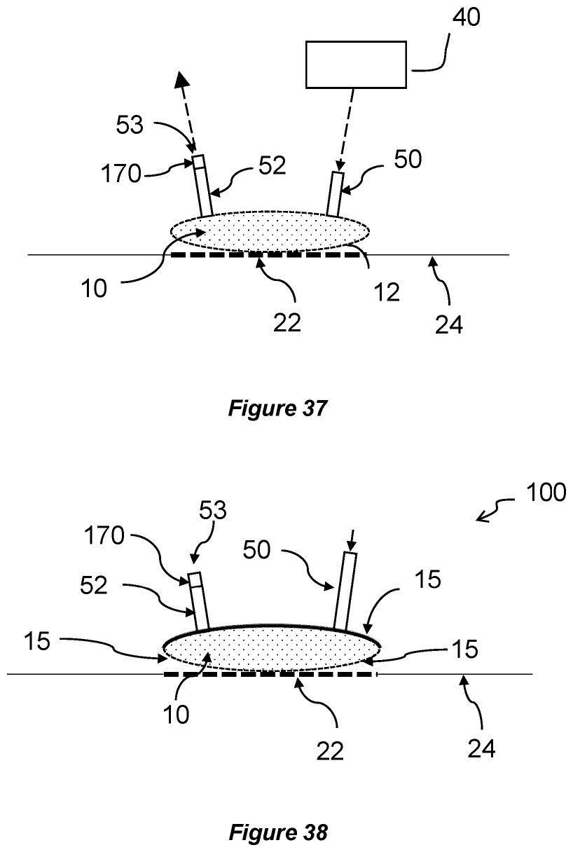

FIG. 37 shows a variation of the embodiment of FIG. 36 in which the cover 30 is not present such that there is no compartment 35. The pressure relief valve 170 is positioned at the fluid outlet 53 of the first fluid outlet conduit 52, in fluid communication with the receptacle 10 as in the embodiment of FIG. 36.

FIG. 38 shows a variation of the embodiment of FIG. 37 in which a wall 15 has a first layer and a second layer. The second layer of the wall 15 of the receptacle 10 faces away from the wound 22 and is made of a material having a thickness that is greater than a thickness of a first layer of the wall 15 that faces toward the wound 22. The thicker material may reduce the loss of fluid through areas of the wall 15 that do not face the wound 22 and may result in an increase in efficiency of the apparatus 100. The first layer of the wall 15 of the receptacle 10 may have the wall thickness as described above for the receptacle 10; that is between about 10 micrometres and about 150 micrometres, for example between approximately 20 micrometres and 140 micrometres, or between approximately 30 micrometres and 130 micrometres, or between approximately 35 micrometres and 100 micrometres, or between approximately 40 micrometres and 80 micrometres, or between approximately 40 micrometres to 70 micrometres, or between approximately 30 micrometres to 60 micrometres, or between approximately 20 to 60 micrometres, or between approximately 45 micrometres and 55 micrometres. In some embodiments, the first layer of the wall 15 may have a wall thickness of approximately 60 micrometres. In some embodiments, the first layer of the wall 15 may have a wall thickness of approximately 50 micrometres. The second layer of the wall 15 may have a wall thickness of between about 75 micrometres and about 1 mm, for example between approximately 80 micrometres and 800 micrometres, or between approximately 85 micrometres and 600 micrometres, or between approximately 90 micrometres and 400 micrometres, or between approximately 95 micrometres and 300 micrometres, or between approximately 100 micrometres and 200 micrometres, or between approximately 125 micrometres and 175 micrometres. In some embodiments, the wall thickness is approximately 150 micrometres. The wall thickness may be greater than 1 mm provided that the second layer remains flexible such that the apparatus 100 inflates and conforms to the underlying patient tissue at the wound 22. It will be apparent to the skilled person that the thicker material of the second layer of the wall 15 may be used with any one of the embodiments of the receptacle 10, 110, 210 310, 410 disclosed herein and this disclosure extends also to those embodiments.

Whilst the pressure regulator of FIG. 36 to FIG. 38 is described in the form of a pressure relief valve 170, the pressure regulator may take other forms to achieve a positive pressure within the receptacle 10. Whilst the pressure regulator is described here with respect to the receptacle 10, it will be apparent to the skilled person that the following applies to all embodiments of the disclosure that include a pressure regulator for regulating fluid pressure within the receptacle 110, 10, including where a pressure regulator is used with the receptacles 210, 210A, 310, 410. The pressure regulator, including the pressure relief valve 170, may be passively and/or mechanically actuable to permit fluid to pass therethrough, i.e. the pressure regulator is not powered. Alternative pressure regulators to the pressure relief valve 170 may include, for example, that the first fluid outlet conduit 52 may have a small internal diameter and/or a long length that would generate a large resistance to fluid flow exiting the receptacle 10 and a resultant positive pressure in the receptacle 10. In practice, the length and the internal diameter of the first fluid outlet conduit 52 will be determined based on the target fluid pressure in the receptacle 10. The skilled person in the art would know to vary the length and/or the internal diameter in order to achieve a target pressure. Similarly, a series of baffles or changes of direction of the first fluid outlet conduit 52 may be used to form a tortuous section of flow path within the first fluid outlet conduit 52 to generate a large resistance to fluid flow, resulting in a positive pressure in the receptacle 10. The tortuous section could be formed in a block of material or by manipulating the shape of the first fluid outlet conduit 52. A further alternative is to utilise a Tesla valve in the flow outlet conduit 52 to generate a large resistance to fluid flow within the first fluid outlet conduit 52.

A calibrated leak orifice similar to the calibrated leak orifice 74 seen in FIG. 8 may be used at the fluid outlet 53 to permit a specified leak, which may result in a positive pressure in the receptacle 10. The calibrated leak orifice may be calibrated to provide a leak out of the receptacle 10 that is small enough to maintain a positive pressure within the receptacle 10, but large enough to reduce the chance of pressure within the receptacle 10 becoming undesirably high.

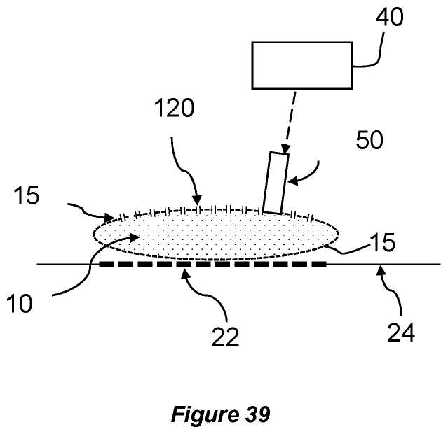

FIG. 39 shows an embodiment of a receptacle 10 in which cycling of fluid within the receptacle 10 may be achieved by incorporating at least one aperture 120 in the second layer of the wall 15, that are configured to permit fluid within the receptacle 10 to pass therethrough to exit the receptacle 10. The at least one aperture 120 may comprise a plurality of apertures. The size and/or number of the at least one aperture 120 may be tailored to allow cycling of fluid from within the receptacle 10 to the atmosphere whilst providing enough resistance to fluid flow to achieve a positive pressure in the receptacle 10. The at least one aperture 120 may also help to prevent the pressure within the receptacle 10 from becoming too high and to relieve pressure within the receptacle 10 during use of the apparatus. The at least one aperture 120 may be used where the apparatus does not include a compartment 35. The at least one aperture 120 functions most effectively in the absence of a negative pressure compartment. In this embodiment, the at least one aperture 120 may be used instead of the first fluid outlet conduit 52.

The at least one aperture 120 may have a border of thicker material to prevent tearing and to maintain the integrity of the material of the second layer surrounding the at least one aperture 120. The border of thicker material may comprise a ring of raised material surrounding the at least one aperture 120 and the border of thicker material may be of any reasonable cross-sectional shape, for example a semi-oval shape, or a rounded rectangular shape or a semi-octagonal shape. The border of thicker material surrounding the at least one aperture 120 may improve the structural integrity of the material of the second layer surrounding the at least one aperture 120 and help to prevent any tear in the at least one aperture 120 from propagating beyond the border.

Whilst the pressure relief valve 170 or other pressure regulator or calibrated leak orifice or the at least one aperture 120 are described with reference to the receptacle 10, any of them may apply also to any of the receptacles 110, 210, 210A, 310, 410 described in this disclosure.

FIGS. 40 to 44 show an embodiment of a receptacle that is similar to the receptacle 110 of FIG. 26, and like features are given like reference numbers. The receptacle 110 has a longitudinal axis X-X that extends through the head portion 93 and the tail portion 90 of the receptacle 110. In the embodiment of the receptacle 110 shown in FIG. 40 and FIG. 41, an internal flow guide 140 in the form of an internal wall. The internal wall extends along the longitudinal axis X-X to form two compartments, one either side of the internal wall. The compartments are arranged in a layered or stacked configuration with a first wall portion of the wall 115 (a lower portion of the wall as viewed in FIG. 40) and a second wall portion of the wall 115 (an upper portion of the wall as viewed in FIG. 40) of the receptacle 110. That is, the internal flow guide 140 has a first flow guide surface 142 that is configured to face the wound 22 and/or healthy skin 24 surrounding the wound 22 during use, and a second flow guide surface 144 that opposes the first flow guide surface 142 and faces away from the wound 22 and/or healthy skin 24 surrounding the wound during use.

Accordingly, a layered or stacked configuration of the compartments refers to a first compartment formed between the first wall portion and the first flow guide surface 142 of the internal flow guide 140 and a second compartment formed between the second flow guide surface 144 and the second wall portion of the wall 115. Whilst the internal flow guide 140 of FIG. 40 extends along the longitudinal axis X-X, in an alternative embodiment it may extend parallel to the longitudinal axis X-X so as to be offset from it. In such an embodiment, the internal flow guide 140 divides the receptacle 110 into two compartments of different sizes. The first wall portion and the second wall portion of the wall 115 are flexible such that, prior to use, the internal flow guide 140 may lie substantially parallel with the first wall portion and the second wall portion of the wall 115 of the receptacle 110, however, in use, of the receptacle 110, the first wall portion and the second wall portion of the wall 115 may be contorted or bent to accommodate a complex wound shape or to fit onto a contoured part of the body, for example, a heel or knee. The internal flow guide 140 extends across the full lateral extent of the receptacle and may be sealed at its periphery to the one or more walls 115 of the receptacle 110. The internal flow guide 140 has a circulation opening 145 towards an end of the internal flow guide 140 that is positioned in the head portion 93 of the receptacle 110. The circulation opening 145 provides a fluid pathway through the receptacle 110 and between the two compartments, as will be described further.

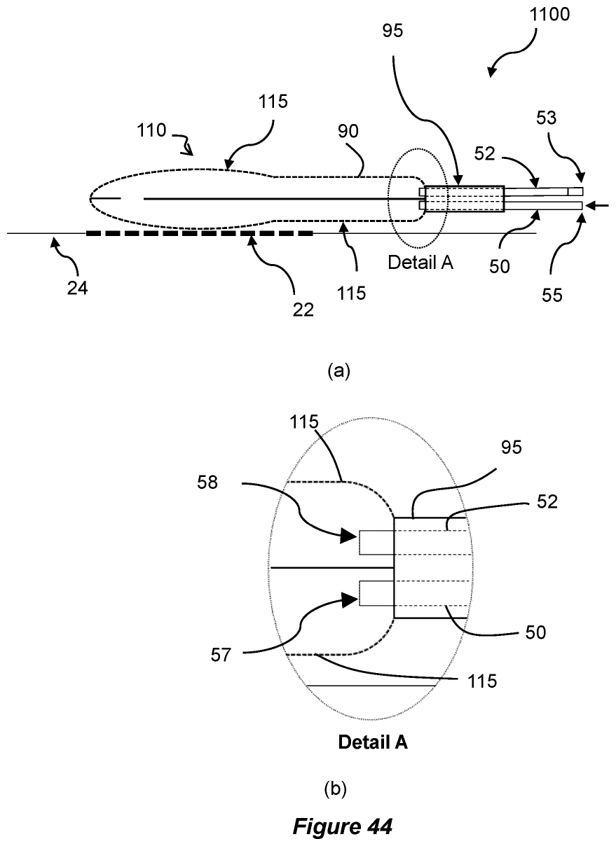

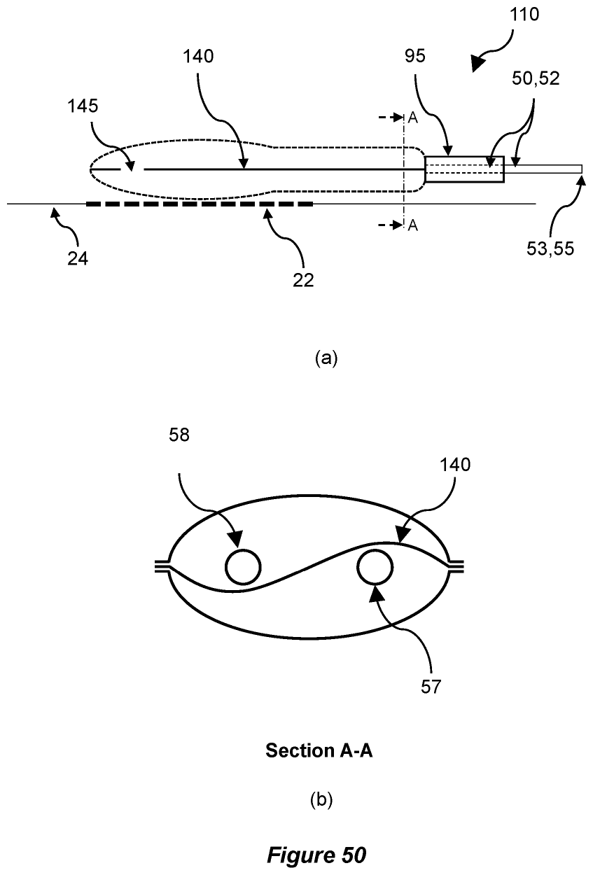

As best seen in FIG. 44, the receptacle 110 has a fluid inlet conduit 50 and a first fluid outlet conduit 52, each of which passes through the pressure spreading device 95 and through which fluid respectively enters and exits the receptacle 110. However, in embodiments of the receptacle 10 that do not include a pressure spreading device 95, the fluid inlet conduit 50 and the first fluid outlet conduit 52 pass directly into the one or more walls 15 of the receptacle 10. In FIG. 40, the fluid inlet conduit 50 is shown beneath the first fluid outlet conduit 52, however the converse arrangement may also be used where the fluid inlet conduit 50 is above the first fluid outlet conduit 52. The fluid inlet conduit 50 and the first fluid outlet conduit 52 may also be disposed side by side, as will be described with respect to FIGS. 50(a) and 50(b). The fluid outlet 53 has a pressure regulator such as a pressure relief valve 170 disposed on it for relieving pressure inside the receptacle 110.

The one or more walls 115, the fluid inlet 55, the fluid outlet 53 and the internal flow guide 140 are configured to define a fluid flow path through the receptacle 110 that extends from the fluid inlet 55, past at least a portion of the internal flow guide 140, and out of the fluid outlet 53. In use of the apparatus 1100, fluid enters the receptacle 110 via the fluid inlet 55 of the fluid inlet conduit 50, and flows along the part of the fluid flow path defined by the major portion of the first flow guide surface 142 that lies upstream of the circulation opening 145 and the first wall portion of the wall 115, through the circulation opening 145 and along the part of the fluid flow path defined by the major part of the second flow guide surface 144 and out of the fluid outlet 53 of the first fluid outlet conduit 52 via the pressure relief valve 170 (or other pressure regulator as described above). As such, the internal flow guide 140 may prolong a residence time in which fluid entering the fluid inlet 55 spends within the receptacle 110 prior to exiting the receptacle 110 through the fluid outlet 53, relative to a receptacle 110 that does not include an internal flow guide 140. The internal flow guide 140 effectively divides the receptacle 110 into first and second compartments that are fluidly connected by the circulation opening 145. The internal flow guide 140 may permit molecules within the fluid within the receptacle 110 to pass through it and may be made of the same material as the one or more walls 115 of the receptacle 110. However, the internal flow guide 140 may be made of a different material and that material may not allow molecules within the fluid within the receptacle 110 to pass through it.

In the embodiment of FIG. 40 and FIG. 41, the fluid inlet conduit 50 and the first fluid outlet conduit 52 are disposed at the same end of the receptacle 110. This arrangement is advantageous in that it facilitates ease of handling a dressing, for example a bandage, and the fluid inlet conduit 50, first fluid outlet conduit 52, and any further tubes that are connected to them, during application of the dressing over a wound 22. In particular, the fluid inlet conduit 50 and the first fluid outlet conduit 52 may be handled in one hand as the receptacle 110 is applied onto the wound 22, leaving the other hand free to apply a dressing or to wind the bandage over the receptacle 110 and the fluid inlet conduit 50 and first fluid outlet conduit 52, and around a limb or other body part having the wound 22. The user must take care when applying the dressing not to trap the fluid inlet conduit 50 and the first fluid outlet conduit 52 such that they dig into the healthy skin 24 surrounding the wound 22 and cause discomfort to the patient. As a result of the fluid inlet conduit 50 and the first fluid outlet conduit 52 being disposed at the same end of the receptacle 110, the fluid inlet conduit 50 and the first fluid outlet conduit 52 can be managed together, minimising the possibility of causing such discomfort. This aspect of the receptacle 110 applies also to the embodiment of the receptacle 10 shown in FIG. 16b, described above, in which the fluid inlet conduit 50, first fluid outlet conduit 52 and the second fluid outlet conduit 54 are arranged adjacent one another at the same section of the receptacle 10. The arrangement also encourages an even dispersal of fluid through the receptacle 110, as fluid must travel from the fluid inlet 55 of the fluid inlet conduit 50, along a majority of the length of the receptacle 110, through the circulation opening 145 and back along the majority of the length of the receptacle 110 before it may exit the fluid outlet 53 and via the first fluid outlet conduit 52.

It is noted that whilst the above description refers to the first flow guide surface 142 being configured to face the wound 22 and/or healthy tissue skin 24 surrounding the wound 22 during use, and the second flow guide surface 144 opposing the first flow guide surface 142 and facing away from the wound 22 and/or healthy skin 24 surrounding the wound during use, the description simply refers to the orientation of the receptacle as it is shown in FIG. 40. In practice, the receptacle 110 may equally be used in an orientation in which the first flow guide surface 142 is configured to face away from the wound 22 and/or healthy tissue skin 24 surrounding the wound 22 during use, and the second flow guide surface 144 opposing the first flow guide surface 142 faces the wound 22 and/or healthy skin 24 surrounding the wound. The one or more walls 115 of the receptacle 110 may also have the same properties regardless of orientation of the receptacle 110. The receptacle 110 may accordingly be used in either orientation in use, making the receptacle 110 user friendly. Furthermore, whilst the features of the internal flow guide 140 are described with reference to the receptacle 10, it will be apparent that one or more of the features of the internal flow guide 140 may apply to any of the receptacles 10, 110, 210, 210A, 310, 410 described in this disclosure.

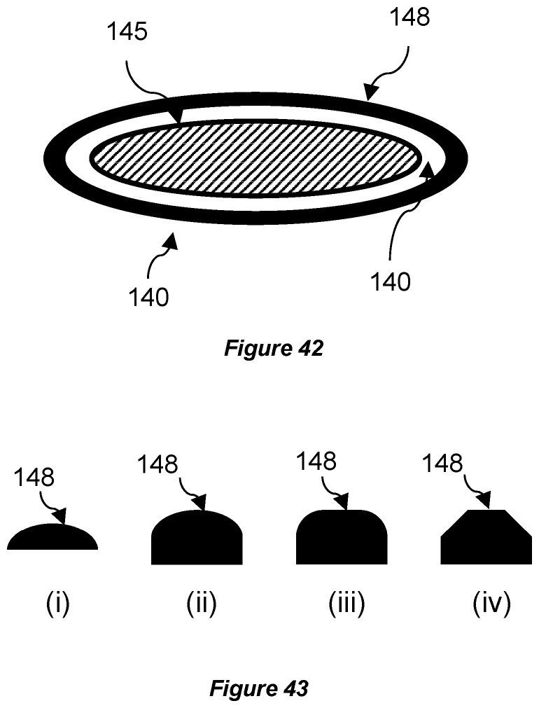

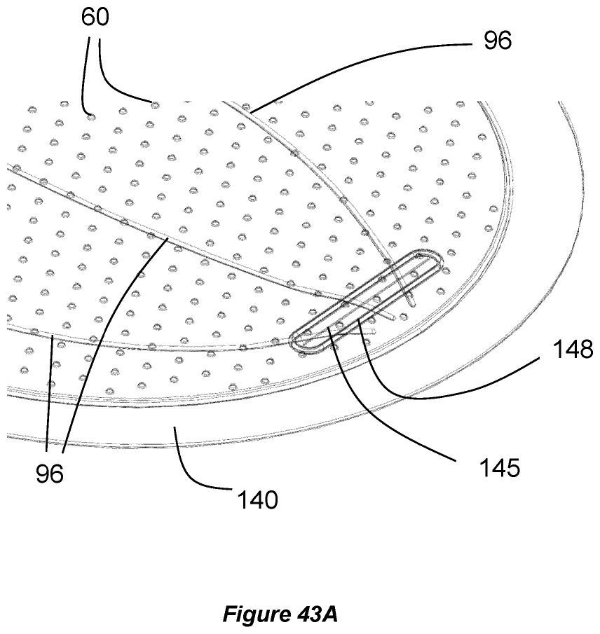

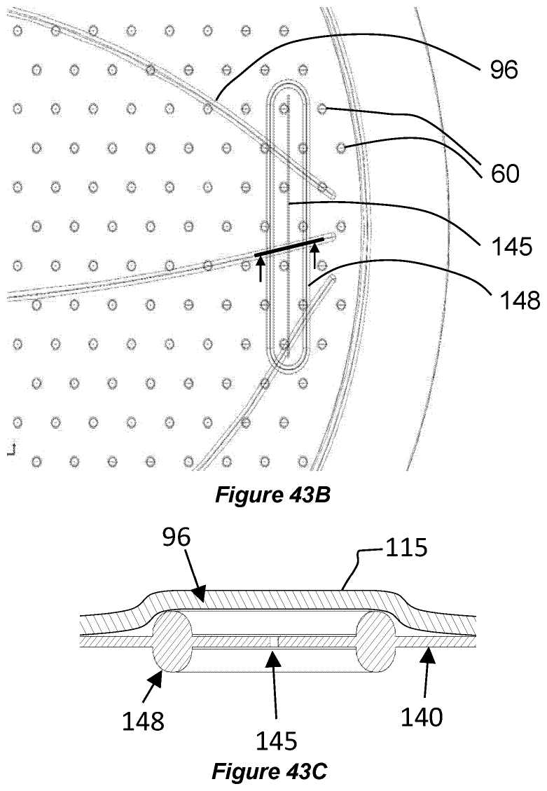

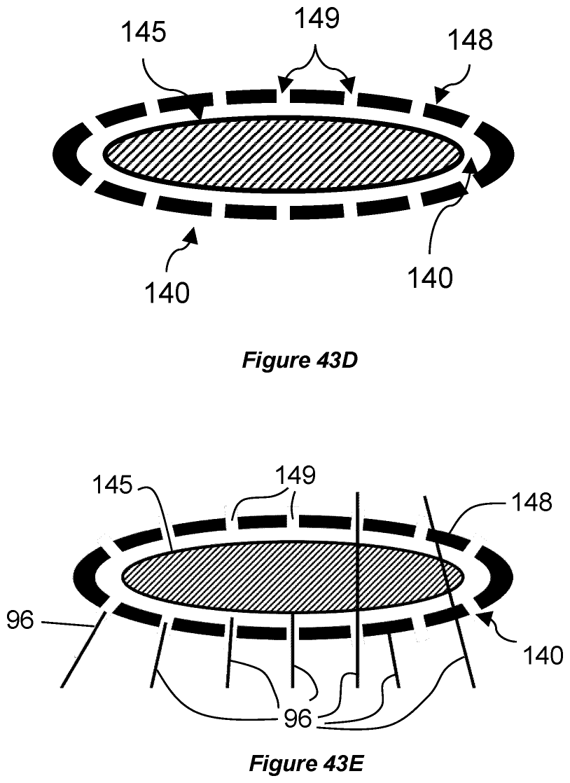

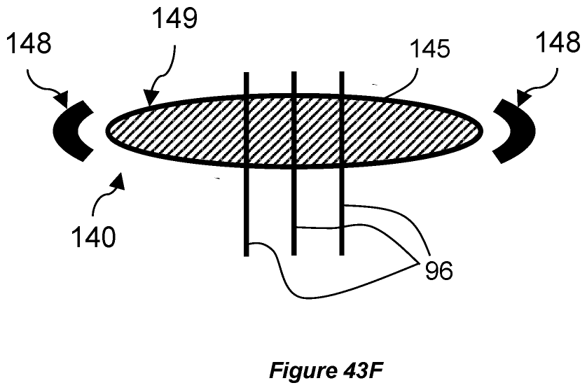

The circulation opening 145 of the internal flow guide 140 is shown in plan view in FIG. 41. In FIG. 41 it has an oval shape, however it may have any reasonable shape such as a circle, rectangle or slit. For example, in FIGS. 43A to 43C, the circulation opening 145 is shaped as a slot. It may have a width dimension that is sufficiently large to allow fluid to pass through it across a significant portion of the interior width of the receptacle 110, across the plurality of ridges 96. A width dimension of the circulation opening 145 may be between 5% and 95% of an interior width of the receptacle 110 when not inflated. In an example, the width of the circulation opening 145 may be between 15% and 85% or between 25% and 75% or between 30% and 60% of the interior width of the receptacle 110 when not inflated. The circulation opening 145 may be positioned at an end of the receptacle 110 that is distal from the fluid inlet conduit 50 and the first fluid outlet conduit 52 as shown in FIG. 41. The circulation opening 145 may be surrounded by an opening surround 148, as seen in FIG. 42. FIG. 42 is an enlarged plan view of the circulation opening 145 and its opening surround 148. The opening surround 148 may comprise a ring of raised material, such as a rib. The raised material may rise from one of the first flow guide surface 142 or the second flow guide surface 144 or both of the first flow guide surface 142 and the second flow guide surface 144. The ring of raised material may have any reasonable cross-sectional shape. Examples of possible cross-sectional shapes of the ring of raised material are shown in FIG. 43. In FIG. 43(i), the ring of raised material of the opening surround 148 has a shallow semi-oval cross-sectional shape. In FIG. 43(ii), the ring of raised material of the opening surround 148 has a deeper ovoid cross-sectional shape. In FIG. 43(iii), the ring of raised material of the opening surround 148 has a rounded rectangular cross-sectional shape. The rounded rectangular cross-sectional shape is easy to manufacture and does not have any sharp corners that may cause discomfort to the patient. In FIG. 43(iv), the ring of raised material of the opening surround 148 has a semi-octagonal cross-sectional shape. A variation of this embodiment may have rounded corners rather than the sharp corners shown in FIG. 43(iv). The opening surround 148 may improve the structural integrity of the material surrounding the circulation opening 145 and help to prevent any tear in the internal flow guide 140 at the circulation opening 145 from propagating too far and substantially increasing the size of the circulation opening 145.

FIG. 43A to FIG. 43C illustrate how the ridges 96 of the first wall portion and/or the second wall portion of the wall 115 may overlap the opening surround 148. The ridges 96 may alternatively or additionally be present on the first flow guide surface 142 and/or the second flow guide surface 144 of the internal flow guide 140. The overlapping of the ridges 96 with the opening surround 148 helps to maintain the fluid flow path inside the receptacle 10, 110 ,210, 210A, 310, 410, and fluid may flow along the side of the ridges 96 as described above and over the opening surround 148 into the circulation opening 145. In FIG. 43A and FIG. 43B, the ridges 96 are shown on the second wall portion of the wall 115. The microstructures 60 are also visible on the second wall portion of the wall 115. The opening surround 148 on the second flow guide surface 144 of the internal flow guide 140 is shown overlapping with some of the ridges 96. FIG. 43C is a view of the circulation opening 145, opening surround 148 and ridge 96 of FIG. 43B and illustrates clearly how the ridge 96 overlaps the opening surround 148 at two points, either side of the circulation opening 145.

The opening surround 148 may have one or more gaps or discontinuities 149 as shown in FIG. 43D to FIG. 43F. The gaps 149 in the opening surround 148 may assist fluid within the receptacle 10,110, 210, 210A, 310, 410 to flow past the opening surround 148 and through the circulation opening 145. The opening surround 148 may include a plurality of the gaps 149. The gaps 149 may be evenly spaced around the opening surround 148 as shown in FIGS. 43D and 43E or they may be unevenly spaced. At least some of the gaps 149 may be aligned with one another across the circulation opening 145.

As shown by way of example in FIG. 43E, some of the ridges 96 of the first wall portion and/or the second wall portion of the wall 115 may terminate at the gaps 149 or at the opening surround 148. Alternatively, some of the ridges 96 may terminate at the circulation opening 145. Some of the ridges 96 may pass through the gaps 149 and continue to the opposite side of the circulation opening 145 and/or may pass through a gap 149 on the opposite side of the opening surround 148. Ridges 96 that pass through gaps 149 in the opening surround 148 may be less prone to creating a pressure point on the tissue of the patient in use. The ridges 96 in the vicinity of any one circulation opening 145 may include a mix of termination points as shown in the example of FIG. 43E.

FIG. 43F shows an embodiment of an opening surround 148 that comprises only sections at the extremities of the circulation opening 145, where the corners are sharp. The sections of the opening surround 148 assist in preventing tears in the material of the internal flow guide 140 at the sharp corners of the circulation opening 145 from propagating through the internal flow guide 140. In this embodiment, one or more of the ridges 96 may extend over the circulation opening 145.

The internal flow guide 140 may include more than one of the circulation openings 145. Having more than one circulation opening 145 can be beneficial in the event that one of the circulation openings 145 becomes blocked, fluid is still able to pass through another circulation opening 145 to reach the fluid outlet 53. The internal flow guide 140 may include a section that is porous such that it provides a flow path through which fluid may flow. The porous section may be distal from the fluid inlet conduit 50.

FIGS. 44a and 44b shows how the fluid inlet conduit 50 and the first fluid outlet conduit 52 attach to the tail portion 90 of the receptacle 110. As shown in FIGS. 40 to 43, the fluid inlet conduit 50 and the first fluid outlet conduit 52 are encapsulated in the pressure spreading device 95 and extend into the tail portion 90 of the receptacle. FIGS. 44a and 44b show further detail of the termination of the fluid inlet conduit 50 and the first fluid outlet conduit 52 at the tail portion 90. The ends of the fluid inlet conduit 50 and the first fluid outlet conduit 52 respectively pass through the first wall portion and the second wall portion of the wall 115 of the receptacle 110 and into the interior of the receptacle 110. In particular, the ends of the fluid inlet conduit 50 and the first fluid outlet conduit 52 are integrally formed, for example moulded, into the first wall portion and the second wall portion of the at least one wall 115 of the receptacle 110 such that an outlet 57 of the fluid inlet conduit 50 and an inlet 58 of the first fluid outlet conduit 52 are disposed inside the tail portion 90 of the receptacle 110. The outlet 57 and the inlet 58 may take the form of a port, or an end of a fluid inlet conduit or first fluid outlet conduit that protrudes into the tail portion 90 of the receptacle 110. Alternatively, the outlet 57 and the inlet 58 may each comprise of an opening that allows delivery of the fluid from the fluid inlet conduit 50 to the receptacle 110, or from the receptacle 110 to the first fluid outlet conduit 52, by maintaining fluid communication between the receptacle 110 and the respective one of the fluid inlet conduit 50 and the first fluid outlet conduit 52. In use, fluid is supplied to the receptacle 110 via the outlet 57 of the fluid inlet conduit 50 and fluid exits the receptacle 110 through the inlet 58 of the first fluid outlet conduit 52. This attachment of the fluid inlet conduit 50 and the first fluid outlet conduit 52 to the receptacle 110 may apply to all embodiments of the receptacle 110 disclosed herein. For embodiments of the receptacle 10, 110, 210, 210A, 310, 410 that may not include a pressure spreading device 95, the fluid inlet conduit 50 and the first fluid outlet conduit 52 may be integrally formed with and pass directly through the first wall portion and the second wall portion of the one or more walls 15 such that their ends extend into the interior of the receptacle 10, 110, 210, 210A, 310, 410. For example, they may be moulded into the one or more walls 15. Alternatively, the fluid inlet conduit 50 and the first fluid outlet conduit 52 may terminate within the pressure spreading device 95 and do not pass through the one or more walls 15 and into the receptacle 10.

A pressure regulator such as a pressure relief valve 170 may be added to the fluid inlet 55 of the fluid inlet conduit 50, either in addition to or instead of at the fluid outlet 53. The embodiment of the apparatus of FIGS. 40 to 44 may be used with or without the cover 30, which if used may form a compartment 35 which can be a negative pressure compartment or it may comprise a bandage or compression bandage.

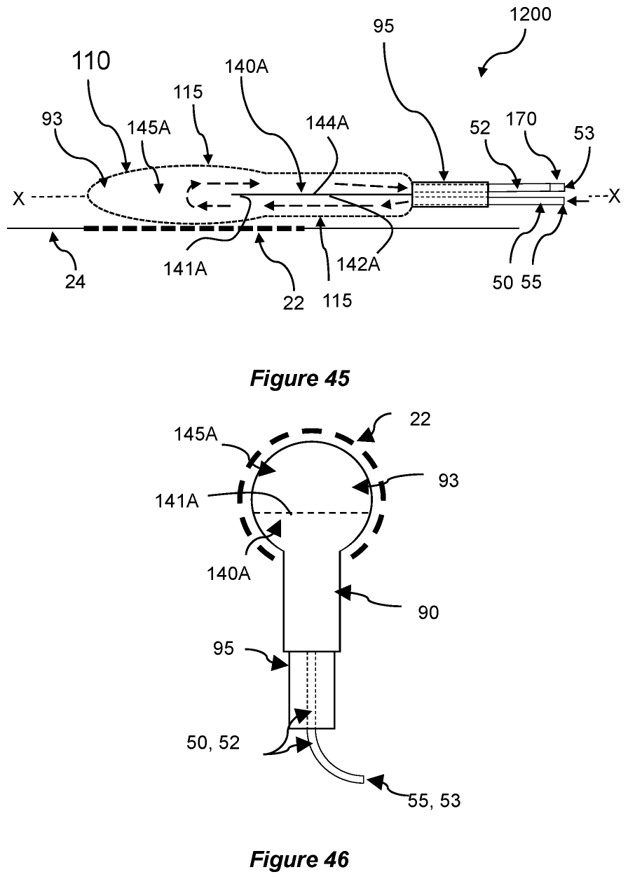

FIG. 45 and FIG. 46 show a variation of the embodiment of FIGS. 41 to 44 in which the circulation opening 145 is replaced with an opening that takes the form of a larger circulation opening or gap 145A. In this embodiment, the internal flow guide 140A does not extend the entire longitudinal axis X-X of the receptacle 110, but extends through the tail portion 90 and part way into the head portion 93 where it terminates. The circulation gap 145A spans the distance between an end 141A of the internal flow guide 140A and the wall 115 of the receptacle 110 at the head portion 93 of the receptacle 110. This embodiment has the benefit of ease of manufacture. The internal flow guide 140A has a first flow guide surface 142A that is configured to face the wound 22 and/or healthy skin 24 surrounding the wound 22 during use, and a second flow guide surface 144A that opposes the first flow guide surface 142A and faces away from the wound 22 and/or healthy tissue skin 24 surrounding the wound 22 during use. Prior to use, the internal flow guide 140A may lie substantially parallel with the first wall portion and the second wall portion of the wall 115 of the receptacle 110. However in use of the receptacle 110, the first wall portion and the second wall portion of the wall 115 is flexible and may be contorted or bent to accommodate a complex wound shape or to fit onto a contoured part of the body, for example a heel or a knee.

The internal flow guide 140A effectively divides the receptacle 110 into first and second compartments that are fluidly connected by the circulation gap 145A. In use of the apparatus 1200, fluid enters the receptacle 110 via the fluid inlet 55 of the fluid inlet conduit 50, and flows along the part of the fluid flow path defined by the first flow guide surface 142A that lies upstream of the circulation gap 145A and the first wall portion of the wall 115, through the circulation gap 145A and along the part of the fluid flow path defined by the part of the second flow guide surface 144A and out of the fluid outlet 53 of the first fluid outlet conduit 52 via the pressure relief valve 170 (or other pressure regulator as described above).

The fluid inlet conduit 50 and the first fluid outlet conduit 52 are disposed at the same end of the receptacle 110. This arrangement is advantageous in that it facilitates ease of handling a dressing, for example a bandage, and the fluid inlet conduit 50, fluid outlet conduit 52, and any further tubes that are connected to them, during application of the dressing over the wound, as in the embodiment of FIG. 41. The arrangement also encourages an even dispersal of fluid through the receptacle 110, as fluid must travel from the fluid inlet 55 of the fluid inlet conduit 50, along a majority of the length of the receptacle 110, through the circulation gap 145A and back along the majority of the length of the receptacle 110 before it may exit the fluid outlet 53 via the first fluid outlet conduit 52. Whilst the features of the internal flow guide 140A are described with reference to the receptacle 110, it will be apparent that one or more of the features of the internal flow guide 140A may apply to any of the receptacles 10, 110, 210, 210A, 310, 410 described in this disclosure.

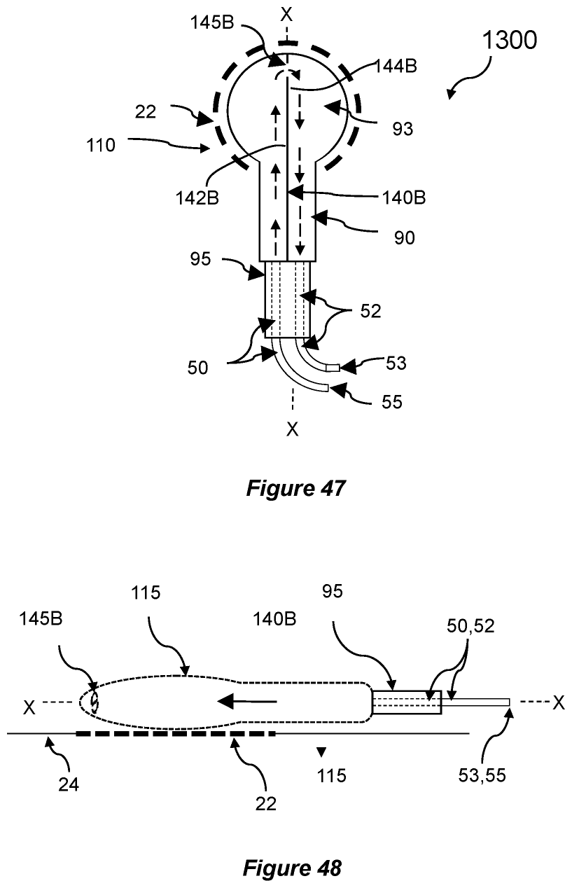

A further variation of the embodiment of FIGS. 40 to 44 is shown in FIGS. 47 and 48. FIG. 47 shows a plan view of a receptacle 110 that has a longitudinal axis X-X that extends through the head portion 93 and the tail portion 90 of the receptacle 110. FIG. 48 is a cross-sectional side view of the receptacle 110. In the embodiment of the receptacle 110 shown in FIG. 47 and FIG. 48, an internal flow guide 140B (best seen in FIG. 47) in the form of an internal wall extends along the longitudinal axis X-X so as to extend between the first wall portion and the second wall portion of the wall 115 of the receptacle 110. The internal flow guide 140B is arranged to form side by side compartments within the receptacle 110 rather than upper and lower compartments and bisects the first wall portion and the second wall portion of the wall 115 at its upper and lower bounds, respectively. That is, the internal flow guide 140B has an upper edge that adjoins the second wall portion of the wall 115 of the receptacle 110 and a lower edge that adjoins a first wall portion of the wall 115 of the receptacle 110 during use. The internal flow guide 140B has a first flow guide surface 142B and a second flow guide surface 144B that opposes the first flow guide surface 142B. Accordingly, a portion of the first wall portion of the wall 115 of the receptacle 110 that faces and may come into contact with the tissue of the patient during use is a tissue facing portion of the first wall portion of the wall 115, and is disposed adjacent both the first flow guide surface 142B and the second flow guide surface 144B such that fluid flowing through the receptacle 110 passes the first wall portion of the wall 115 as it passes the first flow guide surface 142B and also the second flow guide surface 144B. The internal flow guide 140B may bisect the first wall portion of the wall 115 into two equal portions, or alternatively the internal flow guide 140B may be disposed parallel to the longitudinal axis X-X so that it is offset from it and thus divides the first wall portion of the wall 115 into two compartments, wherein the compartments are different sizes. The internal flow guide 140B may also be disposed at an angle to the longitudinal axis X-X such that the two compartments are not symmetrical about the longitudinal axis X-X.

The internal flow guide 140B has a circulation opening 145B that is the same as the circulation opening 145. The circulation opening 145B is positioned towards an end of the internal flow guide 140B that is positioned in the head portion 93 of the receptacle 110. The circulation opening 145B provides a fluid pathway through the receptacle 110 in the same manner as the circulation opening 145.

The receptacle 110 has a fluid inlet conduit 50 and a first fluid outlet conduit 52, each of which passes through the pressure spreading device 95 and through which fluid respectively enters and exits the receptacle 110. In FIG. 47, the fluid inlet conduit 50 is shown adjacent the first fluid outlet conduit 52 such that they lie side by side. The first fluid outlet conduit 52 may have a pressure regulator such as pressure relief valve 170 disposed on it for relieving pressure inside the receptacle 110.

The internal flow guide 140B effectively divides the receptacle 110 into first and second compartments that are disposed side by side and that are fluidly connected by the circulation opening 145B. In use of the apparatus 1300, fluid enters the receptacle 110 via the fluid inlet 55 of the fluid inlet conduit 50, and flows along the part of the fluid flow path defined by the first flow guide surface 142B that lies upstream of the circulation opening 145B and the first wall portion and second wall portion of the wall 115 of the receptacle 110, through the circulation opening 145B and along the part of the fluid flow path defined by the major part of the second flow guide surface 144BA and the first wall portion and the second wall portion of the wall 115 out of the fluid outlet 53 of the first fluid outlet conduit 52 via the pressure relief valve 170 (or other pressure regulator as described above). Whilst the features of the internal flow guide 140B are described with reference to the receptacle 110, it will be apparent that one or more of the features of the internal flow guide 140B may apply to any of the receptacles 10, 110, 210, 210A, 310, 410 described in this disclosure.

In use of the receptacle 110 according to the embodiments of FIG. 47 and FIG. 48, the fluid flow path extends past the first wall portion of the wall 115 of the receptacle 110 both upon entering the receptacle 110 and when exiting it, which may result in prolonging a period of time that the fluid within the receptacle 110 spends adjacent the membrane of the wall 115 receptacle 110.

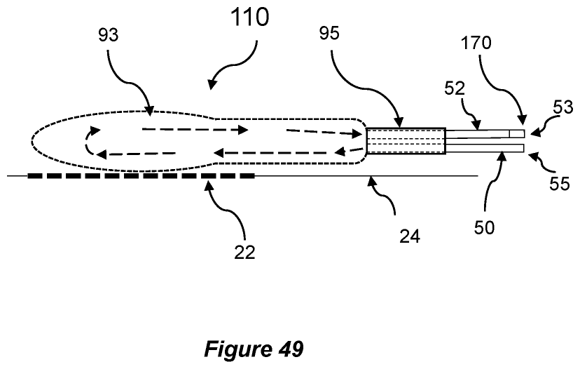

In each of the embodiments of the receptacle 110 shown in FIGS. 40 to 48, the internal flow guide 140, 140A, 140B prevents fluid entering the receptacle 110 from simply exiting through the fluid outlet 53 before it has travelled over a significant portion of the internal wall area, thereby allowing fluid to diffuse through a significant portion of the membrane of the wall 115 to more of the wound area 20. However, it is also envisaged that the receptacle 110 may have no internal flow guide 140, 140A, 140B as shown in the embodiment of FIG. 49. The fluid inlet conduit 50 and the first fluid outlet conduit 52 are disposed at the same end of the receptacle 110. This arrangement allows ease of handling the fluid inlet conduit 50 and the first fluid outlet conduit 52 and of applying a dressing over the fluid inlet conduit 50, the first fluid outlet conduit 52, and any further tubes that are connected to them, as with the embodiments of FIGS. 40 to 48. A pressure regulator such as a pressure relief valve 170 is disposed at the fluid outlet 53 for relieving pressure within the receptacle 110 as has been described in relation to the embodiments of FIGS. 36 to 39.

FIG. 50(a) and FIG. 50(b) show an example of a receptacle 110 that is similar to that of the embodiment of FIGS. 40 to 44, and which has an internal flow guide 140 configured in a layered or stacked configuration. However, in the embodiment of FIGS. 50(a) and (b), the fluid inlet conduit 50 and the first fluid outlet conduit 52 are arranged in a side by side configuration such that the outlet 57 of the fluid inlet conduit 50 and the inlet 58 of the first fluid outlet conduit 52 lie side by side within the interior of the receptacle 110 and/or within the pressure spreading device 95. The internal flow guide 140 separates the outlet 57 from the inlet 58. As shown in FIG. 50(b), this is achieved by positioning the internal flow guide 140 to lie underneath the inlet 58 of the fluid inlet conduit 52 and over the outlet 57 of the first fluid outlet conduit 50. However, it may also be achieved by positioning the internal flow guide 140 to lie over the inlet 58 and underneath the outlet 57. The side by side arrangement of the fluid inlet conduit 50 and the first fluid outlet conduit 52 spreads the forces applied by the fluid inlet conduit 50 and the first fluid outlet conduit 52 on the tissue of the patient over a larger area of tissue than if they are arranged one on top of the other, and thus reduces the pressure applied to one portion of the tissue. As a result, the overall pressure experienced by the patient from the fluid inlet conduit 50 and the first fluid outlet conduit 52 is reduced as it is spread over a wider area, which may result in a more comfortable experience for the patient. Whilst the features of the internal flow guide 140 are described with reference to the receptacle 110, it will be apparent that one or more of the features of the internal flow guide 140 may apply to any of the receptacles 10, 110, 210, 210A, 310, 410 described in this disclosure.

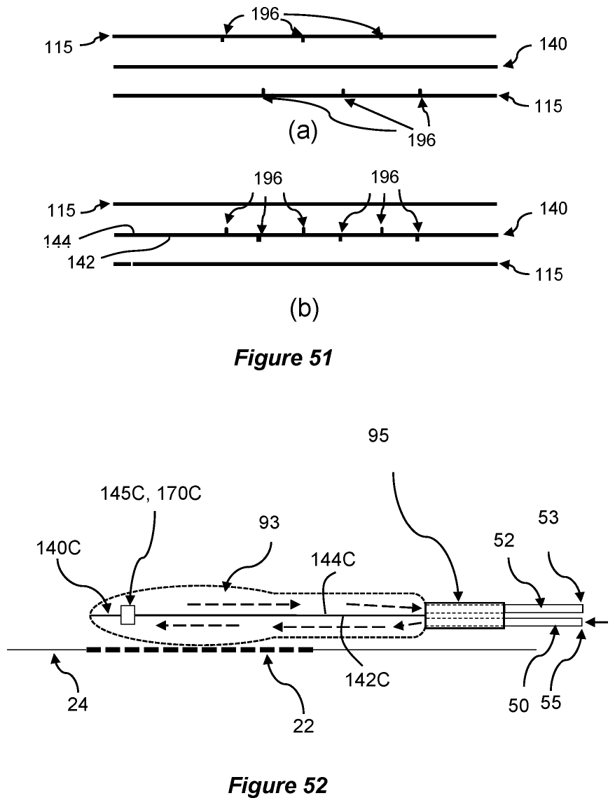

In any one of the embodiments shown in FIGS. 40 to 49, the receptacle 110 may include a plurality of ridges as described in relation to FIGS. 31 to 34 above. The disclosure in relation to the embodiments described in relation to FIGS. 31 to 34 applies also to the embodiments of FIGS. 41 to 49. FIG. 51(a) and FIG. 51(b) each show a cross sectional view through the head portion 93, such as at line A-A of FIG. 41, of an embodiment of the inside surface of at least one wall 115 of a receptacle 110. As shown in FIGS. 51(a) and 51(b), the receptacle 110 includes the internal flow guide 140 of the embodiment of FIGS. 40 to 44, however it may equally be the internal flow guide 140A of FIGS. 45 and 46 or the internal flow guide 140B of the embodiments of FIGS. 47 and 48. The inner surface of the wall 115 includes one or more small, soft, protruding ridges 196. The ridges 196 have the same properties as the ridges 96 described in relation to FIGS. 31 to 34 above and the same disclosure applies.

The ridges 196 may be positioned on opposing inner surfaces of the first wall portion and second wall portion of the one or more walls 115 as shown in FIG. 51(a), such that when they are pressed together, the internal flow guide 140 opposing the ridge 196 drapes over the ridge 196, leaving gaps on either side of it in the manner described above in relation to FIG. 31b. Alternatively, the ridges 196 may be positioned on the first flow guide surface 142 and the second flow guide surface 144 of the internal flow guide 140 as shown in FIG. 51(b). As the first wall portion and the second wall portion of the one or more walls 115 are pressed together, they will drape over the ridge 196 on the internal flow guide 140, leaving gaps either side of it. The gaps will generally remain even when tight dressings are applied over the receptacle 110 in use or regardless of patient movement. Whilst not shown in FIG. 51, a further alternative configuration is for the ridges 196 to be positioned on an inner surface of the first wall portion of the one or more walls 115 and on the first flow guide surface 142. A yet further alternative configuration is for the ridges 196 to be positioned on an inner surface of the second wall portion of the one or more walls 115 and on the second flow guide surface 144.

The ridges 196 may be offset from one another on opposing first wall portion and second wall portion of the one or more walls 115 as shown in FIG. 51(a) or on opposing first flow guide surfaces 142, and second flow guide surface 144 as shown in FIG. 51(b). However, other arrangements of the ridges 196 may also be used. Each ridge 196 may have a height of between approximately 50 to 500 micrometres, for example 75 to 250 micrometres, for example 100 to 200 micrometres, for example between approximately 125 to 175 micrometres. An embodiment of the ridge has a height of 150 micrometres. The height of the ridge 196 is a balance of the requirement for a taller ridge to help maintain a sufficient gap and thereby prevent blockage of the flow path, whilst avoiding an overly tall ridge that may cause indentation to the tissue of the patient and which may render the ridge susceptible to buckling. Each ridge 196 may have a width of between approximately 50 to 500 micrometres, for example 100 to 400 micrometres, for example between approximately 250 to 350 micrometres, or between approximately 280 to 330 micrometres. Whilst the features of the ridges 196 are described with reference to the receptacle 110, it will be apparent that one or more of the features of the ridges 196 may apply to any of the receptacles 10, 110, 210, 210A, 310, 410 described in this disclosure.

FIG. 52 shows an embodiment of the receptacle 110 that is similar to the embodiment of FIG. 40. In particular, the receptacle 110 includes an internal flow guide 140C that is configured in the same way as the internal flow guide 140 and has a first flow guide surface 142C and an opposing second flow guide surface 144C. However, in the embodiment of FIG. 52, the circulation opening 145C has a pressure regulator such as a pressure relief valve 170C disposed in it, such that fluid flowing through the flow path inside the receptacle 110 must pass through the pressure relief valve 170C before it can exit the receptacle 110 through the fluid outlet 53. Accordingly, there may be no pressure relief valve or other form of pressure regulator at the fluid outlet 53. The pressure relief valve 170C may be used instead of (or in addition to) the pressure relief valve 170 also in the embodiment of FIGS. 47 and 48 in which the internal flow guide 140B is arranged to form side by side compartments within the receptacle rather the upper and lower compartments. An alternative pressure regulation device, such as any of the pressure regulators disclosed herein, may be used instead of the pressure relief valve 170C to achieve the same effect. Whilst the features of the internal flow guide 140C are described with reference to the receptacle 110, it will be apparent that one or more of the features of the internal flow guide 140C may apply to any of the receptacles 10, 110, 210, 210A, 310, 410 described in this disclosure.

The internal flow guides 140, 140B and 140C may each be roughened on either or both of the first flow guide surface 142, 142A, 142B, 142C, and the second flow guide surface 144, 144A, 144B, 144C. The surface roughening may take the same form as is described in relation to the embodiment of the receptacle 310 shown in FIG. 29 and FIG. 30. The roughened surface(s) of the internal flow guide 140, 140B, 140C may be in addition to, or instead of, roughening of the internal surface of the receptacle 110 in the manner described in relation to the embodiment of the receptacle 310 shown in FIG. 29 and FIG. 30.

In each of the embodiments shown in FIGS. 40, 41, 45 to 50 and 52, the fluid inlet conduit 50 and the first fluid outlet conduit 52 can be encapsulated in the pressure spreading device 95 of the receptacle 110 so as to extend therefrom in the same direction, as further illustrated in FIG. 52. The fluid inlet conduit 50 and the first fluid outlet conduit 52 may be arranged to be disposed substantially adjacent one another. “Substantially adjacent” may include that the fluid inlet conduit 50 and the first fluid outlet conduit 52 are disposed such that they may be joined together, but need not be joined together, or that a space between the fluid inlet conduit 50 and the first fluid outlet conduit 52 may be such that a user of the receptacle 110 can apply a bandage or other dressing over the receptacle 110 at the wound 22 with the one hand as explained earlier in this disclosure, whilst handling both of the fluid inlet conduit 50 and the first fluid outlet conduit 52 with the other hand. For example, in some embodiments, a space between the fluid inlet conduit 50 and the first fluid outlet conduit 52 may be between approximately 0 mm to 20 mm, or between approximately 1 mm to 15 mm, or between approximately 1.5 mm to 10 mm, or between approximately 2 mm to 7 mm, or between approximately 2.5 mm to 5 mm, or between approximately 3 mm to 4 mm. In an example of a lower leg wound, the user may typically wrap the dressing over the receptacle 10 from the ankle to the knee in one direction. Arranging the fluid inlet conduit 50 and the first fluid outlet conduit 52 substantially adjacent one another may help to prevent the possibility of one conduit becoming trapped beneath the compression dressing as the leg is being wrapped up, which could cause a pressure injury to the patient. In some embodiments, the space between the fluid inlet conduit 50 and the first fluid outlet conduit 52 may be large, e.g. greater than about 20 mm as long as the fluid inlet conduit 50 and first fluid outlet conduit 52 can be handled with one hand to allow the dressing to be applied in this manner.

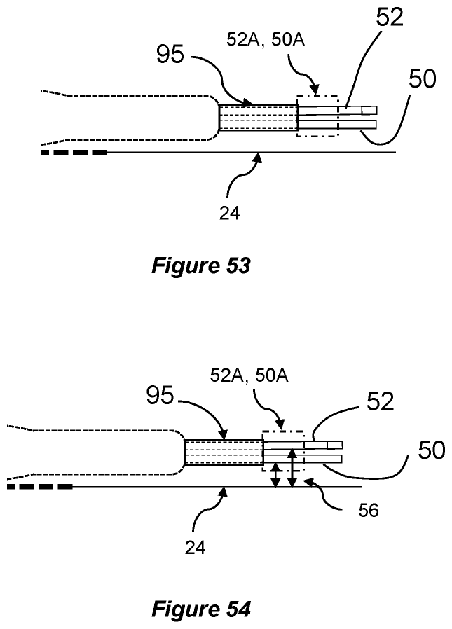

Furthermore, at least an end portion 50A, 52A (shown in FIG. 53) of each of the fluid inlet conduit 50 and the first fluid outlet conduit 52 that are closest to the pressure spreading device 95 may be arranged to protrude from the pressure spreading device 95 in a direction substantially aligned with the longitudinal axis of the receptacle 110, prior to use, as shown in FIG. 53. The end portions 50A, 52A of the fluid inlet conduit 50 and the first fluid outlet conduit 52 may be arranged substantially parallel with one another. The fluid inlet conduit 50 and the first fluid outlet conduit 52 may be at least partially adjoined so as to extend from the receptacle 110 in a common direction. In the embodiment of FIGS. 26 and 27, the fluid inlet conduit 50 is encapsulated in a pressure spreading device 95 that is attached to the receptacle 110. As an alternative to encapsulating the end portions 50A, 52A of the fluid inlet conduit 50 and the first fluid outlet conduit 52 in the pressure spreading device 95, the fluid inlet conduit 50 and the first fluid outlet conduit 52 may be at least partially adjoined or held together with tape, ties, glue, and/or clips (not shown). Such devices could be used to hold the fluid inlet conduit 50 and the first fluid outlet conduit 52 together at the end portions 50A, 52A that are closest to the receptacle 110, or over most of their length, or anything in between. However, in some embodiments, the end portions 50A, 52A of the fluid inlet conduit 50 and the first fluid outlet conduit 52 need not be held together.

At least partially adjoining or holding together the end portions 50A, 52A of the fluid inlet conduit 50 and the first fluid outlet conduit 52 may also allow application of a bandage or other dressing over the receptacle 110 and the fluid inlet conduit 50 and the first fluid outlet conduit 52 as described above, as the end portions 50A, 52A protrude from the pressure spreading device 95 parallel with one another. The end portions 50A, 52A of the fluid inlet conduit 50 and first fluid outlet conduit 52 may be arranged to protrude from the pressure spreading device 95 such that there is a relatively small vertical distance between the end portions 50A, 52A of the fluid inlet conduit 50 and first fluid outlet conduit 52, and a lowermost surface of the receptacle 110. This in turn means that the end portions 50A, 52A are disposed in close spaced relation from the tissue of the patient during use. In this context, “in close spaced relation” may include that the end portions 50A, 52A lie close to the tissue of the patient during use of the receptacle 110 so as to be almost but not quite touching the tissue, as illustrated by the arrows 56 in FIG. 54. This may also allow easier application of a bandage or other dressing over the receptacle 110 and the fluid inlet conduit 50 and the first fluid outlet conduit 52, in the manner described above. In an alternative embodiment, the end portions 50A, 52A of the fluid inlet conduit 50 and first fluid outlet conduit 52 may be arranged to protrude from the pressure spreading device 95 so as to be disposed in contact with the tissue of the patient during use. This disclosure may apply to any of the receptacles 10, 110, 210, 210A, 310, 410 described in this disclosure having a pressure spreading device 95, a fluid inlet conduit 50 and a first fluid outlet conduit 52.

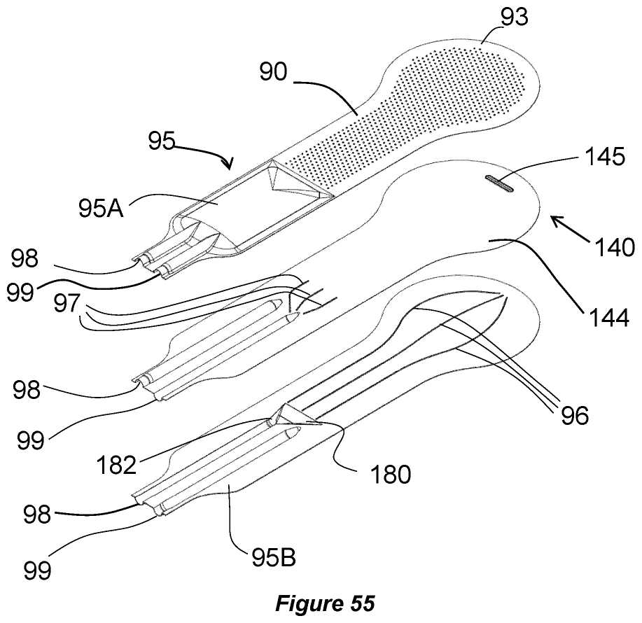

Referring to the embodiment of the receptacle 110 shown in FIGS. 55 to 58, the pressure spreading device 95 attaches to the tail portion 90, and the fluid inlet conduit 50 and the first fluid outlet conduit 52 may terminate within the pressure spreading device 95. This arrangement may help to prevent pressure injuries in use of the receptacle 110 from the fluid inlet conduit 50 and the first fluid outlet conduit 52 protruding into the tail portion 90 and creating a pressure point on the tissue of the patient.

As shown in the embodiment of FIG. 55 to FIG. 57, the pressure spreading device 95 has an upper portion 95A and a lower portion 95B that together form the pressure spreading device 95. The upper portion 95A and the lower portion 95B are the same as one another, however as best seen in FIG. 55, one is flipped relative to the other to form the pressure spreading device 95. An exterior surface of the upper portion 95A and the lower portion 95B are smooth, as seen for the upper portion 95A in FIG. 55. For example, the pressure spreading device 95 may be made of a smooth silicone material for contact with the tissue of the patient. The smooth exterior surface of the pressure spreading device 95 may assist with patient comfort as it contacts the tissue of the patient. The lower portion 95B and the upper portion 95A of the pressure spreading device 95 house a first cavity 180 and a second cavity 181 therein that in use enable fluid communication between the fluid inlet conduit 50 and the first fluid outlet conduit 52 and the tail portion 90, as will be described further below. The lower portion 95B of the pressure spreading device 95 has a fluid inlet conduit channel 98 integrally formed therein. The upper portion 95A of the pressure spreading device 95 has a first fluid outlet conduit channel 99 integrally formed therein. The fluid inlet conduit channel 98 and the first fluid outlet conduit channel 99 serve to guide the positioning of the fluid inlet conduit 50 and the first fluid outlet conduit 52 (not shown) inside the pressure spreading device 95. Consistent positioning of the fluid inlet conduit 50 and the first fluid outlet conduit 52 helps to achieve consistency of flow path length between receptacles 110 and, therefore, consistency of flow characteristics for each receptacle 110.

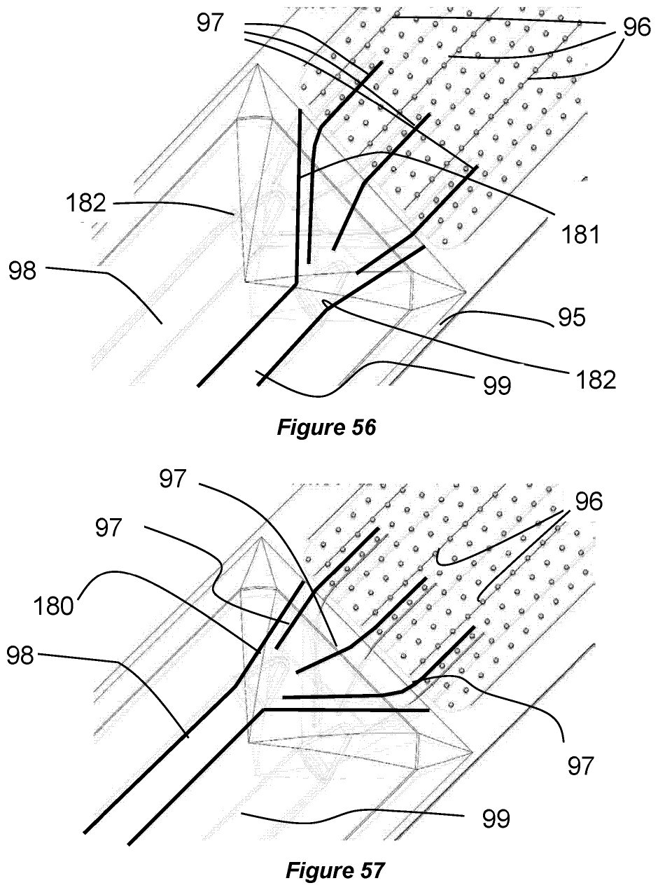

As seen in FIG. 55 and FIG. 57, the lower portion 95B of the pressure spreading device 95 may include the first cavity 180 that extends from the fluid inlet conduit channel 98 to the tail portion 90 of the receptacle 110, to enable fluid communication from the fluid inlet conduit 50 into the tail portion 90 of the receptacle 110. The upper portion 95A of the pressure spreading device 95 includes the second cavity 181, seen in FIG. 56, that extends from the tail portion 90 of the receptacle 110 to the first fluid outlet conduit channel 99 to enable fluid communication from the tail portion 90 of the receptacle 110 to the first fluid outlet conduit 52. The first cavity 180 is formed integrally within the lower portion 95B of the pressure spreading device 95. The second cavity 181 is formed integrally within the upper portion 95A of the pressure spreading device 95.

The pressure spreading device 95 includes an internal flow guide 140 in the form of an internal wall, best seen in FIG. 55. The internal flow guide 140 includes a portion of the fluid inlet conduit channel 98 on one side thereof and a portion of the first fluid outlet conduit channel 99 on an opposing side thereof. The first cavity 180 and the second cavity 181 are separated by the internal flow guide 140 within the pressure spreading device 95, as are the fluid inlet conduit channel 98 and the first fluid outlet conduit channel 99. In use, fluid may pass from the first cavity 180 into the tail portion 90 of the receptacle 110, into the head portion 93 of the receptacle 110 and through the circulation opening 145 (seen in FIG. 55) of the internal flow guide 140, as is described in respect of the embodiment of FIG. 50 above. Once fluid passes through the circulation opening 145, it passes back through the head portion 93, into the tail portion 90 and into the second cavity 181 of the pressure spreading device 95.

The first cavity 180 and the second cavity 181 each have a shape that gradually expands from a narrow end 182 at the respective fluid inlet conduit channel 98 or the first fluid outlet conduit channel 99 to the tail portion 90. With reference to FIG. 55, the shape of the first cavity 180 may assist in distributing fluid entering the first cavity 180 across the width of the tail portion 90, encouraging the fluid to reach each of the plurality of ridges 96 in the tail portion 90. To further assist the fluid entering the first cavity 180 to reach each of the plurality of ridges 96, the internal flow guide 140 may also have a series of cavity ridges 97 on the first flow guide surface 142 that faces the first cavity 180 (not seen in FIGS. 55 to 58). In FIG. 57, three cavity ridges 97 are schematically shown for ease of explanation. The cavity ridges 97 on the first flow guide surface 142 (the underside of the internal flow guide 140) help to maintain a fluid flow path from the fluid inlet conduit 50 to the tail portion 90 even if the first cavity 180 is crushed during use. Similarly, the series of cavity ridges 97 on the second flow guide surface 144 of the internal flow guide 140 (the upper surface of the internal flow guide 140, seen in FIG. 55) help to maintain a fluid flow path from the tail portion 90 to the first fluid outlet conduit 52 even if the second cavity 181 is crushed during use. In FIG. 56, three cavity ridges 97 are shown on the second flow guide surface 144. The cavity ridges 97 are arranged to taper towards each other at the narrow end 182 of the first cavity 180 of the fluid inlet conduit 50 and at the narrow end 182 of the second cavity 181 of the first fluid outlet conduit 52, as shown in FIG. 55 and also in FIGS. 56 and 57, following the shape of the respective cavity in which they are positioned. The cavity ridges 97 may be formed on the upper portion 95A and/or lower portion 95B of the pressure spreading device 95 instead of, or in addition to, those formed on the internal flow guide 140. The ridges 96 and the cavity ridges 97 are positioned so as to be offset from one another, to avoid creating pressure points that may lead to patient discomfort. However, the cavity ridges 97 continue into the tail portion 90 beyond the starting point of the ridges 96. This longitudinal overlap of the ridges 96 and the cavity ridges 97 helps to maintain a fluid flow path between the first cavity 180 and the tail portion 90 and the tail portion 90 and the second cavity 181 even if the first cavity 180 and/or the second cavity 181 is crushed.

In an alternative embodiment, the ridges 96 of the tail portion 90 of the receptacle 110 may extend into the first cavity 180 and the second cavity 181, terminating towards the narrow end 182 of the respective first cavity 180 or second cavity 181. In this embodiment, the cavity ridges 97 are not required.

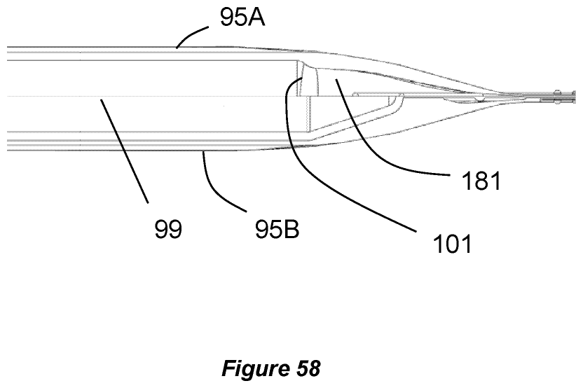

Each of the fluid inlet conduit channel 98 and the first fluid outlet conduit channel 99 may have a conduit locating feature 101, shown in FIG. 58. The conduit locating feature 101 is in the form of a stop. For example, the conduit locating feature 101 may be formed as an inwardly directed ring or partial ring that prevents the fluid inlet conduit 50 or first fluid outlet conduit 52 from being over inserted into the respective one of the fluid inlet conduit channel 98 and the first fluid outlet conduit channel 99. The fluid inlet conduit 50 or the first fluid outlet conduit 52 is positioned in the fluid inlet conduit channel 98 or the first fluid outlet conduit channel 99 so that its end contacts the conduit locating feature 101. In this way, the conduit locating feature 101 facilitates appropriate termination of the fluid inlet conduit 50 and the first fluid outlet conduit 52 at or near the narrow end 182 of the respective first cavity 180 or second cavity 181, preventing over insertion of the fluid inlet conduit 50 or the first fluid outlet conduit 52 which may cause additional pressure points on the tissue of the patient, increasing patient discomfort.

In an alternative embodiment, the conduit locating feature 101 may be omitted. In another alternative embodiment, the fluid inlet conduit 50 and the first fluid outlet conduit 52 may be configured to terminate in the respective one of the fluid inlet conduit channel 98 and the first fluid outlet conduit channel 99 prior to reaching the first cavity 180 or the second cavity 181, for example at the start of the fluid inlet conduit channel 98 and the first fluid outlet conduit channel 99 or part way along the respective channel. In such embodiments, the conduit locating feature 101 may be omitted, or it could be positioned at an appropriate point along the fluid inlet conduit channel 98 and/or the first fluid outlet channel 99. In a still further embodiment, the fluid inlet conduit 50 and the first fluid outlet conduit 52 may be configured to terminate in the first cavity 180 or the second cavity 181.

It will be appreciated that any of the receptacles 10, 110, 210, 210A, 310 or 410 that have a pressure spreading device 95 and a tail portion 90 may have one or more of the features of the first cavity 180, second cavity 181, fluid inlet conduit channel 98, fluid outlet conduit channel 99 or conduit locating feature 101 described above.

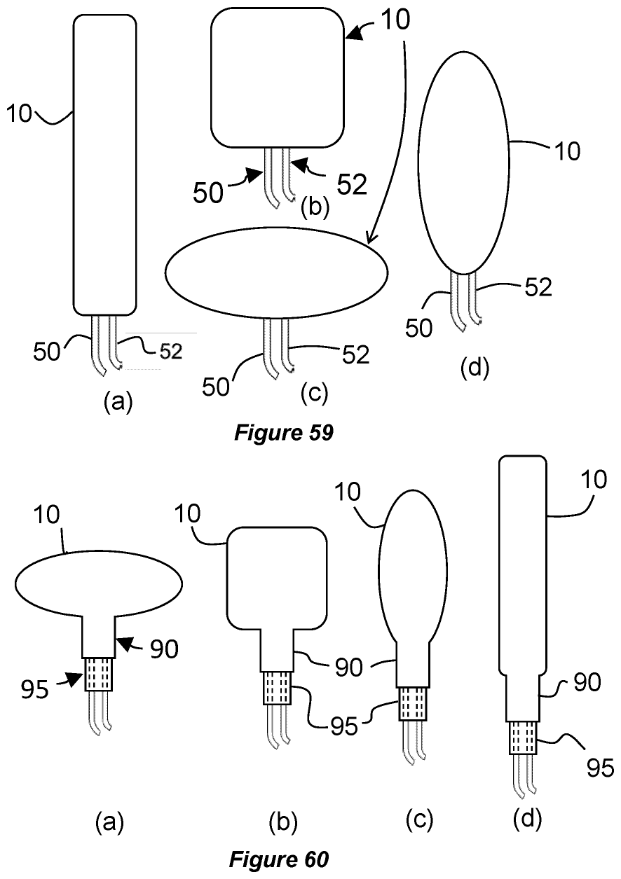

The receptacles 10, 110, 210, 210A, 310, 410 disclosed herein may have a shape that is different to those disclosed in the accompanying Figures and/or the specification. Examples of possible alternative receptacle shapes are shown in FIG. 59 and FIG. 60, however other shapes are also possible. FIG. 59(a) shows a receptacle 10 having a generally elongate rectangular shape. FIG. 59(b) shows a receptacle 10 having a generally square shape, with the fluid inlet conduit 50 and the first fluid outlet conduit 52 both extending from or near a mid-point of one side of the receptacle 10. FIG. 59(c) shows a receptacle 10 having a generally oval shape with the fluid inlet conduit 50 and the first fluid outlet conduit 52 both extending from a mid-point of an elongate side of the receptacle 10. FIG. 59(d) shows another receptacle 10 having a generally oval shape, with the fluid inlet conduit 50 and the first fluid outlet conduit 52 both extending from one end of the receptacle 10. It will be appreciated that the positions of the fluid inlet conduit 50 and the first fluid outlet conduit 52 relative to the receptacle 10 may also be changed from the positions shown in FIG. 59 without departing from the scope of the disclosure, as illustrated by the examples of the oval shaped receptacles of FIG. 59(c) and FIG. 59(d).

Each of the possible alternate shapes of the receptacle 10 may include a tail portion 90 and/or a pressure spreading device 95 as shown in FIG. 60(a) to (d). Whilst the embodiments shown in Figures FIG. 60(a) to (d) have both a tail portion 90 and a pressure spreading device 95, they may alternatively have a tail portion 90 but not a pressure spreading device 95, or they may have a pressure spreading device 95 but not a tail portion 90. The tail portion 90 and/or pressure spreading device 95 may extend from or near a mid-point of an elongate side of the receptacle 10 as shown in the embodiments of FIG. 60(a) and FIG. 60(b) or they may extend from one end of the receptacle 10 as shown in FIG. 60(c) and FIG. 60(d). Whilst the receptacles 10 shown in FIG. 60 have a sharp corner or squared transition between the tail portion 90 and the head portion 93, they may alternatively have a rounded or gradual transition between the tail portion 90 and the head portion 93 to avoid a sharp corner at the transition.

The flow rate of fluid entering the receptacles 10, 110, 210, 310, 410 from the fluid source 40 may be between approximately >0 mL/min to 20 mL/min, or between approximately 5 mL/min to 15 mL/min, or between approximately 7 to 13 mL/min, or between approximately 9 mL/min to 11 mL/min. In an embodiment, the flow rate of fluid entering the receptacle may be approximately 10 mL/min. For each of the embodiments of the receptacle 10, 110, 210, 210A, 310, 410 described in this disclosure, the amount of fluid leaving the interior of the receptacle 10, 110, 210, 210A, 310, 410 as a result of molecules within the fluid passing through the one or more walls of the receptacle 10, 110, 210, 210A, 310, 410 (e.g. via diffusion) may be approximately 0.5 to 2.5 mL/hr/cm2, or 1 to 2 mL/hr/cm2, or 1.2 to 1.6 mL/hr/cm2, or 1.5 mL/hr/cm2, as measured using a measuring system. In some embodiments, the amount of fluid leaving the interior of the receptacle 10, 110, 210, 210A, 310, 410 as a result of the molecules within the fluid passing through the one or more walls of the receptacle 10, 110, 210, 210A, 310, 410 (e.g. via diffusion) may be below about 0.5 mL/hr/cm2, as measured using the measuring system.

The measuring system includes a jig into which a receptacle, e.g. receptacle 10, is placed. The jig allows diffused fluid molecules to escape from the interior of the receptacle 10. The fluid inlet 55 of the receptacle 10 is fluidly connected to the fluid source 40 via an inlet line. An inlet flow meter is fluidly connected to the inlet line between the fluid source 40 and the fluid inlet 55. An outlet flow meter is fluidly connected to the fluid outlet 53 of the receptacle 10. The measurements are taken as follows. To measure the amount of fluid leaving the interior of the receptacle 10, the volumetric flow rate (standardised for temperature and pressure) at the fluid inlet 55 of the receptacle 10 is measured with the inlet flow meter and the volumetric flow rate at the fluid outlet 53 of the receptacle 10 (standardised for temperature and pressure) is measured with the outlet flow meter. The operator must wait until the system has reached equilibrium before taking this data. A difference between the inlet flow rate and the outlet flow rate is calculated. The value obtained corresponds to the amount of fluid leaving the interior of the receptacle 10 as a result of the molecules within the fluid passing through the one or more walls 15 of the receptacle (e.g. via diffusion). The measuring system is leak tested before the measurements are taken to ensure the difference in the two flow meters 507, 511 is not due to a leak but to the molecules within the fluid passing through the one or more walls 15 of the receptacle 10 (e.g. via diffusion).

The apparatus disclosed herein may be provided in various kit forms. For example, where the cover 30 is in the form of a compression bandage, the kit may comprise one or more receptacles 10, 110, 210, 210A, 310, 410 and the compression bandage. The kit may further comprise a fluid source 40, for example a portable oxygen source such as an oxygen bottle. Another kit may include one or more receptacles 10, 110, 210, 210A, 310, 410 and instructions to a user to apply a compression bandage for use with the one or more receptacles 10, 110, 210, 210A, 310, 410. A further kit may include one or more receptacles 10, 110, 210, 210A, 310, 410 and a fluid source 40, for example a portable oxygen source such as an oxygen bottle. The receptacles 10, 110, 210, 210A, 310, 410 may be supplied with one or more of the fluid inlet conduit 50, the first fluid outlet conduit 52 and one or more of the pressure relief valves 70, 170 or other pressure regulator.

Where the cover 30 is in the form of an adhesive dressing, the kit may comprise one or more of the receptacles 10, 110, 210, 210A, 310, 410 and the adhesive dressing. The kit may further comprise a fluid source 40, for example a portable oxygen source such as an oxygen bottle. In one form, the kit may comprise one or more of the receptacles 10, 110, 210, 210A, 310, 410. In each of the kits, the receptacles 10, 110, 210, 210A, 310, 410 may be supplied with one or more of the fluid inlet conduit 50 and the pressure relief valve 70, the first fluid outlet conduit 52 and the pressure relief valve 170 or other pressure regulator. A further kit may include a sealing cover 30 and a negative pressure source 45, for example a diaphragm or peristaltic pump. A canister (not shown) may also be supplied for attachment to the pump 45 for exudate collection.

It will be appreciated by the skilled person that the features and aspects of the various receptacles of the apparatus described herein are generally described in a form prior to use on a patient, unless stated otherwise. It will further be appreciated by the skilled person that the apparatus described herein may sometimes be used with an additional layer between the receptacle and the wound, for example a gauze layer. Reference to a tissue facing layer, wound facing surface, tissue facing surface of the receptacle or to applying the receptacle at the tissue or wound site, or placing the receptacle in contact with the wound or tissue, includes the possibility of using the apparatus with such an additional layer as well as use directly on the wound or tissue.

It will be appreciated by persons skilled in the art that numerous variations and/or modifications may be made to the above-described embodiments, without departing from the broad general scope of the present disclosure, including the following clauses. The present embodiments are, therefore, to be considered in all respects as illustrative and not restrictive.

Clause 1. An apparatus for supplying fluid to tissue of a patient, the apparatus comprising: an inflatable receptacle positionable at a tissue area, the receptacle comprising one or more walls and being adapted to receive a fluid, the one or more walls comprising at least one section adapted to allow molecules within the fluid to diffuse from the receptacle to the tissue area; and a cover positionable over the receptacle to form a compartment substantially bounded by the cover, the receptacle and the tissue area.

Clause 2. The apparatus of clause 1, wherein the receptacle is moveable within the compartment bounded by the cover, the receptacle and the tissue area.

Clause 3. An apparatus for supplying fluid to a tissue area, the apparatus comprising: a receptacle positionable at a tissue area, the receptacle comprising one or more walls and being adapted to receive a fluid, the one or more walls comprising at least one section adapted to allow molecules within the fluid to diffuse from the receptacle to the tissue area; and a cover positionable over the receptacle to form a compartment substantially bounded by the cover, the receptacle and the tissue area, wherein the receptacle is moveable within the compartment substantially bounded by the cover, the receptacle and the tissue area.

Clause 4. The apparatus of clause 3, wherein the receptacle is inflatable.

Clause 5. The apparatus of any one of clauses 1 to 4, wherein the receptacle is in the form of a bag.

Clause 6. The apparatus of any one of the preceding clauses, wherein the receptacle is configured to contact the tissue area.

Clause 7. The apparatus of any one of the preceding clauses, wherein at least the at least one section of the receptacle is conformable with a surface of the tissue area.

Clause 8. The apparatus of any one of the preceding clauses, wherein the one or more walls of the receptacle are adapted to allow molecules within the fluid to diffuse from the receptacle to the tissue area.

Clause 9. The apparatus of any one of the preceding clauses, wherein the at least one section and/or the one or more walls comprise a membrane.

Clause 10. The apparatus of any one of the preceding clauses, wherein the at least one section of the receptacle is configured to have an undulating outer surface when at least partially inflated.

Clause 11. The apparatus of any one of the preceding clauses, wherein the at least one section of the receptacle comprises a stretchable material.

Clause 12. The apparatus of any one of the preceding clauses, wherein the receptacle is connected to the cover at a single connection point or at two connection points.

Clause 13. The apparatus of any one of the preceding clauses, wherein the apparatus further comprises a fluid inlet conduit arranged in fluid communication with the receptacle for delivery of fluid to the receptacle.

Clause 14. The apparatus of clause 13, wherein the cover has a first opening through which the fluid inlet conduit passes or to which the fluid inlet conduit is connected.

Clause 15. The apparatus of clause 13 or clause 14, wherein the receptacle is coupled to the cover via the fluid inlet conduit.

Clause 16. The apparatus of clause 15, wherein the compartment is further bounded by the fluid inlet conduit.

Clause 17. The apparatus of any one of the preceding clauses, further comprising a fastener for securing the receptacle in place at the tissue area.

Clause 18. The apparatus of clause 17, wherein the fastener comprises adhesive.

Clause 19. The apparatus of any one of clause 13 or clause 14 or clause 15, wherein the fluid inlet conduit includes a pressure relief valve.

Clause 20. The apparatus of any one of the preceding clauses, wherein the cover comprises one or more walls and wherein the one or more walls of the cover are independent of the one or more walls of the receptacle.

Clause 21. The apparatus of any one of clauses 1 to 19, wherein the receptacle and the cover share a common wall.

Clause 22. The apparatus of any one of the preceding clauses, wherein the receptacle is configured to receive fluid during use of the apparatus.

Clause 23. The apparatus of any one of clauses 1 to 21, wherein the receptacle is pre-filled with the fluid.

Clause 24. The apparatus of any one of the preceding clauses, wherein the one or more walls of the receptacle may define one or more through-holes through the receptacle.

Clause 25. The apparatus of any one of the preceding clauses, including a plurality of the receptacles.

Clause 26. The apparatus of any one of the preceding clauses, further comprising structures associated with the at least one section of the receptacle, the structures being configured to contact the tissue area.

Clause 27. The apparatus of clause 26, wherein the structures are configured for encouraging cell growth.

Clause 28. The apparatus of clause 26, wherein the structures are microstructures.

Clause 29. The apparatus of clause 27, wherein the microstructures comprise formations which protrude from the one or more walls of the receptacle.

Clause 30. The apparatus of clause 27 or clause 28 or clause 29, wherein molecules diffuse through the at least one section of the receptacle and the microstructures into the tissue area.

Clause 31. The apparatus of clause 28 or clause 29 or clause 30, wherein the microstructures protrude from a tissue facing surface of the one or more walls of the receptacle.

Clause 32. The apparatus of any one of clauses 29 to 31, wherein the formations have one or more of a parabolic, trapezoidal, semi-spherical, spherical, pyramidal, conical, domed or frustoconical shape.

Clause 33. The apparatus of any one of clauses 28 to 32, wherein the microstructures have rounded edges.

Clause 34. The apparatus of clause 9, wherein the membrane adapted to allow molecules to diffuse from the receptacle to the tissue area is substantially pore-free.

Clause 35. The apparatus of clause 34, wherein the membrane comprises substantially no discontinuities or pores that are visible with an optical or electron microscope at a specified resolution.

Clause 36. The apparatus of clause 35, wherein the specified resolution is on the order of 1 micrometre.

Clause 37. The apparatus of any one of clauses 34 to 36, wherein the membrane adapted to allow molecules to diffuse from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 38. The apparatus of any one of clauses 34 to 37, wherein the membrane adapted to allow molecules to diffuse from the receptacle to the tissue area is configured to contact the tissue area.

Clause 39. The apparatus of any one of clauses 34 to 38, wherein the one or more walls of the receptacle, other than the at least one section, comprise a material adapted to allow molecules to pass through via molecular diffusion at only a negligible rate of diffusion.

Clause 40. The apparatus of any one of the preceding clauses, wherein the at least one section is conformable to the surface of the tissue area.

Clause 41. The apparatus of any one of the preceding clauses, wherein the at least one section has a wall thickness of between about 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micropmetres to 70 micrometres, or between about 30 micrometres to 60 micrometres, or between about 20 micrometres to 60 micrometres, or between about 45 micrometres and 55 micrometres.

Clause 42. The apparatus of clause 40 or clause 41, wherein the at least one section has a wall thickness of approximately 50 micrometres or of approximately 60 micrometres.

Clause 43. The apparatus of any one of clauses 33 to 41, wherein the membrane is impermeable to bulk transport of fluid.

Clause 44. The apparatus of any one of the preceding clauses, wherein the at least one section of the one or more walls of the receptacle are hydrophobic.

Clause 45. The apparatus of any one of the preceding clauses, wherein the cover is configured to secure the receptacle to the tissue area.

Clause 46. The apparatus of any one of the preceding clauses, wherein the cover is configured to press the receptacle onto or into the tissue area.

Clause 47. The apparatus of any one of the preceding clauses, wherein the cover comprises a dressing.

Clause 48. The apparatus of clause 47, wherein the dressing is a bandage is wrapped around a body part of the patient to form the cover.

Clause 49. The apparatus of clause 47 or clause 48, wherein the tissue area comprises a wound area, and wherein the cover comprises an absorbent material for absorbing exudate from the wound area.

Clause 50. The apparatus of clause 49, wherein the absorbent material is placed upon or within the wound area of a patient and the bandage is wrapped over the absorbent material to form the cover.

Clause 51. The apparatus of any one of clauses 45-48, wherein the cover comprises an adhesive dressing.

Clause 52. The apparatus of any one of clauses 48 to 51, wherein the bandage is a compression bandage.

Clause 53. The apparatus of any one of the preceding clauses, wherein the cover is integrally formed with the receptacle.

Clause 54. The apparatus of clause 13, wherein the fluid is deliverable to the receptacle via the first opening of the cover.

Clause 55. The apparatus of clause 54, wherein the first opening is adapted for receiving the fluid inlet conduit there through.

Clause 56. The apparatus of clause 53 or clause 55, wherein the cover has a second opening via which the fluid is receivable from the receptacle.

Clause 57. The apparatus of clause 56, further comprising a first fluid outlet conduit arranged in fluid communication with the receptacle, wherein the second opening is adapted for receiving the first fluid outlet conduit.

Clause 58. The apparatus of clause 57, wherein the compartment is further bounded by the first fluid outlet conduit.

Clause 59. The apparatus of clause 56, wherein the cover has a third opening via which the fluid is receivable from the compartment substantially bounded by the cover, the receptacle, and the tissue area.

Clause 60. The apparatus of clause 53 or clause 59, further comprising a second fluid outlet conduit arranged in fluid communication with the compartment substantially bounded by the cover, the receptacle, and the wound area, wherein the third opening is adapted for receiving the second fluid outlet conduit.

Clause 61. The apparatus of any one of clauses 54 to 60, further comprising a pressure relief valve in communication with the first opening and/or the receptacle and operable to relieve pressure within the receptacle.

Clause 62. The apparatus of any one of clauses 54 to 61, further comprising a first fluid outlet conduit arranged in fluid communication with the receptacle and a second fluid outlet conduit arranged in fluid communication with the compartment substantially bounded by the cover, the receptacle and the wound area, wherein at least two of the fluid inlet conduit, the first fluid outlet conduit, and the second fluid outlet conduit are formed as a combined conduit.

Clause 63. The apparatus of clause 62, wherein the at least two of the fluid inlet conduit, the first fluid outlet conduit, and the second fluid outlet conduit are formed as a coaxial conduit.

Clause 64. The apparatus of clause 62 or clause 63, wherein the combined conduit or the coaxial conduit pass through a single opening in the cover.

Clause 65. The apparatus of clause 13, further comprising a fluid inlet conduit arranged in fluid communication with the receptacle, wherein at least one of the fluid inlet conduit and the first fluid outlet conduit passes into the compartment past an outer edge of the cover.

Clause 66. The apparatus of any one of the preceding clauses, wherein the cover is sealable to the tissue area.

Clause 67. The apparatus of clause 66, wherein the cover is hermetically sealable to the tissue area.

Clause 68. The apparatus of clause 63 or clause 64, wherein the cover is sealable to the tissue area by one of an adhesive seal or a suction seal.

Clause 69. The apparatus of any one of clauses 66 to 68, wherein the tissue area comprises a wound area including a wound and skin adjacent the wound, wherein the cover is sealable to the skin adjacent the wound.

Clause 70. The apparatus of any one of clauses 1 to 68, wherein the tissue area comprises a wound area.

Clause 71. The apparatus of any one of the preceding clauses, wherein the cover comprises one or more walls that are adapted to allow molecules to pass through via molecular diffusion at only a negligible rate of diffusion.

Clause 72. The apparatus of any one of the preceding clauses, wherein the compartment substantially bounded by the cover, the receptacle and the tissue area is pressurised during use to form a negative pressure compartment.

Clause 73. The apparatus of any one of the preceding clauses, comprising a negative pressure source arranged in fluid communication with the compartment substantially bounded by the cover, the receptacle and the tissue area for applying a negative pressure to the compartment.

Clause 74. The apparatus of clause 73, wherein the negative pressure source is a pump.

Clause 75. The apparatus of any one of clause 72 to clause 74, wherein the pressure in the compartment is altered to be between approximately 50 mmHg to 150 mmHg or between approximately 80 mmHg to 125 mmHg or between 90 mmHg to 110 mmHg below atmospheric pressure.

Clause 76. The apparatus of any one of the preceding clauses, wherein the fluid comprises a therapeutic gas.

Clause 77. The apparatus of clause 76, wherein the gas comprises oxygen and/or carbon dioxide and/or carbon monoxide.

Clause 78. The apparatus of any one of the preceding clauses, wherein the apparatus further comprises a fluid source.

Clause 79. The apparatus of clause 78, wherein the fluid source comprises a wall source, gas bottle, gas concentrator, or other gas generator.

Clause 80. The apparatus of any one of the preceding clauses, wherein the receptacle forms a reservoir adapted for storing fluid therein.

Clause 81. The apparatus of clause 80, wherein the reservoir is inflatable.

Clause 82. The apparatus of clause 80 or clause 81, wherein the reservoir is fillable with the fluid.

Clause 83. The apparatus of clause 82, wherein the reservoir is adapted to be filled with fluid during use of the apparatus.

Clause 84. The apparatus of clause 82, wherein the reservoir is adapted to be filled with fluid prior to positioning the reservoir at the tissue area.

Clause 85. The apparatus of clause 84 wherein the reservoir is pre-filled with fluid during manufacture of the apparatus.

Clause 86. The apparatus of any one of clauses 80 to 84, wherein the reservoir is configured to provide fluid to the tissue upon disconnection of the reservoir from the fluid source.

Clause 87 The apparatus of any one of clauses 1 to 86, wherein the cover comprises a cover outlet via which fluid is receivable from the compartment substantially bounded by the cover, the receptacle and the tissue area.

Clause 88 The apparatus of clause 87, wherein the cover outlet comprises an outer edge of the cover.

Clause 89 The apparatus of any one of clauses 1 to 88, wherein the receptacle is substantially hollow.

Clause 90 The apparatus of any one of clause 13 or any one of clauses 14 to 89 when dependent on clause 13, wherein the receptacle comprises a head portion that is configured to be positioned over the tissue area, and a tail portion that is configured to extend over and/or away from the tissue area.

Clause 91. The apparatus of clause 90, wherein a fluid inlet conduit is in fluid communication with and/or connected to the tail portion.

Clause 92. The apparatus of clause 90 or 91, wherein the head portion is in fluid communication with the tail portion such that the fluid inlet conduit is configured to supply fluid to the head portion via the tail portion.

Clause 93. The apparatus of any one of clauses 90 to 92, wherein the tail portion is longer than it is wide.

Clause 94. The apparatus of any one of clauses 90 to 93, wherein the tail portion has a width that is less than the width of the head portion.

Clause 95. The apparatus of any one of clauses 1 to 94, further comprising a pressure spreading device adapted for contacting tissue of the patient.

Clause 96. The apparatus of clause 95 when dependent on claim 13 and claim 57, wherein the pressure spreading device encapsulates at least a portion of the fluid inlet conduit and/or the first fluid outlet conduit.

Clause 97. The apparatus of clause 95 when dependent on claim 13 and claim 57, or clause 96, wherein the pressure spreading device is configured to spread a force applied to the tissue of the patient by the fluid inlet conduit and/or the first fluid outlet conduit across a width of the pressure spreading device so as to mitigate patient discomfort.

Clause 98. The apparatus of any one of clauses 95 to 97 when dependent on any one of clauses 90 to 94, wherein the pressure spreading device adjoins with the tail portion of the receptacle.

Clause 99. The apparatus of clause 98, wherein the pressure spreading device is formed integrally with the tail portion of the receptacle.

Clause 100. The apparatus of clause 98, wherein the pressure spreading device is formed as a separate component from the tail portion of the receptacle.

Clause 101. The apparatus of any one of clauses 95 to 100, wherein the pressure spreading device has a shape configured to reduce pressure exerted on the patient by the fluid inlet conduit and/or the first fluid outlet conduit.

Clause 102 The apparatus of any one of clauses 95 to 101, wherein the pressure spreading device has a width that is greater than an external diameter of the fluid inlet conduit.

Clause 103. The apparatus of any one of clauses 95 to 102, wherein the pressure spreading device has a generally elliptical cross-sectional shape.

Clause 104. The apparatus of any one of clauses 1 to 103, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 105. The apparatus of clause 104, wherein the rough surface is irregular.

Clause 106. The apparatus of any one of clauses 104 to 105, wherein the rough surface varies in height.

Clause 107. The apparatus of any one of clauses 104 to 106, wherein the rough surface includes a plurality of raised and/or recessed elements that vary in width.

Clause 108. The apparatus of any one of clauses 104 to 107, wherein the rough surface is a non-wound contacting surface of the receptacle.

Clause 109. The apparatus of any one of clauses 104 to 108 when dependent on one of clauses 90 to 94, wherein the head portion includes the rough surface.

Clause 110. The apparatus of any one of clauses 104 to 109 when dependent on one of clauses 90 to 94, wherein the tail portion includes the rough surface.

Clause 111. The apparatus of clause 57 or any one of clauses 58 to 110 when dependent on clause 57, wherein the first fluid outlet conduit is configured for cycling fluid out of the receptacle.

Clause 112. The apparatus of clause 111, wherein the first fluid outlet conduit is configured to allow cycling of the fluid out of the receptacle to maintain a concentration of the fluid within the receptacle.

Clause 113. The apparatus of any one of clauses 1 to 112, comprising: a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 114. The apparatus of clause 113, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 115. The apparatus of clause 113, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 116. The apparatus of any one of clauses 113 to clause 115, wherein the internal flow guide comprises an internal wall of the receptacle.

Clause 117. The apparatus of any one of clauses 113 to 116, wherein the one or more walls of the receptacle include at least a section comprising a membrane adapted to allow molecules within the fluid to pass from the receptacle to the tissue of the patient.

Clause 118. The apparatus of clause 117, wherein the receptacle is sealed such that fluid may enter the receptacle substantially only through the fluid inlet; and fluid may exit the receptacle substantially only through the fluid outlet or as a result of molecules within the fluid passing through the membrane.

Clause 119: The apparatus of any one of clauses 95 to 103, wherein the pressure spreading device comprises at least one cavity providing fluid communication between the fluid inlet conduit and the tail portion of the receptacle and/or the tail portion of the receptacle and the first fluid outlet conduit.

Clause 120: The apparatus of clause 119, wherein the cavity has a width dimension that increases from the fluid inlet end of the cavity and/or fluid outlet end of the cavity to the tail portion end of the cavity.

Clause 121: The apparatus of clause 120, wherein the width dimension of the cavity increases gradually from the fluid inlet end of the cavity and/or fluid outlet end of the cavity to the tail portion end of the cavity.

Clause 122: The apparatus of clause 119 or clause 120 or claim 121, wherein the cavity has one or more cavity ridges extending substantially from a fluid inlet end of the cavity to a tail portion end of the cavity or from a fluid outlet end of the cavity to the tail portion end of the cavity.

Clause 123: The apparatus of clause 122, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 124: The apparatus of any one of clause 122 to clause 123, wherein the one or more cavity ridges are positioned so as to be offset from the at least one ridge of the tail portion.

Clause 125: The apparatus of clause 122 to clause 124, wherein the one or more cavity ridges overlap longitudinally with the at least one ridge of the tail portion.

Clause 126: The apparatus of any one of clauses 122 to 125, wherein the one or more cavity ridges comprise an extension of the at least one ridge of the tail portion into the cavity.

Clause 127: The apparatus of any one of clause 119 to clause 126, wherein the pressure spreading device is adapted to receive an end portion of the fluid inlet conduit and an end portion of the first fluid outlet conduit therein, such that the end portions terminate inside the pressure spreading device.

Clause 128: The apparatus of any one of clause 119 to clause 126, wherein the pressure spreading device is adapted to receive an end portion of the fluid inlet conduit and an end portion of the first fluid outlet conduit therein, such that the end portions are permitted to terminate inside the tail portion.

Clause 129: The apparatus of clause 127 or clause 128, wherein the pressure spreading device includes an inlet conduit channel and an outlet conduit channel adapted for receiving the respective fluid inlet conduit or the first fluid outlet conduit therein.

Clause 130: The apparatus of clause 127 or clause 129, therein the pressure spreading device comprises at least one conduit locating feature adapted for locating an end of the fluid inlet conduit or first fluid outlet conduit.

Clause 131: The apparatus of clause 130, wherein the conduit locating feature comprises a stop.

Clause 132: The apparatus of clause 130 or clause 131, wherein the conduit locating feature is positioned in the inlet conduit channel and/or the outlet conduit channel.

Clause 133: The apparatus of clause 132, wherein the conduit locating feature is positioned at the fluid inlet end of the cavity and/or the fluid outlet end of the cavity.

Clause 134: The apparatus of any one of clauses 113 to 118, wherein the internal flow guide has a fluid flow path feature adapted for allowing fluid to flow from the first compartment to the second compartment.

Clause 135: The apparatus of clause 134, wherein the fluid flow path feature is one or more of an opening, an aperture, or a perforation.

Clause 136: The apparatus of clause 134 or clause 135 wherein the fluid flow path feature includes an opening surround.

Clause 137: The apparatus of clause 136, wherein the opening surround comprises a continuous surround of the opening, aperture or perforation.

Clause 138: The apparatus of clause 136, wherein the opening surround comprises at least one discontinuity.

Clause 139: The apparatus of any one of clauses 136 to 138, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 140: The apparatus of clause 139, wherein the at least one ridge is arranged to overlap with the opening surround of the internal wall.

Clause 141: The apparatus of claim 140, wherein the at least one ridge comprises two or more ridges, at least one of which is arranged to overlap with the opening surround.

Clause 142: The apparatus of any one of clauses 139 to 141 when dependent on clause 138, wherein the at least one ridge is arranged to extend through the at least one discontinuity in the opening surround.

Clause 143. The apparatus of claim 65, wherein the cover comprises an outer edge past which at least one of the fluid inlet conduit and the first fluid outlet conduit pass to deliver fluid to or from the receptacle.

Clause 144. An apparatus for supplying fluid to a tissue area, the apparatus comprising: a receptacle positionable at a tissue area, the receptacle comprising one or more walls and being adapted to receive a fluid, the one or more walls comprising at least one section adapted to allow the fluid to pass from the receptacle to the tissue area; and a cover positionable over the receptacle to form a compartment substantially bounded by the cover, the receptacle and the tissue area; a first opening in the cover via which fluid is deliverable to the receptacle; a second opening in the cover via which fluid is receivable from the receptacle; and a third opening in the cover via which fluid is receivable from the compartment substantially bounded by the cover, the receptacle and the tissue area.

Clause 145. The apparatus of clause 144, further comprising a fluid inlet conduit arranged in fluid communication with the receptacle for delivery of fluid to the receptacle.

Clause 146. The apparatus of clause 145, wherein the first opening is adapted for receiving the fluid inlet conduit there through.

Clause 147. The apparatus of any one of clauses 145 to 146, further comprising a first fluid outlet conduit arranged in fluid communication with the receptacle, wherein the second opening is adapted for receiving the first fluid outlet conduit.

Clause 148. The apparatus of any one of clauses 145 to 147, further comprising a second fluid outlet conduit arranged in fluid communication with the compartment substantially bounded by the cover, the receptacle, and the tissue area, wherein the third opening is adapted for receiving the second fluid outlet conduit.

Clause 149. The apparatus of any one of clauses 144 to 148, wherein the compartment substantially bounded by the cover, the receptacle, and the tissue area is further bounded by one or more of the first inlet conduit and the first fluid outlet conduit.

Clause 150. The apparatus of any one of clauses 145 to 149, further comprising a pressure relief valve in communication with the first opening and/or the receptacle and operable to relieve pressure within the first opening and/or receptacle.

Clause 151. The apparatus of any one of clauses 148 to 150, wherein at least one of the fluid inlet conduit, the first fluid outlet conduit and the second fluid outlet conduit passes into the compartment past an outer edge of the cover.

Clause 152. An apparatus for supplying fluid to a tissue area, the apparatus comprising: a receptacle positionable at a tissue area, the receptacle comprising one or more walls and being adapted to receive a fluid, the one or more walls comprising at least one section adapted to allow the fluid to pass from the receptacle to the tissue area; an inlet via which fluid is deliverable to the receptacle; an outlet via which fluid is receivable from the receptacle; and a cover positionable over the receptacle to form a compartment substantially bounded by the cover, the receptacle and the tissue area, the cover having a cover outlet via which fluid is receivable from the compartment substantially bounded by the cover, the receptacle and the tissue area.

Clause 153. The apparatus of clause 152, further comprising a fluid inlet conduit arranged in fluid communication with the receptacle for delivery of fluid to the receptacle.

Clause 154. The apparatus of clause 153, wherein the inlet is adapted for receiving the fluid inlet conduit there through.

Clause 155. The apparatus of any one of clauses 152 to 153, further comprising a first fluid outlet conduit arranged in fluid communication with the receptacle, wherein the outlet is adapted for receiving the first fluid outlet conduit.

Clause 156. The apparatus of any one of clauses 152 to 155, further comprising a second fluid outlet conduit arranged in fluid communication with the compartment substantially bounded by the cover, the receptacle, and the tissue area, wherein the cover outlet is adapted for receiving the second fluid outlet conduit.

Clause 157. The apparatus of any one of clauses 152 to 156, wherein the compartment substantially bounded by the cover, the receptacle, and the tissue area is further bounded by one or more of the first inlet conduit and the first fluid outlet conduit.

Clause 158. The apparatus of any one of clauses 152 to 157, further comprising a pressure relief valve in communication with the receptacle and operable to relieve pressure within the receptacle.

Clause 159. The apparatus of any of clauses 156 to 158, wherein at least one of the fluid inlet conduit, the first fluid outlet conduit and the second fluid outlet conduit passes into the compartment past an outer edge of the cover.

Clause 160. The apparatus of any one of the preceding clauses, wherein the cover is sealable to the tissue area.

Clause 161. The apparatus of clause 160, wherein the cover is hermetically sealable to the tissue area.

Clause 162. The apparatus of clause 160 or clause 161, wherein the cover is sealable to the tissue area by one of an adhesive seal or a suction seal.

Clause 163. The apparatus of any one of clauses 160 to clause 162 wherein the tissue area comprises a wound area including a wound and skin adjacent the wound, wherein the cover is sealable to the skin adjacent the wound.

Clause 164. The apparatus of any one of the preceding clauses, wherein the cover comprises one or more walls that are adapted to allow molecules to pass through via molecular diffusion at only a negligible rate of diffusion.

Clause 165. The apparatus of any one of clauses 144 to 164, wherein the cover is configured to secure the receptacle to the tissue area.

Clause 166. The apparatus of any one of clauses 144 to 165, wherein the cover is configured to press the receptacle onto or into the tissue area.

Clause 167. The apparatus of any one of clauses 143 to 166, wherein the cover comprises an absorbent material for absorbing exudate from the wound area.

Clause 168. The apparatus of any one of the preceding clauses, wherein the cover is integrally formed with the receptacle.

Clause 169. The apparatus of any one of clauses 144 to 168, wherein the receptacle is inflatable.

Clause 170. The apparatus of any one of clauses 87 to 104, wherein the receptacle is in the form of a bag.

Clause 171. The apparatus of any one of clauses 144 to 170, wherein the receptacle is configured to contact the tissue area.

Clause 172. The apparatus of any one of clauses 143 to 171, wherein the receptacle is moveable within the compartment substantially bounded by the cover, the receptacle and the tissue area.

Clause 173. The apparatus of any one of clauses 144 to 172, wherein at least the at least one section of the receptacle is conformable with a surface of the tissue area.

Clause 174. The apparatus of any one of clauses 144 to 173, wherein the at least one section is adapted to allow molecules within the fluid to diffuse from the receptacle to the tissue area.

Clause 175. The apparatus of any one of clauses 144 to 174, wherein the at least one section is adapted to allow the fluid to pass from the receptacle to the tissue area by pore flow.

Clause 176. The apparatus of any one of clauses 144 to 175, wherein the at least one section of the receptacle is configured to have an undulating outer surface when at least partially inflated.

Clause 177. The apparatus of any one of clauses 144 to 176, wherein the at least one section of the receptacle comprises a stretchable material.

Clause 178. The apparatus of any one of clauses 144 to 177, wherein the receptacle is connected to the cover at a single connection point or at two connection points.

Clause 179. The apparatus of clause 145 or 153, or any of clauses 146 to 152 when dependent on clause 151 or any one of clauses 154 to 159 when dependent on clause 153, wherein the fluid inlet conduit passes through or is connected to the first opening.

Clause 180. The apparatus of clause 179, wherein the receptacle is connected to the cover via the fluid inlet conduit.

Clause 181. The apparatus of any one of clauses 145 to 180, further comprising a fastener for securing the receptacle in place at the tissue area.

Clause 182. The apparatus of clause 181, wherein the fastener comprises adhesive.

Clause 183. The apparatus of any one of clauses 145 to 180, wherein the fluid inlet conduit includes a pressure relief valve.

Clause 184. The apparatus of any one of clauses 145 to 183, wherein the cover comprises one or more walls and wherein the one or more walls are independent of the one or more walls of the receptacle.

Clause 185. The apparatus of any one of clauses 145 to 183, wherein the receptacle and the cover share a common wall.

Clause 186. The apparatus of any one of clauses 145 to 185, wherein the receptacle is fillable with the fluid during use of the apparatus.

Clause 187. The apparatus of any one of clauses 145 to 185, wherein the receptacle is adapted to be filled with fluid prior to positioning the receptacle at the tissue area.

Clause 188. The apparatus of any one of clauses 88 to 120, wherein the receptacle is pre-filled with the fluid during manufacture of the apparatus.

Clause 189. The apparatus of any one of clauses 145 to 185, wherein the one or more walls of the receptacle may define one or more through-holes through the receptacle.

Clause 190. The apparatus of any one of the preceding clauses, including a plurality of the receptacles.

Clause 191. The apparatus of any one of clauses 144 to 190, further comprising structures associated with the at least one section of the receptacle, the structures being configured to contact the tissue area.

Clause 192. The apparatus of clause 191, wherein the structures are configured for encouraging cell growth.

Clause 193. The apparatus of clause 191 or 192, wherein the structures are microstructures.

Clause 194. The apparatus of clause 193, wherein the microstructures comprise formations which protrude from the one or more walls of the receptacle.

Clause 195. The apparatus of clause 193 or clause 194, wherein the microstructures allow molecules within the fluid to diffuse through the at least one section of the receptacle into the tissue area.

Clause 196. The apparatus of clause 194 or clause 195, wherein the microstructures protrude from a tissue facing surface of the one or more walls of the receptacle.

Clause 197. The apparatus of any one of clauses 194 to 196, wherein the formations have one or more of a parabolic, trapezoidal, semi-spherical, spherical, pyramidal, conical, domed or frustoconical shape.

Clause 198. The apparatus of any one of clauses 193 to 197, wherein the microstructures have rounded edges.

Clause 199. The apparatus of any one of clauses 144 to 198, wherein the at least one section of the receptacle comprises a membrane adapted to allow molecules within the fluid to diffuse from the receptacle to the tissue area.

Clause 200. The apparatus of any one of clauses 144 to 199, wherein the receptacle comprises a membrane adapted to allow molecules within the fluid to diffuse from the receptacle to the tissue area.

Clause 201. The apparatus of clause 199 or clause 200, wherein the membrane is adapted to allow the fluid to pass from the receptacle to the tissue area by pore flow.

Clause 202. The apparatus of any one of clauses 199 to 200, wherein the membrane adapted to allow molecules to diffuse from the receptacle to the tissue area is substantially pore-free.

Clause 203. The apparatus of any one of clauses 199 to 200 or clause 202, wherein the membrane comprises substantially no discontinuities or pores that are visible with an optical or electron microscope at a specified resolution.

Clause 204. The apparatus of clause 203, wherein the specified resolution is on the order of 1 micrometre.

Clause 205. The apparatus of any one of clauses 199 to 204, wherein the membrane adapted to allow molecules to diffuse from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 206. The apparatus of any one of clauses 199 to 205, wherein the membrane adapted to allow molecules to diffuse from the receptacle to the tissue area is configured to contact the tissue area.

Clause 207. The apparatus of any one of clauses 144 to 206, wherein the one or more walls of the receptacle, other than the at least one section, comprise a material adapted to allow molecules to pass through via molecular diffusion at only a negligible rate of diffusion.

Clause 208. The apparatus of any one of clauses 144 to 207, wherein the at least one section is conformable to the surface of the tissue area.

Clause 209. The apparatus of any one of clauses 144 to 208, wherein the at least one section has a wall thickness of between about 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between approximately 20 micrometres to 60 micrometres, or between about 45 micrometres and 55 micrometres.

Clause 210. The apparatus of clause 209, wherein the at least one section has a wall thickness of approximately 50 micrometres or of approximately 60 micrometres.

Clause 211. The apparatus of any one of clauses 200 to 208, wherein the membrane is impermeable to bulk transport of fluid.

Clause 212. The apparatus of any one of the preceding clauses, wherein the at least one section of the one or more walls of the receptacle are hydrophobic.

Clause 213. The apparatus of any one of clauses 144 to 212, wherein the compartment substantially bounded by the cover, the receptacle and the tissue area is pressurised during use to form a negative pressure compartment.

Clause 214. The apparatus of any one of clauses 144 to 213, comprising a negative pressure source arranged in fluid communication with the compartment substantially bounded by the cover, the receptacle and the tissue area for applying a negative pressure to the compartment.

Clause 215. The apparatus of clause 214, wherein the negative pressure source is a pump.

Clause 216. The apparatus of clause 213, wherein the pressure in the compartment is altered to be between approximately 50 mmHg to 150 mmHg or between approximately 80 mmHg to 125 mmHg or between 90 mmHg to 110 mmHg below atmospheric pressure.

Clause 217. The apparatus of any one of clauses 144 to 216, wherein the fluid comprises a therapeutic gas.

Clause 218. The apparatus of clause 217, wherein the gas comprises oxygen and/or carbon dioxide and/or carbon monoxide.

Clause 219. The apparatus of any one of clauses 144 to 215, wherein the apparatus further comprises a fluid source.

Clause 220. The apparatus of clause 219, wherein the fluid source comprises a wall source, gas bottle, gas concentrator, or other gas generator.

Clause 221. The apparatus of any one clauses 144 to 220, wherein the receptacle comprises a tubular receptacle having a fluid inlet and a fluid outlet.

Clause 222. The apparatus of any one of clauses 144 to 221, wherein the tissue area comprises a wound area.

Clause 223. The apparatus of any one of clauses 144 to 222, further comprising a pressure regulator adapted for passage of the fluid receivable from the receptacle there through and operable to regulate pressure within the second opening and/or the receptacle.

Clause 224. The apparatus of clause 223, wherein the pressure regulator is a pressure relief valve.

Clause 225. The apparatus of clause 223 or clause 224, wherein the pressure regulator is configurable to relieve pressure within the receptacle when the pressure within the receptacle reaches a threshold value.

Clause 226. The apparatus of clause 225, wherein the pressure regulator is configurable to adjust the pressure threshold value.

Clause 227. The apparatus of any one of clauses 225 to 226, wherein the pressure threshold value is an upper bound of the pressure.

Clause 228. The apparatus of any one of clauses 223 to 227, wherein the pressure regulator is passively and/or mechanically actuable to permit fluid to pass there through.

Clause 229. The apparatus of any one of clauses 223 to 227, wherein the pressure regulator is actively actuable to permit fluid to pass there through.

Clause 230. The apparatus of any one of clauses 223 to 229, wherein the pressure regulator is positioned in spaced relation from the cover and/or the receptacle.

Clause 231. The apparatus of any one of claims 223 to 230, wherein the pressure regulator is positioned distally from the receptacle so as to be outside of the cover.

Clause 232. The apparatus of clause 230 or 231 when dependent on at least clause 223 and one of clause 147 or clause 155, wherein the pressure regulator is disposed on the first fluid outlet conduit.

Clause 233. The apparatus of any one of clauses 223 to 232, wherein the pressure regulator is selectively actuable to an at least partially open position to permit fluid to pass there through, and is configured, when in the at least partially open position, to vent the fluid passing there through to the atmosphere.

Clause 234 The apparatus of any one of clauses 144 to 233, wherein the cover comprises an outer edge via which fluid from within the receptacle and/or fluid from within the compartment is receivable from the compartment substantially bounded by the cover, the receptacle and the tissue area.

Clause 235 The apparatus of any one of clauses 144 to 234, wherein the receptacle is substantially hollow.

Clause 236 The apparatus of any one of clause 145 or 153 or any one of clauses 146 to 152 when dependent on clause 145 or any one of clauses 153 to 235 when dependent on clause 153, wherein the receptacle comprises a head portion that is configured to be positioned over the tissue area, a tail portion that is configured to extend over and/or away from the tissue area.

Clause 237. The apparatus of clause 236, comprising a fluid inlet conduit arranged in fluid communication with the tail portion and/or connected to the tail portion.

Clause 238. The apparatus of clause 236 or 237, wherein the head portion is in fluid communication with the tail portion such that the fluid inlet conduit is configured to supply fluid to the head portion via the tail portion.

Clause 239. The apparatus of any one of clauses 236 to 238, wherein the tail portion is longer than it is wide.

Clause 240. The apparatus of any one of clauses 236 to 239, wherein the tail portion has a width that is less than the width of the head portion.

Clause 241. The apparatus of any one of clauses 144 to 240, further comprising a pressure spreading device adapted for contacting tissue of the patient.

Clause 242. The apparatus of clause 241 when dependent on clause 145 and clause 147, or clause 153 and clause 155 wherein the pressure spreading device encapsulates at least a portion of the fluid inlet conduit and/or the first fluid outlet conduit.

Clause 243. The apparatus of clause 242 when dependent on claim 145 and claim 147, or clause 153 and clause 155, wherein the pressure spreading device is configured to spread a force applied to the tissue of the patient by the fluid inlet conduit and/or the first fluid outlet conduit across a width of the pressure spreading device so as to mitigate patient discomfort.

Clause 244. The apparatus of any one of clauses 241 to 243 when dependent on any one of clauses 90 to 94, wherein the pressure spreading device adjoins with the tail portion of the receptacle.

Clause 245. The apparatus of clause 244, wherein the pressure spreading device is formed integrally with the tail portion of the receptacle.

Clause 246. The apparatus of clause 244, wherein the pressure spreading device is formed as a separate component from the tail portion of the receptacle.

Clause 247. The apparatus of any one of clauses 241 to 246, wherein the pressure spreading device has a shape configured to reduce pressure exerted on the patient by the fluid inlet conduit and/or the first fluid outlet conduit.

Clause 248. The apparatus of any one of clauses 241 to 247, wherein the pressure spreading device has a width that is greater than an external diameter of the fluid inlet conduit.

Clause 249. The apparatus of any one of clauses 241 to 248, wherein the pressure spreading device has a generally elliptical cross-sectional shape.

Clause 250. The apparatus of any one of clauses 144 to 249, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 251. The apparatus of clause 250, wherein the rough surface is irregular.

Clause 252. The apparatus of any one of clauses 250 to 251, wherein the rough surface varies in height.

Clause 253. The apparatus of any one of clauses 250 to 252, wherein the rough surface includes a plurality of raised and/or recessed elements that vary in width.

Clause 254. The apparatus of any one of clauses 250 to 253, wherein the rough surface is a non-wound contacting surface of the receptacle.

Clause 255. The apparatus of any one of clauses 250 to 254 when dependent on one of clauses 236 to 240, wherein the head portion includes the rough surface.

Clause 256. The apparatus of any one of clauses 250 to 255 when dependent on one of clauses 236 to 240, wherein the tail portion includes the rough surface.

Clause 257. The apparatus of clause 147 or clause 155 wherein the first fluid outlet conduit is configured for cycling fluid out of the receptacle.

Clause 258. The apparatus of clause 257, wherein the first fluid outlet conduit is configured to allow cycling of the fluid out of the receptacle to maintain a concentration of the fluid within the receptacle.

Clause 259. The apparatus of any one of clauses 144 to 258, comprising: a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 260. The apparatus of clause 259, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 261. The apparatus of clause 259, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 262. The apparatus of any one of clauses 259 to clause 261, wherein the internal flow guide comprises an internal wall of the receptacle.

Clause 263. The apparatus of any one of clauses 259 to 262, wherein the one or more walls of the receptacle include at least a section comprising a membrane adapted to allow molecules within the fluid to pass from the receptacle to the tissue of the patient.

Clause 264. The apparatus of clause 263, wherein the receptacle is sealed such that fluid may enter the receptacle substantially only through the fluid inlet; and fluid may exit the receptacle substantially only through the fluid outlet or as a result of molecules within the fluid passing through the membrane.

Clause 265: The apparatus of any one of clauses 241 to 249, wherein the pressure spreading device comprises at least one cavity providing fluid communication between the fluid inlet conduit and the tail portion of the receptacle and/or the tail portion of the receptacle and the first fluid outlet conduit.

Clause 266: The apparatus of clause 265, wherein the cavity has a width dimension that increases from the fluid inlet end of the cavity and/or fluid outlet end of the cavity to the tail portion end of the cavity.

Clause 267: The apparatus of clause 266, wherein the width dimension of the cavity increases gradually from the fluid inlet end of the cavity and/or fluid outlet end of the cavity to the tail portion end of the cavity.

Clause 268: The apparatus of clause 265 or clause 266 or claim 267, wherein the cavity has one or more cavity ridges extending substantially from a fluid inlet end of the cavity to a tail portion end of the cavity or from a fluid outlet end of the cavity to the tail portion end of the cavity.

Clause 269: The apparatus of clause 268, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 270: The apparatus of any one of clause 268 to clause 269, wherein the one or more cavity ridges are positioned so as to be offset from the at least one ridge of the tail portion.

Clause 271: The apparatus of clause 268 to clause 270, wherein the one or more cavity ridges overlap longitudinally with the at least one ridge of the tail portion.

Clause 272: The apparatus of any one of clauses 268 to 271, wherein the one or more cavity ridges comprise an extension of the at least one ridge of the tail portion into the cavity.

Clause 273: The apparatus of any one of clause 265 to clause 272, wherein the pressure spreading device is adapted to receive an end portion of the fluid inlet conduit and an end portion of the first fluid outlet conduit therein, such that the end portions terminate inside the pressure spreading device.

Clause 274: The apparatus of any one of clause 265 to clause 273, wherein the pressure spreading device is adapted to receive an end portion of the fluid inlet conduit and an end portion of the first fluid outlet conduit therein, such that the end portions are permitted to terminate inside the tail portion.

Clause 275: The apparatus of clause 273 or clause 274, wherein the pressure spreading device includes an inlet conduit channel and an outlet conduit channel adapted for receiving the respective fluid inlet conduit or the first fluid outlet conduit therein.

Clause 276: The apparatus of clause 273 or clause 275, therein the pressure spreading device comprises at least one conduit locating feature adapted for locating an end of the fluid inlet conduit or first fluid outlet conduit.

Clause 277: The apparatus of clause 276, wherein the conduit locating feature comprises a stop.

Clause 278: The apparatus of clause 276 or clause 277, wherein the conduit locating feature is positioned in the inlet conduit channel and/or the outlet conduit channel.

Clause 279: The apparatus of clause 278, wherein the conduit locating feature is positioned at the fluid inlet end of the cavity and/or the fluid outlet end of the cavity.

Clause 280: The apparatus of any one of clauses 259 to 264, wherein the internal flow guide has a fluid flow path feature adapted for allowing fluid to flow from the first compartment to the second compartment.

Clause 281: The apparatus of clause 280, wherein the fluid flow path feature is one or more of an opening, an aperture, or a perforation.

Clause 282: The apparatus of clause 281 wherein the fluid flow path feature includes an opening surround.

Clause 283: The apparatus of clause 282, wherein the opening surround comprises a continuous surround of the opening, aperture or perforation.

Clause 284: The apparatus of clause 283, wherein the opening surround comprises at least one discontinuity.

Clause 285: The apparatus of any one of clauses 282 to 284, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 286: The apparatus of clause 285, wherein the at least one ridge is arranged to overlap with the opening surround of the internal wall.

Clause 287: The apparatus of claim 286, wherein the at least one ridge comprises two or more ridges, at least one of which is arranged to overlap with the opening surround.

Clause 288: The apparatus of clause 285 when dependent on clause 284, wherein the at least one ridge is arranged to extend through the at least one discontinuity in the opening surround.

Clause 289. An apparatus for supplying fluid to a tissue area, the apparatus comprising: a plurality of receptacles positionable at a tissue area, each receptacle comprising one or more walls and being adapted to receive a fluid, the one or more walls comprising at least one section adapted to allow the fluid to pass from the receptacle to the tissue area.

Clause 290. The apparatus of clause 25 or clause 190 or clause 289, wherein the one or more walls of each of the plurality of receptacles are independent of the one or more walls of the other receptacles.

Clause 291. The apparatus of clause 289 or clause 290, wherein each receptacle of the plurality of receptacles is individually positionable at the tissue area.

Clause 292. The apparatus of any one of clauses 289 to 291, wherein each receptacle of the plurality of receptacles is associated with a separate fluid supply.

Clause 293. The apparatus of any one of clauses 289 to 292, wherein the apparatus comprises at least one manifold for providing fluid to each receptacle of the plurality of receptacles.

Clause 294. The apparatus of any one of clauses 289 to 293, further comprising a plurality of fluid inlet conduits, wherein each receptacle of the plurality of receptacles is in fluid communication with a respective fluid inlet conduit of the plurality of fluid inlet conduits, via which fluid is supplied to the respective receptacle.

Clause 295. The apparatus of any one of clauses 289 to 294, wherein each of the receptacles is arranged in fluid communication with at least one other of the receptacles.

Clause 296. The apparatus of any one of clauses 289 to 295, wherein one or more of the receptacles of the plurality of receptacles has a plurality of microstructures formed thereon.

Clause 297. The apparatus of any one of clauses 290 to 296, further comprising a cover positionable over the plurality of receptacles to form a compartment substantially bounded by the cover, the plurality of receptacles and the tissue area.

Clause 298. The apparatus of clause 297 when dependent on clause 294, wherein the compartment substantially bounded by the cover, the receptacle and the tissue area is further bounded by the plurality of fluid inlet conduits.

Clause 299. The apparatus of clause 297 when dependent on clause 174, wherein the plurality of fluid inlet conduits are in fluid communication with the manifold and wherein the manifold has a fluid supply conduit that passes through a single opening in the cover.

Clause 300. The apparatus of clause 299, wherein the compartment substantially bounded by the cover, the receptacle and the tissue area is further bounded by the plurality of fluid inlet conduits, the manifold and the fluid supply conduit.

Clause 301. The apparatus of clause 297 or clause 298 when dependent upon clause 294, wherein each fluid inlet conduit of the plurality of fluid inlet conduits passes through a respective opening in the cover.

Clause 302. The apparatus of clause 297, when dependent on clause 294, wherein each fluid inlet conduit of the plurality of fluid inlet conduits extends around an outer edge of the cover from an associated fluid supply.

Clause 303. The apparatus of clause 302, wherein each fluid inlet conduit of the plurality of fluid inlet conduits passes into the compartment past an outer edge of the cover from an associated fluid supply.

Clause 304. The apparatus of any one of clauses 289 to 302, wherein the at least one section of the receptacle comprises a membrane adapted to allow molecules within the fluid to diffuse from the receptacle to the tissue area.

Clause 305. The apparatus of any one of clauses 289 to 304, wherein the receptacle comprises a membrane adapted to allow molecules within the fluid to diffuse from the receptacle to the tissue area.

Clause 306. The apparatus of any one of clauses 304 to 305, wherein the membrane is adapted to allow the fluid to pass from the receptacle to the tissue area by pore flow.

Clause 307. The apparatus of any one of clauses 304 to 305, wherein the membrane adapted to allow molecules to diffuse from the receptacle to the tissue area is substantially pore-free.

Clause 308. The apparatus of any one of clauses 304 to 305 or clause 307, wherein the membrane comprises substantially no discontinuities or pores that are visible with an optical or electron microscope at a specified resolution.

Clause 309. The apparatus of clause 308, wherein the specified resolution is on the order of 1 micrometre.

Clause 310. The apparatus of any one of clauses 304 to 309, wherein the membrane adapted to allow molecules to diffuse from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 311. The apparatus of any one of clauses 304 to 310, wherein the membrane is impermeable to bulk transport of fluid.

Clause 312. The apparatus of any one of the preceding clauses, wherein the at least one section of the one or more walls of the receptacle are hydrophobic.

Clause 313. The apparatus of any one of clauses 289 to 312, wherein the fluid comprises a therapeutic gas.

Clause 314. The apparatus of clause 313, wherein the gas comprises oxygen and/or carbon dioxide and/or carbon monoxide.

Clause 315. The apparatus of any one of clauses 289 to 314, wherein the tissue area comprises a wound area.

Clause 316. The apparatus of any one of clauses 289 to 315, wherein each of the plurality of receptacles is substantially hollow.

Clause 317 The apparatus of any one of clauses 289 to 316, wherein the cover comprises an outer edge, via which fluid from within the receptacle and/or fluid from within the compartment is receivable from the compartment substantially bounded by the cover, the receptacle and the tissue area.

Clause 318. The apparatus of any one of clause 294 or any one of clauses 295 to 317 when dependent on clause 294, wherein each receptacle comprises a head portion that is configured to be positioned over the tissue area, a tail portion that is configured to extend over and/or away from the tissue area.

Clause 319. The apparatus of clause 318, comprising a fluid inlet conduit arranged in fluid communication with the tail portion and/or connected to the tail portion.

Clause 320. The apparatus of clause 318 or 319, wherein the head portion is in fluid communication with the tail portion such that the fluid inlet conduit is configured to supply fluid to the head portion via the tail portion.

Clause 321. The apparatus of any one of clauses 318 to 320, wherein the tail portion is longer than it is wide.

Clause 322. The apparatus of any one of clauses 318 to 321, wherein the tail portion has a width that is less than the width of the head portion.

Clause 323. The apparatus of any one of clauses 289 to 321, further comprising a pressure spreading device adapted for contacting tissue of the patient.

Clause 324. The apparatus of clause 323 when dependent on claim 294, wherein each receptacle of the plurality of receptacles comprises a first fluid outlet conduit, wherein the pressure spreading device encapsulates at least a portion of the fluid inlet conduit and/or the first fluid outlet conduit.

Clause 325. The apparatus of clause 324 when dependent on claim 294, wherein the pressure spreading device is configured to spread a force applied to the tissue of the patient by the fluid inlet conduit and/or the first fluid outlet conduit across a width of the pressure spreading device so as to mitigate patient discomfort.

Clause 326. The apparatus of any one of clauses 324 to 325 when dependent on any one of clauses 318 to 322, wherein the pressure spreading device adjoins with the tail portion of the receptacle.

Clause 327. The apparatus of clause 326, wherein the pressure spreading device is formed integrally with the tail portion of the receptacle.

Clause 328. The apparatus of clause 326, wherein the pressure spreading device is formed as a separate component from the tail portion of the receptacle.

Clause 329. The apparatus of any one of clauses 323 to 328, wherein the pressure spreading device has a shape configured to reduce pressure exerted on the patient by the fluid inlet conduit and/or the first fluid outlet conduit.

Clause 330 The apparatus of any one of clauses 323 to 329, wherein the pressure spreading device has a width that is greater than an external diameter of the fluid inlet conduit.

Clause 331. The apparatus of any one of clauses 323 to 330, wherein the pressure spreading device has a generally elliptical cross-sectional shape.

Clause 332. The apparatus of any one of clauses 289 to 331, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 333. The apparatus of clause 332, wherein the rough surface is irregular.

Clause 334. The apparatus of any one of clauses 332 to 333, wherein the rough surface varies in height.

Clause 335. The apparatus of any one of clauses 332 to 334, wherein the rough surface includes a plurality of raised and/or recessed elements that vary in width.

Clause 336. The apparatus of any one of clauses 332 to 335, wherein the rough surface is a non-wound contacting surface of the receptacle.

Clause 337. The apparatus of any one of clauses 332 to 336 when dependent on one of clauses 318 to 322, wherein the head portion includes the rough surface.

Clause 338. The apparatus of any one of clauses 332 to 337 when dependent on one of clauses 318 to 322, wherein the tail portion includes the rough surface.

Clause 339. The apparatus of clause 325, wherein the first fluid outlet conduit is configured for cycling fluid out of the receptacle.

Clause 340. The apparatus of clause 339, wherein the first fluid outlet conduit is configured to allow cycling of the fluid out of the receptacle to maintain a concentration of the fluid within the receptacle.

Clause 341. The apparatus of any one of clauses 289 to 340, comprising, for each receptacle of the plurality of receptacles: a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 342. The apparatus of clause 341, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 343. The apparatus of clause 341, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 344. The apparatus of any one of clauses 341 to clause 343, wherein the internal flow guide comprises an internal wall of the receptacle.

Clause 345. The apparatus of any one of clauses 341 to 344, wherein the one or more walls of the receptacle include at least a section comprising a membrane adapted to allow molecules within the fluid to pass from the receptacle to the tissue of the patient.

Clause 346. The apparatus of clause 345, wherein the receptacle is sealed such that fluid may enter the receptacle substantially only through the fluid inlet; and fluid may exit the receptacle substantially only through the fluid outlet or as a result of molecules within the fluid passing through the membrane.

Clause 347: The apparatus of any one of clauses 323 to 331, wherein the pressure spreading device comprises at least one cavity providing fluid communication between the fluid inlet conduit and the tail portion of the receptacle and/or the tail portion of the receptacle and the first fluid outlet conduit.

Clause 348: The apparatus of clause 347, wherein the cavity has a width dimension that increases from the fluid inlet end of the cavity and/or fluid outlet end of the cavity to the tail portion end of the cavity.

Clause 349: The apparatus of clause 348, wherein the width dimension of the cavity increases gradually from the fluid inlet end of the cavity and/or fluid outlet end of the cavity to the tail portion end of the cavity.

Clause 350: The apparatus of clause 347 or clause 348 or claim 349, wherein the cavity has one or more cavity ridges extending substantially from a fluid inlet end of the cavity to a tail portion end of the cavity or from a fluid outlet end of the cavity to the tail portion end of the cavity.

Clause 351: The apparatus of clause 350, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 352: The apparatus of any one of clause 350 to clause 351, wherein the one or more cavity ridges are positioned so as to be offset from the at least one ridge of the tail portion.

Clause 353: The apparatus of clause 350 to clause 352, wherein the one or more cavity ridges overlap longitudinally with the at least one ridge of the tail portion.

Clause 354: The apparatus of any one of clauses 350 to 353, wherein the one or more cavity ridges comprise an extension of the at least one ridge of the tail portion into the cavity.

Clause 355: The apparatus of any one of clause 347 to clause 354, wherein the pressure spreading device is adapted to receive an end portion of the fluid inlet conduit and an end portion of the first fluid outlet conduit therein, such that the end portions terminate inside the pressure spreading device.

Clause 356: The apparatus of any one of clause 347 to clause 354, wherein the pressure spreading device is adapted to receive an end portion of the fluid inlet conduit and an end portion of the first fluid outlet conduit therein, such that the end portions are permitted to terminate inside the tail portion.

Clause 357: The apparatus of clause 355 or clause 356, wherein the pressure spreading device includes an inlet conduit channel and an outlet conduit channel adapted for receiving the respective fluid inlet conduit or the first fluid outlet conduit therein.

Clause 358: The apparatus of clause 355 or clause 357, therein the pressure spreading device comprises at least one conduit locating feature adapted for locating an end of the fluid inlet conduit or first fluid outlet conduit.

Clause 359: The apparatus of clause 358, wherein the conduit locating feature comprises a stop.

Clause 360: The apparatus of clause 358 or clause 359, wherein the conduit locating feature is positioned in the inlet conduit channel and/or the outlet conduit channel.

Clause 361: The apparatus of clause 360, wherein the conduit locating feature is positioned at the fluid inlet end of the cavity and/or the fluid outlet end of the cavity.

Clause 362: The apparatus of any one of clauses 341 to 346, wherein the internal flow guide has a fluid flow path feature adapted for allowing fluid to flow from the first compartment to the second compartment.

Clause 363: The apparatus of clause 362, wherein the fluid flow path feature is one or more of an opening, an aperture, or a perforation.

Clause 364: The apparatus of clause 363 wherein the fluid flow path feature includes an opening surround.

Clause 365: The apparatus of clause 364, wherein the opening surround comprises a continuous surround of the opening, aperture or perforation.

Clause 366: The apparatus of clause 364, wherein the opening surround comprises at least one discontinuity.

Clause 367: The apparatus of any one of clauses 365 to 366, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 368: The apparatus of clause 367, wherein the at least one ridge is arranged to overlap with the opening surround of the internal wall.

Clause 369: The apparatus of claim 368, wherein the at least one ridge comprises two or more ridges, at least one of which is arranged to overlap with the opening surround.

Clause 370: The apparatus of clause 367 when dependent on clause 366, wherein the at least one ridge is arranged to extend through the at least one discontinuity in the opening surround.

Clause 371. An apparatus for supplying fluid to a tissue, the apparatus comprising: a receptacle positionable at a tissue area, the receptacle comprising one or more walls and adapted to receive a fluid, the one or more walls comprising at least one section adapted to allow molecules within the fluid to pass from the receptacle to the tissue area; a fluid inlet in fluid communication with the receptacle; and a pressure relief valve in fluid communication with the fluid inlet and/or the receptacle and operable to relieve pressure within the fluid inlet and/or receptacle.

Clause 372. The apparatus of clause 371, wherein the receptacle is inflatable.

Clause 373. The apparatus of any one of clauses 371 to 372, further comprising a cover positionable over the receptacle to form a compartment substantially bounded by the cover, the receptacle and the tissue area, wherein the pressure relief valve is positioned inside the compartment such that the compartment is further bounded by the pressure relief valve.

Clause 374. The apparatus of any one of clauses 371 to 372, further comprising a cover positionable over the receptacle to form a compartment substantially bounded by the cover, the receptacle and the wound area, wherein the pressure relief valve is positioned outside of the compartment.

Clause 375. The apparatus of any one of clauses 371 to 374, wherein the fluid inlet comprises an opening formed in the cover.

Clause 376. The apparatus of any one of clauses 371 to 375, wherein the at least one section of the receptacle comprises a membrane adapted to allow molecules within the fluid to diffuse from the receptacle to the tissue area.

Clause 377. The apparatus of any one of clauses 371 to 376, wherein the receptacle comprises a membrane adapted to allow molecules within the fluid to diffuse from the receptacle to the tissue area.

Clause 378. The apparatus of clause 376 or clause 377, wherein the membrane is adapted to allow the fluid to pass from the receptacle to the tissue area by pore flow.

Clause 379. The apparatus of any one of clauses 376 to 377, wherein the membrane adapted to allow molecules to diffuse from the receptacle to the tissue area is substantially pore-free.

Clause 380. The apparatus of any one of clauses 376 to 377 or 379, wherein the membrane comprises substantially no discontinuities or pores that are visible with an optical or electron microscope at a specified resolution.

Clause 381. The apparatus of clause 380, wherein the specified resolution is on the order of 1 micrometre.

Clause 382. The apparatus of any one of clauses 376 to 381, wherein the membrane adapted to allow molecules to diffuse from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 383. The apparatus of any one of clauses 376 to 382, wherein the membrane is impermeable to bulk transport of fluid.

Clause 384. The apparatus of any one of clauses 371 to 383, wherein the at least one section of the one or more walls of the receptacle are hydrophobic.

Clause 385. The apparatus of any one of the preceding clauses, wherein the one or more walls comprises at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 386. The apparatus of clause 385, wherein the rough surface varies in height.

Clause 387. The apparatus of clause 385 or clause 386, wherein the rough surface includes a plurality of raised and/or recessed elements that vary in width.

Clause 388. The apparatus of any one of clauses 385 to 387, wherein the rough surface is a non-wound contacting surface of the receptacle.

Clause 389. The apparatus of any one of the preceding clauses, wherein the one or more walls comprises a first wall portion and a second wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 391 The apparatus of any one of the preceding clauses, wherein the one or more walls include at least one flexible wall including a first wall portion and a second wall portion, and at least one ridge disposed on an inner surface of at least one of the first wall portion and the second wall portion.

Clause 392 The apparatus of clause 391, wherein the at least one ridge is configured to at least partially maintain the fluid flow path upon a pressing force being applied to the apparatus.

Clause 393. The apparatus of any one of clauses 389 to 392, wherein the at least one ridge has one or more properties configured to at least partially maintain the fluid flow path whilst mitigating indentation of the at least one ridge on the tissue area of the patient, upon a pressing force being applied to the apparatus.

Clause 394. The apparatus of any one of clauses 389 to 393, wherein the at least one ridge is substantially straight.

Clause 395. The apparatus of any one of clauses 389 to 393, wherein the at least one ridge has a longitudinal form, when viewed in plan view, that is substantially non-straight.

Clause 396. The apparatus of clause 395, wherein the substantially non-straight longitudinal form of the at least one ridge, when viewed in plan view, is one or more of curved, zigzag or irregular.

Clause 397. The apparatus of any one of clauses 393 to 396, wherein the one or more properties include that the at least one ridge has a height dimension of between about 100 to 200 micrometres, or between about 125 to 175 micrometres, or approximately 150 micrometres.

Clause 398. The apparatus of any one of clauses 393 to 397, wherein the one or more properties include that the at least one ridge is formed from a yielding material.

Clause 399. The apparatus of any one of clauses 393 to 398, wherein the one or more properties include that the at least one ridge comprises two or more ridges arranged substantially parallel to one another.

Clause 400. The apparatus of any one of clauses 393 to 399, wherein the one or more properties comprise at least one ridge on the inner surface of the first wall portion of the receptacle and at least one ridge on the inner surface of the second wall portion of the receptacle.

Clause 401. The apparatus of clause 400, wherein the one or more properties include that the at least one ridge on the inner surface of the first wall portion is offset from the at least one ridge on the inner surface of the second wall portion.

Clause 402. The apparatus of clause 401, wherein the at least one ridge adjoins the inner surface of the respective first wall portion or second wall portion with a substantially non-filleted corner.

Clause 403. The apparatus of clause 402, wherein the substantially non-filleted corner is a sharp corner.

Clause 404. The apparatus of clause 402, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 405. The apparatus of any one of clauses 393 to 404, wherein a thickness dimension of the first wall portion and/or the second wall portion is less than or approximately equal to a height dimension of the ridge.

Clause 406. The apparatus of any one of clauses 393 to 404, wherein a thickness dimension of the first wall portion and/or the second wall portion is greater than a height dimension of the ridge.

Clause 407. The apparatus of any one of clauses 393 to 406, wherein the first wall portion and/or the second wall portion has a wall thickness of between about 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micrometres and 70 micrometres, or between about 30 micrometres to 60 micrometres, or between about 20 to 60 micrometres, or between about 45 micrometres and 55 micrometres.

Clause 408. The apparatus of clause 407, wherein the first wall portion and/or the second wall portion has a wall thickness of approximately 50 micrometres or of approximately 60 micrometres.

Clause 409. The apparatus of any one of clauses 393 to 408, wherein the inner surface of the at least one of the first wall portion and/or the second wall portion comprises a plurality of the ridges.

Clause 410. The apparatus of any one of clauses 393 to 409, wherein the first wall portion and the second wall portion are part of a single common wall.

Clause 411. The apparatus of any one of clauses 393 to 409 wherein the first wall portion is a portion of a first wall and the second wall portion is a portion of a second wall.

Clause 412. The apparatus of any one of clauses 371 to 411, wherein the receptacle has a plurality of microstructures formed thereon.

Clause 413. The apparatus of any one of clauses 371 to 411, wherein the fluid comprises a therapeutic gas.

Clause 414. The apparatus of clause 413, wherein the gas comprises oxygen and/or carbon dioxide and/or carbon monoxide.

Clause 415. The apparatus of any one of clauses 371 to 414, wherein the tissue area comprises a wound area.

Clause 416. The apparatus of any one of clauses 371 to 415, further comprising a fluid outlet in fluid communication with the receptacle.

Clause 417. The apparatus of clause 416, further comprising a further pressure relief valve in fluid communication with the fluid outlet and/or the receptacle and operable to relieve pressure within the fluid outlet and/or receptacle.

Clause 418. The apparatus of any one of clauses 371 to 417, wherein the receptacle is maintained at a positive pressure.

Clause 419. The apparatus of any one of clauses 417 to 418, wherein the further pressure relief valve is configurable to relieve pressure within the receptacle when the pressure within the receptacle reaches a threshold value.

Clause 420. The apparatus of clause 419, wherein the further pressure relief valve is configurable to adjust the pressure threshold value.

Clause 421. The apparatus of any one of clauses 419 to 420, wherein the pressure threshold value is an upper bound of the pressure.

Clause 422. The apparatus of any one of clauses 417 to 421, wherein the further pressure relief valve is passively and/or mechanically actuable to permit fluid to pass there through.

Clause 423. The apparatus of any one of clauses 417 to 421, wherein the further pressure relief valve is actively actuable to permit fluid to pass there through.

Clause 424. The apparatus of any one of clauses 417 to 423, when dependent on any clause when dependent on clause 375 or clause 376 and clause 374, wherein the further pressure relief valve is positioned in spaced relation from the cover and/or the receptacle.

Clause 425. The apparatus of clause 424 when dependent on clause 417, further comprising a first fluid outlet conduit arranged in fluid communication with the receptacle, wherein the further pressure relief valve is disposed on the first fluid outlet conduit.

Clause 426. The apparatus of any one of clauses 417 to 425, wherein the further pressure relief valve is selectively actuable to an at least partially open position to permit fluid to pass there through, and is configured, when in the at least partially open position, to vent the fluid passing there through to the atmosphere.

Clause 427 The apparatus of any one of clauses 371 to 426, wherein the cover comprises an outer edge via which fluid from within the receptacle and/or fluid from within the compartment is receivable from the compartment substantially bounded by the cover, the receptacle and the tissue area.

Clause 428 The apparatus of any one of clauses 371 to 427, wherein the receptacle is substantially hollow.

Clause 429 The apparatus of any one of clause 371 to 428, wherein the receptacle comprises a head portion that is configured to be positioned over the tissue area, a tail portion that is configured to extend over and/or away from the tissue area.

Clause 430. The apparatus of clause 429, comprising a fluid inlet conduit arranged in fluid communication with the tail portion and/or connected to the tail portion.

Clause 431. The apparatus of clause 429 or 430, wherein the head portion is in fluid communication with the tail portion such that the fluid inlet conduit is configured to supply fluid to the head portion via the tail portion.

Clause 432. The apparatus of any one of clauses 429 to 431, wherein the tail portion is longer than it is wide.

Clause 433. The apparatus of any one of clauses 429 to 432, wherein the tail portion has a width that is less than the width of the head portion.

Clause 434. The apparatus of any one of clauses 371 to 433, further comprising a pressure spreading device adapted for contacting tissue of the patient.

Clause 435. The apparatus of clause 434 when dependent on claim 425 and claim 429, wherein the pressure spreading device encapsulates at least a portion of the fluid inlet conduit and/or the first fluid outlet conduit.

Clause 436. The apparatus of clause 434 when dependent on claim 425 and 429, wherein the pressure spreading device is configured to spread a force applied to the tissue of the patient by the fluid inlet conduit and/or the first fluid outlet conduit across a width of the pressure spreading device so as to mitigate patient discomfort.

Clause 437. The apparatus of any one of clauses 434 to 426 when dependent on any one of clauses 429 to 433, wherein the pressure spreading device adjoins with the tail portion of the receptacle.

Clause 438. The apparatus of clause 437, wherein the pressure spreading device is formed integrally with the tail portion of the receptacle.

Clause 439. The apparatus of clause 437, wherein the pressure spreading device is formed as a separate component from the tail portion of the receptacle.

Clause 440. The apparatus of any one of clauses 434 to 439, wherein the pressure spreading device has a shape configured to reduce pressure exerted on the patient by the fluid inlet conduit and/or the first fluid outlet conduit.

Clause 441 The apparatus of any one of clauses 434 to 439, wherein the pressure spreading device has a width that is greater than an external diameter of the fluid inlet conduit.

Clause 442. The apparatus of any one of clauses 434 to 441, wherein the pressure spreading device has a generally elliptical cross-sectional shape.

Clause 443. The apparatus of any one of clauses 371 to 442, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 444. The apparatus of clause 443, wherein the rough surface is irregular.

Clause 445. The apparatus of any one of clauses 443 to 444, wherein the rough surface varies in height.

Clause 446. The apparatus of any one of clauses 443 to 445, wherein the rough surface includes a plurality of raised and/or recessed elements that vary in width.

Clause 447. The apparatus of any one of clauses 443 to 446, wherein the rough surface is a non-wound contacting surface of the receptacle.

Clause 448. The apparatus of any one of clauses 443 to 447 when dependent on one of clauses 429 to 433, wherein the head portion includes the rough surface.

Clause 449. The apparatus of any one of clauses 443 to 448 when dependent on one of clauses 429 to 433, wherein the tail portion includes the rough surface.

Clause 450. The apparatus of clause 425 or any one of clauses 426 to 449 when dependent on clause 425, wherein the first fluid outlet conduit is configured for cycling fluid out of the receptacle.

Clause 451. The apparatus of clause 450, wherein the first fluid outlet conduit is configured to allow cycling of the fluid out of the receptacle to maintain a concentration of the fluid within the receptacle.

Clause 452. The apparatus of any one of clauses 371 to 451, comprising: a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 453. The apparatus of clause 452, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 454. The apparatus of clause 453, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 455. The apparatus of any one of clauses 452 to clause 454, wherein the internal flow guide comprises an internal wall of the receptacle.

Clause 456. The apparatus of any one of clauses 453 to 457, wherein the one or more walls of the receptacle include at least a section comprising a membrane adapted to allow molecules within the fluid to pass from the receptacle to the tissue of the patient.

Clause 457. The apparatus of clause 456, wherein the receptacle is sealed such that fluid may enter the receptacle substantially only through the fluid inlet; and fluid may exit the receptacle substantially only through the fluid outlet or as a result of molecules within the fluid passing through the membrane.

Clause 458: The apparatus of any one of clauses 434 to 442, wherein the pressure spreading device comprises at least one cavity providing fluid communication between the fluid inlet conduit and the tail portion of the receptacle and/or the tail portion of the receptacle and the first fluid outlet conduit.

Clause 459: The apparatus of clause 458, wherein the cavity has a width dimension that increases from the fluid inlet end of the cavity and/or fluid outlet end of the cavity to the tail portion end of the cavity.

Clause 460: The apparatus of clause 459, wherein the width dimension of the cavity increases gradually from the fluid inlet end of the cavity and/or fluid outlet end of the cavity to the tail portion end of the cavity.

Clause 461: The apparatus of clause 458 or clause 459 or claim 460, wherein the cavity has one or more cavity ridges extending substantially from a fluid inlet end of the cavity to a tail portion end of the cavity or from a fluid outlet end of the cavity to the tail portion end of the cavity.

Clause 462: The apparatus of clause 461, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 463: The apparatus of any one of clause 461 to clause 462, wherein the one or more cavity ridges are positioned so as to be offset from the at least one ridge of the tail portion.

Clause 464: The apparatus of clause 461 to clause 463, wherein the one or more cavity ridges overlap longitudinally with the at least one ridge of the tail portion.

Clause 465: The apparatus of any one of clauses 461 to 464, wherein the one or more cavity ridges comprise an extension of the at least one ridge of the tail portion into the cavity.

Clause 466: The apparatus of any one of clause 458 to clause 465, wherein the pressure spreading device is adapted to receive an end portion of the fluid inlet conduit and an end portion of the first fluid outlet conduit therein, such that the end portions terminate inside the pressure spreading device.

Clause 467: The apparatus of any one of clause 458 to clause 465, wherein the pressure spreading device is adapted to receive an end portion of the fluid inlet conduit and an end portion of the first fluid outlet conduit therein, such that the end portions are permitted to terminate inside the tail portion.

Clause 468: The apparatus of clause 466 or clause 467, wherein the pressure spreading device includes an inlet conduit channel and an outlet conduit channel adapted for receiving the respective fluid inlet conduit or the first fluid outlet conduit therein.

Clause 469: The apparatus of clause 466 or clause 468, therein the pressure spreading device comprises at least one conduit locating feature adapted for locating an end of the fluid inlet conduit or first fluid outlet conduit.

Clause 470: The apparatus of clause 469, wherein the conduit locating feature comprises a stop.

Clause 471: The apparatus of clause 469 or clause 470, wherein the conduit locating feature is positioned in the inlet conduit channel and/or the outlet conduit channel.

Clause 472: The apparatus of clause 471, wherein the conduit locating feature is positioned at the fluid inlet end of the cavity and/or the fluid outlet end of the cavity.

Clause 473: The apparatus of any one of clauses 452 to 457, wherein the internal flow guide has a fluid flow path feature adapted for allowing fluid to flow from the first compartment to the second compartment.

Clause 474: The apparatus of clause 473, wherein the fluid flow path feature is one or more of an opening, an aperture, or a perforation.

Clause 475: The apparatus of clause 474 wherein the fluid flow path feature includes an opening surround.

Clause 476: The apparatus of clause 475, wherein the opening surround comprises a continuous surround of the opening, aperture or perforation.

Clause 477: The apparatus of clause 475, wherein the opening surround comprises at least one discontinuity.

Clause 478: The apparatus of any one of clauses 475 to 477, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 479: The apparatus of clause 478, wherein the at least one ridge is arranged to overlap with the opening surround of the internal wall.

Clause 480: The apparatus of claim 479, wherein the at least one ridge comprises two or more ridges, at least one of which is arranged to overlap with the opening surround.

Clause 481: The apparatus of clause 478 when dependent on clause 477, wherein the at least one ridge is arranged to extend through the at least one discontinuity in the opening surround.

Clause 482. An apparatus for supplying fluid to a tissue, the apparatus comprising: a fluid receptacle positionable at a tissue area, the fluid receptacle comprising one or more walls, a fluid inlet for receiving fluid into the fluid receptacle, and a fluid outlet for the passage of fluid from the fluid receptacle; and a pressure regulator associated with the fluid outlet.

Clause 483. The apparatus of clause 482, wherein the pressure regulator is positioned in spaced relation from the fluid receptacle. Clause 484. The apparatus of clause 482 or 483, further comprising a first fluid outlet conduit arranged in fluid communication with the fluid receptacle, wherein the pressure regulator is disposed on the first fluid outlet conduit.

Clause 485. The apparatus of any one of clauses 482 to 484, wherein the pressure regulator is selectively actuable to an at least partially open position to permit fluid to pass there through, and is configured, when in the at least partially open position, to vent the fluid passing there through to the atmosphere.

Clause 486. The apparatus of any one of clauses 482 to 485, wherein a portion of the fluid receptacle is a tissue facing portion.

Clause 487. The apparatus of any one of clauses 482 to 486, wherein a fluid flow path through the fluid receptacle is defined by the one or more walls of the fluid receptacle, the fluid inlet and the fluid outlet.

Clause 488. The apparatus of clause any one of clauses 482 to 485, wherein the one or more walls of the fluid receptacle are made of a polymer material.

Clause 489. The apparatus of any one of clauses 487 to 487-488, wherein a boundary of the fluid flow path through the fluid receptacle is independent of the tissue of the patient in use.

Clause 490. The apparatus of clause 487-489, wherein the one or more walls is present between the fluid flow path and the tissue of the patient in use.

Clause 491. The apparatus of clause 486, wherein at least a portion of the tissue facing portion comprises a membrane adapted to allow fluid within the receptacle to pass through the membrane to the tissue of the patient.

Clause 492. The apparatus of clause 486 or clause 491, wherein at least a portion of the tissue facing portion comprises a membrane adapted to allow molecules within the fluid to diffuse through the membrane to the tissue of the patient.

Clause 493. The apparatus of any one of clauses 482 to 492, wherein the fluid inlet is adapted for providing a positive pressure within the fluid receptacle.

Clause 494. The apparatus of any one of clauses 482 or 493, wherein the one or more walls of the fluid receptacle define a positive pressure receptacle.

Clause 495. The apparatus of any one of clauses 486 to 494, wherein the fluid receptacle is inflatable.

Clause 496. The apparatus of any one of clauses 482 to 495, wherein the pressure regulator comprises a pressure relief valve.

Clause 497. The apparatus of any one of clauses 482 to 496, wherein the pressure regulator is configurable to relieve pressure within the receptacle when the pressure within the receptacle reaches a threshold value.

Clause 498. The apparatus of any one of clauses 482 to 497, wherein the pressure regulator is configurable to adjust the pressure threshold value.

Clause 499. The apparatus of any one of clauses 493 to 498, wherein the pressure threshold value is an upper bound of the pressure.

Clause 500. The apparatus of any one of clauses 493 to 499, wherein the pressure regulator is passively and/or mechanically actuable and/or actively actuable to permit fluid to pass there through.

Clause 501. The apparatus of any one of clauses 493 to 500, wherein the pressure regulator is actively actuable to permit fluid to pass there through.

Clause 502. The apparatus of any one of clauses 482 to 501, wherein the fluid receptacle comprises multiple layers, comprising a first layer configured to form a tissue facing layer and a second layer.

Clause 503. The apparatus of clause 502, wherein the second layer has a first thickness and the first layer has a second thickness, the first thickness being greater than the second thickness.

Clause 504. The apparatus of clause 502 or clause 503, wherein the second layer and the first layer are permeable, the second layer having a lower permeability than the first layer.

Clause 505. The apparatus of any one of clauses 502 to 504 when dependent on any one of clauses 482 to 495 or 497 to 501, wherein the second layer includes one or more apertures configured to permit fluid within the fluid receptacle to pass there through to exit the fluid receptacle.

Clause 506. The apparatus of clause 505, wherein the one or more apertures are configured to provide pressure relief within the fluid receptacle.

Clause 507. The apparatus of any one of clauses 482 to 506, wherein the fluid receptacle is adapted to allow fluid to enter into the fluid receptacle at the fluid inlet.

Clause 508. The apparatus of clause 507, further comprising a fluid source in fluid communication with the fluid inlet and adapted to provide a substantially continuous supply of fluid to the fluid receptacle.

Clause 509. The apparatus of any one of clauses 482 to 508, further adapted to apply a negative pressure to the tissue of the patient.

Clause 510. The apparatus of clause 509, further comprising a cover positionable over the fluid receptacle at the tissue area of the patient to form a compartment substantially bounded by the cover, the receptacle and the tissue area, the cover comprising a negative pressure port.

Clause 511. The apparatus of clause 483, wherein wherein the pressure regulator is positioned distally from the receptacle so as to be positioned outside of the cover.

Clause 512. The apparatus of any one of clauses 492 or clauses 493 to 511 when dependent on clause 492, wherein the membrane is adapted to allow molecules within the fluid to diffuse from the receptacle to the tissue of the patient.

Clause 513. The apparatus of clause 512, wherein the membrane adapted to allow molecules to diffuse from the receptacle to the tissue area is substantially pore free.

Clause 514. The apparatus of clause 513, wherein the membrane comprises substantially no discontinuities or pores that are visible with an optical or electron microscope at a specified resolution.

Clause 515. The apparatus of clause 514, wherein the specified resolution is on the order of 1 micrometre.

Clause 516. The apparatus of any one of clauses 512 to 515, wherein the membrane adapted to allow molecules to diffuse from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 517. The apparatus of any one of clauses 492 or clauses 512 to 516, wherein the membrane is impermeable to bulk transport of fluid.

Clause 518. The apparatus of clause 492 or any one of clauses 512 to 517, wherein the membrane is hydrophobic.

Clause 519. The apparatus of any one of clauses 510 to 518, wherein the compartment substantially bounded by the cover, the receptacle and the tissue area is pressurised during use to form a negative pressure compartment.

Clause 520. The apparatus of any one of clauses 510 to 519, comprising a negative pressure source arranged in fluid communication with the compartment substantially bounded by the cover, the receptacle and the tissue area for applying a negative pressure to the compartment.

Clause 521. The apparatus of clause 520, wherein the negative pressure source is a pump.

Clause 522. The apparatus of any one of clauses 519 to 521, wherein the pressure in the compartment is altered to be between approximately 50 mmHg to 150 mmHg or between approximately 80 mmHg to 125 mmHg or between 90 mmHg to 110 mmHg below atmospheric pressure.

Clause 523. The apparatus of any one of clauses 482 to 522, wherein the fluid comprises a therapeutic gas.

Clause 524. The apparatus of clause 523, wherein the gas comprises oxygen and/or carbon dioxide and/or carbon monoxide.

Clause 525. The apparatus of any one of clauses 482 to 524, further comprising a fluid source.

Clause 526. The apparatus of clause 525, wherein the fluid source comprises a wall source, gas bottle, gas concentrator, or other gas generator.

Clause 527. The apparatus of clause 492 or any one of clauses 493 to 510 or clauses 516 to 526 when dependent on clause 492, wherein the membrane is adapted to allow molecules within the fluid to pass from the receptacle to the tissue of the patient by pore flow.

Clause 528. The apparatus of any one of clauses 482 to 527, wherein the receptacle is substantially hollow.

Clause 529. The apparatus of clause 486 or any one of clauses 487 to 528 when dependent on clause 486, further comprising structures associated with the tissue facing portion of the receptacle, the structures being configured to face the tissue of the patient.

Clause 530. The apparatus of clause 529, wherein the structures are configured for encouraging cell growth.

Clause 531. The apparatus of clause 529 or 530, wherein the structures are microstructures.

Clause 532. The apparatus of clause 531, wherein the structures comprise formations which protrude from the one or more walls of the receptacle.

Clause 533. The apparatus of clause 531 or 532, wherein molecules within the fluid diffuse through the tissue facing portion of the receptacle and the structures into the tissue area.

Clause 534. The apparatus of any one of clauses 532 to 533, wherein the formations have one or more of a parabolic, trapezoidal, semi-spherical, spherical, pyramidal, conical, domed or frustoconical shape.

Clause 535. The apparatus of any one of clauses 529 to 534, wherein the structures have rounded edges.

Clause 536. The apparatus of any one of clauses 482 to 535, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion.

Clause 537. The apparatus of clause 536, wherein the at least one ridge is configured to at least partially maintain the fluid flow path upon a pressing force being applied to the apparatus.

Clause 538. The apparatus of clause 536 or 537, wherein the at least one ridge has one or more properties configured to at least partially maintain the fluid flow path whilst mitigating indentation of the at least one ridge on the tissue area of the patient, upon a pressing force being applied to the apparatus.

Clause 539. The apparatus of any one of clauses 536 to 538, wherein the at least one ridge is substantially straight.

Clause 540. The apparatus of any one of clauses 536 to 538, wherein the at least one ridge has a longitudinal form, when viewed in plan view, that is substantially non-straight.

Clause 541. The apparatus of clause 540, wherein the substantially non-straight longitudinal form of the at least one ridge, when viewed in plan view, is one or more of curved, zigzag or irregular.

Clause 542. The apparatus of any one of clauses 538 to 541, wherein the one or more properties include that the at least one ridge has a height dimension of between about 100 to 200 micrometres, or between about 125 to 175 micrometres, or approximately 150 micrometres.

Clause 543. The apparatus of any one of clauses 538 to 542, wherein the one or more properties include that the at least one ridge is formed from a yielding material.

Clause 544. The apparatus of any one of clauses 538 to 543, wherein the one or more properties include that the at least one ridge comprises two or more ridges arranged substantially parallel to one another.

Clause 545. The apparatus of any one of clauses 538 to 544, wherein the one or more properties comprise at least one ridge on the inner surface of the first wall portion of the receptacle and at least one ridge on the inner surface of the second wall portion of the receptacle.

Clause 546. The apparatus of clause 545, wherein the one or more properties include that the at least one ridge on the inner surface of the first wall portion is offset from the at least one ridge on the inner surface of the second wall portion.

Clause 547. The apparatus of clause 545 or 546, wherein the at least one ridge adjoins the inner surface of the respective first wall portion or second wall portion with a substantially non-filleted corner.

Clause 548. The apparatus of clause 547, wherein the substantially non-filleted corner is a sharp corner.

Clause 549. The apparatus of clause 546, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 550. The apparatus of any one of clauses 536 to 549, wherein a thickness dimension of the first wall portion and/or the second wall portion is less than or approximately equal to a height dimension of the ridge.

Clause 551. The apparatus of any one of clauses 536 to 549, wherein a thickness dimension of the first wall portion and/or the second wall portion is greater than a height dimension of the ridge.

Clause 552. The apparatus of any one of clauses 536 to 551, wherein the first wall portion and/or the second wall portion has a wall thickness of between 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between about 20 micrometres to 60 micrometres, or between about 45 micrometres and 55 micrometres.

Clause 553. The apparatus of clause 552, wherein the first wall portion and/or the second wall portion has a wall thickness of approximately 50 micrometres or of approximately 60 micrometres.

Clause 554. The apparatus of any one of clauses 536 to 553, wherein the inner surface of the at least one of the first wall portion and/or the second wall portion comprises a plurality of the ridges.

Clause 555. The apparatus of any one of clauses 536 to 554, wherein the first wall portion and the second wall portion are part of a single common wall.

Clause 556. The apparatus of any one of clauses 536 to 554 wherein the first wall portion is a portion of a first wall and the second wall portion is a portion of a second wall.

Clause 557. The apparatus of any one of clauses 531 to 556, wherein the receptacle has a plurality of microstructures formed thereon.

Clause 558. The apparatus of any one of clauses 482 to 557, wherein the tissue area comprises a wound area.

Clause 559. The apparatus of any one of clauses 482 to 558, wherein the cover comprises an outer edge via which fluid from within the receptacle and/or fluid from within the compartment is receivable from the compartment substantially bounded by the cover, the receptacle and the tissue area.

Clause 560. The apparatus of any one of clauses 482 to 559, wherein the receptacle comprises a head portion that is configured to be positioned over the tissue area, a tail portion that is configured to extend over and/or away from the tissue area.

Clause 561. The apparatus of clause 560, and a fluid inlet conduit arranged in fluid communication with the tail portion and/or connected to the tail portion.

Clause 562. The apparatus of clause 560 or 561, wherein the head portion is in fluid communication with the tail portion such that the fluid inlet conduit is configured to supply fluid to the head portion via the tail portion.

Clause 563. The apparatus of any one of clauses 560 to 562, wherein the tail portion is longer than it is wide.

Clause 564. The apparatus of any one of clauses 560 to 563, wherein the tail portion has a width that is less than the width of the head portion.

Clause 565. The apparatus of any one of clauses 482 to 564, further comprising a pressure spreading device adapted for contacting tissue of the patient.

Clause 566. The apparatus of clause 565 when dependent on claim 13 and claim 57, wherein the pressure spreading device encapsulates at least a portion of the fluid inlet conduit and/or the first fluid outlet conduit.

Clause 567. The apparatus of clause 565 when dependent on claim 560 and claim 484, wherein the pressure spreading device is configured to spread a force applied to the tissue of the patient by the fluid inlet conduit and/or the first fluid outlet conduit across a width of the pressure spreading device so as to mitigate patient discomfort.

Clause 568. The apparatus of any one of clauses 565 to 567 when dependent on any one of clauses 560 to 564, wherein the pressure spreading device adjoins with the tail portion of the receptacle.

Clause 569. The apparatus of clause 568, wherein the pressure spreading device is formed integrally with the tail portion of the receptacle.

Clause 570. The apparatus of clause 568, wherein the pressure spreading device is formed as a separate component from the tail portion of the receptacle.

Clause 571. The apparatus of any one of clauses 565 to 570, wherein the pressure spreading device has a shape configured to reduce pressure exerted on the patient by the fluid inlet conduit and/or the first fluid outlet conduit.

Clause 572 The apparatus of any one of clauses 565 to 571, wherein the pressure spreading device has a width that is greater than an external diameter of the fluid inlet conduit.

Clause 573. The apparatus of any one of clauses 565 to 572, wherein the pressure spreading device has a generally elliptical cross-sectional shape.

Clause 574. The apparatus of any one of clauses 482 to 573, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 575. The apparatus of clause 574, wherein the rough surface is irregular.

Clause 576. The apparatus of any one of clauses 574 to 575, wherein the rough surface varies in height.

Clause 577. The apparatus of any one of clauses 574 to 576, wherein the rough surface includes a plurality of raised and/or recessed elements that vary in width.

Clause 578. The apparatus of any one of clauses 574 to 577, wherein the rough surface is a non-wound contacting surface of the receptacle.

Clause 579. The apparatus of any one of clauses 574 to 578 when dependent on one of clauses 560 to 564, wherein the head portion includes the rough surface.

Clause 580. The apparatus of any one of clauses 574 to 579 when dependent on one of clauses 560 to 564, wherein the tail portion includes the rough surface.

Clause 581. The apparatus of clause 484 or any one of clauses 485 to 580 when dependent on clause 484, wherein the first fluid outlet conduit is configured for cycling fluid out of the receptacle.

Clause 582. The apparatus of clause 581, wherein the first fluid outlet conduit is configured to allow cycling of the fluid out of the receptacle to maintain a concentration of the fluid within the receptacle.

Clause 583. The apparatus of any one of clauses 482 to 582, comprising: a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 584. The apparatus of clause 583, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 585. The apparatus of clause 583, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 586. The apparatus of any one of clauses 583 to clause 115, wherein the internal flow guide comprises an internal wall of the receptacle.

Clause 587. The apparatus of any one of clauses 583 to 586, wherein the one or more walls of the receptacle include at least a section comprising a membrane adapted to allow molecules within the fluid to pass from the receptacle to the tissue of the patient.

Clause 588. The apparatus of clause 587, wherein the receptacle is sealed such that fluid may enter the receptacle substantially only through the fluid inlet; and fluid may exit the receptacle substantially only through the fluid outlet or as a result of molecules within the fluid passing through the membrane.

Clause 589: The apparatus of any one of clauses 565 to 573, wherein the pressure spreading device comprises at least one cavity providing fluid communication between the fluid inlet conduit and the tail portion of the receptacle and/or the tail portion of the receptacle and the first fluid outlet conduit.

Clause 590: The apparatus of clause 589, wherein the cavity has a width dimension that increases from the fluid inlet end of the cavity and/or fluid outlet end of the cavity to the tail portion end of the cavity.

Clause 591: The apparatus of clause 590, wherein the width dimension of the cavity increases gradually from the fluid inlet end of the cavity and/or fluid outlet end of the cavity to the tail portion end of the cavity.

Clause 592: The apparatus of clause 589 or clause 590 or claim 591, wherein the cavity has one or more cavity ridges extending substantially from a fluid inlet end of the cavity to a tail portion end of the cavity or from a fluid outlet end of the cavity to the tail portion end of the cavity.

Clause 593: The apparatus of clause 592, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 594: The apparatus of any one of clause 592 to clause 593, wherein the one or more cavity ridges are positioned so as to be offset from the at least one ridge of the tail portion.

Clause 595: The apparatus of clause 592 to clause 594, wherein the one or more cavity ridges overlap longitudinally with the at least one ridge of the tail portion.

Clause 596: The apparatus of any one of clauses 592 to 595, wherein the one or more cavity ridges comprise an extension of the at least one ridge of the tail portion into the cavity.

Clause 597: The apparatus of any one of clause 589 to clause 596, wherein the pressure spreading device is adapted to receive an end portion of the fluid inlet conduit and an end portion of the first fluid outlet conduit therein, such that the end portions terminate inside the pressure spreading device.

Clause 598: The apparatus of any one of clause 589 to clause 596, wherein the pressure spreading device is adapted to receive an end portion of the fluid inlet conduit and an end portion of the first fluid outlet conduit therein, such that the end portions are permitted to terminate inside the tail portion.

Clause 599: The apparatus of clause 597 or clause 598, wherein the pressure spreading device includes an inlet conduit channel and an outlet conduit channel adapted for receiving the respective fluid inlet conduit or the first fluid outlet conduit therein.

Clause 600: The apparatus of clause 598 or clause 599, therein the pressure spreading device comprises at least one conduit locating feature adapted for locating an end of the fluid inlet conduit or first fluid outlet conduit.

Clause 601: The apparatus of clause 600, wherein the conduit locating feature comprises a stop.

Clause 602: The apparatus of clause 600 or clause 601, wherein the conduit locating feature is positioned in the inlet conduit channel and/or the outlet conduit channel.

Clause 603: The apparatus of clause 602, wherein the conduit locating feature is positioned at the fluid inlet end of the cavity and/or the fluid outlet end of the cavity.

Clause 604: The apparatus of any one of clauses 583 to 588, wherein the internal flow guide has a fluid flow path feature adapted for allowing fluid to flow from the first compartment to the second compartment.

Clause 605: The apparatus of clause 604, wherein the fluid flow path feature is one or more of an opening, an aperture, or a perforation.

Clause 606: The apparatus of clause 604 wherein the fluid flow path feature includes an opening surround.

Clause 607: The apparatus of clause 604, wherein the opening surround comprises a continuous surround of the opening, aperture or perforation.

Clause 608: The apparatus of clause 607, wherein the opening surround comprises at least one discontinuity.

Clause 609: The apparatus of any one of clauses 606 to 608, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 610: The apparatus of clause 609, wherein the at least one ridge is arranged to overlap with the opening surround of the internal wall.

Clause 611: The apparatus of claim 610, wherein the at least one ridge comprises two or more ridges, at least one of which is arranged to overlap with the opening surround.

Clause 612: The apparatus of clause 609 when dependent on clause 608, wherein the at least one ridge is arranged to extend through the at least one discontinuity in the opening surround.

Clause 613. An apparatus, comprising: a receptacle comprising one or more walls, a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 614. The apparatus of clause 613, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 615. The apparatus of clause 613, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 616. The apparatus of any one of clauses 613 to clause 615, wherein the internal flow guide comprises an internal wall of the receptacle.

Clause 617. The apparatus of any one of clauses 613 to 616, wherein the one or more walls of the receptacle include at least a section comprising a membrane adapted to allow molecules within the fluid to pass from the receptacle to the tissue of the patient.

Clause 618. The apparatus of clause 617, wherein the receptacle is sealed such that fluid may enter the receptacle substantially only through the fluid inlet; and fluid may exit the receptacle substantially only through the fluid outlet or as a result of molecules within the fluid passing through the membrane.

Clause 619. The apparatus of clause 617 or clause 618, wherein the membrane is adapted to allow molecules within the fluid to diffuse from the receptacle to the tissue of the patient.

Clause 620. The apparatus of clause 619, wherein the membrane adapted to allow molecules to diffuse from the receptacle to the tissue area is substantially pore free.

Clause 621. The apparatus of clause 620, wherein the membrane comprises substantially no discontinuities or pores that are visible with an optical or electron microscope at a specified resolution.

Clause 622. The apparatus of clause 621, wherein the specified resolution is on the order of 1 micrometre.

Clause 623. The apparatus of any one of clauses 617 to 622, wherein the membrane adapted to allow molecules to pass from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 624. The apparatus of any one of clauses 617 to 623, wherein the membrane is impermeable to bulk transport of fluid.

Clause 625. The apparatus of any one of clauses 617 to 624, wherein the membrane is hydrophobic.

Clause 626. The apparatus of any one of clauses 613 to 625, wherein the membrane comprises a tissue facing portion, configured to allow fluid within the receptacle to pass through the tissue facing portion to the tissue of the patient.

Clause 627. The apparatus of clause 626, wherein at least a section of the one or more walls of the receptacle forms the tissue facing portion.

Clause 628. The apparatus of any one of clauses 613 to 627, wherein at least a portion of the internal flow guide has a length, wherein the fluid flow path extends along the length and fluid flows along the fluid flow path in contact with the tissue facing portion such that molecules from the fluid can pass through the tissue facing portion.

Clause 629. The apparatus of any one of clauses 613 to 628, wherein the internal flow guide is configured to prolong a residence time in which fluid entering the fluid inlet spends within the receptacle prior to exiting the receptacle through the fluid outlet.

Clause 630. The apparatus of any one of clauses 614 to 629 having a longitudinal axis, wherein the internal flow guide is configured to extend at least partially along or substantially parallel to the longitudinal axis.

Clause 631. The apparatus of clause 615 wherein the internal flow guide is disposed substantially orthogonal to the tissue facing portion.

Clause 632. The apparatus of any one of clauses 613 to 631, wherein the internal flow guide divides the receptacle into a first compartment and a second compartment, the first compartment and the second compartment being fluidly interconnected.

Clause 633. The apparatus of any one of clauses 613 to 632, wherein the internal flow guide has a fluid flow path feature adapted for allowing fluid to flow from the first compartment to the second compartment.

Clause 634. The apparatus of clause 633, wherein the fluid flow path feature is one or more of an opening, an aperture, a permeable membrane or a perforation.

Clause 635. The apparatus of any one of clauses 613 to 634, wherein the fluid inlet and the fluid outlet are disposed substantially adjacent one another.

Clause 636. The apparatus of any one of clauses 632 to 635, wherein the fluid inlet is disposed to allow fluid to enter into the first compartment, the fluid outlet is disposed to allow fluid in the second compartment to exit the receptacle, and wherein the internal flow guide is configured to substantially separate the first compartment and the second compartment.

Clause 637. The apparatus of clause 635 or 636 when dependent on clause 635, wherein the internal flow guide is configured to cause fluid within the first compartment to flow past at least a portion of the internal flow guide prior to exiting the receptacle at the outlet.

Clause 638. The apparatus of any one of clauses 613 to 636, wherein the internal flow guide is configured to permit fluid within the receptacle to pass through it.

Clause 639. The apparatus of any one of clauses 613 to 638, wherein the internal flow guide has a thickness that is one of greater than or substantially the same as a thickness of the one or more walls of the receptacle.

Clause 640. The apparatus of any one of clauses 613 to 637 or clause 639, wherein the internal flow guide comprises a material adapted to substantially prevent molecules within the fluid from passing through it.

Clause 641. The apparatus of any one of clauses 613 to 640, wherein the receptacle is inflatable.

Clause 642. The apparatus of clause 641, wherein the internal flow guide is disposed inside the inflatable receptacle.

Clause 643. The apparatus of any one of clauses 613 to 642, wherein the receptacle is divided into a first compartment and a second compartment, wherein the first and second compartments of the receptacle are substantially hollow.

Clause 644. The apparatus of any one of clauses 613 to 643, further comprising a pressure regulator associated with the fluid outlet.

Clause 645. The apparatus of clause 644, wherein the pressure regulator is a pressure relief valve.

Clause 646. The apparatus of clause 644 or 645, wherein the pressure regulator is configurable to relieve pressure within the receptacle when the pressure within the receptacle reaches a threshold value.

Clause 647. The apparatus of any one of clauses 644 to 646, wherein the pressure regulator is passively and/or mechanically and/or actively actuable to permit fluid to pass there through.

Clause 648. The apparatus of any one of clauses 644 to 646, wherein the pressure regulator is disposed in spaced relation from the fluid receptacle.

Clause 649. The apparatus of any one of clauses 644 to 648, wherein the pressure regulator is selectively actuable to an at least partially open position to permit fluid to pass there through, and is configured, when in the at least partially open position, to vent the fluid passing there through to the atmosphere.

Clause 650. The apparatus of any one of clauses 613 to 648, wherein a fluid flow path through the fluid receptacle is defined by the one or more walls of the fluid receptacle, the internal flow guide, the fluid inlet and the fluid outlet.

Clause 651. The apparatus of any one of clauses 613 to 649, wherein the receptacle is in the form of a bag.

Clause 652. The apparatus of any one of clauses 613 to 651, wherein the receptacle is configured to face the tissue of the patient in use.

Clause 653. The apparatus of any one of clauses 613 to 652, wherein at least the at least one section of the receptacle is conformable with a surface of the tissue of the patient.

Clause 654. The apparatus of any one of clauses 613 to 653, wherein the one or more walls of the receptacle are adapted to allow molecules within the fluid to diffuse from the receptacle to the tissue of the patient.

Clause 655. The apparatus of any one of clauses 650 to 654, wherein the one or more walls comprise a membrane.

Clause 656. The apparatus of any one of clauses 613 to 655, wherein the at least one section of the receptacle is configured to have an undulating outer surface when at least partially inflated.

Clause 657. The apparatus of any one of clauses 613 to 656, wherein the at least one section of the receptacle comprises a stretchable material.

Clause 658. The apparatus of any one of clauses 613 to 657, further comprising a cover positionable over the receptacle to form a compartment substantially bounded by the cover, the receptacle and the tissue of the patient.

Clause 659. The apparatus of any one of clauses 613 to 658, wherein a boundary of the fluid flow path through the fluid receptacle is independent of the tissue of the patient, optionally wherein the one or more walls is present between the fluid flow path and the tissue of the patient in use.

Clause 660. The apparatus of clause 658 or clause 659, wherein the compartment substantially bounded by the cover, the receptacle and the tissue area is pressurised during use to form a negative pressure compartment.

Clause 661. The apparatus of any one of clauses 658 to 660, comprising a negative pressure source arranged in fluid communication with the compartment substantially bounded by the cover, the receptacle and the tissue area for applying a negative pressure to the compartment.

Clause 662. The apparatus of clause 661, wherein the negative pressure source is a pump.

Clause 663. The apparatus of any one of clauses 660 to 662, wherein the pressure in the compartment is altered to be between approximately 50 mmHg to 150 mmHg or between approximately 80 mmHg to 125 mmHg or between 90 mmHg to 110 mmHg below atmospheric pressure.

Clause 664. The apparatus of any of clauses 658 to 663 when dependent on clause 664, wherein the pressure regulator is positioned distally from the receptacle so as to be positioned outside of the cover.

Clause 665. The apparatus of any one of clauses 613 to 6642, wherein the fluid comprises a therapeutic gas.

Clause 666. The apparatus of clause 665, wherein the gas comprises oxygen and/or carbon dioxide and/or carbon monoxide.

Clause 667. The apparatus of any one of clauses 613 to 666, further comprising a fluid source.

Clause 668. The apparatus of clause 667, wherein the fluid source comprises a wall source, gas bottle, gas concentrator, or other gas generator.

Clause 669. The apparatus of clause 617, wherein the membrane is adapted to allow the molecules within the fluid to pass from the receptacle to the tissue of the patient by pore flow.

Clause 670. The apparatus of any one of clauses 613 to 668, wherein the fluid outlet is configured to allow cycling of fluid out of the fluid receptacle.

Clause 671. The apparatus of any one of clauses 613 to 669, configured for delivery of fluid to tissue of a patient.

Clause 672. The apparatus of any one of clauses 613 to 671, wherein the tissue of the patient comprises a wound area.

Clause 673. The apparatus of clause 617 or any one of clauses 618 to 672 when dependent on clause 617, further comprising structures associated with the at least one section of the receptacle, the structures being configured to face the tissue of the patient.

Clause 674. The apparatus of clause 673, wherein the structures are configured for encouraging cell growth.

Clause 675. The apparatus of clause 673 or 674, wherein the structures are microstructures.

Clause 676. The apparatus of clause 675, wherein the structures comprise formations which protrude from the one or more walls of the receptacle.

Clause 677. The apparatus of clause 675 or 676, wherein molecules within the fluid diffuse through the tissue facing portion of the receptacle and the structures into the tissue. Clause 678. The apparatus of any one of clauses 673 to 677 wherein the structures protrude from a tissue facing surface of the one or more walls of the receptacle. Clause 679. The apparatus of any one of clauses 676 to 678, wherein the formations have one or more of a parabolic, trapezoidal, semi-spherical, spherical, pyramidal, conical, domed or frustoconical shape.

Clause 680. The apparatus of any one of clauses 673 to 678, wherein the structures have rounded edges.

Clause 681. The apparatus of any one of clauses 613 to 678, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion.

Clause 682. The apparatus of clause 681, wherein the at least one ridge is configured to at least partially maintain the fluid flow path upon a pressing force being applied to the apparatus.

Clause 683. The apparatus of clause 681 or 682, wherein the at least one ridge has one or more properties configured to at least partially maintain the fluid flow path whilst mitigating indentation of the at least one ridge on the tissue of the patient, upon a pressing force being applied to the apparatus.

Clause 684. The apparatus of clause 681 or clause 682 or 683, wherein the at least one ridge is substantially straight.

Clause 685. The apparatus of clause 681 or clause 684, wherein the at least one ridge has a longitudinal form, when viewed in plan view, that is substantially non-straight.

Clause 686. The apparatus of clause 685, wherein the substantially non-straight longitudinal form of the at least one ridge, when viewed in plan view, is one or more of curved, zigzag or irregular.

Clause 687. The apparatus of any one of clauses 683 to 686, wherein the one or more properties include that the at least one ridge has a height dimension of between about 100 to 200 micrometres, or between about 125 to 175 micrometres, or approximately 150 micrometres.

Clause 688. The apparatus of any one of clauses 683 to 687, wherein the one or more properties include that the at least one ridge is formed from a yielding material.

Clause 689. The apparatus of any one of clauses 683 to 688, wherein the one or more properties include that the at least one ridge comprises two or more ridges arranged substantially parallel to one another.

Clause 690. The apparatus of any one of clauses 683 to 689, wherein the one or more properties comprise at least one ridge on the inner surface of the first wall portion of the receptacle and at least one ridge on the inner surface of the second wall portion of the receptacle.

Clause 691. The apparatus of clause 690, wherein the one or more properties include that the at least one ridge on the inner surface of the first wall portion is offset from the at least one ridge on the inner surface of the second wall portion.

Clause 692. The apparatus of clause 691, wherein the at least one ridge adjoins the inner surface of the respective first wall portion or second wall portion with a substantially non-filleted corner.

Clause 693. The apparatus of clause 692, wherein the substantially non-filleted corner is a sharp corner.

Clause 694. The apparatus of clause 693, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 695. The apparatus of any one of clauses 381 to 694, wherein a thickness dimension of the first wall portion and/or the second wall portion is less than or approximately equal to a height dimension of the ridge.

Clause 696. The apparatus of any one of clauses 681 to 695, wherein a thickness dimension of the first wall portion and/or the second wall portion is greater than a height dimension of the ridge.

Clause 697. The apparatus of any one of clauses 681 to 696, wherein the first wall portion and/or the second wall portion has a wall thickness of between about 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between about 20 micrometres to 60 micrometres, or between about 45 micrometres and 55 micrometres.

Clause 698. The apparatus of clause 697, wherein the first wall portion and/or the second wall portion has a wall thickness of approximately 50 micrometres or of approximately 60 micrometres.

Clause 699. The apparatus of any one of clauses 681 to 405, wherein the inner surface of the at least one of the first wall portion and/or the second wall portion comprises a plurality of the ridges.

Clause 700. The apparatus of any one of clauses 681 to 699, wherein the first wall portion and the second wall portion are part of a single common wall.

Clause 701. The apparatus of any one of clauses 681 to 699, wherein the first wall portion is a portion of a first wall and the second wall portion is a portion of a second wall.

Clause 702. The apparatus of any one of clauses 673 to 701, wherein the one or more walls of the receptacle has a plurality of microstructures formed thereon.

Clause 703 The apparatus of any one of clauses 613 to 702, wherein the cover comprises an outer edge via which fluid from within the receptacle and/or fluid from within the compartment is receivable from the compartment substantially bounded by the cover, the receptacle and the tissue area.

Clause 704. The apparatus of any one of clause 613 to 703, wherein the receptacle comprises a head portion that is configured to be positioned over the tissue area, a tail portion that is configured to extend over and/or away from the tissue area.

Clause 705. The apparatus of clause 704, and a fluid inlet conduit arranged in fluid communication with the tail portion and/or connected to the tail portion.

Clause 706. The apparatus of clause 704 or 705, wherein the head portion is in fluid communication with the tail portion such that the fluid inlet conduit is configured to supply fluid to the head portion via the tail portion.

Clause 707. The apparatus of any one of clauses 704 to 706, wherein the tail portion is longer than it is wide.

Clause 708. The apparatus of any one of clauses 704 to 707, wherein the tail portion has a width that is less than the width of the head portion.

Clause 709. The apparatus of any one of clauses 613 to 708, further comprising a pressure spreading device adapted for contacting tissue of the patient.

Clause 710. The apparatus of clause 709 when dependent on clause 704, further comprising a first fluid outlet conduit, wherein the pressure spreading device encapsulates at least a portion of the fluid inlet conduit and/or the first fluid outlet conduit.

Clause 711. The apparatus of clause 710, wherein the pressure spreading device is configured to spread a force applied to the tissue of the patient by the fluid inlet conduit and/or the first fluid outlet conduit across a width of the pressure spreading device so as to mitigate patient discomfort.

Clause 712. The apparatus of any one of clauses 709 to 711 when dependent on any one of clauses 704 to 708, wherein the pressure spreading device adjoins with the tail portion of the receptacle.

Clause 713. The apparatus of clause 712, wherein the pressure spreading device is formed integrally with the tail portion of the receptacle.

Clause 714. The apparatus of clause 713, wherein the pressure spreading device is formed as a separate component from the tail portion of the receptacle.

Clause 715. The apparatus of any one of clauses 709 to 714, wherein the pressure spreading device has a shape configured to reduce pressure exerted on the patient by the fluid inlet conduit and/or the first fluid outlet conduit.

Clause 716 The apparatus of any one of clauses 709 to 715, wherein the pressure spreading device has a width that is greater than an external diameter of the fluid inlet conduit.

Clause 717. The apparatus of any one of clauses 709 to 716, wherein the pressure spreading device has a generally elliptical cross-sectional shape.

Clause 718. The apparatus of any one of clauses 613 to 717, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 719. The apparatus of clause 718, wherein the rough surface is irregular.

Clause 720. The apparatus of any one of clauses 718 to 719, wherein the rough surface varies in height.

Clause 721. The apparatus of any one of clauses 718 to 720, wherein the rough surface includes a plurality of raised and/or recessed elements that vary in width.

Clause 722. The apparatus of any one of clauses 718 to 721, wherein the rough surface is a non-wound contacting surface of the receptacle.

Clause 723. The apparatus of any one of clauses 718 to 722 when dependent on one of clauses 90 to 94, wherein the head portion includes the rough surface.

Clause 724. The apparatus of any one of clauses 718 to 723 when dependent on one of clauses 704 to 708, wherein the tail portion includes the rough surface.

Clause 725: The apparatus of any one of clauses 709 to 717, wherein the pressure spreading device comprises at least one cavity providing fluid communication between the fluid inlet conduit and the tail portion of the receptacle and/or the tail portion of the receptacle and the first fluid outlet conduit.

Clause 726: The apparatus of clause 725, wherein the cavity has a width dimension that increases from the fluid inlet end of the cavity and/or fluid outlet end of the cavity to the tail portion end of the cavity.

Clause 727: The apparatus of clause 726, wherein the width dimension of the cavity increases gradually from the fluid inlet end of the cavity and/or fluid outlet end of the cavity to the tail portion end of the cavity.

Clause 728: The apparatus of clause 725 or clause 726 or claim 727, wherein the cavity has one or more cavity ridges extending substantially from a fluid inlet end of the cavity to a tail portion end of the cavity or from a fluid outlet end of the cavity to the tail portion end of the cavity.

Clause 729: The apparatus of clause 728, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 730: The apparatus of any one of clause 728 to clause 729, wherein the one or more cavity ridges are positioned so as to be offset from the at least one ridge of the tail portion.

Clause 731: The apparatus of clause 728 to clause 730, wherein the one or more cavity ridges overlap longitudinally with the at least one ridge of the tail portion.

Clause 732: The apparatus of any one of clauses 728 to 731, wherein the one or more cavity ridges comprise an extension of the at least one ridge of the tail portion into the cavity.

Clause 733: The apparatus of any one of clause 725 to clause 732, wherein the pressure spreading device is adapted to receive an end portion of the fluid inlet conduit and an end portion of the first fluid outlet conduit therein, such that the end portions terminate inside the pressure spreading device.

Clause 734: The apparatus of any one of clause 725 to clause 733, wherein the pressure spreading device is adapted to receive an end portion of the fluid inlet conduit and an end portion of the first fluid outlet conduit therein, such that the end portions are permitted to terminate inside the tail portion.

Clause 735: The apparatus of clause 733 or clause 734, wherein the pressure spreading device includes an inlet conduit channel and an outlet conduit channel adapted for receiving the respective fluid inlet conduit or the first fluid outlet conduit therein.

Clause 736: The apparatus of clause 733 or clause 735, therein the pressure spreading device comprises at least one conduit locating feature adapted for locating an end of the fluid inlet conduit or first fluid outlet conduit.

Clause 737: The apparatus of clause 736, wherein the conduit locating feature comprises a stop.

Clause 738: The apparatus of clause 736 or clause 737, wherein the conduit locating feature is positioned in the inlet conduit channel and/or the outlet conduit channel.

Clause 739: The apparatus of clause 738, wherein the conduit locating feature is positioned at the fluid inlet end of the cavity and/or the fluid outlet end of the cavity.

Clause 740: The apparatus of clause 634 wherein the fluid flow path feature includes an opening surround.

Clause 741: The apparatus of clause 740, wherein the opening surround comprises a continuous surround of the opening, aperture or perforation.

Clause 742: The apparatus of clause 741, wherein the opening surround comprises at least one discontinuity.

Clause 743: The apparatus of any one of clauses 740 to 742, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 744: The apparatus of clause 743, wherein the at least one ridge is arranged to overlap with the opening surround of the internal wall.

Clause 745: The apparatus of claim 744, wherein the at least one ridge comprises two or more ridges, at least one of which is arranged to overlap with the opening surround.

Clause 746: The apparatus of clause 743 when dependent on clause 742, wherein the at least one ridge is arranged to extend through the at least one discontinuity in the opening surround.

Clause 747. An apparatus for supplying fluid to tissue of a patient, the apparatus comprising: a fluid receptacle positionable at a tissue area, the fluid receptacle comprising one or more walls defining a first compartment and a second compartment of the fluid receptacle; a fluid inlet disposed for receiving fluid into the first compartment of the fluid receptacle, and a fluid outlet disposed for the passage of fluid from the second compartment of the fluid receptacle to exit the receptacle; wherein the one or more walls of the receptacle, the fluid inlet and the fluid outlet are configured to define a fluid flow path whereby fluid entering the receptacle at the fluid inlet is caused to pass from the first compartment into the second compartment.

Clause 748. The apparatus of clause 747, the one or more walls of the fluid receptacle including an internal wall configured to divide the receptacle into the first compartment and the second compartment.

Clause 749. The apparatus of clause 747 or clause 748, wherein the one or more walls of the receptacle include at least a section of each of the first compartment and/or the second compartment comprising a membrane adapted to allow molecules within the fluid to pass from the receptacle to the tissue of the patient.

Clause 750. The apparatus of clause 749, wherein the membrane is adapted to allow molecules within the fluid to diffuse from the receptacle to the tissue of the patient.

Clause 751. The apparatus of clause 750, wherein the membrane adapted to allow molecules to diffuse from the receptacle to the tissue area is substantially pore free.

Clause 752. The apparatus of clause 751, wherein the membrane comprises substantially no discontinuities or pores that are visible with an optical or electron microscope at a specified resolution.

Clause 753. The apparatus of clause 752, wherein the specified resolution is on the order of 1 micrometre.

Clause 754. The apparatus of any one of clauses 747 to 753, wherein the membrane adapted to allow molecules to pass from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 755. The apparatus of any one of clauses 747 to 754, wherein the membrane is impermeable to bulk transport of fluid.

Clause 756. The apparatus of any one of clauses 747 to 755, wherein the membrane is hydrophobic.

Clause 757. The apparatus of any one of clauses 747 to 756, wherein the receptacle is sealed such that fluid may enter the receptacle substantially only through the fluid inlet; and fluid may exit the receptacle substantially only through the fluid outlet or as a result of molecules within the fluid passing through the membrane.

Clause 758. The apparatus of any one of clauses 747 to 757, wherein the membrane comprises a tissue facing portion, configured to face the tissue of the patient in use.

Clause 759. The apparatus of clause 758, wherein at least a section of the one or more walls of the receptacle forms the tissue facing portion.

Clause 760. The apparatus of any one of clauses 758 to 759 when dependent on clause 748, wherein at least a portion of the internal wall has a length, and the fluid flow path extends along the length and fluid flows along the fluid flow path in contact with the tissue facing surface such that molecules from the fluid can pass through the tissue facing portion prior to exiting the receptacle.

Clause 761. The apparatus of any one of clauses 747 to 760, wherein the internal wall is configured to prolong a residence time in which fluid entering the fluid inlet spends within the receptacle prior to exiting the receptacle through the fluid outlet.

Clause 762. The apparatus of any one of clauses 747 to 761 having a longitudinal axis, wherein the internal wall is configured to extend at least partially along or substantially parallel to the longitudinal axis.

Clause 763. The apparatus of clause 759, or any one of clauses 760 to 762 when dependent on clause 759, wherein the internal wall has a first flow guide surface and a second opposing flow guide surface and is disposed such that the tissue facing portion is disposed adjacent each of the first flow guide surface and the second flow guide surface.

Clause 764. The apparatus of clause 762 when dependent on clause 759, wherein the internal wall has a first flow guide surface and a second opposing flow guide surface; wherein the internal wall is either disposed in the receptacle such that only its first flow guide surface faces the tissue facing portion.

Clause 765. The apparatus of any one of clauses 747 to 764, wherein the fluid outlet is configured for cycling fluid out of the fluid receptacle.

Clause 766. The apparatus of any one of clauses 748 to 749, wherein at least a periphery of each of the first compartment and the second compartment is adjoining and forms the internal wall.

Clause 767. The apparatus of any one of clauses 747 to 766, wherein the internal wall has a fluid flow path feature adapted for allowing fluid to flow from the first compartment to the second compartment.

Clause 768. The apparatus of clause 767, wherein the fluid flow path feature is one or more of an opening, an aperture, a permeable membrane, a connector or a perforation.

Clause 769. The apparatus of any one of clauses 747 to 768, wherein the fluid inlet and the fluid outlet are disposed substantially adjacent one another.

Clause 770. The apparatus of clause 769 when dependent on clause 748, wherein the internal wall is configured to cause fluid within the first compartment to flow past at least a portion of the internal wall prior to exiting the receptacle via the adjacent outlet.

Clause 771. The apparatus of any one of clauses 748 to 770, wherein the internal wall is configured to permit fluid within the receptacle to pass through it.

Clause 772. The apparatus of any one of clauses 748 to 771, wherein the internal wall has a thickness that is one of greater than or substantially the same as a thickness of the one or more walls of the receptacle.

Clause 773. The apparatus of any one of clauses 748 to 770 or clause 772, wherein the internal wall comprises a material adapted to substantially prevent molecules within the fluid from passing through it.

Clause 774. The apparatus of any one of clauses 747 to 773, wherein the first and second compartments are inflatable.

Clause 775. The apparatus of clause 774, wherein the internal wall is disposed inside the inflatable receptacle.

Clause 776. The apparatus of any one of clauses 748 to 775, wherein the first and second compartments of the receptacle are substantially hollow.

Clause 777. The apparatus of any one of clauses 747 to 776, further comprising a pressure regulator associated with the fluid outlet.

Clause 778. The apparatus of clause 777, wherein the pressure regulator is a pressure relief valve.

Clause 779. The apparatus of clause 777 or 778, wherein the pressure regulator is configurable to relieve pressure within the receptacle when the pressure within the receptacle reaches a threshold value.

Clause 780. The apparatus of any one of clauses 777 to 779, wherein the pressure regulator is passively and/or mechanically actuable and/or actively actuable to permit fluid to pass there through.

Clause 781. The apparatus of any one of clauses 777 to 780, wherein the pressure regulator is disposed in spaced relation from the fluid receptacle.

Clause 782. The apparatus of any of clauses 777 to 781, wherein the pressure regulator is selectively actuable to an at least partially open position to permit fluid to pass there through, and is configured, when in the at least partially open position, to vent the fluid passing there through to the atmosphere.

Clause 783. The apparatus of any one of clauses 747 to 781, wherein a fluid flow path through the fluid receptacle is defined by the one or more walls of the fluid receptacle, the at least a portion of the internal wall, the fluid inlet and the fluid outlet.

Clause 784. The apparatus of any one of clauses 747 to 782, wherein the receptacle is in the form of a bag.

Clause 785. The apparatus of any one of clauses 747 to 784, wherein the receptacle is configured to contact the tissue of the patient in use.

Clause 786. The apparatus of any one of clauses 747 to 785, wherein the one or more walls of the receptacle are conformable with a surface of the tissue of the patient.

Clause 787. The apparatus of any one of clauses 747 to 786, wherein the one or more walls of the receptacle are adapted to allow molecules within the fluid to diffuse from the receptacle to the tissue of the patient.

Clause 788. The apparatus of any one of clauses 747 to 787, wherein the one or more walls comprise a membrane.

Clause 789. The apparatus of any one of clauses 747 to 788, wherein the at least one section of the receptacle is configured to have an undulating outer surface when at least partially inflated.

Clause 790. The apparatus of any one of clauses 747 to 789, wherein the at least one section of the receptacle comprises a stretchable material.

Clause 791. The apparatus of any one of clauses 747 to 790, further comprising a cover positionable over the receptacle to form a compartment substantially bounded by the cover, the receptacle and the tissue of the patient.

Clause 792. The apparatus of clause 791, wherein the compartment substantially bounded by the cover, the receptacle and the tissue area is pressurised during use to form a negative pressure compartment.

Clause 793. The apparatus of any one of clauses 792 to 792, comprising a negative pressure source arranged in fluid communication with the compartment substantially bounded by the cover, the receptacle and the tissue of the patient for applying a negative pressure to the compartment.

Clause 794. The apparatus of clause 793, wherein the negative pressure source is a pump.

Clause 795. The apparatus of any one of clauses 791 to 794, wherein the pressure in the compartment is altered to be between approximately 50 mmHg to 150 mmHg or between approximately 80 mmHg to 125 mmHg or between 90 mmHg to 110 mmHg below atmospheric pressure.

Clause 796. The apparatus of clause 791 to 795 when dependent on clause 777, wherein the pressure regulator is positioned distally from the receptacle so as to be positioned outside of the cover.

Clause 797. The apparatus of any one of clauses 747 to 795, wherein the fluid comprises a therapeutic gas.

Clause 798. The apparatus of clause 797, wherein the gas comprises oxygen and/or carbon dioxide and/or carbon monoxide.

Clause 799. The apparatus of any one of clauses 747 to 798, further comprising a fluid source.

Clause 800. The apparatus of clause 799, wherein the fluid source comprises a wall source, gas bottle, gas concentrator, or other gas generator.

Clause 801. The apparatus of clause 748, wherein the membrane is adapted to allow molecules within the fluid to pass from the receptacle to the tissue of the patient by pore flow.

Clause 802. The apparatus of any one of clauses 747 to 801, wherein the tissue of the patient comprises a wound area.

Clause 803. The apparatus of clause 749 or any one of clauses 750 to 802 when dependent on clause 749, further comprising structures associated with the at least one section of the receptacle, the structures being configured to contact the tissue of the patient.

Clause 804. The apparatus of clause 803, wherein the structures are configured for encouraging cell growth.

Clause 805. The apparatus of clause 803 or 804, wherein the structures are microstructures.

Clause 806. The apparatus of clause 805, wherein the structures comprise formations which protrude from the one or more walls of the receptacle.

Clause 807. The apparatus of clause 805 or 806, wherein molecules within the fluid diffuse through the tissue facing portion of the receptacle and the structures into the tissue of the patient.

Clause 808. The apparatus of any one of clauses 803 to 807 wherein the structures protrude from a tissue facing surface of the one or more walls of the receptacle.

Clause 809. The apparatus of any one of clauses 806 to 808, wherein the formations have one or more of a parabolic, trapezoidal, semi-spherical, spherical, pyramidal, conical, domed or frustoconical shape.

Clause 810. The apparatus of any one of clauses 803 to 809, wherein the structures have rounded edges.

Clause 811. The apparatus of any one of clauses 747 to 810, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion.

Clause 812. The apparatus of clause 811, wherein the at least one ridge is configured to at least partially maintain the fluid flow path upon a pressing force being applied to the apparatus.

Clause 813. The apparatus of clause 811 or 812, wherein the at least one ridge has one or more properties configured to at least partially maintain the fluid flow path whilst mitigating indentation of the at least one ridge on the tissue of the patient, upon a pressing force being applied to the apparatus.

Clause 814. The apparatus of any one of clauses 811 to 813, wherein the one or more properties include that the at least one ridge is substantially straight.

Clause 815. The apparatus of clause 811 or clause 812 or clause 813, wherein the at least one ridge has a longitudinal form, when viewed in plan view, that is substantially non-straight.

Clause 816. The apparatus of clause 815, wherein the substantially non-straight longitudinal form of the at least one ridge, when viewed in plan view, is one or more of curved, zigzag or irregular.

Clause 817. The apparatus of any one of clauses 811 to 816, wherein the one or more properties include that the at least one ridge has a height dimension of between about 100 to 200 micrometres, or between about 125 to 175 micrometres, or approximately 150 micrometres.

Clause 818. The apparatus of any one of clauses 811 to 817, wherein the one or more properties include that the at least one ridge is formed from a yielding material.

Clause 819. The apparatus of any one of clauses 811 to 818, wherein the one or more properties include that the at least one ridge comprises two or more ridges arranged substantially parallel to one another.

Clause 820. The apparatus of any one of clauses 811 to 819, wherein the one or more properties comprise at least one ridge on the inner surface of the first wall portion of the receptacle and at least one ridge on the inner surface of the second wall portion of the receptacle.

Clause 821. The apparatus of clause 820, wherein the one or more properties include that the at least one ridge on the inner surface of the first wall portion is offset from the at least one ridge on the inner surface of the second wall portion.

Clause 822. The apparatus of clause 821, wherein the at least one ridge adjoins the inner surface of the respective first wall portion or second wall portion with a substantially non-filleted corner.

Clause 823. The apparatus of clause 822, wherein the substantially non-filleted corner is a sharp corner.

Clause 824. The apparatus of clause 823, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 825. The apparatus of any one of clauses 811 to 824, wherein a thickness dimension of the first wall portion and/or the second wall portion is less than or approximately equal to a height dimension of the ridge.

Clause 826. The apparatus of any one of clauses 811 to 825, wherein a thickness dimension of the first wall portion and/or the second wall portion is greater than a height dimension of the ridge.

Clause 827. The apparatus of any one of clauses 811 to 826, wherein the first wall portion and/or the second wall portion has a wall thickness of between about 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres or between about 40 micrometres and 70 micrometres, or between about 30 micrometres and 60 micrometres, or between about 20 micrometres and 60 micrometres, or between about 45 micrometres and 55 micrometres.

Clause 828. The apparatus of clause 827, wherein the first wall portion and/or the second wall portion has a wall thickness of approximately 50 micrometres or of approximately 60 micrometres.

Clause 829. The apparatus of any one of clauses 811 to 828, wherein the inner surface of the at least one of the first wall portion and/or the second wall portion comprises a plurality of the ridges.

Clause 830. The apparatus of any one of clauses 811 to 829, wherein the first wall portion and the second wall portion are part of a single common wall.

Clause 831. The apparatus of any one of clauses 811 to 830, wherein the first wall portion is a portion of a first wall and the second wall portion is a portion of a second wall.

Clause 832. The apparatus of any one of clauses 806 to 831, wherein the receptacle has a plurality of microstructures formed thereon.

Clause 833. The apparatus of any one of clauses 747 to 832, wherein the cover comprises an outer edge via which fluid from within the receptacle and/or fluid from within the compartment is receivable from the compartment substantially bounded by the cover, the receptacle and the tissue area.

Clause 834. The apparatus of any one of clauses 747 to 833, wherein the receptacle comprises a head portion that is configured to be positioned over the tissue area, a tail portion that is configured to extend over and/or away from the tissue area.

Clause 835. The apparatus of clause 834, and a fluid inlet conduit arranged in fluid communication with the tail portion and/or connected to the tail portion.

Clause 836. The apparatus of clause 834 or 835, wherein the head portion is in fluid communication with the tail portion such that the fluid inlet conduit is configured to supply fluid to the head portion via the tail portion.

Clause 837. The apparatus of any one of clauses 834 to 836, wherein the tail portion is longer than it is wide.

Clause 838. The apparatus of any one of clauses 834 to 837, wherein the tail portion has a width that is less than the width of the head portion.

Clause 839. The apparatus of any one of clauses 747 to 838, further comprising a pressure spreading device adapted for contacting tissue of the patient.

Clause 840. The apparatus of clause 839 when dependent on clause 834, comprising a first fluid outlet conduit, wherein the pressure spreading device encapsulates at least a portion of the fluid inlet conduit and/or the first fluid outlet conduit.

Clause 841. The apparatus of clause 840 when dependent on clause 834, wherein the pressure spreading device is configured to spread a force applied to the tissue of the patient by the fluid inlet conduit and/or the first fluid outlet conduit across a width of the pressure spreading device so as to mitigate patient discomfort.

Clause 842. The apparatus of any one of clauses 839 to 841 when dependent on any one of clauses 834 to 838, wherein the pressure spreading device adjoins with the tail portion of the receptacle.

Clause 843. The apparatus of clause 842, wherein the pressure spreading device is formed integrally with the tail portion of the receptacle.

Clause 844. The apparatus of clause 842, wherein the pressure spreading device is formed as a separate component from the tail portion of the receptacle.

Clause 845. The apparatus of any one of clauses 840 to 844, when dependent on clause 834, wherein the pressure spreading device has a shape configured to reduce pressure exerted on the patient by the fluid inlet conduit and/or the first fluid outlet conduit.

Clause 846 The apparatus of any one of clauses 839 to 845, wherein the pressure spreading device has a width that is greater than an external diameter of the fluid inlet conduit.

Clause 847. The apparatus of any one of clauses 839 to 846, wherein the pressure spreading device has a generally elliptical cross-sectional shape.

Clause 848. The apparatus of any one of clauses 747 to 847, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 849. The apparatus of clause 848, wherein the rough surface is irregular.

Clause 850. The apparatus of any one of clauses 848 to 849, wherein the rough surface varies in height.

Clause 851. The apparatus of any one of clauses 848 to 850, wherein the rough surface includes a plurality of raised and/or recessed elements that vary in width.

Clause 852. The apparatus of any one of clauses 848 to 851, wherein the rough surface is a non-wound contacting surface of the receptacle.

Clause 853. The apparatus of any one of clauses 848 to 852 when dependent on one of clauses 90 to 94, wherein the head portion includes the rough surface.

Clause 854. The apparatus of any one of clauses 104 to 109 when dependent on one of clauses 834 to 838, wherein the tail portion includes the rough surface.

Clause 855: The apparatus of any one of clauses 840 to 847, when dependent on 834, wherein the pressure spreading device comprises at least one cavity providing fluid communication between the fluid inlet conduit and the tail portion of the receptacle and/or the tail portion of the receptacle and the first fluid outlet conduit.

Clause 856: The apparatus of clause 855, wherein the cavity has a width dimension that increases from the fluid inlet end of the cavity and/or fluid outlet end of the cavity to the tail portion end of the cavity.

Clause 857: The apparatus of clause 856, wherein the width dimension of the cavity increases gradually from the fluid inlet end of the cavity and/or fluid outlet end of the cavity to the tail portion end of the cavity.

Clause 858: The apparatus of clause 855 or clause 856 or claim 857, wherein the cavity has one or more cavity ridges extending substantially from a fluid inlet end of the cavity to a tail portion end of the cavity or from a fluid outlet end of the cavity to the tail portion end of the cavity.

Clause 859: The apparatus of clause 858, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 860: The apparatus of any one of clause 858 to clause 859, wherein the one or more cavity ridges are positioned so as to be offset from the at least one ridge of the tail portion.

Clause 861: The apparatus of clause 858 to clause 860, wherein the one or more cavity ridges overlap longitudinally with the at least one ridge of the tail portion.

Clause 862: The apparatus of any one of clauses 858 to 861, wherein the one or more cavity ridges comprise an extension of the at least one ridge of the tail portion into the cavity.

Clause 863: The apparatus of any one of clause 858 to clause 862, wherein the pressure spreading device is adapted to receive an end portion of the fluid inlet conduit and an end portion of the first fluid outlet conduit therein, such that the end portions terminate inside the pressure spreading device.

Clause 864: The apparatus of any one of clause 858 to clause 863, wherein the pressure spreading device is adapted to receive an end portion of the fluid inlet conduit and an end portion of the first fluid outlet conduit therein, such that the end portions are permitted to terminate inside the tail portion.

Clause 865: The apparatus of clause 863 or clause 864, wherein the pressure spreading device includes an inlet conduit channel and an outlet conduit channel adapted for receiving the respective fluid inlet conduit or the first fluid outlet conduit therein.

Clause 866: The apparatus of clause 863 or clause 865, wherein the pressure spreading device comprises at least one conduit locating feature adapted for locating an end of the fluid inlet conduit or first fluid outlet conduit.

Clause 867: The apparatus of clause 866, wherein the conduit locating feature comprises a stop.

Clause 868: The apparatus of clause 865 or clause 866, wherein the conduit locating feature is positioned in the inlet conduit channel and/or the outlet conduit channel.

Clause 869: The apparatus of clause 868, wherein the conduit locating feature is positioned at the fluid inlet end of the cavity and/or the fluid outlet end of the cavity.

Clause 870: The apparatus of clause 767, wherein the fluid flow path feature is one or more of an opening, an aperture, or a perforation.

Clause 871: The apparatus of clause 870, wherein the fluid flow path feature includes an opening surround.

Clause 872: The apparatus of clause 871, wherein the opening surround comprises a continuous surround of the opening, aperture or perforation.

Clause 873: The apparatus of clause 871, wherein the opening surround comprises at least one discontinuity.

Clause 874: The apparatus of any one of clauses 871 to 873, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 875: The apparatus of clause 874, wherein the at least one ridge is arranged to overlap with the opening surround of the internal wall.

Clause 876: The apparatus of claim 875, wherein the at least one ridge comprises two or more ridges, at least one of which is arranged to overlap with the opening surround.

Clause 877: The apparatus of clause 874 when dependent on clause 873, wherein the at least one ridge is arranged to extend through the at least one discontinuity in the opening surround.

Clause 878. An apparatus for supplying fluid to tissue of a patient, the apparatus comprising: a fluid receptacle comprising one or more outer walls and adapted to receive a fluid, and having an internal wall configured to divide the receptacle into a first region and a second region, the first region and the second region being fluidly interconnected; and a fluid inlet disposed for receiving fluid into the first region of the fluid receptacle, and a fluid outlet disposed for the passage of fluid from the second region of the fluid receptacle, wherein the one or more outer walls of the receptacle, the internal wall, the fluid inlet and the fluid outlet are configured to define a fluid flow path whereby fluid entering the receptacle at the fluid inlet is caused to pass from the first region into the second region and to exit the second region of the receptacle at the fluid outlet.

Clause 879. The apparatus of clause 878, wherein the internal wall includes a fluid flow path feature adapted for allowing fluid to flow from the first region to the second region.

Clause 880. The apparatus of clause 879, wherein the fluid flow path feature is one or more of an opening, an aperture, a permeable membrane, connector or a perforation.

Clause 881. The apparatus of any one of clauses 878 to 880, wherein the one or more outer walls are adapted to allow molecules within the fluid to pass from the receptacle to the tissue of the patient.

Clause 882. The apparatus of clause 881, wherein the one or more outer walls are adapted to allow molecules within the fluid to diffuse from the receptacle to the tissue of the patient.

Clause 883. The apparatus of clause 881 or 882, wherein the one or more outer walls adapted to allow molecules to diffuse from the receptacle to the tissue area are substantially pore free.

Clause 884. The apparatus of clause 883, wherein the one or more outer walls comprise substantially no discontinuities or pores that are visible with an optical or electron microscope at a specified resolution.

Clause 885. The apparatus of clause 884, wherein the specified resolution is on the order of 1 micrometre.

Clause 886. The apparatus of any one of clauses 881 to 885, wherein the one or more outer walls adapted to allow molecules to pass from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 887. The apparatus of any one of clauses 878 to 886, wherein the one or more outer walls are impermeable to bulk transport of fluid.

Clause 888. The apparatus of any one of clauses 878 to 887, wherein the one or more outer walls are hydrophobic.

Clause 889. The apparatus of any one of clauses 878 to 888, wherein the one or more outer walls comprise a membrane.

Clause 890. The apparatus of any one of clauses 878 to 889, wherein at least one section of the one or more outer walls forms a tissue facing portion.

Clause 891. The apparatus of clause 890, wherein the receptacle is sealed such that fluid may enter the receptacle substantially only through the fluid inlet; and fluid may exit the receptacle substantially only through the fluid outlet or as a result of molecules within the fluid passing through the tissue facing portion.

Clause 892. The apparatus of any one of clauses 890 to 891, wherein the tissue facing portion is configured to allow molecules within the fluid within the receptacle to pass through the tissue facing portion to the tissue of the patient.

Clause 893. The apparatus of any one of clauses 889 to 892, wherein at least a portion of the internal wall has a length, the fluid flow path extends along the length, and wherein fluid within the receptacle is caused to pass along the length and in fluid communication with the tissue of the patient via the tissue facing portion.

Clause 894. The apparatus of any one of clauses 878 to 893, wherein the one or more outer walls of the receptacle comprise first and second walls.

Clause 895. The apparatus of clause 894, wherein the first wall forms the tissue facing portion.

Clause 896. The apparatus of any one of clauses 894 to 895, wherein the internal wall is a flow guide.

Clause 897. The apparatus of any one of clauses 894 to 896, wherein a periphery of the second wall is substantially sealed against a periphery of the first wall to form a substantially sealed receptacle.

Clause 898. The apparatus of clause 897, wherein the internal wall at least partially divides the sealed receptacle into the first region and the second region.

Clause 899. The apparatus of clause 896, wherein the flow guide is configured to prolong a residence time in which fluid entering the fluid inlet spends within the receptacle prior to exiting the receptacle through the fluid outlet.

Clause 900. The apparatus of any one of clauses 896 to 899 having a longitudinal axis, wherein the flow guide is configured to extend at least partially along or substantially parallel to the longitudinal axis.

Clause 901. The apparatus of clause 896 when dependent on clause 890 or any one of clauses 897 to 900 when dependent on clause 896 and clause 890 wherein the flow guide has a first flow guide surface and an opposing second flow guide surface, and wherein the flow guide is disposed such that the tissue facing portion is disposed adjacent each of the first flow guide surface and the second flow guide surface.

Clause 902. The apparatus of clause 900 when dependent on clause 896 and clause 890, wherein the flow guide has a first flow guide surface and a second opposing flow guide surface; wherein the flow guide is disposed in the receptacle such that only its first flow guide surface faces the tissue facing portion.

Clause 903. The apparatus of any one of clauses 878 to 902, wherein the fluid inlet and the fluid outlet are disposed substantially adjacent one another.

Clause 904. The apparatus of any one of clauses 878 to 903, wherein the fluid inlet is disposed to allow fluid to enter into the first region, the fluid outlet is disposed to allow fluid in the second region to exit the receptacle, and wherein the internal wall is configured to substantially separate the first region and the second region.

Clause 905. The apparatus of clause 903 or 904 when dependent on clause 903, wherein the internal wall is configured to require fluid entering the receptacle at the fluid inlet in the first region to flow past the internal flow guide prior to exiting the receptacle via the outlet.

Clause 906. The apparatus of any one of clauses 878 to 905, wherein the internal wall is configured to allow molecules within the fluid within the receptacle to pass through it.

Clause 907. The apparatus of any one of clauses 878 to 906, wherein the internal wall has a thickness that is one of greater than or substantially the same as a thickness of the one or more walls of the receptacle.

Clause 908. The apparatus of any one of clauses 878 to 905 or clause 907, wherein the internal wall comprises a material is adapted to substantially prevent molecules within the fluid from passing through it.

Clause 909. The apparatus of any one of clauses 878 to 908, wherein the receptacle is inflatable.

Clause 910. The apparatus of any one of clauses 878 to 909, wherein the receptacle is substantially hollow.

Clause 911. The apparatus of any one of clauses 878 to 910, further comprising a pressure regulator associated with the fluid outlet.

Clause 912. The apparatus of clause 911, wherein the pressure regulator is a pressure relief valve.

Clause 913. The apparatus of any one of clauses 878 to 912, wherein the receptacle is in the form of a bag.

Clause 914. The apparatus of any one of clauses 878 to 913, wherein the receptacle is configured to contact the tissue of the patient in use.

Clause 915. The apparatus of any one of clauses 878 to 914, wherein at least the at least one section of the receptacle is conformable with a surface of the tissue of the patient.

Clause 916. The apparatus of any one of clauses 878 to 915, wherein the one or more outer walls of the receptacle are adapted to allow molecules within the fluid to diffuse from the receptacle to the tissue of the patient.

Clause 917. The apparatus of any one of clauses 878 to 916, wherein the one or more outer walls comprise a membrane.

Clause 918. The apparatus of any one of clauses 879 to 917, wherein the at least one portion of the receptacle is configured to have an undulating outer surface when at least partially inflated.

Clause 919. The apparatus of any one of clauses 879 to 918, wherein the at least one portion of the receptacle comprises a stretchable material.

Clause 920. The apparatus of any one of clauses 878 to 919, further comprising a cover positionable over the receptacle to form a compartment substantially bounded by the cover, the receptacle and the tissue of the patient.

Clause 921. The apparatus of clause 920, wherein the compartment substantially bounded by the cover, the receptacle and the tissue of the patient is pressurised during use to form a negative pressure compartment.

Clause 922. The apparatus of clause 920 or 921, comprising a negative pressure source arranged in fluid communication with the compartment substantially bounded by the cover, the receptacle and the tissue of the patient for applying a negative pressure to the compartment.

Clause 923. The apparatus of clause 922, wherein the negative pressure source is a pump.

Clause 924. The apparatus of any one of clauses 920 to 923, wherein the pressure in the compartment is altered to be between approximately 50 mmHg to 150 mmHg or between approximately 80 mmHg to 125 mmHg or between 90 mmHg to 110 mmHg below atmospheric pressure.

Clause 925. The apparatus of any one of clauses 878 to 924, wherein the fluid comprises a therapeutic gas.

Clause 926. The apparatus of clause 925, wherein the gas comprises oxygen and/or carbon dioxide and/or carbon monoxide.

Clause 927. The apparatus of any one of clauses 878 to 926, further comprising a fluid source.

Clause 928. The apparatus of clause 927, wherein the fluid source comprises a wall source, gas bottle, gas concentrator, or other gas generator.

Clause 929. The apparatus of clause 878, wherein the one or more outer walls are adapted to allow the molecules within the fluid to pass from the receptacle to the tissue of the patient by pore flow.

Clause 930. The apparatus of any one of clauses 878 to 929, wherein the tissue of the patient comprises a wound area.

Clause 931. The apparatus of clause 890 or any one of clauses 891 to 930 when dependent on clause 890, further comprising structures associated with the at least one section of the receptacle, the structures being configured to contact the tissue of the patient.

Clause 932. The apparatus of clause 931, wherein the structures are configured for encouraging cell growth.

Clause 933. The apparatus of clause 931 or 932, wherein the structures are microstructures.

Clause 934. The apparatus of clause 932, wherein the structures comprise formations which protrude from the one or more walls of the receptacle.

Clause 935. The apparatus of clause 931 or 932, wherein molecules within the fluid diffuse through the tissue facing portion of the receptacle and the structures into the tissue of the patient.

Clause 936. The apparatus of any one of clauses 931 to 935 wherein the structures protrude from a tissue facing surface of the one or more walls of the receptacle.

Clause 937. The apparatus of any one of clauses 931 to 936, wherein the formations have one or more of a parabolic, trapezoidal, semi-spherical, spherical, pyramidal, conical, domed or frustoconical shape.

Clause 938. The apparatus of any one of clauses 931 to 937, wherein the structures have rounded edges.

Clause 939. The apparatus of any one of clauses 878 to 938, wherein the one or more outer walls include a first wall portion and a second wall portion opposite the first wall portion, the first wall portion and the second wall portion forming a fluid flow path there between, at least one ridge disposed on an inner surface of at least one of the first wall portion and the second wall portion.

Clause 940. The apparatus of clause 939, wherein the at least one ridge is configured to at least partially maintain the fluid flow path upon a pressing force being applied to the apparatus.

Clause 941. The apparatus of clause 939, wherein the at least one ridge has one or more properties configured to at least partially maintain the fluid flow path whilst mitigating indentation of the at least one ridge on the tissue area of the patient, upon a pressing force being applied to the apparatus.

Clause 942. The apparatus of clause 939 or clause 940, wherein the at least one ridge is substantially straight.

Clause 943. The apparatus of clause 939 or clause 940, wherein the at least one ridge has a longitudinal form, when viewed in plan view, that is substantially non-straight.

Clause 944. The apparatus of clause 943, wherein the substantially non-straight longitudinal form of the at least one ridge, when viewed in plan view, is one or more of curved, zigzag or irregular.

Clause 945. The apparatus of any one of clauses 941 to 944, wherein the one or more properties include that the at least one ridge has a height dimension of between about 100 to 200 micrometres, or between about 125 to 175 micrometres, or approximately 150 micrometres.

Clause 946. The apparatus of any one of clauses 941 to 945, wherein the one or more properties include that the at least one ridge is formed from a yielding material.

Clause 947. The apparatus of any one of clauses 941 to 946, wherein the one or more properties include that the at least one ridge comprises two or more ridges arranged substantially parallel to one another.

Clause 948. The apparatus of any one of clauses 941 to 947, wherein the one or more properties comprise at least one ridge on the inner surface of the first wall portion of the receptacle and at least one ridge on the inner surface of the second wall portion of the receptacle.

Clause 949. The apparatus of clause 948, wherein the one or more properties include that the at least one ridge on the inner surface of the first wall portion is offset from the at least one ridge on the inner surface of the second wall portion.

Clause 950. The apparatus of clause 949, wherein the at least one ridge adjoins the inner surface of the respective first wall portion or second wall portion with a substantially non-filleted corner.

Clause 951. The apparatus of clause 950, wherein the substantially non-filleted corner is a sharp corner.

Clause 952. The apparatus of clause 951, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 953. The apparatus of any one of clauses 939 to 952, wherein a thickness dimension of the first wall portion and/or the second wall portion is less than or approximately equal to a height dimension of the ridge.

Clause 954. The apparatus of any one of clauses 939 to 953, wherein a thickness dimension of the first wall portion and/or the second wall portion is greater than a height dimension of the ridge.

Clause 955. The apparatus of any one of clauses 939 to 954, wherein the first wall portion and/or the second wall portion has a wall thickness of between about 10 micrometres and 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between about 20 micrometres to 60 micrometres, or between about 45 micrometres and 55 micrometres.

Clause 956. The apparatus of clause 955, wherein the first wall portion and/or the second wall portion has a wall thickness of approximately 50 micrometres or of approximately 60 micrometres.

Clause 957. The apparatus of any one of clauses 939 to 956, wherein the inner surface of the at least one of the first wall portion and/or the second wall portion comprises a plurality of the ridges.

Clause 958. The apparatus of any one of clauses 939 to 957, wherein the first wall portion and the second wall portion are part of a single common wall.

Clause 959. The apparatus of any one of clauses 939 to 958, wherein the first wall portion is a portion of a first wall and the second wall portion is a portion of a second wall.

Clause 960. The apparatus of any one of clauses 878 to 959, wherein the one or more outer walls of the receptacle has a plurality of microstructures formed thereon.

Clause 961 The apparatus of any one of clauses 878 to 960, wherein the cover comprises an outer edge via which fluid from within the receptacle and/or fluid from within the compartment is receivable from the compartment substantially bounded by the cover, the receptacle and the tissue area.

Clause 962. The apparatus of any one of clause 878 to 961, wherein the receptacle comprises a head portion that is configured to be positioned over the tissue area, a tail portion that is configured to extend over and/or away from the tissue area.

Clause 963. The apparatus of clause 962, comprising a fluid inlet conduit arranged in fluid communication with the tail portion and/or connected to the tail portion.

Clause 964. The apparatus of clause 962 or 963, wherein the head portion is in fluid communication with the tail portion such that the fluid inlet conduit is configured to supply fluid to the head portion via the tail portion.

Clause 965. The apparatus of any one of clauses 962 to 964, wherein the tail portion is longer than it is wide.

Clause 966. The apparatus of any one of clauses 962 to 965, wherein the tail portion has a width that is less than the width of the head portion.

Clause 967. The apparatus of any one of clauses 878 to 966, further comprising a pressure spreading device adapted for contacting tissue of the patient.

Clause 968. The apparatus of clause 967 when dependent on claim 13 and claim 57, wherein the pressure spreading device encapsulates at least a portion of the fluid inlet conduit and/or the first fluid outlet conduit.

Clause 969. The apparatus of clause 967 when dependent on claim 13 and claim 57, or clause 96, wherein the pressure spreading device is configured to spread a force applied to the tissue of the patient by the fluid inlet conduit and/or the first fluid outlet conduit across a width of the pressure spreading device so as to mitigate patient discomfort.

Clause 970. The apparatus of any one of clauses 967 to 969 when dependent on any one of clauses 962 to 966, wherein the pressure spreading device adjoins with the tail portion of the receptacle.

Clause 971. The apparatus of clause 970, wherein the pressure spreading device is formed integrally with the tail portion of the receptacle.

Clause 972. The apparatus of clause 971, wherein the pressure spreading device is formed as a separate component from the tail portion of the receptacle.

Clause 973. The apparatus of any one of clauses 967 to 972, wherein the pressure spreading device has a shape configured to reduce pressure exerted on the patient by the fluid inlet conduit and/or the first fluid outlet conduit.

Clause 974. The apparatus of any one of clauses 967 to 973, wherein the pressure spreading device has a width that is greater than an external diameter of the fluid inlet conduit.

Clause 975. The apparatus of any one of clauses 967 to 974, wherein the pressure spreading device has a generally elliptical cross-sectional shape.

Clause 976. The apparatus of any one of clauses 878 to 975, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 977. The apparatus of clause 976, wherein the rough surface is irregular.

Clause 978. The apparatus of any one of clauses 976 to 977, wherein the rough surface varies in height.

Clause 979. The apparatus of any one of clauses 976 to 978, wherein the rough surface includes a plurality of raised and/or recessed elements that vary in width.

Clause 980. The apparatus of any one of clauses 976 to 979, wherein the rough surface is a non-wound contacting surface of the receptacle.

Clause 981. The apparatus of any one of clauses 976 to 980 when dependent on one of clauses 962 to 966, wherein the head portion includes the rough surface.

Clause 982. The apparatus of any one of clauses 976 to 981 when dependent on one of clauses 962 to 966, wherein the tail portion includes the rough surface.

Clause 983: The apparatus of any one of clauses 967 to 975, when dependent on clause 962, comprising a first fluid outlet conduit, and wherein the pressure spreading device comprises at least one cavity providing fluid communication between the fluid inlet conduit and the tail portion of the receptacle and/or the tail portion of the receptacle and the first fluid outlet conduit.

Clause 984: The apparatus of clause 983, wherein the cavity has a width dimension that increases from the fluid inlet end of the cavity and/or fluid outlet end of the cavity to the tail portion end of the cavity.

Clause 985: The apparatus of clause 984, wherein the width dimension of the cavity increases gradually from the fluid inlet end of the cavity and/or fluid outlet end of the cavity to the tail portion end of the cavity.

Clause 986: The apparatus of clause 983 or clause 984 or claim 985, wherein the cavity has one or more cavity ridges extending substantially from a fluid inlet end of the cavity to a tail portion end of the cavity or from a fluid outlet end of the cavity to the tail portion end of the cavity.

Clause 987: The apparatus of clause 986, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 988: The apparatus of any one of clause 986 to clause 987, wherein the one or more cavity ridges are positioned so as to be offset from the at least one ridge of the tail portion.

Clause 989: The apparatus of clause 986 to clause 987, wherein the one or more cavity ridges overlap longitudinally with the at least one ridge of the tail portion.

Clause 990: The apparatus of any one of clauses 986 to 989, wherein the one or more cavity ridges comprise an extension of the at least one ridge of the tail portion into the cavity.

Clause 991: The apparatus of any one of clause 983 or clause 984 to 989 when dependent on clause 983, wherein the pressure spreading device is adapted to receive an end portion of the fluid inlet conduit and an end portion of the first fluid outlet conduit therein, such that the end portions terminate inside the pressure spreading device.

Clause 992: The apparatus of any one of clause 983 or clause 984 to 990, wherein the pressure spreading device is adapted to receive an end portion of the fluid inlet conduit and an end portion of the first fluid outlet conduit therein, such that the end portions are permitted to terminate inside the tail portion.

Clause 993: The apparatus of clause 991 or clause 992, wherein the pressure spreading device includes an inlet conduit channel and an outlet conduit channel adapted for receiving the respective fluid inlet conduit or the first fluid outlet conduit therein.

Clause 994: The apparatus of clause 991 or clause 993, therein the pressure spreading device comprises at least one conduit locating feature adapted for locating an end of the fluid inlet conduit or first fluid outlet conduit.

Clause 995: The apparatus of clause 994, wherein the conduit locating feature comprises a stop.

Clause 996: The apparatus of clause 994 or clause 995, wherein the conduit locating feature is positioned in the inlet conduit channel and/or the outlet conduit channel.

Clause 997: The apparatus of clause 996, wherein the conduit locating feature is positioned at the fluid inlet end of the cavity and/or the fluid outlet end of the cavity.

Clause 998: The apparatus of clause 879 or 880 wherein the fluid flow path feature includes an opening surround.

Clause 999: The apparatus of clause 998, wherein the opening surround comprises a continuous surround of the opening, aperture or perforation.

Clause 1000: The apparatus of clause 998, wherein the opening surround comprises at least one discontinuity.

Clause 1001: The apparatus of any one of clauses 998 to 1000, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 1002: The apparatus of clause 1001, wherein the at least one ridge is arranged to overlap with the opening surround of the internal wall.

Clause 1003: The apparatus of claim 1002, wherein the at least one ridge comprises two or more ridges, at least one of which is arranged to overlap with the opening surround.

Clause 1004: The apparatus of clause 1001 when dependent on clause 1000, wherein the at least one ridge is arranged to extend through the at least one discontinuity in the opening surround.

Clause 1005. An apparatus, comprising: a fluid receptacle comprising one or more walls and adapted to receive a fluid therein, the one or more walls having a fluid inlet for receiving fluid into the receptacle and a fluid outlet through which fluid within the receptacle may pass upon exiting the receptacle; wherein the fluid inlet and the fluid outlet are disposed substantially adjacent one another.

Clause 1006. The apparatus of clause 1005, further comprising a fluid inlet conduit arranged in fluid communication with the fluid inlet and a first fluid outlet conduit arranged in fluid communication with the fluid outlet, and substantially parallel to the fluid inlet conduit, wherein the fluid inlet conduit and the first fluid outlet conduit adjoin the receptacle.

Clause 1007. The apparatus of clause 1005 or clause 1006, wherein an end portion of the fluid inlet conduit and an end portion of the first fluid outlet conduit are disposed substantially adjacent one another at a portion of the one or more walls of the receptacle.

Clause 1008. The apparatus of clause 1007, wherein the fluid inlet conduit and the first fluid outlet conduit are arranged substantially parallel with one another at the portion of the one or more walls of the receptacle.

Clause 1009. The apparatus of clause 1008, wherein the fluid inlet conduit and the first fluid outlet conduit are at least partially adjoined so as to extend from the portion of the one or more walls of the receptacle in a common direction.

Clause 1010 The apparatus of clause 1009, wherein the fluid inlet conduit and the first fluid outlet conduit are at least partially adjoined at or near the end portions thereof.

Clause 1011. The apparatus of any one of clauses 1008 to 1010, wherein at least the end portion of the fluid inlet conduit and the first fluid outlet conduit are configured to lie substantially parallel with the tissue of a patient during use.

Clause 1012. The apparatus of clause 1010 or 1011, wherein at least the end portion of the fluid inlet conduit and the first fluid outlet conduit are configured to lie substantially adjacent and in close spaced relation from the tissue of a patient during use.

Clause 1013. The apparatus of clause 1011, wherein at least the end portion of the fluid inlet conduit and the first fluid outlet conduit are configured to lie substantially adjacent and in contact with the tissue of a patient during use.

Clause 1014. The apparatus of any one of clauses 1008 to clause 1013, wherein the receptacle has a longitudinal axis, and at least the end portion of the fluid inlet conduit and the first fluid outlet conduit are arranged to extend from the receptacle in a direction along or substantially parallel to the longitudinal axis.

Clause 10155. The apparatus of any one of clauses 1005 to 1014, wherein the receptacle is inflatable.

Clause 1016. The apparatus of any one of clauses 1005 to 1015, wherein the fluid outlet is configured for cycling fluid out of the fluid receptacle.

Clause 1017. The apparatus of clause 1016, wherein the fluid outlet is configured to allow cycling of the fluid out of the receptacle to maintain a concentration of the fluid within the receptacle.

Clause 1018. The apparatus of any one of clauses 1005 to 1017, wherein the receptacle comprises an internal wall configured to divide the receptacle into a first region and a second region, wherein the fluid inlet is disposed to allow fluid to enter into the first region and the fluid outlet is disposed to allow fluid to exit the receptacle from the second region.

Clause 1019. The apparatus of clause 1018, wherein the internal wall is configured to substantially separate the first region and the second region.

Clause 1020. The apparatus of clause 1018 or 1019, wherein the internal wall is configured to cause fluid entering the receptacle at the fluid inlet in the first region to flow past at least a portion of the internal wall prior to exiting the receptacle via the adjacent outlet.

Clause 1021. The apparatus of any one of clauses 1005 to 1020, wherein at least a section of the one or more walls forms a tissue facing portion.

Clause 1022. The apparatus of clause 1021, wherein the tissue facing portion is adapted to allow molecules within the fluid to pass from the receptacle to the tissue of the patient.

Clause 1023. The apparatus of clause 1022, wherein the tissue facing portion is adapted to allow molecules within the fluid to diffuse from the receptacle to the tissue of the patient.

Clause 1024. The apparatus of clause 1023, wherein the tissue facing portion adapted to allow molecules to diffuse from the receptacle to the tissue area is substantially pore free.

Clause 1025. The apparatus of clause 1024, wherein the tissue facing portion comprises substantially no discontinuities or pores that are visible with an optical or electron microscope at a specified resolution.

Clause 1026. The apparatus of clause 1026, wherein the specified resolution is on the order of 1 micrometre.

Clause 1027. The apparatus of any one of clauses 1023 to 1026, wherein the tissue facing portion adapted to allow molecules to pass from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 1028. The apparatus of any one of clauses 1023 to 1027, wherein the tissue facing portion is impermeable to bulk transport of fluid.

Clause 1029. The apparatus of any one of clauses 1023 to 1027, wherein the tissue facing portion is hydrophobic.

Clause 1030. The apparatus of any one of clauses 1023 to 1029, wherein the tissue facing portion is a membrane.

Clause 1031. The apparatus of any one of clauses 1023 to 1030, wherein the receptacle is sealed such that fluid may enter the receptacle substantially only through the fluid inlet; and fluid may exit the receptacle substantially only through the fluid outlet or as a result of molecules within the fluid passing through the tissue facing portion.

Clause 1032. The apparatus of any one of clauses 1005 to 1031, wherein a fluid flow path through the fluid receptacle is defined by the one or more walls of the fluid receptacle, the at least a portion of the internal wall, the fluid inlet and the fluid outlet.

Clause 1033. The apparatus of clause 1032, wherein a boundary of the fluid flow path through the fluid receptacle is independent of tissue of the patient.

Clause 1034. The apparatus of clause 1033, wherein the one or more walls is present between the fluid flow path and the tissue of the patient in use.

Clause 1035. The apparatus of any one of clauses 1005 to 1033, further comprising a pressure spreading device adapted for contacting tissue of the patient.

Clause 1036. The apparatus of clause 1035, comprising a fluid inlet conduit arranged in fluid communication with the fluid inlet and a first fluid outlet conduit arranged in fluid communication with the fluid outlet, wherein the pressure spreading device encapsulates at least a portion of the fluid inlet conduit and/or the first fluid outlet conduit.

Clause 1037. The apparatus of clause 1036, wherein the pressure spreading device is configured to spread a force applied to the tissue of the patient by the fluid inlet conduit and/or the first fluid outlet conduit across a width of the pressure spreading device so as to mitigate patient discomfort.

Clause 1038. The apparatus of any one of clauses 1005 to 1036, wherein the receptacle comprises a head portion and a tail portion, wherein the fluid inlet and the fluid outlet are disposed at or near the tail portion.

Clause 1039. The apparatus of clause 1038 when dependent on clause 1036 or 1037, wherein the fluid inlet conduit and the first fluid outlet conduit each have a tail end that terminates at or near the tail portion of the receptacle to provide fluid communication between the respective one of the fluid inlet conduit and the first fluid outlet conduit and the receptacle.

Clause 1040. The apparatus of any one of clauses 1038 to 1039, wherein the pressure spreading device adjoins with the tail portion of the receptacle.

Clause 1041. The apparatus of any one of clauses 1038-1039, wherein the pressure spreading device is formed integrally with the tail portion of the receptacle.

Clause 1042. The apparatus of any one of clauses 1038-1041, wherein the pressure spreading device is formed as a separate component from the tail portion of the receptacle.

Clause 1043. The apparatus of any one of clauses 1035 to 1041, wherein the pressure spreading device has a shape configured to reduce pressure exerted on the patient by the fluid inlet conduit and/or the first fluid outlet conduit.

Clause 1044 The apparatus of any one of clauses 1035 to 1043, wherein the pressure spreading device has a width that is greater than an external diameter of the fluid inlet conduit.

Clause 1045. The apparatus of any one of clauses 1035 to 1042, wherein the pressure spreading device has a generally elliptical cross-sectional shape.

Clause 1046. The apparatus of any one of clauses 1005 to 1045, further comprising a pressure regulator associated with the fluid outlet.

Clause 1047. The apparatus of clause 1046, wherein the pressure regulator is a pressure relief valve.

Clause 1048. The apparatus of any one of clauses 1005 to 1047, wherein the receptacle is in the form of a bag.

Clause 1049. The apparatus of any one of clauses 1005 to 1048, wherein the receptacle is configured to contact the tissue of the patient in use.

Clause 1050. The apparatus of any one of clauses 1005 to 1049, wherein at least the at least one section of the receptacle is conformable with a surface of the tissue of the patient.

Clause 1051. The apparatus of any one of clauses 1005 to 1050, wherein the one or more walls of the receptacle are adapted to allow molecules within the fluid to diffuse from the receptacle to the tissue of the patient.

Clause 1052. The apparatus of any one of clauses 1005 to 1051, wherein the one or more walls comprise a membrane.

Clause 1053. The apparatus of clause 1021, wherein the at least a portion of the receptacle is configured to have an undulating outer surface when at least partially inflated.

Clause 1054. The apparatus of any one of clauses 1021 to 1053, wherein the at least a portion of the receptacle comprises a stretchable material.

Clause 1055. The apparatus of any one of clauses 1005 to 1054, further comprising a cover positionable over the receptacle to form a compartment substantially bounded by the cover, the receptacle and the tissue of the patient.

Clause 1056. The apparatus of clause 1055, wherein the compartment substantially bounded by the cover, the receptacle and the tissue of the patient is pressurised during use to form a negative pressure compartment.

Clause 1057. The apparatus of clause 1055 or 1056, comprising a negative pressure source arranged in fluid communication with the compartment substantially bounded by the cover, the receptacle and the tissue of the patient for applying a negative pressure to the compartment.

Clause 1058. The apparatus of clause 1057, wherein the negative pressure source is a pump.

Clause 1059. The apparatus of any one of clauses 1055 to 1057, wherein the pressure in the compartment is altered to be between approximately 50 mmHg to 150 mmHg or between approximately 80 mmHg to 125 mmHg or between 90 mmHg to 110 mmHg below atmospheric pressure.

Clause 1060. The apparatus of any one of clauses 1005 to 1059, wherein the fluid comprises a therapeutic gas.

Clause 1061. The apparatus of clause 1060, wherein the gas comprises oxygen and/or carbon dioxide and/or carbon monoxide.

Clause 1062. The apparatus of any one of clauses 1005 to 1061, further comprising a fluid source.

Clause 1063. The apparatus of clause 1062, wherein the fluid source comprises a wall source, gas bottle, gas concentrator, or other gas generator.

Clause 1064. The apparatus of any one of clauses 1052 to 1063, wherein the membrane is adapted to allow the fluid to pass from the receptacle to the tissue of the patient by pore flow.

Clause 1065. The apparatus of any one of clauses 1005 to 1064, wherein the receptacle is substantially hollow.

Clause 1066. The apparatus of any one of clauses 1005 to 1064, wherein the tissue of the patient comprises a wound area.

Clause 1067. The apparatus of clause 1021 or any one of clauses 1022 to 1066 when dependent on clause 1021, further comprising structures associated with the at least one section of the receptacle, the structures being configured to contact the tissue of the patient.

Clause 1068. The apparatus of clause 1067, wherein the structures are configured for encouraging cell growth.

Clause 1069. The apparatus of clause 1067 or 1068, wherein the structures are microstructures.

Clause 1070. The apparatus of any one of clauses 1067 to 1069, wherein the structures comprise formations which protrude from the one or more walls of the receptacle.

Clause 1071. The apparatus of clause 1069 or 1070, wherein molecules within the fluid diffuse through the tissue facing portion of the receptacle and the structures into the tissue of the patient.

Clause 1072. The apparatus of any one of clauses 1067 to 1071 wherein the structures protrude from a tissue facing surface of the one or more walls of the receptacle.

Clause 1073. The apparatus of any one of clauses 1070 to 1071, wherein the formations have one or more of a parabolic, trapezoidal, semi-spherical, spherical, pyramidal, conical, domed or frustoconical shape.

Clause 1074. The apparatus of any one of clauses 1067 to 1073, wherein the structures have rounded edges.

Clause 1075. The apparatus of any one of clauses 1005 to 1074, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion.

Clause 1076. The apparatus of clause 1075, wherein the at least one ridge is configured to at least partially maintain the fluid flow path upon a pressing force being applied to the apparatus.

Clause 1077. The apparatus of clause 1075 or 1076, wherein the at least one ridge has one or more properties configured to at least partially maintain the fluid flow path whilst mitigating indentation of the at least one ridge on the tissue area of the patient, upon a pressing force being applied to the apparatus.

Clause 1078. The apparatus of clause 1075 or clause 1076 or clause 1077, wherein the at least one ridge is substantially straight.

Clause 1079. The apparatus of clause 1075 or clause 1076 or clause 1077, wherein the at least one ridge has a longitudinal form, when viewed in plan view, that is substantially non-straight.

Clause 1080. The apparatus of clause 1079, wherein the substantially non-straight longitudinal form of the at least one ridge, when viewed in plan view, is one or more of curved, zigzag or irregular.

Clause 1081. The apparatus of any one of clauses 1077 to 1080, wherein the one or more properties include that the at least one ridge has a height dimension of between about 100 to 200 micrometres, or between about 125 to 175 micrometres, or approximately 150 micrometres.

Clause 1082. The apparatus of any one of clauses 1077 to 1081, wherein the one or more properties include that the at least one ridge is formed from a yielding material.

Clause 1083. The apparatus of any one of clauses 1077 to 1082, wherein the one or more properties include that the at least one ridge comprises two or more ridges arranged substantially parallel to one another.

Clause 1084. The apparatus of any one of clauses 1077 to 1083, wherein the one or more properties comprise at least one ridge on the inner surface of the first wall portion of the receptacle and at least one ridge on the inner surface of the second wall portion of the receptacle.

Clause 1085. The apparatus of clause 1084, wherein the one or more properties include that the at least one ridge on the inner surface of the first wall portion is offset from the at least one ridge on the inner surface of the second wall portion.

Clause 1086. The apparatus of clause 1085, wherein the at least one ridge adjoins the inner surface of the respective first wall portion or second wall portion with a substantially non-filleted corner.

Clause 1087. The apparatus of clause 1086, wherein the substantially non-filleted corner is a sharp corner.

Clause 1088. The apparatus of clause 1085, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 1089. The apparatus of any one of clauses 1075 to 1088, wherein a thickness dimension of the first wall portion and/or the second wall portion is less than or approximately equal to a height dimension of the ridge.

Clause 1090. The apparatus of any one of clauses 1075 to 1089, wherein a thickness dimension of the first wall portion and/or the second wall portion is greater than a height dimension of the ridge.

Clause 1091. The apparatus of any one of clauses 1075 to 1090, wherein the first wall portion and/or the second wall portion has a wall thickness of between about 10 micrometres to 150 micrometres, or between 20 micrometres and 140 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between about 20 micrometres and 60 micrometres, or between about 45 micrometres and 55 micrometres.

Clause 1092. The apparatus of clause 1091, wherein the first wall portion and/or the second wall portion has a wall thickness of approximately 50 micrometres or of approximately 60 micrometres.

Clause 1093. The apparatus of any one of clauses 1075 to 1092, wherein the inner surface of the at least one of the first wall portion and/or the second wall portion comprises a plurality of the ridges.

Clause 1094. The apparatus of any one of clauses 1075 to 1093, wherein the first wall portion and the second wall portion are part of a single common wall.

Clause 1095. The apparatus of any one of clauses 1075 to 1094, wherein the first wall portion is a portion of a first wall and the second wall portion is a portion of a second wall.

Clause 1096. The apparatus of any one of clauses 1005 to 1095, wherein the receptacle has a plurality of microstructures formed thereon.

Clause 1097. The apparatus of clause 1039, wherein the head portion is configured to be positioned over the tissue area, and the tail portion is configured to extend over and/or away from the tissue area.

Clause 1098. The apparatus of clause 1097, and a fluid inlet conduit arranged in fluid communication with the tail portion and/or connected to the tail portion.

Clause 1099. The apparatus of clause 1097 or 1098, wherein the head portion is in fluid communication with the tail portion such that the fluid inlet conduit is configured to supply fluid to the head portion via the tail portion.

Clause 1100. The apparatus of any one of clauses 1097 to 1099, wherein the tail portion is longer than it is wide.

Clause 1101. The apparatus of any one of clauses 1097 to 1100, wherein the tail portion has a width that is less than the width of the head portion.

Clause 1102. The apparatus of any one of clauses 1005 to 1101, wherein the one or more walls comprise at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 1103. The apparatus of clause 1102, wherein the rough surface is irregular.

Clause 1104. The apparatus of any one of clauses 1102 to 1103, wherein the rough surface varies in height.

Clause 1105. The apparatus of any one of clauses 1102 to 1104, wherein the rough surface includes a plurality of raised and/or recessed elements that vary in width.

Clause 1106. The apparatus of any one of clauses 1102 to 1105, wherein the rough surface is a non-wound contacting surface of the receptacle.

Clause 1107. The apparatus of any one of clauses 1102 to 1106 when dependent on one of clauses 90 to 94, wherein the head portion includes the rough surface.

Clause 1108. The apparatus of any one of clauses 104 to 109 when dependent on one of clauses 1038 to 1039, wherein the tail portion includes the rough surface.

Clause 1109: The apparatus of any one of clauses 1036 to 1045, wherein the pressure spreading device comprises at least one cavity providing fluid communication between the fluid inlet conduit and the tail portion of the receptacle and/or the tail portion of the receptacle and the first fluid outlet conduit.

Clause 1110: The apparatus of clause 1109, wherein the cavity has a width dimension that increases from the fluid inlet end of the cavity and/or fluid outlet end of the cavity to the tail portion end of the cavity.

Clause 1111: The apparatus of clause 1110, wherein the width dimension of the cavity increases gradually from the fluid inlet end of the cavity and/or fluid outlet end of the cavity to the tail portion end of the cavity.

Clause 1112: The apparatus of clause 1109 or clause 1110 or claim 1111, wherein the cavity has one or more cavity ridges extending substantially from a fluid inlet end of the cavity to a tail portion end of the cavity or from a fluid outlet end of the cavity to the tail portion end of the cavity.

Clause 1113: The apparatus of clause 1112, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 1114: The apparatus of any one of clause 1112 to clause 1113, wherein the one or more cavity ridges are positioned so as to be offset from the at least one ridge of the tail portion.

Clause 1115: The apparatus of clause 1112 to clause 1114, wherein the one or more cavity ridges overlap longitudinally with the at least one ridge of the tail portion.

Clause 1116: The apparatus of any one of clauses 1112 to 1115, wherein the one or more cavity ridges comprise an extension of the at least one ridge of the tail portion into the cavity.

Clause 1117: The apparatus of any one of clause 1109 to clause 1116, wherein the pressure spreading device is adapted to receive an end portion of the fluid inlet conduit and an end portion of the first fluid outlet conduit therein, such that the end portions terminate inside the pressure spreading device.

Clause 1118: The apparatus of any one of clause 1110 to clause 1117, wherein the pressure spreading device is adapted to receive an end portion of the fluid inlet conduit and an end portion of the first fluid outlet conduit therein, such that the end portions are permitted to terminate inside the tail portion.

Clause 1119: The apparatus of clause 1117 or clause 1118, wherein the pressure spreading device includes an inlet conduit channel and an outlet conduit channel adapted for receiving the respective fluid inlet conduit or the first fluid outlet conduit therein.

Clause 1120: The apparatus of clause 1117 or clause 1119, therein the pressure spreading device comprises at least one conduit locating feature adapted for locating an end of the fluid inlet conduit or first fluid outlet conduit.

Clause 1121: The apparatus of clause 1120, wherein the conduit locating feature comprises a stop.

Clause 1122: The apparatus of clause 1120 or clause 1121, wherein the conduit locating feature is positioned in the inlet conduit channel and/or the outlet conduit channel.

Clause 1123: The apparatus of clause 1122, wherein the conduit locating feature is positioned at the fluid inlet end of the cavity and/or the fluid outlet end of the cavity.

Clause 1124: The apparatus of any one of clauses 1018 to 1020, wherein the internal wall includes a fluid flow path feature adapted for allowing fluid to flow from the first region to the second region.

Clause 1125: The apparatus of clause 1124, wherein the fluid flow path feature is one or more of an opening, an aperture, or a perforation.

Clause 1126: The apparatus of clause 1125 wherein the fluid flow path feature includes an opening surround.

Clause 1127: The apparatus of clause 1126, wherein the opening surround comprises a continuous surround of the opening, aperture or perforation.

Clause 1128: The apparatus of clause 1127, wherein the opening surround comprises at least one discontinuity.

Clause 1129: The apparatus of any one of clauses 1126 to 1128, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 1130: The apparatus of clause 1129, wherein the at least one ridge is arranged to overlap with the opening surround of the internal wall.

Clause 1131: The apparatus of claim 1130, wherein the at least one ridge comprises two or more ridges, at least one of which is arranged to overlap with the opening surround.

Clause 1132: The apparatus of clause 1129 when dependent on clause 1128, wherein the at least one ridge is arranged to extend through the at least one discontinuity in the opening surround.

Clause 1133. A tissue care dressing, comprising: a receptacle comprising at least one flexible wall and at least one inner surface; wherein at least a portion of the at least one inner surface comprises a rough surface adapted for reducing adherence of the at least a portion of the at least one inner surface to another portion of the inner surface and/or to another inner surface of the receptacle.

Clause 1134. The tissue care dressing of clause 1133, wherein the receptacle is inflatable.

Clause 1135. The tissue care dressing of clause 1133 or clause 1134, wherein the rough surface is irregular.

Clause 1136. The tissue care dressing of any one of clauses 1133 to 1135, wherein the rough surface varies in height.

Clause 1137. The tissue care dressing of any one of clauses 1133 to 1136, wherein the rough surface includes a plurality of raised and/or recessed elements that vary in width.

Clause 1138. The tissue care dressing of any one of clauses 1133 to 1137, wherein the rough surface is a non-wound contacting surface of the receptacle.

Clause 1139. The tissue care dressing of any one of clauses 1133 to 1137, wherein the receptacle is adapted for connection to a fluid source.

Clause 1140. The tissue care dressing of clause 1139, wherein the receptacle is configured to deliver therapy to a wound upon connection of the receptacle to the fluid source.

Clause 1141. The tissue care dressing of any one of clauses 1139 to 1140, the receptacle being positionable at a tissue area and adapted to receive a fluid, the at least one flexible wall comprising at least one section adapted to allow the fluid to pass from the receptacle to the tissue area.

Clause 1142. The tissue care dressing of clause 1141, wherein the at least one section of the at least one flexible wall is adapted to allow molecules within the fluid to diffuse from the receptacle to the tissue area.

Clause 1143. The tissue care dressing of clause 1142, wherein the at least one section of the at least one flexible wall is adapted to allow the fluid to pass from the receptacle to the tissue area by pore flow.

Clause 1144. The apparatus of clause 1142, wherein the at least one section of the at least one flexible wall adapted to allow molecules to diffuse from the receptacle to the tissue area is substantially pore free.

Clause 1145. The apparatus of clause 1144, wherein the tissue facing portion comprises substantially no discontinuities or pores that are visible with an optical or electron microscope at a specified resolution.

Clause 1146. The apparatus of clause 1145, wherein the specified resolution is on the order of 1 micrometre.

Clause 1147. The apparatus of any one of clauses 1142 to 1146, wherein the tissue facing portion adapted to allow molecules to pass from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 1148. The apparatus of any one of clauses 1142 to 1147, wherein a tissue facing portion of the at least one flexible wall is impermeable to bulk transport of fluid.

Clause 1149. The apparatus of any one of clauses 1142 to 1148, wherein a tissue facing portion of the at least one flexible wall is hydrophobic.

Clause 1150. The tissue care dressing of any one of clauses 1133 to 1149, wherein the receptacle is in the form of a bag.

Clause 1151. The tissue care dressing of any one of clauses 1133 to 1150, wherein the receptacle is configured to contact the tissue area.

Clause 1152. The tissue care dressing of any one of clauses 1141 to 1151, wherein the at least one section of the receptacle is conformable with a surface of the tissue area.

Clause 1153. The tissue care dressing of any one of clauses 1141 to 1152, wherein the at least one section of the at least one flexible wall comprises a stretchable material.

Clause 1154. The tissue care dressing of any one of any one of clauses 1141 to 1153, wherein the at least one section of the at least one flexible wall comprises a membrane.

Clause 1155. The tissue care dressing of clause 1154, wherein the membrane is adapted to allow molecules to diffuse from the receptacle to the tissue area.

Clause 1156. The tissue care dressing of clause 1155, wherein the membrane is substantially pore-free.

Clause 1157. The tissue care dressing of any one of clauses 1154 to clause 1156, wherein the membrane is impermeable to bulk transport of fluid.

Clause 1158. The tissue care dressing of any one of clauses 1141 to 1157, wherein the at least one section of the receptacle is configured to have an undulating outer surface when at least partially inflated.

Clause 1159. The tissue care dressing of any one of clauses 1141 to 1157, further comprising structures associated with the at least one section of the receptacle, the structures being configured to contact the tissue area.

Clause 1160. The tissue care dressing of clause 1159, wherein the structures are configured for encouraging cell growth.

Clause 1161. The tissue care dressing of clause 1159 or clause 1160 wherein the structures are microstructures.

Clause 1162. The tissue care dressing of any one of clauses 1159 to 1161, wherein the structures comprise formations which protrude from the one or more walls of the receptacle.

Clause 1163. The tissue care dressing of clause 1161 or 1162, wherein molecules within the fluid diffuse through the tissue facing portion of the receptacle and the structures into the tissue of the patient.

Clause 1164. The tissue care dressing of any one of clauses 1159 to 1163 wherein the structures protrude from a tissue facing surface of the one or more walls of the receptacle.

Clause 1165. The tissue care dressing of any one of clauses 1162 to 1164, wherein the formations have one or more of a parabolic, trapezoidal, semi-spherical, spherical, pyramidal, conical, domed or frustoconical shape.

Clause 1166. The tissue care dressing of any one of clauses 1159 to 1165, wherein the structures have rounded edges.

Clause 1167. The tissue care dressing of any one of clauses 1133 to 1166, wherein the tissue care dressing comprises a head portion that is configured to be positioned over the tissue area, a tail portion that is configured to extend over and/or away from the tissue area.

Clause 1168. The tissue care dressing of clause 1167, and a fluid inlet conduit arranged in fluid communication with the tail portion and/or connected to the tail portion.

Clause 1169. The tissue care dressing of clause 1168, wherein the head portion is in fluid communication with the tail portion such that the fluid inlet conduit is configured to supply fluid to the head portion via the tail portion.

Clause 1170. The tissue care dressing of any one of clauses 1167 to 1169, wherein the tail portion is longer than it is wide.

Clause 1171. The tissue care dressing of any one of clauses 1168 to 1170, wherein the tail portion has a width that is less than the width of the head portion.

Clause 1172. The tissue care dressing of any one of clauses 1168 to 1171, wherein the head portion includes the rough surface.

Clause 1173. The tissue care dressing of any one of clauses 1168 to 1172, wherein the tail portion includes the rough surface.

Clause 1174. The tissue care dressing of any one of clauses 1133 to 1173, wherein the at least one flexible wall including a first wall portion and a second wall portion opposite the first wall portion, the first wall portion and the second wall portion forming a fluid flow path there between, at least one ridge disposed on an inner surface of at least one of the first wall portion and the second wall portion.

Clause 1175 The tissue care dressing of clause 1174, wherein the at least one ridge is configured to at least partially maintain the fluid flow path upon a pressing force being applied to the tissue care dressing.

Clause 1176. The tissue care dressing of clause 1174 or 1175, wherein the at least one ridge has one or more properties configured to at least partially maintain the fluid flow path whilst mitigating indentation of the at least one ridge on the tissue area of the patient, upon a pressing force being applied to the tissue care dressing.

Clause 1177. The tissue care dressing of clause 1174 or clause 1175 or clause 1176, wherein the at least one ridge is substantially straight.

Clause 1178. The tissue care dressing of clause 1174 or clause 1175 or clause 1176, wherein the at least one ridge has a longitudinal form, when viewed in plan view, that is substantially non-straight.

Clause 1179. The tissue care dressing of clause 1178, wherein the substantially non-straight longitudinal form of the at least one ridge, when viewed in plan view, is one or more of curved, zigzag or irregular.

Clause 1180. The tissue care dressing of clause 1176 or clauses 1177 to 1179 when dependent on clause 1176, wherein the one or more properties include that the at least one ridge has a height dimension of between about 100 to 200 micrometres, or between about 125 to 175 micrometres, or approximately 150 micrometres.

Clause 1181. The tissue care dressing of any one of clauses 1176 to 1180, wherein the one or more properties include that the at least one ridge is formed from a yielding material.

Clause 1182. The tissue care dressing of any one of clauses 1176 to 1181, wherein the one or more properties include that the at least one ridge comprises two or more ridges arranged substantially parallel to one another.

Clause 1183. The tissue care dressing of any one of clauses 1176 to 1182, wherein the one or more properties comprise at least one ridge on the inner surface of the first wall portion of the receptacle and at least one ridge on the inner surface of the second wall portion of the receptacle.

Clause 1184. The tissue care dressing of clause 1183, wherein the one or more properties include that the at least one ridge on the inner surface of the first wall portion is offset from the at least one ridge on the inner surface of the second wall portion.

Clause 1185. The tissue care dressing of clause 1184, wherein the at least one ridge adjoins the inner surface of the respective first wall portion or second wall portion with a substantially non-filleted corner.

Clause 1186. The tissue care dressing of clause 1185, wherein the substantially non-filleted corner is a sharp corner.

Clause 1187. The tissue care dressing of clause 1185, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 1188. The tissue care dressing of any one of clauses 1174 to 1187, wherein a thickness dimension of the first wall portion and/or the second wall portion is less than or approximately equal to a height dimension of the ridge.

Clause 1189. The tissue care dressing of any one of clauses 1174 to 1188, wherein a thickness dimension of the first wall portion and/or the second wall portion is greater than a height dimension of the ridge.

Clause 1190. The tissue care dressing of any one of clauses 1174 to 1189, wherein the first wall portion and/or the second wall portion has a wall thickness of between about 10 micrometres to 150 micrometres, or between about 20 micrometres and 140 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between about 20 micrometres to 60 micrometres, or between about 45 micrometres and 55 micrometres.

Clause 1191. The tissue care dressing of clause 1190, wherein the first wall portion and/or the second wall portion has a wall thickness of approximately 50 micrometres or of approximately 60 micrometres.

Clause 1192. The tissue care dressing of any one of clauses 1174 to 1191, wherein the inner surface of the at least one of the first wall portion and/or the second wall portion comprises a plurality of the ridges.

Clause 1193. The tissue care dressing of any one of clauses 1174 to 1192, wherein the first wall portion and the second wall portion are part of a single common wall.

Clause 1194. The tissue care dressing of any one of clauses 1174 to 1192 wherein the first wall portion is a portion of a first wall and the second wall portion is a portion of a second wall.

Clause 1195. The tissue care dressing of any one of clauses 1174 to 1194, wherein the tissue area comprises a wound area.

Clause 1196. The tissue care dressing of any one of clauses 1174 to 1194, comprising a plurality of the receptacles.

Clause 1197. The tissue care dressing of any one of clauses 1174 to 1196, wherein the fluid comprises a therapeutic gas.

Clause 1198. The tissue care dressing of clause 711, wherein the gas comprises oxygen and/or carbon dioxide and/or carbon monoxide.

Clause 1199. The tissue care dressing of any one of clauses 1174 to 1198, further comprising a fluid source.

Clause 1200. The tissue care dressing of clause 1199, wherein the fluid source comprises a wall source, gas bottle, gas concentrator, or other gas generator.

Clause 1201. The tissue care dressing of any one of clauses 1133 to 1200, further comprising a cover positionable over the receptacle to form a compartment substantially bounded by the cover, the receptacle and the tissue of the patient.

Clause 1202. The tissue care dressing of clause 1201, wherein the compartment substantially bounded by the cover, the receptacle and the tissue of the patient is pressurised during use to form a negative pressure compartment.

Clause 1203. The tissue care dressing of clause 1201 or 1202, comprising a negative pressure source arranged in fluid communication with the compartment substantially bounded by the cover, the receptacle and the tissue of the patient for applying a negative pressure to the compartment.

Clause 1204. The tissue care dressing of clause 1203, wherein the negative pressure source is a pump.

Clause 1205. The tissue care dressing of any one of clauses 1201 to 1204, wherein the pressure in the compartment is altered to be between approximately 50 mmHg to 150 mmHg or between approximately 80 mmHg to 125 mmHg or between 90 mmHg to 110 mmHg below atmospheric pressure.

Clause 1206. The tissue care dressing of any one of clauses 1133 to 1205, further comprising a fluid outlet for the passage of fluid from the fluid receptacle; and a pressure regulator associated with the fluid outlet.

Clause 1207. The tissue care dressing of clause 1206, wherein the pressure regulator comprises a pressure relief valve.

Clause 1208. The tissue care dressing of any one of clauses 1206 or 1207, wherein the pressure regulator is configurable to relieve pressure within the receptacle when the pressure within the receptacle reaches a threshold value.

Clause 1209. The tissue care dressing of any one of clauses 1208, wherein the pressure regulator is configurable to adjust the pressure threshold value.

Clause 1210. The tissue care dressing of any one of clauses 1208 to 1209, wherein the pressure threshold value is an upper bound of the pressure.

Clause 1211. The tissue care dressing of any one of clauses 1206 to 1210, wherein the pressure regulator is passively and/or mechanically actuable and/or actively actuable to permit fluid to pass there through.

Clause 1212. The tissue care dressing of any one of clauses 1206 to 1211, wherein the pressure regulator is actively actuable to permit fluid to pass there through.

Clause 1213. The tissue care dressing of any one of clauses 1133 to 1212, further comprising a pressure spreading device adapted for contacting tissue of the patient.

Clause 1214. The tissue care dressing of clause 1213 when dependent on clause 1168 or any one of clauses 1169 to 1212 when dependent on clause 1168, further comprising a first fluid outlet conduit wherein the pressure spreading device encapsulates at least a portion of the fluid inlet conduit and/or the first fluid outlet conduit.

Clause 1215. The tissue care dressing of either clause 1213 when dependent on clause 1168 or any one of clauses 1169 to 1212 when dependent on clause 1168, or clause 1214, wherein the pressure spreading device is configured to spread a force applied to the tissue of the patient by the fluid inlet conduit and/or the first fluid outlet conduit across a width of the pressure spreading device so as to mitigate patient discomfort.

Clause 1216. The tissue care dressing of any one of clauses 1213 to 1215 when dependent on any one of clauses 1167 to 1171, wherein the pressure spreading device adjoins with the tail portion of the receptacle.

Clause 1217. The tissue care dressing of clause 1216, wherein the pressure spreading device is formed integrally with the tail portion of the receptacle.

Clause 1218. The tissue care dressing of clause 1216, wherein the pressure spreading device is formed as a separate component from the tail portion of the receptacle.

Clause 1219. The tissue care dressing of any one of clauses 1213 to 1218, wherein the pressure spreading device has a shape configured to reduce pressure exerted on the patient by the fluid inlet conduit and/or the first fluid outlet conduit.

Clause 1220. The tissue care dressing of any one of clauses 1213 to 1219, wherein the pressure spreading device has a width that is greater than an external diameter of the fluid inlet conduit.

Clause 1221. The tissue care dressing of any one of clauses 1213 to 1210, wherein the pressure spreading device has a generally elliptical cross-sectional shape.

Clause 1222. The tissue care dressing of any one of clauses 1133 to 1221, comprising: a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 1223. The tissue care dressing of clause 1222, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 1224. The tissue care dressing of clause 1223, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 1225. The tissue care dressing of any one of clauses 1222 to clause 1224, wherein the internal flow guide comprises an internal wall of the receptacle.

Clause 1226. The tissue care dressing of any one of clauses 1222 to 1225, wherein the one or more walls of the receptacle include at least a section comprising a membrane adapted to allow molecules within the fluid to pass from the receptacle to the tissue of the patient.

Clause 1227. The tissue care dressing of clause 1226, wherein the receptacle is sealed such that fluid may enter the receptacle substantially only through the fluid inlet; and fluid may exit the receptacle substantially only through the fluid outlet or as a result of molecules within the fluid passing through the membrane.

Clause 1228: The tissue care dressing of clause 1214 to 1221, wherein the pressure spreading device comprises at least one cavity providing fluid communication between the fluid inlet conduit and the tail portion of the receptacle and/or the tail portion of the receptacle and the first fluid outlet conduit.

Clause 1229: The tissue care dressing of clause 1228, wherein the cavity has a width dimension that increases from the fluid inlet end of the cavity and/or fluid outlet end of the cavity to the tail portion end of the cavity.

Clause 1230: The tissue care dressing of clause 1229, wherein the width dimension of the cavity increases gradually from the fluid inlet end of the cavity and/or fluid outlet end of the cavity to the tail portion end of the cavity.

Clause 1231: The tissue care dressing of clause 1228 or clause 1229 or claim 1230, wherein the cavity has one or more cavity ridges extending substantially from a fluid inlet end of the cavity to a tail portion end of the cavity or from a fluid outlet end of the cavity to the tail portion end of the cavity.

Clause 1232: The tissue care dressing of clause 1231, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 1233: The tissue care dressing of any one of clause 1231 to clause 1232, wherein the one or more cavity ridges are positioned so as to be offset from the at least one ridge of the tail portion.

Clause 1234: The tissue care dressing of clause 1231 to clause 1233, wherein the one or more cavity ridges overlap longitudinally with the at least one ridge of the tail portion.

Clause 1235: The tissue care dressing of any one of clauses 1231 to 1234, wherein the one or more cavity ridges comprise an extension of the at least one ridge of the tail portion into the cavity.

Clause 1236: The tissue care dressing of any one of clause 1228 to clause 1235, wherein the pressure spreading device is adapted to receive an end portion of the fluid inlet conduit and an end portion of the first fluid outlet conduit therein, such that the end portions terminate inside the pressure spreading device.

Clause 1237: The tissue care dressing of any one of clause 1228 to clause 1236, wherein the pressure spreading device is adapted to receive an end portion of the fluid inlet conduit and an end portion of the first fluid outlet conduit therein, such that the end portions are permitted to terminate inside the tail portion.

Clause 1238: The tissue care dressing of clause 1236 or clause 1237, wherein the pressure spreading device includes an inlet conduit channel and an outlet conduit channel adapted for receiving the respective fluid inlet conduit or the first fluid outlet conduit therein.

Clause 1239: The tissue care dressing of clause 1236 or clause 1238, therein the pressure spreading device comprises at least one conduit locating feature adapted for locating an end of the fluid inlet conduit or first fluid outlet conduit.

Clause 1240: The tissue care dressing of clause 1239, wherein the conduit locating feature comprises a stop.

Clause 1241: The tissue care dressing of clause 1239 or clause 1240, wherein the conduit locating feature is positioned in the inlet conduit channel and/or the outlet conduit channel.

Clause 1242: The tissue care dressing of clause 1239, wherein the conduit locating feature is positioned at the fluid inlet end of the cavity and/or the fluid outlet end of the cavity.

Clause 1243: The tissue care dressing of clause 1222 to 1225, wherein the internal flow guide has a fluid flow path feature adapted for allowing fluid to flow from the first compartment to the second compartment.

Clause 1244: The tissue care dressing of clause 1243, wherein the fluid flow path feature is one or more of an opening, an aperture, or a perforation.

Clause 1245: The tissue care dressing of clause 1244 wherein the fluid flow path feature includes an opening surround.

Clause 1246: The tissue care dressing of clause 1245, wherein the opening surround comprises a continuous surround of the opening, aperture or perforation.

Clause 1247: The tissue care dressing of clause 1245, wherein the opening surround comprises at least one discontinuity.

Clause 1248: The tissue care dressing of any one of clauses 1245 to 1247, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 1249: The tissue care dressing of clause 1248, wherein the at least one ridge is arranged to overlap with the opening surround of the internal wall.

Clause 1250: The tissue care dressing of claim 1249, wherein the at least one ridge comprises two or more ridges, at least one of which is arranged to overlap with the opening surround.

Clause 1251: The tissue care dressing of clause 1248 when dependent on clause 1247, wherein the at least one ridge is arranged to extend through the at least one discontinuity in the opening surround.

Clause 1252. A tissue care dressing, comprising: a receptacle being positionable at a tissue area of a patient and comprising at least one flexible wall including a first wall portion and a second wall portion, the first wall portion and the second wall portion forming a fluid flow path there between, at least one ridge disposed on an inner surface of at least one of the first wall portion and the second wall portion.

Clause 1253. The tissue care dressing of clause 1252, wherein the at least one ridge is configured to at least partially maintain the fluid flow path upon a pressing force being applied to the tissue care dressing.

Clause 1254. The tissue care dressing of clause 1252 or 1253, wherein the at least one ridge is configured to at least partially maintain the fluid flow path whilst mitigating indentation of the at least one ridge on the tissue area of the patient, upon a pressing force being applied to the tissue care dressing.

Clause 1255. The tissue care dressing of clause 1252 or clause 1253 or clause 1254, wherein the receptacle comprises a head portion that is configured to be positioned over the tissue area, and a tail portion in fluid communication with the head portion and that is configured to extend away from the tissue area.

Clause 1256. The tissue care dressing of clause 1255, wherein the at least one ridge is positioned in the tail portion and/or in the head portion.

Clause 1257. The tissue care dressing of any one of clauses 1252 to 1256, wherein the at least one ridge has one or more properties configured to maintain the fluid flow path at least partially open upon compression of the tail portion and/or the head portion of the receptacle in use.

Clause 1258. The tissue care dressing of any one of clauses 1252 to 1257, wherein the at least one ridge extends along a longitudinal axis of the tail portion of the receptacle.

Clause 1259. The tissue care dressing of any one of clauses 1252 to 1258, wherein the at least one ridge extends along a longitudinal axis of the head portion of the receptacle.

Clause 1260. The tissue care dressing of any one of clauses 1252 to 1259, wherein the at least one ridge is substantially straight.

Clause 1261. The tissue care dressing of any one of clauses 1252 to 1260, wherein the at least one ridge has a longitudinal form, when viewed in plan view, that is substantially non-straight.

Clause 1262. The tissue care dressing of clause 1261, wherein the substantially non-straight longitudinal form of the at least one ridge, when viewed in plan view, is one or more of curved, zigzag or irregular.

Clause 1263. The tissue care dressing of clause 1257, wherein the one or more properties include that the at least one ridge has a height dimension of between about 100 to 200 micrometres, or between about 125 to 175 micrometres, or approximately 150 micrometres.

Clause 1264. The tissue care dressing of any one of clauses 1257 to 1263, wherein the one or more properties include that the at least one ridge is formed from a yielding material.

Clause 1265. The tissue care dressing of any one of clauses 1257 to 1264, wherein the one or more properties include that the at least one ridge comprises two or more ridges arranged substantially parallel to one another.

Clause 1266. The tissue care dressing of any one of clauses 1257 to 1265, wherein the one or more properties comprise at least one ridge on the inner surface of the first wall portion of the receptacle and at least one ridge on the inner surface of the second wall portion of the receptacle.

Clause 1267. The tissue care dressing of clause 1266, wherein the one or more properties include that the at least one ridge on the inner surface of the first wall portion is offset from the at least one ridge on the inner surface of the second wall portion.

Clause 1268. The tissue care dressing of any one of clauses 1257 to 1267, wherein the at least one ridge adjoins the inner surface of the respective first wall portion or second wall portion with a substantially non-filleted corner.

Clause 1269. The tissue care dressing of clause 1268, wherein the substantially non-filleted corner is a sharp corner.

Clause 1270. The tissue care dressing of clause 1268, wherein the at least one ridge adjoins the inner surface of the respective first wall portion or second wall portion with a fillet having a fillet radius of less than or equal to about 30% or less than or equal to about 25% or less than or equal to about 20% or less than or equal to about 10% of a height dimension of the ridge.

Clause 1271. The tissue care dressing of any one of clauses 1252 to 1270, wherein a thickness dimension of the first wall portion and/or the second wall portion is less than or approximately equal to a height dimension of the ridge.

Clause 1272. The tissue care dressing of any one of clauses 1252 to 1270, wherein a thickness dimension of the first wall portion and/or the second wall portion is greater than a height dimension of the ridge.

Clause 1273. The tissue care dressing of any one of clauses 1252 to 1272, wherein the first wall portion and/or the second wall portion has a wall thickness of between about 10 micrometres and 150 micrometres, or between about 20 micrometres and 150 micrometres, or between about 30 micrometres and 130 micrometres, or between about 35 micrometres and 100 micrometres, or between about 40 micrometres and 80 micrometres, or between about 40 micrometres to 70 micrometres, or between about 30 micrometres to 60 micrometres, or between about 20 micrometres and 60 micrometres, or between about 45 micrometres and 55 micrometres.

Clause 1274. The tissue care dressing of clause 1273, wherein the first wall portion and/or the second wall portion has a wall thickness of approximately 50 micrometres or of approximately 60 micrometres.

Clause 1275. The tissue care dressing of any one of clauses 1252 to 1274, wherein the inner surface of the at least one of the first wall portion and/or the second wall portion comprises a plurality of the ridges.

Clause 1276. The tissue care dressing of any one of clauses 1252 to 1275, wherein the first wall portion and the second wall portion are part of a single common wall.

Clause 1277. The tissue care dressing of any one of clauses 1252 to 1275 wherein the first wall portion is a portion of a first wall and the second wall portion is a portion of a second wall.

Clause 1278. The tissue care dressing of any one of clauses 1252 to 1277, wherein the first wall portion and/or the second wall portion comprises a rough surface adapted for reducing adherence of the first wall portion to the second wall portion.

Clause 1279. The tissue care dressing of any one of clauses 1252 to 1278, wherein the receptacle is inflatable.

Clause 1280. The tissue care dressing of any one of clauses 1252 to 1278, wherein the receptacle is adapted for connection to a fluid source.

Clause 1281. The tissue care dressing of clause 1280, wherein the receptacle is configured to deliver therapy to a wound upon connection of the receptacle to the fluid source.

Clause 1282. The tissue care dressing of any one of clauses 1252 to 1281, the receptacle being adapted to receive a fluid, the at least one flexible wall comprising at least one section adapted to allow the fluid to pass from the receptacle to the tissue area.

Clause 1283. The tissue care dressing of clause 1282, wherein the at least one section of the at least one flexible wall is adapted to allow molecules within the fluid to diffuse from the receptacle to the tissue area.

Clause 1284. The tissue care dressing of clause 1282, wherein the at least one section of the at least one flexible wall is adapted to allow the fluid to pass from the receptacle to the tissue area by pore flow.

Clause 1285. The tissue care dressing of any one of clauses 1252 to 1284, wherein the receptacle is in the form of a bag.

Clause 1286. The tissue care dressing of any one of clauses 1252 to 1284, wherein the receptacle is configured to contact the tissue area.

Clause 1287. The tissue care dressing of any one of clauses 1282 to 1286, wherein the at least one section of the receptacle is conformable with a surface of the tissue area.

Clause 1288. The tissue care dressing of any one of clauses 1282 to 1287, wherein the at least one section of the at least one flexible wall and/or the first wall portion and/or the second wall portion comprises a stretchable material.

Clause 1289. The tissue care dressing of any one of any one of clauses 1282 to 1286, wherein the at least one section and/or the first wall portion and/or the second wall portion of the at least one flexible wall comprises a membrane.

Clause 1290. The tissue care dressing of clause 1289, wherein the membrane is adapted to allow molecules to diffuse from the receptacle to the tissue area.

Clause 1291. The tissue care dressing of clause 1289, wherein the membrane is substantially pore-free.

Clause 1292. The tissue care dressing of any one of clauses 1289 to 1291, wherein the membrane is impermeable to bulk transport of fluid.

Clause 1293. The tissue care dressing of any one of clauses 1282 to 1292, wherein the at least one section of the receptacle is configured to have an undulating outer surface when at least partially inflated.

Clause 1294. The tissue care dressing of any one of clauses 1282 to 1293, further comprising structures associated with the at least one section of the receptacle, the structures being configured to contact the tissue area.

Clause 1295. The tissue care dressing of clause 1294, wherein the structures are configured for encouraging cell growth.

Clause 1296. The tissue care dressing of clause 1294 or clause 1295 wherein the structures are microstructures.

Clause 1297. The tissue care dressing of any one of clauses 1294 to 1296, wherein the structures comprise formations which protrude from the one or more walls of the receptacle.

Clause 1298. The tissue care dressing of any one of clauses 1294 to 1297, wherein molecules within the fluid diffuse through the at least one section of the receptacle and the structures into the tissue of the patient.

Clause 1299. The tissue care dressing of any one of clauses 1294 to 1298 wherein the structures protrude from a tissue facing surface of the one or more walls of the receptacle.

Clause 1300. The tissue care dressing of any one of clauses 1294 to 1299, wherein the formations have one or more of a parabolic, trapezoidal, semi-spherical, spherical, pyramidal, conical, domed or frustoconical shape.

Clause 1301. The tissue care dressing of any one of clauses 1294 to 1300, wherein the structures have rounded edges.

Clause 1302. The tissue care dressing of any one of clauses 1252 to 1301, wherein the tissue care dressing comprises a head portion that is configured to be positioned over the tissue area, a tail portion that is configured to extend over and/or away from the tissue area.

Clause 1303. The tissue care dressing of clause 1302, comprising a fluid inlet conduit arranged in fluid communication with the tail portion and/or connected to the tail portion.

Clause 1304. The tissue care dressing of clause 1303, wherein the head portion is in fluid communication with the tail portion such that the fluid inlet conduit is configured to supply fluid to the head portion via the tail portion.

Clause 1305. The tissue care dressing of any one of clauses 1302 to 1303, wherein the tail portion is longer than it is wide.

Clause 1306. The tissue care dressing of any one of clauses 1302 to 1305, wherein the tail portion has a width that is less than the width of the head portion.

Clause 1307. The tissue care dressing of any one of clauses 1302 to 1305, when dependent on clause 1278, wherein the head portion includes the rough surface.

Clause 1308. The tissue care dressing of any one of clauses 1302 to 1307, when dependent on clause 1278, wherein the tail portion includes the rough surface.

Clause 1309. The tissue care dressing of any one of clauses 1252 to 1308, wherein the tissue area comprises a wound area.

Clause 1310. The tissue care dressing of any one of clauses 1252 to 1309, comprising a plurality of the receptacles.

Clause 1311. The tissue care dressing of any one of clauses 1252 to 1310, wherein the fluid comprises a therapeutic gas.

Clause 1312. The tissue care dressing of clause 1311, wherein the gas comprises oxygen and/or carbon dioxide and/or carbon monoxide.

Clause 1313. The tissue care dressing of any one of clauses 1252 to 1312, further comprising a fluid source.

Clause 1314. The tissue care dressing of clause 1313, wherein the fluid source comprises a wall source, gas bottle, gas concentrator, or other gas generator.

Clause 1315. The tissue care dressing of any one of clauses 1252 to 1314, further comprising a cover positionable over the receptacle to form a compartment substantially bounded by the cover, the receptacle and the tissue of the patient.

Clause 1316. The tissue care dressing of clause 1315, wherein the compartment substantially bounded by the cover, the receptacle and the tissue of the patient is pressurised during use to form a negative pressure compartment.

Clause 1317. The tissue care dressing of clause 1315 or 1316, comprising a negative pressure source arranged in fluid communication with the compartment substantially bounded by the cover, the receptacle and the tissue of the patient for applying a negative pressure to the compartment.

Clause 1318. The tissue care dressing of clause 1318, wherein the negative pressure source is a pump.

Clause 1319. The tissue care dressing of any one of clauses 1315 to 1318, wherein the pressure in the compartment is altered to be between approximately 50 mmHg to 150 mmHg or between approximately 80 mmHg to 125 mmHg or between 90 mmHg to 110 mmHg below atmospheric pressure.

Clause 1320. The tissue care dressing of any one of clauses 1252 to 1319, further comprising a fluid outlet for the passage of fluid from the fluid receptacle; and a pressure regulator associated with the fluid outlet.

Clause 1321. The tissue care dressing of clause 1320, wherein the pressure regulator comprises a pressure relief valve.

Clause 1322. The tissue care dressing of any one of clauses 1320 or 1321, wherein the pressure regulator is configurable to relieve pressure within the receptacle when the pressure within the receptacle reaches a threshold value.

Clause 1323. The tissue care dressing of any one of clauses 1320 to 1322, wherein the pressure regulator is configurable to adjust the pressure threshold value.

Clause 1324. The tissue care dressing of any one of clauses 1322 to 1323, wherein the pressure threshold value is an upper bound of the pressure.

Clause 1325. The tissue care dressing of any one of clauses 1320 to 1324, wherein the pressure regulator is passively and/or mechanically actuable and/or actively actuable to permit fluid to pass there through.

Clause 1326. The tissue care dressing of any one of clauses 1320 to 1325, wherein the pressure regulator is actively actuable to permit fluid to pass there through.

Clause 1327. The tissue care dressing of any one of clauses 1252 to 1326, further comprising a pressure spreading device adapted for contacting tissue of the patient.

Clause 1328. The tissue care dressing of clause 1327 when dependent on clause 1303, further comprising a fluid inlet arranged in fluid communication with the receptacle and a first fluid outlet conduit arranged in fluid communication with the receptacle, wherein the pressure spreading device encapsulates at least a portion of the fluid inlet conduit and/or the first fluid outlet conduit.

Clause 1329. The tissue care dressing of clause 1328, wherein the pressure spreading device is configured to spread a force applied to the tissue of the patient by the fluid inlet conduit and/or the first fluid outlet conduit across a width of the pressure spreading device so as to mitigate patient discomfort.

Clause 1330. The tissue care dressing of any one of clauses 1327 to 1329 when dependent on any one of clauses 1302 to 1308, wherein the pressure spreading device adjoins with the tail portion of the receptacle.

Clause 1331. The tissue care dressing of clause 1330, wherein the pressure spreading device is formed integrally with the tail portion of the receptacle.

Clause 1332. The tissue care dressing of clause 1330, wherein the pressure spreading device is formed as a separate component from the tail portion of the receptacle.

Clause 1333. The tissue care dressing of clause 1328 or any one of clauses 1329 to 1332 when dependent on 1328, wherein the pressure spreading device has a shape configured to reduce pressure exerted on the patient by the fluid inlet conduit and/or the first fluid outlet conduit.

Clause 1334. The tissue care dressing of any one of clauses 1328 to 1333, wherein the pressure spreading device has a width that is greater than an external diameter of the fluid inlet conduit.

Clause 1335. The tissue care dressing of any one of clauses 1328 to 1334, wherein the pressure spreading device has a generally elliptical cross-sectional shape.

Clause 1336. The tissue care dressing of any one of clauses 1252 to 1335, comprising: a fluid inlet for receiving fluid into the receptacle, and a fluid outlet for the passage of fluid out of the receptacle; and an internal flow guide; wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

Clause 1337. The tissue care dressing of clause 1336, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

Clause 1338. The tissue care dressing of clause 1335, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

Clause 1339. The tissue care dressing of any one of clauses 1336 to clause 1338, wherein the internal flow guide comprises an internal wall of the receptacle.

Clause 1340. The tissue care dressing of any one of clauses 1336 to 1339, wherein the one or more walls of the receptacle include at least a section comprising a membrane adapted to allow molecules within the fluid to pass from the receptacle to the tissue of the patient.

Clause 1341. The tissue care dressing of clause 1340, wherein the receptacle is sealed such that fluid may enter the receptacle substantially only through the fluid inlet; and fluid may exit the receptacle substantially only through the fluid outlet or as a result of molecules within the fluid passing through the membrane.

Clause 1342: The tissue care dressing of any one of clauses 1327 to 1335, wherein the pressure spreading device comprises at least one cavity providing fluid communication between the fluid inlet conduit and the tail portion of the receptacle and/or the tail portion of the receptacle and the first fluid outlet conduit.

Clause 1343: The tissue care dressing of clause 1342, wherein the cavity has a width dimension that increases from the fluid inlet end of the cavity and/or fluid outlet end of the cavity to the tail portion end of the cavity.

Clause 1344: The tissue care dressing of clause 1343, wherein the width dimension of the cavity increases gradually from the fluid inlet end of the cavity and/or fluid outlet end of the cavity to the tail portion end of the cavity.

Clause 1345: The tissue care dressing of clause 1342 or clause 1343 or claim 1344, wherein the cavity has one or more cavity ridges extending substantially from a fluid inlet end of the cavity to a tail portion end of the cavity or from a fluid outlet end of the cavity to the tail portion end of the cavity.

Clause 1346: The tissue care dressing of clause 1345, wherein the one or more cavity ridges comprises a plurality of the cavity ridges disposed in a configuration such that the spacing between adjacent ridges increases from the fluid inlet end or fluid outlet end of the cavity to the tail portion end of the cavity.

Clause 1347: The tissue care dressing of any one of clause 1345 to clause 1346, wherein the one or more cavity ridges are positioned so as to be offset from the at least one ridge of the tail portion.

Clause 1348: The tissue care dressing of clause 1345 to clause 1347, wherein the one or more cavity ridges overlap longitudinally with the at least one ridge of the tail portion.

Clause 1349: The tissue care dressing of any one of clauses 1345 to 1348, wherein the one or more cavity ridges comprise an extension of the at least one ridge of the tail portion into the cavity.

Clause 1350: The tissue care dressing of any one of clause 1342 to clause 1349, wherein the pressure spreading device is adapted to receive an end portion of the fluid inlet conduit and an end portion of the first fluid outlet conduit therein, such that the end portions terminate inside the pressure spreading device.

Clause 1351: The tissue care dressing of any one of clause 1342 to clause 1350, wherein the pressure spreading device is adapted to receive an end portion of the fluid inlet conduit and an end portion of the first fluid outlet conduit therein, such that the end portions are permitted to terminate inside the tail portion.

Clause 1352: The tissue care dressing of clause 1350 or clause 1351, wherein the pressure spreading device includes an inlet conduit channel and an outlet conduit channel adapted for receiving the respective fluid inlet conduit or the first fluid outlet conduit therein.

Clause 1353: The tissue care dressing of clause 1350 or clause 1352, therein the pressure spreading device comprises at least one conduit locating feature adapted for locating an end of the fluid inlet conduit or first fluid outlet conduit.

Clause 1354: The tissue care dressing of clause 1353, wherein the conduit locating feature comprises a stop.

Clause 1355: The tissue care dressing of clause 1353 or clause 1354, wherein the conduit locating feature is positioned in the inlet conduit channel and/or the outlet conduit channel.

Clause 1356: The tissue care dressing of clause 1355, wherein the conduit locating feature is positioned at the fluid inlet end of the cavity and/or the fluid outlet end of the cavity.

Clause 1357: The tissue care dressing of any one of clauses 1336 to 1339, wherein the internal flow guide has a fluid flow path feature adapted for allowing fluid to flow from the first compartment to the second compartment.

Clause 1358: The tissue care dressing of clause 1357, wherein the fluid flow path feature is one or more of an opening, an aperture, or a perforation.

Clause 1359: The tissue care dressing of clause 1357 or 1358 wherein the fluid flow path feature includes an opening surround.

Clause 1360: The tissue care dressing of clause 1359, wherein the opening surround comprises a continuous surround of the opening, aperture or perforation.

Clause 1361: The tissue care dressing of clause 1359, wherein the opening surround comprises at least one discontinuity.

Clause 1362: The tissue care dressing of any one of clauses 1359 to 1361, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion, wherein the at least one ridge is configured to at least partially maintain the fluid flow path upon a pressing force being applied to the apparatus.

Clause 1363: The tissue care dressing of clause 1362, wherein the at least one ridge is arranged to overlap with the opening surround of the internal wall.

Clause 1364: The tissue care dressing of claim 1363, wherein the at least one ridge comprises two or more ridges, at least one of which is arranged to overlap with the opening surround.

Clause 1365: The tissue care dressing of clause 1362 when dependent on clause 1361, wherein the at least one ridge is arranged to extend through the at least one discontinuity in the opening surround.

Clause 1366: An apparatus, comprising a receptacle having:
one or more walls comprising a first wall portion and a second wall portion opposite the first wall portion;
an internal wall extending between the first wall portion and the second wall portion;
the internal wall having an opening for passage of fluid there through;
a fluid inlet; a fluid outlet;
wherein the internal wall is configured as a barrier between the fluid inlet and the fluid outlet.

Clause 1367: The apparatus of clause 1366, wherein the internal wall is configured to extend at least partially along a longitudinal axis of the receptacle.

Clause 1368: The apparatus of clause 1366 or clause 1367, wherein the internal wall is configured to extend from an inner surface of the first wall portion to an inner surface of the second wall portion.

Clause 1369: The apparatus of any one of clauses 1366 to 1368, wherein the internal wall is configured to divide the receptacle into a first compartment and a second compartment.

Clause 1370: The apparatus of any one of clauses 1366 to 1369, wherein the fluid inlet and the fluid outlet are disposed substantially adjacent one another.

Clause 1371: The apparatus of clause 1366 or clause 1370 when dependent on clause 4, wherein the fluid inlet is disposed to allow fluid to enter into the first compartment and the fluid outlet is disposed to allow fluid in the second compartment to exit the receptacle.

Clause 1372: The apparatus of any one of clauses 1366 to 1372, wherein the opening of the internal wall is positioned distally of the fluid inlet.

Clause 1373: The apparatus of any one of clause 1369 to clause 1372, configured to provide a fluid flow path from the fluid inlet into the first compartment, through the opening in the internal wall, into the second compartment and out of the fluid outlet.

Clause 1374: The apparatus of any one of clauses 1366 to 1373, wherein the receptacle is inflatable.

Clause 1375: The apparatus of any one of clauses 1366 to 1374, comprising a fluid inlet conduit arranged in fluid communication with the receptacle, and a first fluid outlet conduit arranged in fluid communication with the receptacle.

Clause 1376: The apparatus of clause 1375, wherein the fluid inlet conduit and the first fluid outlet conduit are arranged substantially parallel with one another at the portion of the one or more walls of the receptacle.

Clause 1377. The apparatus of clause 1376, wherein the fluid inlet conduit and the first fluid outlet conduit are at least partially adjoined so as to extend from the portion of the one or more walls of the receptacle in a common direction.

Clause 1378 The apparatus of clause 1377, wherein the fluid inlet conduit and the first fluid outlet conduit are at least partially adjoined at or near end portions thereof.

Clause 1379. The apparatus of 1378, wherein at least the end portion of the fluid inlet conduit and the first fluid outlet conduit are configured to lie substantially parallel with the tissue of a patient during use.

Clause 1380. The apparatus of clause 1378 or 1379, wherein at least the end portion of the fluid inlet conduit and the first fluid outlet conduit are configured to lie substantially adjacent and in close spaced relation from the tissue of a patient during use.

Clause 1381. The apparatus of clause 1378 or 1379, wherein at least the end portion of the fluid inlet conduit and the first fluid outlet conduit are configured to lie substantially adjacent and in contact with the tissue of a patient during use.

Clause 1382. The apparatus of any one of clauses 1375 to clause 1381, wherein the receptacle has a longitudinal axis, and at least the end portion of the fluid inlet conduit and the first fluid outlet conduit are arranged to extend from the receptacle in a direction along or substantially parallel to the longitudinal axis.

Clause 1383. The apparatus of any one of clauses 1366 to 1382, wherein the fluid outlet is configured for cycling fluid out of the fluid receptacle.

Clause 1384. The apparatus of clause 1383, wherein the fluid outlet is configured to allow cycling of the fluid out of the receptacle to maintain a concentration of the fluid within the receptacle.

Clause 1385. The apparatus of any one of clauses 1367 to 1384, wherein at least a section of the one or more walls forms a tissue facing portion.

Clause 1386. The apparatus of clause 1385, wherein the tissue facing portion is adapted to allow molecules within the fluid to pass from the receptacle to the tissue of the patient.

Clause 1387. The apparatus of clause 1385 or 1386, wherein the tissue facing portion is adapted to allow molecules within the fluid to diffuse from the receptacle to the tissue of the patient.

Clause 1388. The apparatus of clause 1387, wherein the tissue facing portion adapted to allow molecules to diffuse from the receptacle to the tissue area is substantially pore free.

Clause 1389. The apparatus of clause 1388, wherein the tissue facing portion comprises substantially no discontinuities or pores that are visible with an optical or electron microscope at a specified resolution.

Clause 1390. The apparatus of clause 1389, wherein the specified resolution is on the order of 1 micrometre.

Clause 1391. The apparatus of any one of clauses 1386 to 1390, wherein the tissue facing portion adapted to allow molecules to pass from the receptacle to the tissue area comprises at least one of the following: liquid silicone rubber, pre-formed silicone sheet, silicone hydrogel, linear low-density polyethylene treated with calcium carbonate, silicone coatings, and high consistency rubber silicone.

Clause 1392. The apparatus of any one of clauses 1386 to 1391, wherein the tissue facing portion is impermeable to bulk transport of fluid.

Clause 1393. The apparatus of any one of clauses 1386 to 1392, wherein the tissue facing portion is hydrophobic.

Clause 1394. The apparatus of any one of clauses 1386 to 1393, wherein the tissue facing portion is a membrane.

Clause 1395. The apparatus of any one of clauses 1386 to 1394, wherein the receptacle is sealed such that fluid may enter the receptacle substantially only through the fluid inlet; and fluid may exit the receptacle substantially only through the fluid outlet or as a result of molecules within the fluid passing through the tissue facing portion.

Clause 1396. The apparatus of clause 1395, wherein a boundary of the fluid flow path through the fluid receptacle is independent of tissue of the patient.

Clause 1397. The apparatus of clause 1396, wherein the one or more walls is present between the fluid flow path and the tissue of the patient in use.

Clause 1398. The apparatus of any one of clauses 1367 to 1397, further comprising a pressure spreading device adapted for contacting tissue of the patient.

Clause 1399. The apparatus of clause 1398 when dependent on 1375, wherein the pressure spreading device encapsulates at least a portion of the fluid inlet conduit and/or the first fluid outlet conduit.

Clause 1400. The apparatus of clause 1399, wherein the pressure spreading device is configured to spread a force applied to the tissue of the patient by the fluid inlet conduit and/or the first fluid outlet conduit across a width of the pressure spreading device so as to mitigate patient discomfort.

Clause 1401. The apparatus of any one of clauses 1366 to 1400, wherein the receptacle comprises a head portion and a tail portion, wherein the fluid inlet and the fluid outlet are disposed at the tail portion.

Clause 1402. The apparatus of clause 1401 when dependent on clause 1375, wherein the fluid inlet conduit and the first fluid outlet conduit each have a tail end that terminates at the tail portion of the receptacle to provide fluid communication between the respective one of the fluid inlet conduit and the first fluid outlet conduit and the receptacle.

Clause 1403. The apparatus of any one of clauses 1401 to 1402 when dependent on any of clauses 1398 to 1400, wherein the pressure spreading device adjoins with the tail portion of the receptacle.

Clause 1404. The apparatus of any one of clauses 1401 to 1403 when dependent on any of clauses 1398 to 1400, wherein the pressure spreading device is formed integrally with the tail portion of the receptacle.

Clause 1405. The apparatus of any one of clauses 1401 to 1403 when dependent on any one of clauses 1398 to 1400, wherein the pressure spreading device is formed as a separate component from the tail portion of the receptacle.

Clause 1406. The apparatus of any one of clauses 1398 to 1405, wherein the pressure spreading device has a shape configured to reduce pressure exerted on the patient by the fluid inlet conduit and/or the first fluid outlet conduit.

Clause 1407 The apparatus of any one of clauses 1399 to 1406, wherein the pressure spreading device has a width that is greater than an external diameter of the fluid inlet conduit.

Clause 1408. The apparatus of any one of clauses 1398 to 1407, wherein the pressure spreading device has a generally elliptical cross-sectional shape.

Clause 1409. The apparatus of any one of clauses 1366 to 1408, further comprising a pressure regulator associated with the fluid outlet.

Clause 1410. The apparatus of clause 1409, wherein the pressure regulator is a pressure relief valve.

Clause 1411. The apparatus of any one of clauses 1366 to 1410, wherein the receptacle is in the form of a bag.

Clause 1412. The apparatus of any one of clauses 1366 to 1411, wherein the receptacle is configured to contact the tissue of the patient in use.

Clause 1413. The apparatus of any one of clauses 1387 to 1412, wherein at least the at least one section of the receptacle is conformable with a surface of the tissue of the patient.

Clause 1414. The apparatus of any one of clauses 1366 to 1413, wherein the one or more walls of the receptacle are adapted to allow molecules within the fluid to diffuse from the receptacle to the tissue of the patient.

Clause 1415. The apparatus of any one of clauses 1366 to 1414, wherein the one or more walls comprise a membrane.

Clause 1416. The apparatus of any one of clauses 1366 to 1415, further comprising a cover positionable over the receptacle to form a compartment substantially bounded by the cover, the receptacle and the tissue of the patient.

Clause 1417. The apparatus of clause 1416, wherein the compartment substantially bounded by the cover, the receptacle and the tissue of the patient is pressurised during use to form a negative pressure compartment.

Clause 1418. The apparatus of clause 1416 or 1417, comprising a negative pressure source arranged in fluid communication with the compartment substantially bounded by the cover, the receptacle and the tissue of the patient for applying a negative pressure to the compartment.

Clause 1419. The apparatus of clause 1418, wherein the negative pressure source is a pump.

Clause 1420. The apparatus of any one of clauses 1416 to 1419, wherein the pressure in the compartment is altered to be between approximately 50 mmHg to 150 mmHg or between approximately 80 mmHg to 125 mmHg or between 90 mmHg to 110 mmHg below atmospheric pressure.

Clause 1421. The apparatus of any one of clauses 1366 to 1420, wherein the fluid comprises a therapeutic gas.

Clause 1422. The apparatus of clause 1421, wherein the gas comprises oxygen and/or carbon dioxide and/or carbon monoxide.

Clause 1423. The apparatus of any one of clauses 1366 to 1422, further comprising a fluid source.

Clause 1424. The apparatus of clause 1423, wherein the fluid source comprises a wall source, gas bottle, gas concentrator, or other gas generator.

Clause 1425. The apparatus of any one of clauses 1415, wherein the membrane is adapted to allow the fluid to pass from the receptacle to the tissue of the patient by pore flow.

Clause 1426. The apparatus of any one of clauses 1366 to 1425, wherein the receptacle is substantially hollow.

Clause 1427. The apparatus of any one of clauses 1366 to 1426, wherein the tissue of the patient comprises a wound area.

Clause 1428. The apparatus of clause 1387 or any one of clauses 1388 to 1427 when dependent on clause 1387, further comprising structures associated with the receptacle, the structures being configured to contact the tissue of the patient.

Clause 1429. The apparatus of clause 1428, wherein the structures are microstructures.

Clause 1430. The apparatus of any one of clauses 1428 to 1429, wherein the structures comprise formations which protrude from the one or more walls of the receptacle.

Clause 1431. The apparatus of clause 1429 or 1430, wherein molecules within the fluid diffuse through the tissue facing portion of the receptacle and the structures into the tissue of the patient.

Clause 1432. The apparatus of any one of clauses 1428 to 1431 wherein the structures protrude from a tissue facing surface of the one or more walls of the receptacle.

Clause 1433. The apparatus of any one of clauses 1429 to 1432, wherein the formations have one or more of a parabolic, trapezoidal, semi-spherical, spherical, pyramidal, conical, domed or frustoconical shape.

Clause 1434. The apparatus of any one of clauses 1428 to 1433, wherein the structures have rounded edges.

Clause 1435. The apparatus of any one of clauses 1366 to 1434, wherein the one or more walls include a first wall portion and a second wall portion opposite the first wall portion, the first wall portion and the second wall portion forming a fluid flow path there between, at least one ridge disposed on an inner surface of at least one of the first wall portion and the second wall portion.

Clause 1436. The apparatus of clause 1435, wherein the at least one ridge is configured to at least partially maintain the fluid flow path upon a pressing force being applied to the apparatus.

Clause 1437. The apparatus of clause 1436, wherein the at least one ridge has one or more properties configured to at least partially maintain the fluid flow path whilst mitigating indentation of the at least one ridge on the tissue area of the patient, upon a pressing force being applied to the apparatus.

Clause 1438. The apparatus of clause 1436 or clause 1437, wherein the at least one ridge is substantially straight.

Clause 1439. The apparatus of clause 1437 or clause 1438, wherein the at least one ridge has a longitudinal form, when viewed in plan view, that is substantially non-straight.

Clause 1440. The apparatus of clause 1439, wherein the non-straight longitudinal form of the at least one ridge, when viewed in plan view, is one or more of curved, zigzag or irregular.

Clause 1441. The apparatus of any one of clauses 1437 to 1440, wherein the one or more properties include that the at least one ridge has a height dimension of between about 100 to 200 micrometres, or between about 125 to 175 micrometres, or approximately 150 micrometres.

Clause 1442. The apparatus of any one of clauses 1437 to 1441, wherein the one or more properties include that the at least one ridge is formed from a yielding material.

Clause 1443. The apparatus of any one of clauses 1437 to 1442, wherein the one or more properties include that the at least one ridge comprises two or more ridges arranged substantially parallel to one another.

Clause 1444. The apparatus of any one of clauses 1437 to 1443, wherein the one or more properties comprise at least one ridge on the inner surface of the first wall portion of the receptacle and at least one ridge on the inner surface of the second wall portion of the receptacle.

Clause 1445. The apparatus of clause 1444, wherein the one or more properties include that the at least one ridge on the inner surface of the first wall portion is offset from the at least one ridge on the inner surface of the second wall portion.

Clause 1446. The apparatus of clause 1444, wherein the at least one ridge adjoins the inner surface of the respective first wall portion or second wall portion with a substantially non-filleted corner.

Clause 1447. The apparatus of clause 1446, wherein the substantially non-filleted corner is a sharp corner.

Clause 1448. The apparatus of clause 1444, wherein the at least one ridge adjoins the inner surface of the respective first wall portion or second wall portion with a fillet having a fillet radius of less than or equal to about 30% or less than or equal to about 20% or less than or equal to about 10% of a height dimension of the ridge.

Clause 1449. The apparatus of any one of clauses 1435 to 1448, wherein a thickness dimension of the first wall portion and/or the second wall portion is less than or approximately equal to a height dimension of the ridge.

Clause 1450. The apparatus of any one of clauses 1435 to 1448, wherein a thickness dimension of the first wall portion and/or the second wall portion is greater than a height dimension of the ridge.

Clause 1451. The apparatus of any one of clauses 1435 to 1450, wherein the first wall portion and/or the second wall portion has a wall thickness of between about 10 micrometres and about 150 micrometres, or between 20 micrometres and 140 micrometres, or between about 30 micrometres and 130 micrometres, or between about 35 micrometres and 100 micrometres, or between about 40 micrometres and 80 micrometres, or between about 40 micrometres and 70 micrometres, or between 30 micrometres and 60 micrometres, or between about 20 micrometres to 60 micrometres, or between about 45 micrometres and 55 micrometres.

Clause 1452. The apparatus of clause 1451, wherein the first wall portion and/or the second wall portion has a wall thickness of approximately 50 micrometres or of approximately 60 micrometres.

Clause 1453. The apparatus of any one of clauses 1435 to 1452, wherein the inner surface of the at least one of the first wall portion and/or the second wall portion comprises a plurality of the ridges.

Clause 1454. The apparatus of any one of clauses 1435 to 1453, wherein the first wall portion and the second wall portion are part of a single common wall.

Clause 1455. The apparatus of any one of clauses 1435 to 1454, wherein the first wall portion is a portion of a first wall and the second wall portion is a portion of a second wall.

Clause 1456. The apparatus of any one of clauses 1366 to 1455, wherein the receptacle has a plurality of microstructures formed thereon.

Clause 1457. A kit of parts, comprising the apparatus of any one of clauses 289 to 315 and a dressing.

Clause 1458. The kit of parts of clause 1457, further comprising a fluid source.

Clause 1459. The kit of parts of clause 1457 or clause 1458, further comprising an absorbent material.

Clause 1460. The kit of parts of clause 1457 or clause 1459, wherein the dressing comprises a compression bandage or an adhesive dressing.

Clause 1461. A kit of parts, comprising the apparatus of any one of clauses 289 to 370 and a fluid source.

Clause 1462. The kit of clause 1458 or clause 1461, wherein the fluid source is a portable fluid source.

Clause 1463. A kit of parts, comprising the apparatus of any one of clauses 1 to 142, a negative pressure source and a fluid pump.

Clause 1464. The kit of parts of clause 1463, further comprising a portable fluid source.

Clause 1465. The apparatus of any one of clauses 73 to 74 or 214 to 215 or 520 to 521, 661 to 662,793 to 794, 922 to 923, 1057 to 1058 or 1418 to 1419 configured for simultaneous delivery of fluid to tissue of a patient via the receptacle and application of a negative pressure to the compartment.

Clause 1466. The tissue care dressing of any one of clauses 1203 to 1204 or 1317 to 1318, configured for simultaneous delivery of fluid to tissue of a patient via the receptacle and application of a negative pressure to the compartment.

Clause 1467. The apparatus of any one of clauses 73 to 74 or 214 to 215 or 520 to 521, 661 to 662,793 to 794, 922 to 923, 1057 to 1058 or 1418 to 1419, wherein the receptacle is adapted for operation at a positive pressure simultaneously as a negative pressure is applied to the compartment.

Clause 1468 The tissue care dressing of any one of clauses 1203 to 1204 or 1317 to 1318, wherein the receptacle is adapted for operation at a positive pressure simultaneously as a negative pressure is applied to the compartment.

Clause 1469. The apparatus of any one of clauses 113 to 116 when dependent on any one of clauses 90 to 92, or any one of clauses 259 to 262 when dependent on any one of clauses 236 to 238, or any one of clauses 341 to 344 when dependent on any one of clauses 318 to 320, or any one of clauses 452 to 455 when dependent on any one of clauses 429 to 431, or any one of clauses 583 to 586 when dependent on any one of clauses 560 to 562, or any one of clauses 704 to 708, wherein the internal flow guide extends at least partially into the head portion of the receptacle.

Clause 1470. The apparatus of any one of clauses 962 to 964, wherein the internal wall extends at least partially into the head portion of the receptacle.

Clause 1471. The tissue care dressing of any one of clauses 1222 to 1225 when dependent on any one of clauses 1167 to 1169, or any one of clauses 1336 to 1339 when dependent on any one of clauses 1302 to 1304, wherein the internal flow guide extends at least partially into the head portion of the receptacle.

1. An apparatus for supplying fluid to tissue of a patient, the apparatus comprising:
an inflatable receptacle positionable at a tissue area, the receptacle comprising one or more walls and being adapted to receive a fluid, the one or more walls comprising at least one section adapted to allow molecules within the fluid to diffuse from the receptacle to the tissue area; and
a cover positionable over the receptacle to form a compartment substantially bounded by the cover, the receptacle and the tissue area.

2. The apparatus of claim 1, wherein the receptacle is moveable within the compartment bounded by the cover, the receptacle and the tissue area.

3. An apparatus for supplying fluid to a tissue area, the apparatus comprising:
a receptacle positionable at a tissue area, the receptacle comprising one or more walls and being adapted to receive a fluid, the one or more walls comprising at least one section adapted to allow molecules within the fluid to diffuse from the receptacle to the tissue area; and
a cover positionable over the receptacle to form a compartment substantially bounded by the cover, the receptacle and the tissue area,
wherein the receptacle is moveable within the compartment substantially bounded by the cover, the receptacle and the tissue area.

4. The apparatus of any one of claims 1 to 3, wherein the receptacle is in the form of a bag.

5. The apparatus of any one of the preceding claims, wherein the receptacle is configured to contact the tissue area.

6. The apparatus of any one of the preceding claims, wherein at least the at least one section of the receptacle is conformable with a surface of the tissue area.

7. The apparatus of any one of the preceding claims, wherein the at least one section and/or the one or more walls comprise a membrane.

8. The apparatus of any one of the preceding claims, wherein the at least one section of the receptacle comprises a stretchable material.

9. The apparatus of any one of the preceding claims, wherein the apparatus further comprises a fluid inlet conduit arranged in fluid communication with the receptacle for delivery of fluid to the receptacle.

10. The apparatus of claim 9, wherein the cover has a first opening through which the fluid inlet conduit passes or to which the fluid inlet conduit is connected.

11. The apparatus of claim 9 or claim 10, wherein the receptacle is coupled to the cover via the fluid inlet conduit.

12. The apparatus of claim 11, wherein the compartment is further bounded by the fluid inlet conduit.

13. The apparatus of any one of claim 9 or claim 10 or claim 11, wherein the fluid inlet conduit includes a pressure relief valve.

14. The apparatus of any one of the preceding claims, wherein the cover comprises one or more walls and wherein the one or more walls of the cover are independent of the one or more walls of the receptacle.

15. The apparatus of any one of claims 1 to 14, wherein the receptacle and the cover share a common wall.

16. The apparatus of any one of the preceding claims, wherein the receptacle is configured to receive fluid during use of the apparatus.

17. The apparatus of any one of claims 1 to 15, wherein the receptacle is pre-filled with the fluid.

18. The apparatus of any one of the preceding claims, wherein the one or more walls of the receptacle may define one or more through-holes through the receptacle.

19. The apparatus of any one of the preceding claims, including a plurality of the receptacles.

20. The apparatus of any one of the preceding claims, further comprising structures associated with the at least one section of the receptacle, the structures being configured to contact the tissue area.

21. The apparatus of claim 20, wherein the structures are configured for encouraging cell growth.

22. The apparatus of claim 20, wherein the structures are microstructures.

23. The apparatus of claim 21, wherein the microstructures comprise formations which protrude from the one or more walls of the receptacle.

24. The apparatus of claim 21 or claim 22 or claim 23, wherein molecules diffuse through the at least one section of the receptacle and the microstructures into the tissue area.

25. The apparatus of claim 22 or claim 23 or claim 24, wherein the microstructures protrude from a tissue facing surface of the one or more walls of the receptacle.

26. The apparatus of any one of claims 23 to 25, wherein the formations have one or more of a parabolic, trapezoidal, semi-spherical, spherical, pyramidal, conical, domed or frustoconical shape.

27. The apparatus of any one of claims 22 to 26, wherein the microstructures have rounded edges.

28. The apparatus of claim 7, wherein the membrane adapted to allow molecules to diffuse from the receptacle to the tissue area is substantially pore-free.

29. The apparatus of claim 28, wherein the membrane comprises substantially no discontinuities or pores that are visible with an optical or electron microscope at a specified resolution.

30. The apparatus of claim 29, wherein the specified resolution is on the order of 1 micrometre.

31. The apparatus of any one of claims 28 to 30, wherein the membrane adapted to allow molecules to diffuse from the receptacle to the tissue area is configured to contact the tissue area.

32. The apparatus of any one of claims 28 to 31, wherein the one or more walls of the receptacle, other than the at least one section, comprise a material adapted to allow molecules to pass through via molecular diffusion at only a negligible rate of diffusion.

33. The apparatus of any one of the preceding claims, wherein the at least one section has a wall thickness of between about 10 micrometres and 150 micrometres, or between about 20 micrometres and 150 micrometres, or between about 20 micrometres and 140 micrometres, or between about 30 micrometres and 130 micrometres, or between about 35 micrometres and 100 micrometres, or between about 40 micrometres and 80 micrometres or between about 40 micrometres and 70 micrometres, or between about 30 micrometres and 60 micrometres, or between about 20 micrometres and 60 micrometres, or between about 45 micrometres and 55 micrometres.

34. The apparatus of claim 33, wherein the at least one section has a wall thickness of approximately 50 micrometres or wherein the at least one section has a wall thickness of approximately 60 micrometres.

35. The apparatus of any one of claims 27 to 34, wherein the membrane is impermeable to bulk transport of fluid.

36. The apparatus of any one of the preceding claims, wherein the at least one section of the one or more walls of the receptacle are hydrophobic.

37. The apparatus of any one of the preceding claims, wherein the cover is configured to secure the receptacle to the tissue area.

38. The apparatus of any one of the preceding claims, wherein the cover is configured to press the receptacle onto or into the tissue area.

39. The apparatus of any one of the preceding claims, wherein the cover comprises one or more of a dressing, a bandage wrapped around a body part of the patient to form the cover, an adhesive dressing and a compression bandage.

40. The apparatus of claim 39, wherein the tissue area comprises a wound area, and wherein the cover comprises an absorbent material for absorbing exudate from the wound area.

41. The apparatus of claim 40, wherein the absorbent material is placed upon or within the wound area of a patient and the cover holds the absorbent material in place.

42. The apparatus of any one of the preceding claims, wherein the cover is integrally formed with the receptacle.

43. The apparatus of claim 9, wherein the receptacle comprises a first fluid outlet conduit via which fluid is receivable from the receptacle.

44. The apparatus of claim 43, wherein the compartment is further bounded by the first fluid outlet conduit.

45. The apparatus of claim 43 or 44, further comprising a second fluid outlet conduit arranged in fluid communication with the compartment substantially bounded by the cover, the receptacle, and the wound area.

46. The apparatus of any one of claims 43 to 45, further comprising a pressure relief valve in communication with the first opening and/or the receptacle and operable to relieve pressure within the receptacle.

47. The apparatus of claim 9, further comprising a first fluid outlet conduit arranged in fluid communication with the receptacle and a second fluid outlet conduit arranged in fluid communication with the compartment substantially bounded by the cover, the receptacle and the wound area, wherein at least two of the fluid inlet conduit, the first fluid outlet conduit, and the second fluid outlet conduit are formed as a combined conduit.

48. The apparatus of claim 47, wherein the at least two of the fluid inlet conduit, the first fluid outlet conduit, and the second fluid outlet conduit are formed as a coaxial conduit.

49. The apparatus of claim 43 or claim 44, wherein the fluid inlet conduit and the first fluid outlet conduit are formed as a co-axial conduit or as a combined conduit.

50. The apparatus of claim 47 or claim 48 or claim 49, wherein the combined conduit or the coaxial conduit pass through a single opening in the cover.

51. The apparatus of any one of claims 45 to 49, wherein at least one of the fluid inlet conduit, the first fluid outlet conduit and the second fluid outlet conduit passes into the compartment adjacent an outer edge of the cover.

52. The apparatus of any one of the preceding claims, wherein the cover is sealable to the tissue area.

53. The apparatus of any one of the preceding claims, wherein the cover comprises one or more walls that are adapted to allow molecules to pass through via molecular diffusion at only a negligible rate of diffusion.

54. The apparatus of any one of the preceding claims, wherein the compartment substantially bounded by the cover, the receptacle and the tissue area is pressurised during use to form a negative pressure compartment.

55. The apparatus of any one of the preceding claims, comprising a negative pressure source arranged in fluid communication with the compartment substantially bounded by the cover, the receptacle and the tissue area for applying a negative pressure to the compartment.

56. The apparatus of any one of the preceding claims, wherein the fluid comprises a therapeutic gas.

57. The apparatus of claim 56, wherein the gas comprises oxygen and/or carbon dioxide and/or carbon monoxide.

58. The apparatus of any one of the preceding claims, wherein the receptacle forms a reservoir adapted for storing fluid therein.

59. The apparatus of claim 58, wherein the reservoir is configured to provide fluid to the tissue upon disconnection of the reservoir from the fluid source.

60. The apparatus of claim 55, configured for simultaneous delivery of fluid to tissue of a patient via the receptacle and application of a negative pressure to the compartment.

61. The apparatus of claim 55 or claim 60, wherein the receptacle is adapted for operation at a positive pressure simultaneously as a negative pressure is applied to the compartment.

62. An apparatus for supplying fluid to a tissue area, the apparatus comprising:
a receptacle positionable at a tissue area, the receptacle comprising one or more walls and being adapted to receive a fluid, the one or more walls comprising at least one section adapted to allow the fluid to pass from the receptacle to the tissue area; and
a cover positionable over the receptacle to form a compartment substantially bounded by the cover, the receptacle and the tissue area;
a first opening in the cover via which fluid is deliverable to the receptacle;
a second opening in the cover via which fluid is receivable from the receptacle; and
a third opening in the cover via which fluid is receivable from the compartment substantially bounded by the cover, the receptacle and the tissue area.

63. An apparatus for supplying fluid to a tissue area, the apparatus comprising:
a plurality of receptacles positionable at a tissue area, each receptacle comprising one or more walls and being adapted to receive a fluid, the one or more walls comprising at least one section adapted to allow the fluid to pass from the receptacle to the tissue area.

64. An apparatus for supplying fluid to a tissue, the apparatus comprising:
a receptacle positionable at a tissue area, the receptacle comprising one or more walls and adapted to receive a fluid, the one or more walls comprising at least one section adapted to allow molecules within the fluid to pass from the receptacle to the tissue area;
a fluid inlet in fluid communication with the receptacle; and
a pressure relief valve in fluid communication with the fluid inlet and/or the receptacle and operable to relieve pressure within the fluid inlet and/or receptacle.

65. An apparatus for supplying fluid to a tissue, the apparatus comprising:
a fluid receptacle positionable at a tissue area, the fluid receptacle comprising one or more walls, a fluid inlet for receiving fluid into the fluid receptacle, and a fluid outlet for the passage of fluid from the fluid receptacle;
and a pressure regulator associated with the fluid outlet.

66. The apparatus of claim 65, wherein the pressure regulator is positioned in spaced relation from the fluid receptacle.

67. The apparatus of claim 65 or 66, further comprising a fluid outlet conduit arranged in fluid communication with the fluid receptacle, wherein the pressure regulator is disposed on the fluid outlet conduit.

68. The apparatus of any one of claims 65 to 67, wherein the pressure regulator is selectively actuable to an at least partially open position to permit fluid to pass there through, and is configured, when in the at least partially open position, to vent the fluid passing there through to the atmosphere.

69. The apparatus of any one of claims 65 to 68, wherein a portion of the fluid receptacle is a tissue facing portion.

70. The apparatus of any one of claims 65 to 69, wherein a fluid flow path through the fluid receptacle is defined by the one or more walls of the fluid receptacle, the fluid inlet and the fluid outlet.

71. The apparatus of claim 69, wherein at least a portion of the tissue facing portion comprises a membrane adapted to allow fluid within the receptacle to pass through the membrane to the tissue of the patient.

72. The apparatus of claim 69 or claim 70, wherein at least a portion of the tissue facing portion comprises a membrane adapted to allow molecules within the fluid to diffuse through the membrane to the tissue of the patient.

73. The apparatus of any one of claims 65 to 72, wherein the fluid inlet is adapted for providing a positive pressure within the fluid receptacle.

74. The apparatus of any one of claim 65 or 73, wherein the one or more walls of the fluid receptacle define a positive pressure receptacle.

75. The apparatus of any one of claims 65 to 74, wherein the fluid receptacle is inflatable.

76. The apparatus of any one of claims 65 to 75, wherein the pressure regulator comprises a pressure relief valve.

77. The apparatus of any one of claims 65 to 76, wherein the pressure regulator is configurable to relieve pressure within the receptacle when the pressure within the receptacle reaches a threshold value.

78. The apparatus of any one of claims 65 to 77, wherein the pressure regulator is configurable to adjust the pressure threshold value.

79. The apparatus of any one of claims 277 to 78, wherein the pressure threshold value is an upper bound of the pressure.

80. The apparatus of any one of claims 65 to 79, wherein the fluid receptacle comprises multiple layers, comprising a first layer configured to form a tissue facing layer and a second layer.

81. The apparatus of claim 80, wherein the second layer has a first thickness and the first layer has a second thickness, the first thickness being greater than the second thickness.

82. The apparatus of claim 80 or claim 81, wherein the second layer and the first layer are permeable, the second layer having a lower permeability than the first layer.

83. The apparatus of any one of claims 80 to 82 when dependent on any one of claims 65 to 75 or 77 to 79, wherein the second layer includes one or more apertures configured to permit fluid within the fluid receptacle to pass there through to exit the fluid receptacle.

84. The apparatus of claim 83, wherein the one or more apertures are configured to provide pressure relief within the fluid receptacle.

85. The apparatus of any one of claims 65 to 84, further comprising a fluid source in fluid communication with the fluid inlet and adapted to provide a substantially continuous supply of fluid to the fluid receptacle.

86. The apparatus of any one of claims 65 to 85, further adapted to apply a negative pressure to the tissue of the patient.

87. The apparatus of any one of claims 65 to 86, wherein the fluid comprises a therapeutic gas.

88. The apparatus of claim 87, wherein the gas comprises oxygen and/or carbon dioxide and/or carbon monoxide.

89. The apparatus of claim 72 or any one of claims 73 to 88 when dependent on claim 72, wherein the membrane is adapted to allow molecules within the fluid to pass from the receptacle to the tissue of the patient by pore flow.

90. The apparatus of any one of claims 65 to 89, wherein the receptacle is substantially hollow.

91. The apparatus of claim 69 or any one of claims 70 to 90 when dependent on claim 69, further comprising structures associated with the tissue facing portion of the receptacle, the structures being configured to face the tissue of the patient.

92. The apparatus of claim 91, wherein the structures are microstructures and/or wherein the structures comprise formations which protrude from the one or more walls of the receptacle.

93. The apparatus of any one of claims 65 to 92, wherein the one or more walls include a first wall portion and a second wall portion opposite the first wall portion, the first wall portion and the second wall portion forming a fluid flow path there between,
at least one ridge disposed on an inner surface of at least one of the first wall portion and the second wall portion,
wherein the at least one ridge is configured to at least partially maintain the fluid flow path upon a pressing force being applied to the apparatus.

94. An apparatus, comprising:
a receptacle comprising one or more walls, a fluid inlet for receiving fluid into the receptacle, and a fluid outlet for the passage of fluid out of the receptacle; and an internal flow guide;
wherein the one or more walls, the fluid inlet, the fluid outlet and the internal flow guide are configured to define a fluid flow path through the receptacle that extends from the fluid inlet, past at least a portion of the internal flow guide, and out of the fluid outlet.

95. The apparatus of claim 94, wherein the one or more walls include a tissue facing portion; and the internal flow guide has a first flow guide surface and a second opposing flow guide surface; wherein the internal flow guide is disposed in the receptacle such that only the first flow guide surface faces the tissue facing portion.

96. The apparatus of claim 94, wherein the one or more walls include a tissue facing portion; and the internal flow guide has a first flow guide surface and a second opposing flow guide surface; wherein the internal flow guide is disposed in the receptacle such that the tissue facing portion is disposed adjacent each of the first flow guide surface and the second flow guide surface.

97. The apparatus of any one of claim 94 to claim 96, wherein the internal flow guide comprises an internal wall of the receptacle.

98. The apparatus of any one of claims 94 to 97, wherein the one or more walls of the receptacle include at least a section comprising a membrane adapted to allow molecules within the fluid to pass from the receptacle to the tissue of the patient.

99. The apparatus of claim 98, wherein the receptacle is sealed such that fluid may enter the receptacle substantially only through the fluid inlet; and fluid may exit the receptacle substantially only through the fluid outlet or as a result of molecules within the fluid passing through the membrane.

100. The apparatus of claim 98 or claim 99, wherein the membrane is adapted to allow molecules within the fluid to diffuse from the receptacle to the tissue of the patient.

101. The apparatus of any one of claims 98 to 100, wherein the membrane is impermeable to bulk transport of fluid.

102. The apparatus of any one of claims 98 to 101, wherein the membrane is hydrophobic.

103. The apparatus of any one of claims 94 to 102, wherein the membrane comprises a tissue facing portion, configured to allow fluid within the receptacle to pass through the tissue facing portion to the tissue of the patient.

104. The apparatus of claim 103, wherein at least a section of the one or more walls of the receptacle forms the tissue facing portion.

105. The apparatus of any one of claims 94 to 104, wherein the at least a portion of the internal flow guide has a length, wherein the fluid flow path extends along the length and fluid flows along the fluid flow path in contact with the tissue facing portion such that molecules from the fluid can pass through the tissue facing portion.

106. The apparatus of any one of claims 94 to 105, wherein the internal flow guide is configured to prolong a residence time in which fluid entering the fluid inlet spends within the receptacle prior to exiting the receptacle through the fluid outlet.

107. The apparatus of any one of claims 95 to 106 having a longitudinal axis, wherein the internal flow guide is configured to extend at least partially along or substantially parallel to the longitudinal axis.

108. The apparatus of claim 96 wherein the internal flow guide is disposed substantially orthogonal to the tissue facing portion.

109. The apparatus of any one of claims 94 to 108, wherein the internal flow guide divides the receptacle into a first compartment and a second compartment, the first compartment and the second compartment being fluidly interconnected.

110. The apparatus of any one of claims 94 to 109, wherein the internal flow guide has a fluid flow path feature adapted for allowing fluid to flow from the first compartment to the second compartment.

111. The apparatus of claim 110, wherein the fluid flow path feature is one or more of an opening, an aperture, a permeable membrane or a perforation.

112. The apparatus of any one of claims 94 to 111, wherein the fluid inlet and the fluid outlet are disposed substantially adjacent one another.

113. The apparatus of any one of claims 109 to 112, wherein the fluid inlet is disposed to allow fluid to enter into the first compartment, the fluid outlet is disposed to allow fluid in the second compartment to exit the receptacle, and wherein the internal flow guide is configured to substantially separate the first compartment and the second compartment.

114. The apparatus of claim 112 or 113 when dependent on claim 112, wherein the internal flow guide is configured to cause fluid within the first compartment to flow past at least a portion of the internal flow guide prior to exiting the receptacle at the outlet.

115. The apparatus of any one of claims 94 to 113, wherein the internal flow guide is configured to permit fluid within the receptacle to pass through it.

116. The apparatus of any one of claims 94 to 115, wherein the internal flow guide has a thickness that is one of greater than or substantially the same as a thickness of the one or more walls of the receptacle.

117. The apparatus of any one of claims 94 to 114 or claim 116, wherein the internal flow guide comprises a material adapted to substantially prevent molecules within the fluid from passing through it.

118. The apparatus of any one of claims 94 to 117, wherein the receptacle is inflatable.

119. The apparatus of claim 118, wherein the internal flow guide is disposed inside the inflatable receptacle.

120. The apparatus of any one of claims 94 to 119, wherein the receptacle is divided into a first compartment and a second compartment, wherein the first and second compartments of the receptacle are substantially hollow.

121. The apparatus of any one of claims 94 to 120, further comprising a pressure regulator associated with the fluid outlet.

122. The apparatus of claim 121, wherein the pressure regulator is a pressure relief valve.

123. The apparatus of claim 121 or 122, wherein the pressure regulator is configurable to relieve pressure within the receptacle when the pressure within the receptacle reaches a threshold value.

124. The apparatus of any one of claims 94 to 123, wherein a fluid flow path through the fluid receptacle is defined by the one or more walls of the fluid receptacle, the internal flow guide, the fluid inlet and the fluid outlet.

125. The apparatus of any one of claims 94 to 124, wherein the receptacle is in the form of a bag.

126. The apparatus of any one of claims 94 to 125, further comprising a cover positionable over the receptacle to form a compartment substantially bounded by the cover, the receptacle and the tissue area.

127. The apparatus of any one of claims 94 to 126, wherein a boundary of the fluid flow path through the fluid receptacle is independent of the tissue of the patient and/or the one or more walls of the receptacle may be present between the fluid flow path and the tissue of the patient in use.

128. The apparatus of claim 126 or claim 127, wherein the compartment substantially bounded by the cover, the receptacle and the tissue area is pressurised during use to form a negative pressure compartment.

129. The apparatus of any one of claims 126 to 128, comprising a negative pressure source arranged in fluid communication with the compartment substantially bounded by the cover, the receptacle and the tissue area for applying a negative pressure to the compartment.

130. The apparatus of any one of claims 94 to 129, wherein the fluid comprises a therapeutic gas.

131. The apparatus of claim 130, wherein the gas comprises oxygen and/or carbon dioxide and/or carbon monoxide.

132. The apparatus of any one of claims 94 to 131, wherein the fluid outlet is configured to allow cycling of fluid out of the fluid receptacle.

133. The apparatus of claim 98 or any one of claims 99 to 132 when dependent on claim 98, further comprising structures associated with the at least one section of the receptacle, the structures being configured to face the tissue of the patient.

134. The apparatus of claim 133, wherein the structures are microstructures.

135. The apparatus of claim 134, wherein the structures comprise formations which protrude from the one or more walls of the receptacle.

136. The apparatus of any one of claims 94 to 135, wherein the one or more walls include a first wall portion and a second wall portion opposite the first wall portion, the first wall portion and the second wall portion forming a fluid flow path there between,
at least one ridge disposed on an inner surface of at least one of the first wall portion and the second wall portion,
wherein the at least one ridge is configured to at least partially maintain the fluid flow path upon a pressing force being applied to the apparatus.

137. The apparatus of claim 136, wherein the at least one ridge is configured to at least partially maintain the fluid flow path whilst mitigating indentation of the at least one ridge on the tissue of the patient, upon a pressing force being applied to the apparatus.

138. The apparatus of any one of claims 136 to 137, wherein a thickness dimension of the first wall portion and/or the second wall portion is less than or approximately equal to a height dimension of the ridge.

139. The apparatus of any one of claims 136 to 138, wherein a thickness dimension of the first wall portion and/or the second wall portion is greater than a height dimension of the ridge.

140. The apparatus of any one of claims 402 to 403, wherein the first wall portion and the second wall portion are part of a single common wall.

141. The apparatus of any one of claims 402 to 403, wherein the first wall portion is a portion of a first wall and the second wall portion is a portion of a second wall.

142. An apparatus for supplying fluid to tissue of a patient, the apparatus comprising:
a fluid receptacle positionable at a tissue area, the fluid receptacle comprising one or more walls defining a first compartment and a second compartment of the fluid receptacle; a fluid inlet disposed for receiving fluid into the first compartment of the fluid receptacle, and a fluid outlet disposed for the passage of fluid from the second compartment of the fluid receptacle to exit the receptacle;
wherein the one or more walls of the receptacle, the fluid inlet and the fluid outlet are configured to define a fluid flow path whereby fluid entering the receptacle at the fluid inlet is caused to pass from the first compartment into the second compartment.

143. An apparatus for supplying fluid to tissue of a patient, the apparatus comprising:
a fluid receptacle comprising one or more outer walls and adapted to receive a fluid, and having an internal wall configured to divide the receptacle into a first region and a second region, the first region and the second region being fluidly interconnected; and
a fluid inlet disposed for receiving fluid into the first region of the fluid receptacle, and a fluid outlet disposed for the passage of fluid from the second region of the fluid receptacle, wherein the one or more outer walls of the receptacle, the internal wall, the fluid inlet and the fluid outlet are configured to define a fluid flow path whereby fluid entering the receptacle at the fluid inlet is caused to pass from the first region into the second region and to exit the second region of the receptacle at the fluid outlet.

144. An apparatus, comprising:
a fluid receptacle comprising one or more walls and adapted to receive a fluid therein, the one or more walls having a fluid inlet for receiving fluid into the receptacle and a fluid outlet through which fluid within the receptacle may pass upon exiting the receptacle; wherein the fluid inlet and the fluid outlet are disposed substantially adjacent one another.

145. A tissue care dressing, comprising:
a receptacle comprising at least one flexible wall and at least one inner surface;
wherein at least a portion of the at least one inner surface comprises a rough surface adapted for reducing adherence of the at least a portion of the at least one inner surface to another portion of the inner surface and/or to another inner surface of the receptacle.

146. A tissue care dressing, comprising:
a receptacle being positionable at a tissue area of a patient and comprising at least one flexible wall including a first wall portion and a second wall portion, the first wall portion and the second wall portion forming a fluid flow path there between,
at least one ridge disposed on an inner surface of at least one of the first wall portion and the second wall portion,
wherein the at least one ridge is configured to at least partially maintain the fluid flow path upon a pressing force being applied to the tissue care dressing.

147. An apparatus, comprising a receptacle having:
one or more walls comprising a first wall portion and a second wall portion opposite the first wall portion;
an internal wall extending between the first wall portion and the second wall portion;
the internal wall having an opening for passage of fluid there through;
a fluid inlet; a fluid outlet;
wherein the internal wall is configured as a barrier between the fluid inlet and the fluid outlet.

148. The apparatus of claim 147, wherein the internal wall is configured to extend at least partially along a longitudinal axis of the receptacle.

149. The apparatus of claim 147 or claim 148, wherein the internal wall is configured to extend from an inner surface of the first wall portion to an inner surface of the second wall portion.

150. The apparatus of any one of claims 147 to 149, wherein the internal wall is configured to divide the receptacle into a first compartment and a second compartment.

151. The apparatus of any one of claims 147 to 150, wherein the fluid inlet and the fluid inlet outlet are disposed substantially adjacent one another.

152. The apparatus of claim 150 or claim 151 when dependent on claim 150, wherein the fluid inlet is disposed to allow fluid to enter into the first compartment and the fluid outlet is disposed to allow fluid in the second compartment to exit the receptacle.

153. The apparatus of any one of claims 147 to 152, wherein the opening of the internal wall is positioned distally of the fluid inlet.

154. The apparatus of any one of clause 150 to clause 153, configured to provide a fluid flow path from the fluid inlet into the first compartment, through the opening in the internal wall, into the second compartment and out of the fluid outlet.

155. The apparatus of any one of claims 147 to 154, wherein the receptacle is inflatable.

156. The apparatus of claim 70, wherein a boundary of the fluid flow path through the fluid receptacle may be independent of the tissue of the patient and/or the one or more walls of the receptacle may be present between the fluid flow path and the tissue of the patient in use.

157. An apparatus for supplying fluid to a tissue area, the apparatus comprising: a receptacle positionable at a tissue area, the receptacle comprising one or more walls and being adapted to receive a fluid, the one or more walls comprising at least one section adapted to allow the fluid to pass from the receptacle to the tissue area; an inlet via which fluid is deliverable to the receptacle; an outlet via which fluid is receivable from the receptacle; and a cover positionable over the receptacle to form a compartment substantially bounded by the cover, the receptacle and the tissue area, the cover having a cover outlet via which fluid is receivable from the compartment substantially bounded by the cover, the receptacle and the tissue area.
